(19) United States
(12) Patent Application Publication
(10) Pub. No.: US 2024/0336585 A1
(43) Pub. Date: Oct. 10, 2024

(54) Organic Compound, Organic Semiconductor Device, and Electronic Appliance

(71) Applicant: Semiconductor Energy Laboratory Co., Ltd., Kanagawa-ken (JP)

(72) Inventors: Sachiko KAWAKAMI, Atsugi (JP); Kazuki Kajiyama, Hadano (JP)

(73) Assignee: Semiconductor Energy Laboratory Co., Ltd., Kanagawa-ken (JP)

(21) Appl. No.: 18/612,700

(22) Filed: Mar. 21, 2024

(30) Foreign Application Priority Data
Mar. 24, 2023 (JP) 2023-048370

(51) Int. Cl. C07D 307/92; H10K 85/60

(52) U.S. Cl. CPC C07D 307/92; H10K 85/6574

(57) ABSTRACT

An organic compound represented by General Formula (G1) is provided. Ar¹ represents an aryl group having 6 to 30 carbon atoms or a heteroaryl group having 2 to 30 carbon atoms; Ar² is a group represented by General Formula (G1-1); R¹ to R¹⁷ each independently represent any of hydrogen, an alkyl group having 1 to 6 carbon atoms, and a cycloalkyl group having 3 to 10 carbon atoms; and n represents an integer of 0 to 3. In General Formula (G1-1), X represents oxygen or sulfur; any one of R²¹ to R³⁰ is bonded to nitrogen in General Formula (G1); and the others of R²¹ to R³⁰ are each independently represent any one of hydrogen, a halogen, a cyano group, an alkyl group having 1 to 6 carbon atoms, an alkenyl group having 2 to 6 carbon atoms, an alkynyl group having 2 to 6 carbon atoms, a cycloalkyl group having 3 to 10 carbon atoms, an alkoxy group having 1 to 6 carbon atoms, a trialkylsilyl group having 3 to 10 carbon atoms, an aryl group having 6 to 30 carbon atoms, and a heteroaryl group having 2 to 30 carbon atoms.

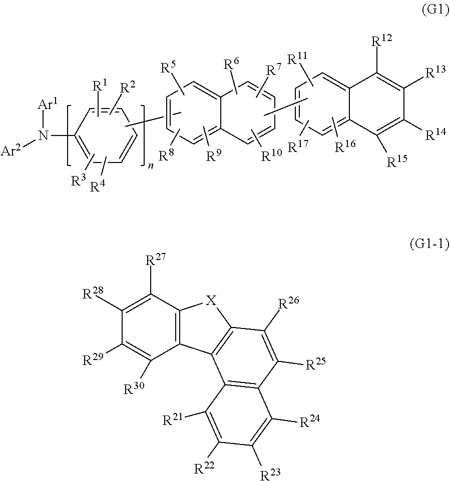

### BACKGROUND OF THE INVENTION

### 1. Field of the Invention

One embodiment of the present invention relates to an organic compound, an organic semiconductor device, a light-emitting device, a light-receiving device, a light-emitting apparatus, a light-receiving apparatus, a display device, an electronic appliance, a lighting device, and an electronic device. Note that one embodiment of the present invention is not limited to the above technical field. The technical field of one embodiment of the invention disclosed in this specification and the like relates to an object, a method, or a manufacturing method. One embodiment of the present invention relates to a process, a machine, manufacture, or a composition of matter. Specifically, examples of the technical field of one embodiment of the present invention disclosed in this specification include a semiconductor apparatus, a display device, a liquid crystal display apparatus, a light-emitting apparatus, a lighting device, a power storage device, a memory device, an imaging device, a driving method thereof, and a manufacturing method thereof.

### 2. Description of the Related Art

In recent years, organic semiconductor devices have been expected to be applied to a variety of uses. Specific examples of the organic semiconductor devices include a light-emitting device such as an organic light-emitting diode (OLED), a photoelectric conversion device such as an organic optical sensor or an organic thin film solar cell, and an organic field-effect transistor. Among them, light-emitting devices utilizing electroluminescence (hereinafter referred to as EL) have features such as ease of reduction in thickness and weight, high-speed response to input signals, and driving with a constant DC voltage power source, and thus have been used in display devices.

In improving device characteristics of the organic semiconductor device, there are many problems which depend on a substance contained in the organic semiconductor device, such as an organic compound, a metal, and a metal compound. In order to solve the problems, improvement of a device structure, development of a substance, and the like have been carried out. For example, Patent Document 1 discloses a hole-transport material, a kind of organic compound that can increase emission efficiency of a light-emitting device, a kind of organic semiconductor device, when used for the light-emitting device.

### REFERENCE

[Patent Document 1] Japanese Published Patent Application No. 2009-298767

### SUMMARY OF THE INVENTION

An object of one embodiment of the present invention is to provide a novel organic compound. Another object of one embodiment of the present invention is to provide a novel carrier-transport material. Another object of one embodiment of the present invention is to provide a novel hole-transport material. Another object of one embodiment of the present invention is to provide a highly heat-resistant carrier-transport material or hole-transport material.

Another object of one embodiment of the present invention is to provide an organic semiconductor device with small change in driving voltage over driving time. Another object of one embodiment of the present invention is to provide an organic semiconductor device having a long driving lifetime. Another object of one embodiment of the present invention is to provide a light-emitting device having high emission efficiency. Another object of one embodiment of the present invention is to provide a photoelectric conversion device having excellent detection sensitivity. Another object of one embodiment of the present invention is to provide an organic semiconductor device, a light-emitting apparatus, an electronic appliance, a display device, and an electronic device each having low power consumption.

Note that the description of these objects does not preclude the existence of other objects. One embodiment of the present invention does not need to achieve all of these objects. Other objects will be apparent from and can be derived from the description of the specification, the drawings, the claims, and the like.

One embodiment of the present invention is an organic compound represented by General Formula (G1).

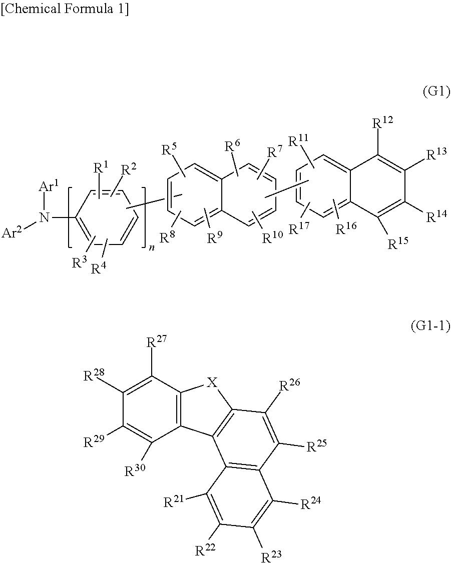

In General Formula (G1), Ar¹ represents a substituted or unsubstituted aryl group having 6 to 30 carbon atoms or a substituted or unsubstituted heteroaryl group having 2 to 30 carbon atoms; Ar² is a group represented by General Formula (G1-1); R¹ to R¹⁷ each independently represent any one of hydrogen (including deuterium), a substituted or unsubstituted alkyl group having 1 to 6 carbon atoms, and a substituted or unsubstituted cycloalkyl group having 3 to 10 carbon atoms; n represents an integer of 0 to 3; and in the case where n is 2 or more, a plurality of R¹ to R⁴ may be the same or different from each other. In General Formula (G1-1), X represents oxygen or sulfur; any one of R²¹ to R³⁰ is bonded to nitrogen in General Formula (G1); and R²¹ to R³⁰, except for the one bonded to the nitrogen, each independently represent any one of hydrogen (including deuterium), a halogen, a cyano group, a substituted or unsubstituted alkyl group having 1 to 6 carbon atoms, a substituted or unsubstituted alkenyl group having 2 to 6 carbon atoms, a substituted or unsubstituted alkynyl group having 2 to 6 carbon atoms, a substituted or unsubstituted cycloalkyl group having 3 to 10 carbon atoms, a substituted or unsubstituted alkoxy group having 1 to 6 carbon atoms, a trialkylsilyl group having 3 to 10 carbon atoms, a substituted or unsubstituted aryl group having 6 to 30 carbon atoms, and a substituted or unsubstituted heteroaryl group having 2 to 30 carbon atoms.

Another embodiment of the present invention is an organic compound represented by General Formula (G2).

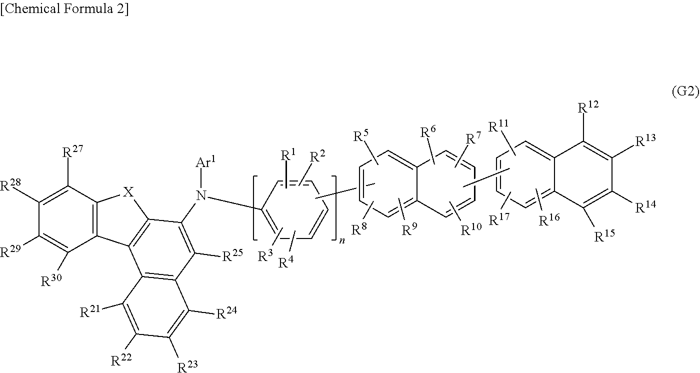

In General Formula (G2), Ar¹ represents a substituted or unsubstituted aryl group having 6 to 30 carbon atoms or a substituted or unsubstituted heteroaryl group having 2 to 30 carbon atoms; R¹ to R¹⁷ each independently represent any one of hydrogen (including deuterium), a substituted or unsubstituted alkyl group having 1 to 6 carbon atoms, and a substituted or unsubstituted cycloalkyl group having 3 to 10 carbon atoms; X represents oxygen or sulfur; R²¹ to R²⁵ and R²⁷ to R³⁰ each independently represent any one of hydrogen (including deuterium), a halogen, a cyano group, a substituted or unsubstituted alkyl group having 1 to 6 carbon atoms, a substituted or unsubstituted alkenyl group having 2 to 6 carbon atoms, a substituted or unsubstituted alkynyl group having 2 to 6 carbon atoms, a substituted or unsubstituted cycloalkyl group having 3 to 10 carbon atoms, a substituted or unsubstituted alkoxy group having 1 to 6 carbon atoms, a trialkylsilyl group having 3 to 10 carbon atoms, a substituted or unsubstituted aryl group having 6 to 30 carbon atoms, and a substituted or unsubstituted heteroaryl group having 2 to 30 carbon atoms; n represents an integer of 0 to 3; and in the case where n is 2 or more, a plurality of R¹ to R⁴ may be the same or different from each other.

Another embodiment of the present invention is an organic compound represented by General Formula (G3).

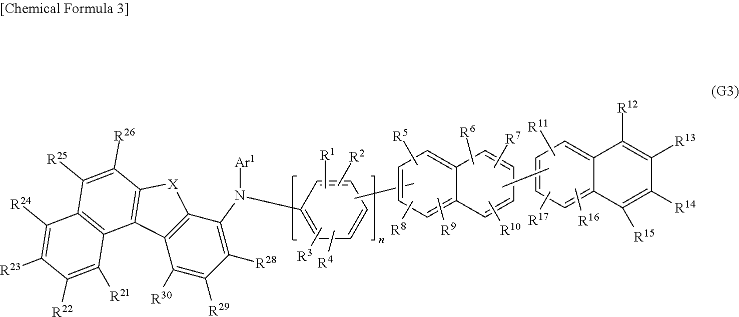

In General Formula (G3), Ar¹ represents a substituted or unsubstituted aryl group having 6 to 30 carbon atoms or a substituted or unsubstituted heteroaryl group having 2 to 30 carbon atoms; R¹ to R¹⁷ each independently represent any one of hydrogen (including deuterium), a substituted or unsubstituted alkyl group having 1 to 6 carbon atoms, and a substituted or unsubstituted cycloalkyl group having 3 to 10 carbon atoms; X represents oxygen or sulfur; R²¹ to R²⁶ and R²⁸ to R³⁰ each independently represent any one of hydrogen (including deuterium), a halogen, a cyano group, a substituted or unsubstituted alkyl group having 1 to 6 carbon atoms, a substituted or unsubstituted alkenyl group having 2 to 6 carbon atoms, a substituted or unsubstituted alkynyl group having 2 to 6 carbon atoms, a substituted or unsubstituted cycloalkyl group having 3 to 10 carbon atoms, a substituted or unsubstituted alkoxy group having 1 to 6 carbon atoms, a trialkylsilyl group having 3 to 10 carbon atoms, a substituted or unsubstituted aryl group having 6 to 30 carbon atoms, and a substituted or unsubstituted heteroaryl group having 2 to 30 carbon atoms; n represents an integer of 0 to 3; and in the case where n is 2 or more, a plurality of R¹ to R⁴ may be the same or different from each other.

Another embodiment of the present invention is an organic compound represented by General Formula (G4).

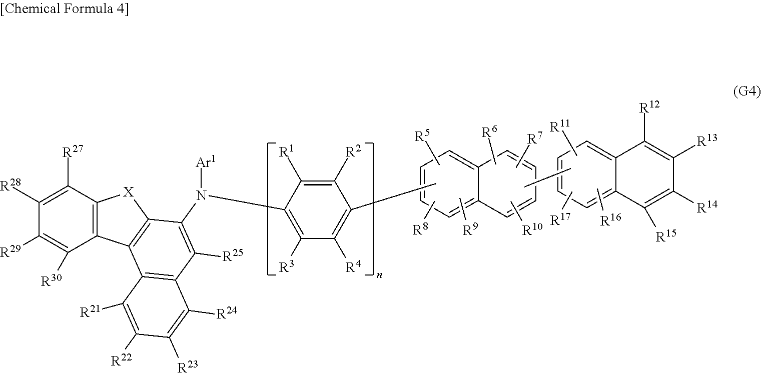

In General Formula (G4), Ar¹ represents a substituted or unsubstituted aryl group having 6 to 30 carbon atoms or a substituted or unsubstituted heteroaryl group having 2 to 30 carbon atoms; R¹ to R¹⁷ each independently represent any one of hydrogen (including deuterium), a substituted or unsubstituted alkyl group having 1 to 6 carbon atoms, and a substituted or unsubstituted cycloalkyl group having 3 to 10 carbon atoms; X represents oxygen or sulfur; R²¹ to R²⁵ and R²⁷ to R³⁰ each independently represent any one of hydrogen (including deuterium), a halogen, a cyano group, a substituted or unsubstituted alkyl group having 1 to 6 carbon atoms, a substituted or unsubstituted alkenyl group having 2 to 6 carbon atoms, a substituted or unsubstituted alkynyl group having 2 to 6 carbon atoms, a substituted or unsubstituted cycloalkyl group having 3 to 10 carbon atoms, a substituted or unsubstituted alkoxy group having 1 to 6 carbon atoms, a trialkylsilyl group having 3 to 10 carbon atoms, a substituted or unsubstituted aryl group having 6 to 30 carbon atoms, and a substituted or unsubstituted heteroaryl group having 2 to 30 carbon atoms; n represents an integer of 1 to 3; and in the case where n is 2 or more, a plurality of R¹ to R⁴ may be the same or different from each other.

Another embodiment of the present invention is an organic compound represented by General Formula (G5).

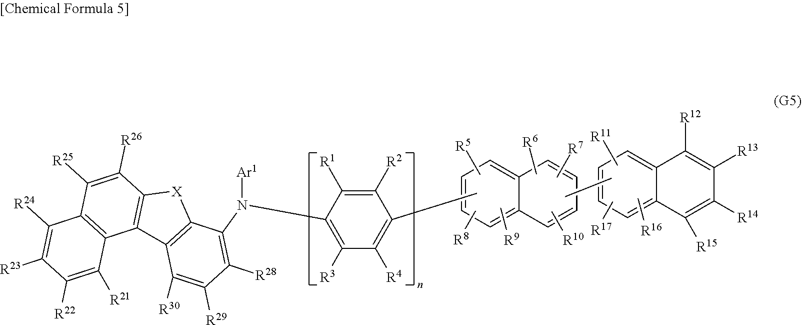

In General Formula (G5), Ar¹ represents a substituted or unsubstituted aryl group having 6 to 30 carbon atoms or a substituted or unsubstituted heteroaryl group having 2 to 30 carbon atoms; R¹ to R¹⁷ each independently represent any one of hydrogen (including deuterium), a substituted or unsubstituted alkyl group having 1 to 6 carbon atoms, and a substituted or unsubstituted cycloalkyl group having 3 to 10 carbon atoms; X represents oxygen or sulfur; R²¹ to R²⁶ and R²⁸ to R³⁰ each independently represent any one of hydrogen (including deuterium), a halogen, a cyano group, a substituted or unsubstituted alkyl group having 1 to 6 carbon atoms, a substituted or unsubstituted alkenyl group having 2 to 6 carbon atoms, a substituted or unsubstituted alkynyl group having 2 to 6 carbon atoms, a substituted or unsubstituted cycloalkyl group having 3 to 10 carbon atoms, a substituted or unsubstituted alkoxy group having 1 to 6 carbon atoms, a trialkylsilyl group having 3 to 10 carbon atoms, a substituted or unsubstituted aryl group having 6 to 30 carbon atoms, and a substituted or unsubstituted heteroaryl group having 2 to 30 carbon atoms; n represents an integer of 1 to 3; and in the case where n is 2 or more, a plurality of R¹ to R⁴ may be the same or different from each other.

Another embodiment of the present invention is an organic compound represented by General Formula (G6).

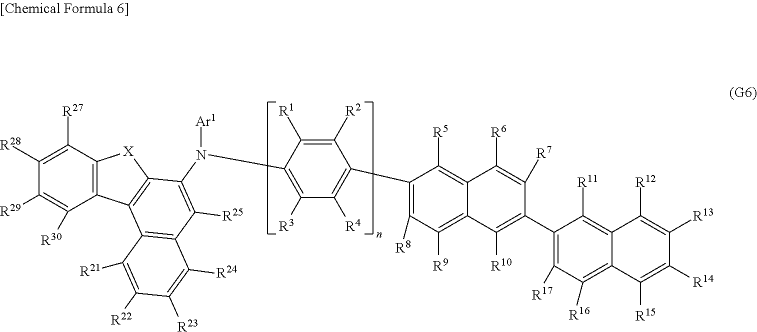

In General Formula (G6), Ar¹ represents a substituted or unsubstituted aryl group having 6 to 30 carbon atoms or a substituted or unsubstituted heteroaryl group having 2 to 30 carbon atoms; R¹ to R¹⁷ each independently represent any one of hydrogen (including deuterium), a substituted or unsubstituted alkyl group having 1 to 6 carbon atoms, and a substituted or unsubstituted cycloalkyl group having 3 to 10 carbon atoms; X represents oxygen or sulfur; R²¹ to R²⁵ and R²⁷ to R³⁰ each independently represent any one of hydrogen (including deuterium), a halogen, a cyano group, a substituted or unsubstituted alkyl group having 1 to 6 carbon atoms, a substituted or unsubstituted alkenyl group having 2 to 6 carbon atoms, a substituted or unsubstituted alkynyl group having 2 to 6 carbon atoms, a substituted or unsubstituted cycloalkyl group having 3 to 10 carbon atoms, a substituted or unsubstituted alkoxy group having 1 to 6 carbon atoms, a trialkylsilyl group having 3 to 10 carbon atoms, a substituted or unsubstituted aryl group having 6 to 30 carbon atoms, and a substituted or unsubstituted heteroaryl group having 2 to 30 carbon atoms; n represents an integer of 0 to 3; and in the case where n is 2 or more, a plurality of R¹ to R⁴ may be the same or different from each other.

Another embodiment of the present invention is an organic compound represented by General Formula (G7).

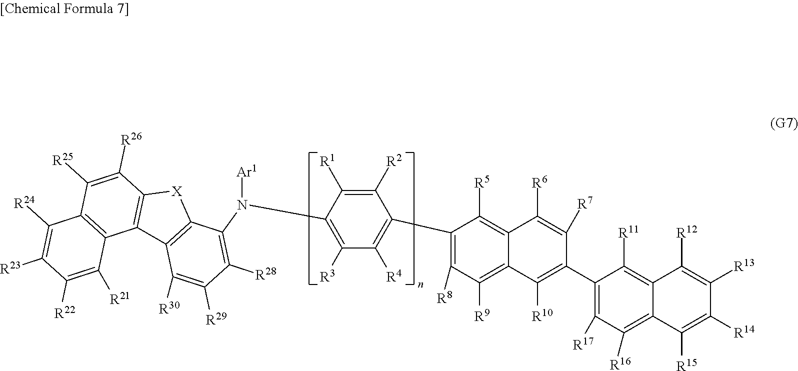

In General Formula (G7), Ar¹ represents a substituted or unsubstituted aryl group having 6 to 30 carbon atoms or a substituted or unsubstituted heteroaryl group having 2 to 30 carbon atoms; R¹ to R¹⁷ each independently represent any one of hydrogen (including deuterium), a substituted or unsubstituted alkyl group having 1 to 6 carbon atoms, and a substituted or unsubstituted cycloalkyl group having 3 to 10 carbon atoms; X represents oxygen or sulfur; R²¹ to R²⁶ and R²⁸ to R³⁰ each independently represent any one of hydrogen (including deuterium), a halogen, a cyano group, a substituted or unsubstituted alkyl group having 1 to 6 carbon atoms, a substituted or unsubstituted alkenyl group having 2 to 6 carbon atoms, a substituted or unsubstituted alkynyl group having 2 to 6 carbon atoms, a substituted or unsubstituted cycloalkyl group having 3 to 10 carbon atoms, a substituted or unsubstituted alkoxy group having 1 to 6 carbon atoms, a trialkylsilyl group having 3 to 10 carbon atoms, a substituted or unsubstituted aryl group having 6 to 30 carbon atoms, and a substituted or unsubstituted heteroaryl group having 2 to 30 carbon atoms; n represents an integer of 0 to 3; and in the case where n is 2 or more, a plurality of R^{t} to R⁴ may be the same or different from each other.

Another embodiment of the present invention is any of the above organic compounds in which n represents an integer of 1 to 3.

Another embodiment of the present invention is an organic compound represented by Structural Formula (103), (108), (117), or (118) below.

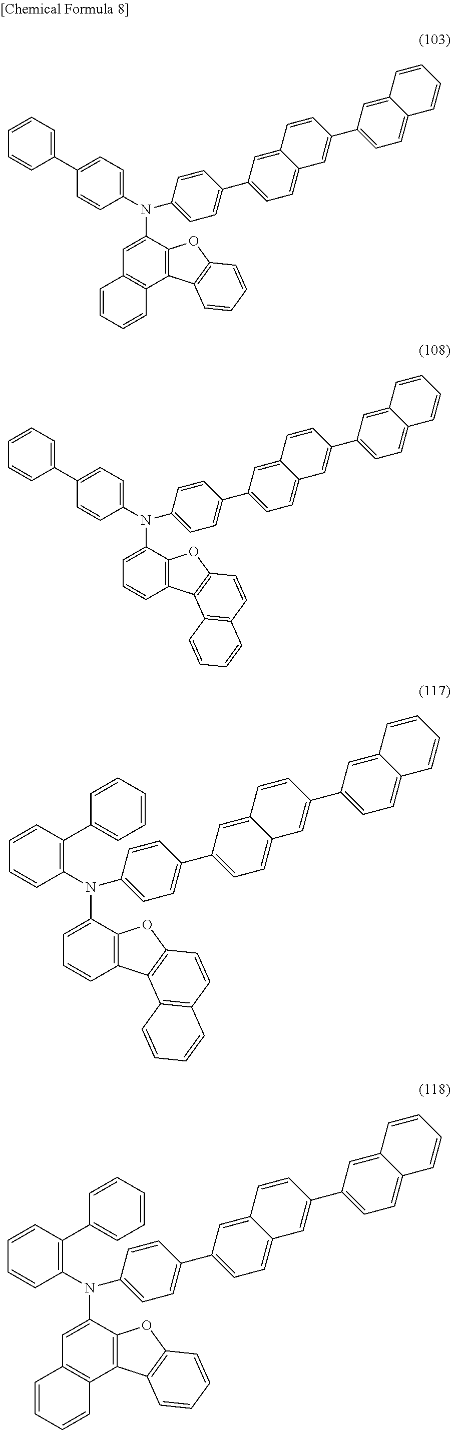 [SMILES: c1ccc(-c2ccc(N(c3ccc(-c4ccc5cc(-c6ccc7ccccc7c6)ccc5c4)cc3)c3cc4ccccc4c4c3oc3ccccc34)cc2)cc1 ; c1ccc(-c2ccc(N(c3ccc(-c4ccc5cc(-c6ccc7ccccc7c6)ccc5c4)cc3)c3cccc4c3oc3ccc5ccccc5c34)cc2)cc1 ; c1ccc(-c2ccccc2N(c2ccc(-c3ccc4cc(-c5ccc6ccccc6c5)ccc4c3)cc2)c2cccc3c2oc2ccc4ccccc4c23)cc1 ; c1ccc(-c2ccccc2N(c2ccc(-c3ccc4cc(-c5ccc6ccccc6c5)ccc4c3)cc2)c2cc3ccccc3c3c2oc2ccccc23)cc1]

Another embodiment of the present invention is an organic semiconductor device including the organic compound having any of the above structures.

Another embodiment of the present invention is an electronic appliance including the organic semiconductor device having the above structure.

According to one embodiment of the present invention, a novel organic compound can be provided. According to one embodiment of the present invention, a novel carrier-transport material can be provided. According to one embodiment of the present invention, a novel hole-transport material can be provided. According to one embodiment of the present invention, a highly heat-resistant carrier-transport material or hole-transport material can be provided.

According to another embodiment of the present invention, an organic semiconductor device with small change in driving voltage over driving time can be provided. According to another embodiment of the present invention, an organic semiconductor device having a long driving lifetime can be provided. According to another embodiment of the present invention, a light-emitting device having high emission efficiency can be provided. According to another embodiment of the present invention, a photoelectric conversion device having excellent detection sensitivity can be provided. According to another embodiment of the present invention, an organic semiconductor device, a light-emitting apparatus, an electronic appliance, a display device, and an electronic device each having low power consumption.

Note that the description of these effects does not preclude the existence of other effects. One embodiment of the present invention does not necessarily have all of these effects. Other effects can be derived from the description of the specification, the drawings, and the claims.

### BRIEF DESCRIPTION OF THE DRAWINGS

In the accompanying drawings:

FIGS. 1A to 1C each illustrate a structure of an organic semiconductor device of an embodiment;

FIGS. 2A and 2B each illustrate a structure of a device of an embodiment;

FIGS. 3A to 3F each illustrate a structure of a light-emitting device of an embodiment;

FIGS. 4A to 4D illustrate a light-emitting and light-receiving apparatus of an embodiment;

FIGS. 5A to 5C illustrate a method for manufacturing a light-emitting and light-receiving apparatus of an embodiment;

FIGS. 6A to 6C illustrate a method for manufacturing a light-emitting and light-receiving apparatus of an embodiment;

FIGS. 7A to 7D illustrate a method for manufacturing a light-emitting and light-receiving apparatus of an embodiment;

FIGS. 8A to 8C illustrate a light-emitting and light-receiving apparatus of an embodiment.

FIGS. 9A to 9F illustrate an apparatus and pixel arrangements of an embodiment;

FIGS. 10A and 10B illustrate pixel circuits of an embodiment and FIG. 10C illustrates a transistor used in the pixel circuits;

FIG. 11 illustrates a light-emitting apparatus of an embodiment;

FIGS. 12A to 12E illustrate electronic appliances of an embodiment;

FIGS. 13A to 13E illustrate electronic appliances of an embodiment;

FIGS. 14A and 14B illustrate electronic appliances of an embodiment;

FIGS. 15A and 15B illustrate a lighting device of an embodiment;

FIG. 16 illustrates a lighting device of an embodiment;

FIG. 17 shows a ¹H NMR spectrum of (βN2)PBiBnf(6);

FIG. 18 shows an absorption spectrum and an emission spectrum of a toluene solution of (βN2)PBiBnf(6);

FIG. 19 shows an absorption spectrum and an emission spectrum of a solid thin film of (βN2)PBiBnf(6);

FIG. 20 shows a ¹H NMR spectrum of (βN2)PBiBnf(8);

FIG. 21 shows an absorption spectrum and an emission spectrum of a toluene solution of (βN2)PBiBnf(8);

FIG. 22 shows an absorption spectrum and an emission spectrum of a solid thin film of (βN2)PBiBnf(8);

FIG. 23 shows a ¹H NMR spectrum of (βN2)PoBiBnf(6);

FIG. 24 shows an absorption spectrum and an emission spectrum of a toluene solution of (βN2)PoBiBnf(6);

FIG. 25 shows an absorption spectrum and an emission spectrum of a solid thin film of (βN2)PoBiBnf(6);

FIG. 26 shows a ¹H NMR spectrum of (βN2)PoBiBnf(8);

FIG. 27 shows an absorption spectrum and an emission spectrum of a toluene solution of (βN2)PoBiBnf(8);

FIG. 28 shows an absorption spectrum and an emission spectrum of a solid thin film of (βN2)PoBiBnf(8); and

FIG. 29 illustrates a light-emitting device of an example.

### DETAILED DESCRIPTION OF THE INVENTION

Embodiments of the present invention will be described in detail below with reference to the drawings. Note that the present invention is not limited to the following description, and the modes and details of the present invention can be modified in various ways without departing from the spirit and scope of the present invention. Thus, the present invention should not be construed as being limited to the description in the following embodiments.

Note that the position, size, range, or the like of each component illustrated in drawings and the like is not accurately represented in some cases for easy understanding. Thus, the disclosed invention is not necessarily limited to the position, size, range, or the like disclosed in the drawings and the like.

Ordinal numbers such as “first” and “second” in this specification and the like are used for convenience and do not denote the order of steps or the stacking order of layers in some cases. Thus, for example, description can be made even when “first” is replaced with “second” or “third”, as appropriate. In addition, the ordinal numbers in this specification and the like are not necessarily the same as those used to specify one embodiment of the present invention.

In the description of structures of the present invention in this specification and the like with reference to the drawings, the same components in different drawings are denoted by the same reference numeral in some cases.

In this specification and the like, the terms “film” and “layer” can be interchanged with each other. For example, the term “conductive layer” can be changed to the term “conductive film” in some cases. For another example, the term “insulating film” can be changed into the term “insulating layer” in some cases.

In this specification and the like, a singlet excited state (S*) refers to a singlet state having excitation energy. An S₁ level means the lowest level of the singlet excitation energy level, that is, the excitation energy level of the lowest singlet excited state. A triplet excited state (T*) refers to a triplet state having excitation energy. A T₁ level means the lowest level of the triplet excitation energy level, that is, the excitation energy level of the lowest triplet excited state.

Note that when the ν=0→ν=0 transition (0→0 band) between vibrational levels of the ground state and the excited state is clearly observed from a fluorescent spectrum or a phosphorescent spectrum, the S₁ level or the T₁ level of an organic compound is preferably calculated using the 0→0 band (see Nicholas J. Turro, V. Ramamurthy, J. C. Scaiano, “MODERN MOLECULAR PHOTOCHEMISTRY OF ORGANIC MOLECULES”, UNIVERSITY SCIENCE BOOKS, 2010.02.10, pp. 204-208). When the 0→0 band is unclear, the S₁ level can be energy of the wavelength at the intersection of the horizontal axis (wavelength) or the base line and a tangent to the fluorescence spectrum at a point where the slope of the spectrum at a peak on the shorter wavelength side has a maximum value, and the T₁ level can be energy of the wavelength at the intersection of the horizontal axis (wavelength) or the base line and a tangent to the phosphorescence spectrum at a point where the slope of the spectrum at a peak on the shorter wavelength side has a maximum value (see Daisaku TANAKA et al., “Ultra High Efficiency Green Organic Light-Emitting Devices”, Japanese Journal of Applied Physics, Vol. 46, No. 1, 2007, pp. L10-L12). In this specification, the latter method is employed to measure the levels. In the case where the levels are compared with each other, those calculated by the same method are used.

In addition, in this specification and the like, the term “substituted” in the expression “substituted or unsubstituted” means that a group has a substituent.

### Embodiment 1

In this embodiment, organic compounds of embodiments of the present invention will be described.

One embodiment of the present invention is an organic compound having a structure in which a first aryl group, a second aryl group, and a third aryl group are bonded to one nitrogen, i.e., triarylamine. The triarylamine excels in hole-transport property because it has a high LUMO (Lowest Unoccupied Molecular Orbital) level and a large gap between the HOMO (Highest Occupied Molecular Orbital) and the LUMO. Note that in this specification, nitrogen at the center of the triarylamine is sometimes referred to as “N^{A}” for short.

In one embodiment of the present invention, two naphthalene rings bonded through a single bond (this structure is referred to as a binaphthyl structure or a binaphthyl skeleton in this specification) are employed for the first aryl group. The naphthalene ring has a structure in which two benzene rings are condensed and thus is stable. In the binaphthyl structure, a conjugated system spreads across the two naphthalene rings, achieving a stable structure. Thus, employing the binaphthyl structure for the first aryl group can increase the stability and the heat resistance of the triarylamine. Accordingly, when deposited by vacuum evaporation, such triarylamine is less likely to be thermally decomposed by heat generated during sublimation, and thus, a film with high purity can be obtained. As a result, a film with a stable quality can be formed. With such triarylamine, a highly reliable organic semiconductor device having high quality can be manufactured.

In the case where at least one of the two naphthalene rings is substituted at its Q-position in the binaphthyl structure, the planarity of the structure is high, and the conjugated system spreads across the two naphthalene rings. It can be said that in the case where both of the two naphthalene rings are substituted at the β-position, the planarity of the structure is highest, and the conjugation easily spreads. Accordingly, in both cases, employing the binaphthyl structure for the first aryl group can spread conjugation between an unshared electron pair of nitrogen at the center of the triarylamine (N^{A}) and the binaphthyl structure, so that a hole-transport property of the triarylamine can be increased. Thus, with such triarylamine, the efficiency of an organic semiconductor device can be increased.

In the case where at least one of the two naphthalene rings is substituted at its α-position in the binaphthyl structure, the two naphthalene rings are likely to have a twisted steric configuration. In the case where both of the two naphthalene rings are bonded at the α-position, the two naphthalene rings have the most twisted steric configuration, i.e., a bulky molecular structure. As a result, the compound can be highly resistant to heat. Accordingly, in both cases, employing the binaphthyl structure for the first aryl group can produce a bulky triarylamine compound that is a material having high heat resistance. Thus, with such triarylamine, an organic semiconductor device having high heat resistance can be provided.

In one embodiment of the present invention, for the second aryl group, a benzo[b]naphtho[1,2-d]furan ring (sometimes referred to as a benzonaphthofuran ring in this specification) or a benzo[b]naphtho[1,2-d]thiophene ring (sometimes referred to as a benzonaphthothiophene ring in this specification) is used. The benzonaphthofuran ring and the benzonaphthothiophene ring each have a naphthalene skeleton in the ring and thus has a property of easily accepting electrons because conjugation spreads in the ring. Thus, by using the benzonaphthofuran ring or the benzonaphthothiophene ring for the second aryl group, the LUMO of the triarylamine is likely to be distributed across the ring. As a result, the LUMO is less likely to be distributed in the other aryl groups of the triarylamine, i.e., the first aryl group and the third aryl group, and thus the resistance to reduction of the triarylamine can be increased. Accordingly, with such triarylamine, change in driving voltage over driving time of an organic semiconductor device can be made small. In addition, the organic semiconductor device can have a long driving lifetime.

More specifically, one embodiment of the present invention is an organic compound represented by General Formula (G1).

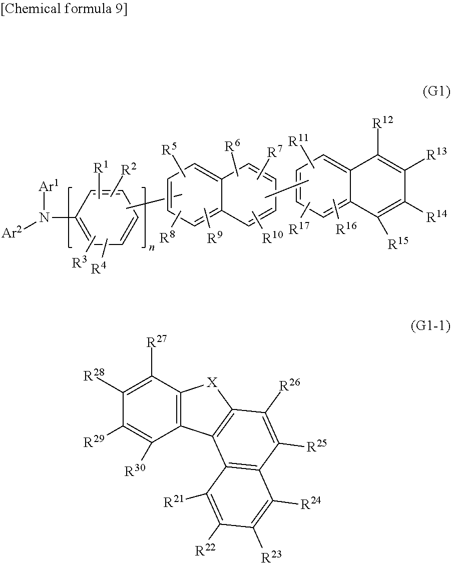

In General Formula (G1), Ar¹ represents a substituted or unsubstituted aryl group having 6 to 30 carbon atoms or a substituted or unsubstituted heteroaryl group having 2 to 30 carbon atoms; Ar² is a group represented by General Formula (G1-1); R¹ to R¹⁷ each independently represent any one of hydrogen (including deuterium), a substituted or unsubstituted alkyl group having 1 to 6 carbon atoms, and a substituted or unsubstituted cycloalkyl group having 3 to 10 carbon atoms; n represents an integer of 0 to 3; and in the case where n is 2 or more, a plurality of R¹ to R⁴ may be the same or different from each other. In General Formula (G1-1), X represents oxygen or sulfur; any one of R²¹ to R³⁰ is bonded to nitrogen (nitrogen at the center of the triarylamine, i.e., N^{A}) in General Formula (G1); and R²¹ to R³⁰, except for the one bonded to the nitrogen, each independently represent any one of hydrogen (including deuterium), a halogen, a cyano group, a substituted or unsubstituted alkyl group having 1 to 6 carbon atoms, a substituted or unsubstituted alkenyl group having 2 to 6 carbon atoms, a substituted or unsubstituted alkynyl group having 2 to 6 carbon atoms, a substituted or unsubstituted cycloalkyl group having 3 to 10 carbon atoms, a substituted or unsubstituted alkoxy group having 1 to 6 carbon atoms, a trialkylsilyl group having 3 to 10 carbon atoms, a substituted or unsubstituted aryl group having 6 to 30 carbon atoms, and a substituted or unsubstituted heteroaryl group having 2 to 30 carbon atoms.

The organic compound represented by General Formula (G1) is triarylamine, an organic compound in which the binaphthyl structure is used for the first aryl group and the benzonaphthofuran ring or the benzonaphthothiophene ring represented by General Formula (G1-1) is used for the second aryl group.

As described above, employing the binaphthyl structure for the first aryl group can increase the stability and the heat resistance of the triarylamine. Accordingly, when deposited by vacuum evaporation, the organic compound represented by General Formula (G1) is less likely to be thermally decomposed by heat generated during sublimation, and thus, a film with high purity can be obtained. As a result, a film with a more stable quality can be formed. Thus, with the organic compound represented by General Formula (G1), a highly reliable organic semiconductor device having high quality can be manufactured.

As described above, the LUMO of the organic compound represented by General Formula (G1) is likely to be distributed around a naphthalene skeleton of the benzonaphthofuran ring or the benzonaphthothiophene ring. Thus, the LUMO is less likely to be distributed in the other aryl groups of the triarylamine, i.e., the first aryl group and the third aryl group, and thus the organic compound represented by General Formula (G1) can be said to be highly resistant to reduction. In the case where such an organic compound is used for a hole-transport layer in contact with the light-emitting layer of a light-emitting device, which is an example of an organic semiconductor device, for example, the hole-transport layer having high resistance to electrons is resistant to electrons passed through the light-emitting layer, and thus the driving lifetime of the device can be increased. As described above, with the organic compound represented by General Formula (G1), the driving lifetime of the organic semiconductor device can be extended; that is, the reliability of the organic semiconductor device can be increased.

Furthermore, part of the HOMO of the organic compound represented by General Formula (G1) is likely to be distributed in a naphthalene skeleton or a benzene skeleton, which is directly bonded to N^{A}, of the benzonaphthofuran ring or the benzonaphthothiophene ring. In General Formula (G1-1), it is preferable that R²⁶ or R²⁷ be bonded to N^{A}, that is, a 6-position or a 8-position of the benzonaphthofuran ring or the benzonaphthothiophene ring be bonded to N^{A}, because the HOMO level of the organic compound is not too high with this structure. An electron-withdrawing effect of oxygen of the benzonaphthofuran ring or sulfur of the benzonaphthothiophene ring can easily reduce the electron density of carbon where R²⁷ is bonded in the benzene skeleton or the electron density of carbon where R²⁷ is bonded in the naphthalene skeleton; accordingly, the HOMO level of the triarylamine can be made low. Thus, with the organic compound represented by General Formula (G1), a material having a desired HOMO level for an organic semiconductor device can be provided.

Note that in the case where n is 1, the positional relation between the binaphthyl skeleton and N^{A} which are bonded to a phenylene group where R¹ to R⁴ are bonded is made to be at a para-position to further reduce steric hindrance around N^{A}, whereby a hole-transport property of triarylamine can be further increased. In particular, a compound having a high hole-injection property and a high hole mobility can be provided. Since the binaphthyl structure is not directly bonded to N^{A} in this structure, the T₁ level of the triarylamine of the organic compound represented by General Formula (G1) can be increased. With this structure, triarylamine having high heat resistance can be provided.

Note that in the case where n is 1, the positional relation between the binaphthyl skeleton and N^{A} which are bonded to a phenylene group where R¹ to R⁴ are bonded is made to be at a meta-position to further reduce steric hindrance around N^{A}, whereby triarylamine which sublimates at a low temperature can be provided. Since solubility in an organic solvent can be potentially increased with this structure, purification by column chromatography or the like is easily conducted, whereby a highly purified triarylamine can be provided. Furthermore, since the binaphthyl structure is not directly bonded to N^{A} in this structure, the T₁ level of the triarylamine can be increased. In particular, in the case where a 1,3-phenylene group (m-phenylene group) crosslinks N^{A} and the binaphthyl structure, conjugation can be prevented from excessively spreading, and the T₁ level of the triarylamine can be potentially increased further more as compared to the case where a 1,4-phenylene group (p-phenylene group) crosslinks N^{A} and the binaphthyl structure.

Note that in the case where n is 1, the positional relation between the binaphthyl skeleton and N^{A} which is bonded to a phenylene group where R¹ to R⁴ are bonded is made to be at an ortho-position, whereby the triarylamine can have a bulky molecular structure without lowering of a hole-transport property, and a compound having high heat resistance can be provided. Since the binaphthyl structure is not directly bonded to N^{A} in this structure, the T₁ level of the triarylamine of the organic compound represented by General Formula (G1) can be increased. In particular, when a 1,2-phenylene group (o-phenylene group) crosslinks N^{A} and the binaphthyl structure, the triarylamine can be inhibited from increasing in crystallinity and can have a structure having high heat resistance.

In General Formula (G1-1), the organic compound in the case where X is oxygen can have a lower refractive index than that in the case where X is sulfur. An organic compound having a low refractive index is preferably used for a light-emitting device or the like, in which case the light extraction efficiency can be increased. Meanwhile, in General Formula (G1-1), the organic compound in the case where X is sulfur can have higher heat resistance (a higher decomposition temperature, melting point, sublimation point, or the like) than that in the case where X is oxygen. An organic compound having high heat resistance is preferably used for a light-emitting device or the like, in which case an organic semiconductor device capable of stably driving in a high-temperature environment can be provided.

In General Formula (G1-1), when R²⁶ or R²⁷ is a substituent other than hydrogen, the organic compound has a bulky molecular structure and its heat resistance can be increased.

In the case where a substituent other than hydrogen is introduced in General Formula (G1-1), its substitution site is preferably R²⁶ or R²⁷. In the case where R²⁶ or R²⁷ in General Formula (G1-1) is hydrogen, because of having relatively high reactivity, the hydrogen can reduce the number of synthesis steps and introduce a substituent at R²⁶ or R²⁷ with a high yield as compared to the case where a substituent is introduced at a substitution site other than R²⁶ and R²⁷. In that case, an effect of reducing production cost can be obtained, and thus an excellent material can be offered at low cost; in other words, this structure excels also in mass production.

Specific examples of the group represented by General Formula (G1-1) are shown in Structural Formulae (Ar²-1) to (Ar²-11) below. Note that specific examples of the group represented by General Formula (G1-1) are not limited thereto.

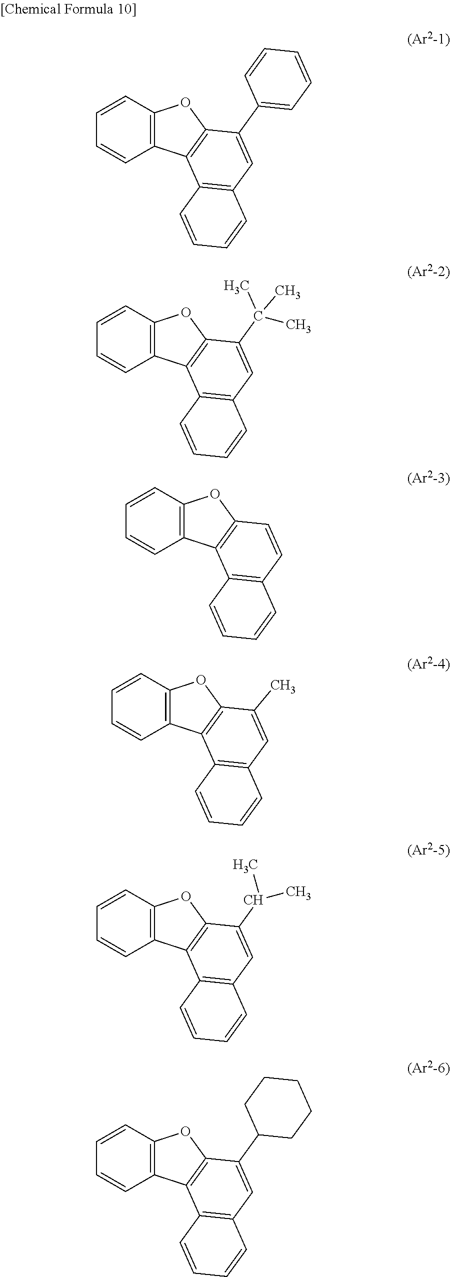

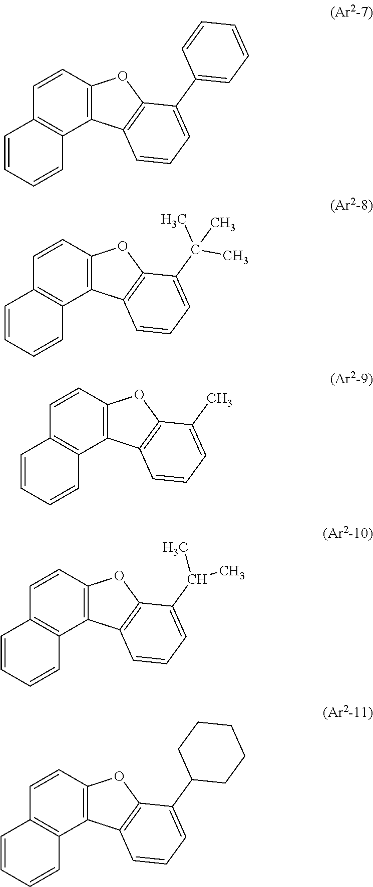

One embodiment of the present invention is an organic compound represented by General Formula (G2). Note that General Formula (G2) is different from General Formula (G1) in that the substitution site of General Formula (G1-1) in General Formula (G1) is fixed to R²⁶. Thus, description of the structure, effect, and the like of the organic compound represented by General Formula (G1) can be applied to General Formula (G2).

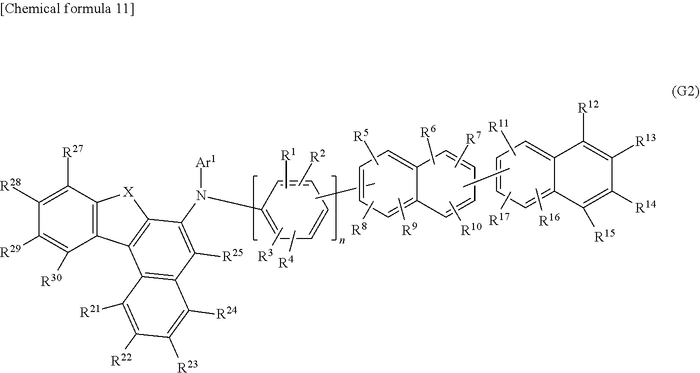

In General Formula (G2), Ar¹ represents a substituted or unsubstituted aryl group having 6 to 30 carbon atoms or a substituted or unsubstituted heteroaryl group having 2 to 30 carbon atoms; R¹ to R¹⁷ each independently represent any one of hydrogen (including deuterium), a substituted or unsubstituted alkyl group having 1 to 6 carbon atoms, and a substituted or unsubstituted cycloalkyl group having 3 to 10 carbon atoms; X represents oxygen or sulfur; R²¹ to R²⁵ and R²⁷ to R³⁰ each independently represent any one of hydrogen (including deuterium), a halogen, a cyano group, a substituted or unsubstituted alkyl group having 1 to 6 carbon atoms, a substituted or unsubstituted alkenyl group having 2 to 6 carbon atoms, a substituted or unsubstituted alkynyl group having 2 to 6 carbon atoms, a substituted or unsubstituted cycloalkyl group having 3 to 10 carbon atoms, a substituted or unsubstituted alkoxy group having 1 to 6 carbon atoms, a trialkylsilyl group having 3 to 10 carbon atoms, a substituted or unsubstituted aryl group having 6 to 30 carbon atoms, and a substituted or unsubstituted heteroaryl group having 2 to 30 carbon atoms; n represents an integer of 0 to 3; and in the case where n is 2 or more, a plurality of R¹ to R⁴ may be the same or different from each other.

As described above, in General Formula (G2), the substitution site of General Formula (G1-1) in General Formula (G1) is fixed to R²⁶. In other words, in General Formula (G2), a 6-position of a benzonaphthofuran ring or a benzonaphthothiophene ring is bonded and fixed to N^{A}. With this structure, the electron density of a naphthalene skeleton directly bonded to N^{A} is easily lowered owing to an influence of oxygen of the benzonaphthofuran ring or sulfur of the benzonaphthothiophene ring; accordingly, the HOMO level of the triarylamine can be made low. Thus, with the organic compound represented by General Formula (G1), a material having a desired HOMO level for an organic semiconductor device can be provided. In General Formula (G2), the 6-position of the benzonaphthofuran ring or the benzonaphthothiophene ring is bonded to N^{A}, whereby the heat resistance of the organic compound can be further increased.

One embodiment of the present invention is an organic compound represented by General Formula (G3). Note that General Formula (G3) is different from General Formula (G1) in that the substitution site of General Formula (G1-1) in General Formula (G1) is fixed to R²⁷. Thus, description of the structure, effect, and the like of the organic compound represented by General Formula (G1) can be applied to General Formula (G3).

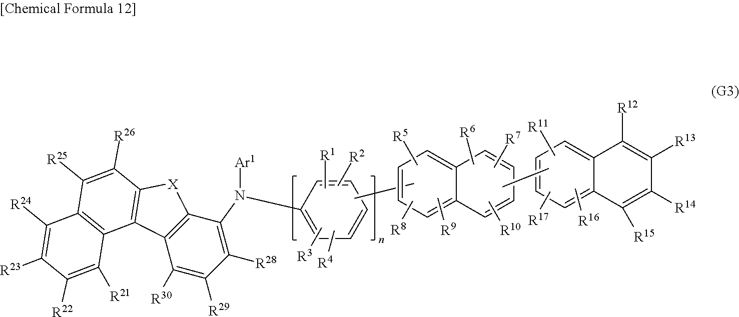

In General Formula (G3), Ar¹ represents a substituted or unsubstituted aryl group having 6 to 30 carbon atoms or a substituted or unsubstituted heteroaryl group having 2 to 30 carbon atoms; R¹ to R¹⁷ each independently represent any one of hydrogen (including deuterium), a substituted or unsubstituted alkyl group having 1 to 6 carbon atoms, and a substituted or unsubstituted cycloalkyl group having 3 to 10 carbon atoms; X represents oxygen or sulfur; R²¹ to R²⁶ and R²⁸ to R³⁰ each independently represent any one of hydrogen (including deuterium), a halogen, a cyano group, a substituted or unsubstituted alkyl group having 1 to 6 carbon atoms, a substituted or unsubstituted alkenyl group having 2 to 6 carbon atoms, a substituted or unsubstituted alkynyl group having 2 to 6 carbon atoms, a substituted or unsubstituted cycloalkyl group having 3 to 10 carbon atoms, a substituted or unsubstituted alkoxy group having 1 to 6 carbon atoms, a trialkylsilyl group having 3 to 10 carbon atoms, a substituted or unsubstituted aryl group having 6 to 30 carbon atoms, and a substituted or unsubstituted heteroaryl group having 2 to 30 carbon atoms; n represents an integer of 0 to 3; and in the case where n is 2 or more, a plurality of R¹ to R⁴ may be the same or different from each other.

As described above, in General Formula (G3), the substitution site of General Formula (G1-1) in General Formula (G1) is R²⁷. In other words, in General Formula (G3), an 8-position of a benzonaphthofuran ring or a benzonaphthothiophene ring is bonded to N^{A}. With this structure, the electron density of a benzene skeleton directly bonded to N^{A} is easily lowered owing to the influence of oxygen of the benzonaphthofuran ring or sulfur of the benzonaphthothiophene ring; accordingly, the HOMO level of the triarylamine can be made low. Thus, with the organic compound represented by General Formula (G3), a material having a desired HOMO level for an organic semiconductor device can be provided.

In the case where the 8-position of the benzonaphthofuran ring or the benzonaphthothiophene ring is bonded to N^{A}, the compound can have a higher phosphorescent level (T₁ level) than that in the case where the naphthalene skeleton of the benzonaphthofuran ring or the benzonaphthothiophene ring (a 1-, 2-, 3-, 4-, 5-, or 6-position of the benzonaphthofuran ring or the benzonaphthothiophene ring) is bonded to N^{A}. Thus, in the case where the organic compound represented by General Formula (G3) is used for a light-emitting layer of a phosphorescent light-emitting device or a layer in contact with the light-emitting layer, such as an electron-blocking layer, for example, the emission efficiency of the device can be increased.

One embodiment of the present invention is an organic compound represented by General Formula (G4). Note that General Formula (G4) is different from General Formula (G2) in that a phenylene group that connects the binaphthyl structure and N^{A} is fixed to a 1,4-phenylene group (p-phenylene group) and n is limited to one or more. Thus, description of the structure, effect, and the like of the organic compound represented by General Formula (G2) can be applied to General Formula (G4).

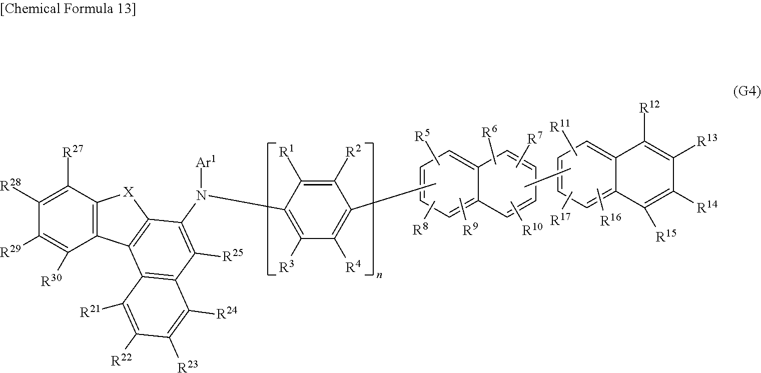

In General Formula (G4), Ar¹ represents a substituted or unsubstituted aryl group having 6 to 30 carbon atoms or a substituted or unsubstituted heteroaryl group having 2 to 30 carbon atoms; R¹ to R¹⁷ each independently represent any one of hydrogen (including deuterium), a substituted or unsubstituted alkyl group having 1 to 6 carbon atoms, and a substituted or unsubstituted cycloalkyl group having 3 to 10 carbon atoms; X represents oxygen or sulfur; R²¹ to R²⁵ and R²⁷ to R³⁰ each independently represent any one of hydrogen (including deuterium), a halogen, a cyano group, a substituted or unsubstituted alkyl group having 1 to 6 carbon atoms, a substituted or unsubstituted alkenyl group having 2 to 6 carbon atoms, a substituted or unsubstituted alkynyl group having 2 to 6 carbon atoms, a substituted or unsubstituted cycloalkyl group having 3 to 10 carbon atoms, a substituted or unsubstituted alkoxy group having 1 to 6 carbon atoms, a trialkylsilyl group having 3 to 10 carbon atoms, a substituted or unsubstituted aryl group having 6 to 30 carbon atoms, and a substituted or unsubstituted heteroaryl group having 2 to 30 carbon atoms; n represents an integer of 1 to 3; and in the case where n is 2 or more, a plurality of R¹ to R⁴ may be the same or different from each other.

Another embodiment of the present invention is an organic compound represented by General Formula (G5). Note that General Formula (G5) is different from General Formula (G3) in that a phenylene group that connects the binaphthyl structure and N^{A} is fixed to a p-phenylene group and n is limited to one or more. Thus, description of the structure, effect, and the like of the organic compound represented by General Formula (G3) can be applied to General Formula (G5).

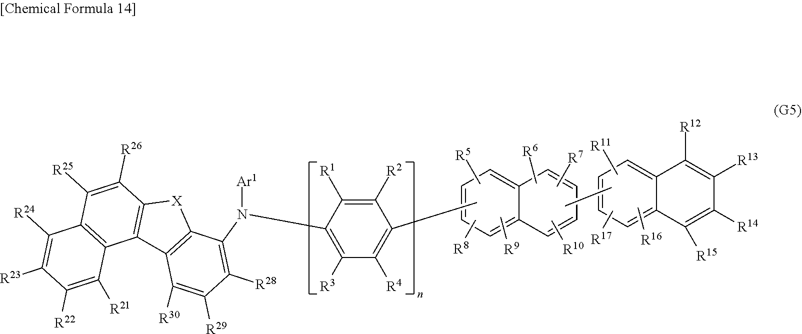

In General Formula (G5), Ar¹ represents a substituted or unsubstituted aryl group having 6 to 30 carbon atoms or a substituted or unsubstituted heteroaryl group having 2 to 30 carbon atoms; R¹ to R¹⁷ each independently represent any one of hydrogen (including deuterium), a substituted or unsubstituted alkyl group having 1 to 6 carbon atoms, and a substituted or unsubstituted cycloalkyl group having 3 to 10 carbon atoms; X represents oxygen or sulfur; R²¹ to R²⁶ and R²⁸ to R³⁰ each independently represent any one of hydrogen (including deuterium), a halogen, a cyano group, a substituted or unsubstituted alkyl group having 1 to 6 carbon atoms, a substituted or unsubstituted alkenyl group having 2 to 6 carbon atoms, a substituted or unsubstituted alkynyl group having 2 to 6 carbon atoms, a substituted or unsubstituted cycloalkyl group having 3 to 10 carbon atoms, a substituted or unsubstituted alkoxy group having 1 to 6 carbon atoms, a trialkylsilyl group having 3 to 10 carbon atoms, a substituted or unsubstituted aryl group having 6 to 30 carbon atoms, and a substituted or unsubstituted heteroaryl group having 2 to 30 carbon atoms; n represents an integer of 1 to 3; and in the case where n is 2 or more, a plurality of R¹ to R⁴ may be the same or different from each other.

As described above, in each of General Formulae (G4) and (G5), a phenylene group that connects the binaphthyl structure and N^{A} in General Formulae (G2) and (G3) is fixed to a p-phenylene group, and n is limited to one or more. With this structure, steric hindrance around N^{A} is reduced, whereby a hole-transport property of triarylamine can be further increased. In particular, a compound having a high hole-injection property and a high hole mobility can be provided. With this structure, the T₁ level of the organic compound represented by General Formula (G4) or General Formula (G5) can be increased. In particular, the organic compound represented by General Formula (G5) is preferable because it has higher a T₁ level than the organic compound represented by General Formula (G4). Thus, in the case where the organic compound represented by General Formula (G4) or General Formula (G5) is used for a phosphorescent light-emitting device or the like, the emission efficiency of the device can be increased. In particular, in the case where the organic compound represented by General Formula (G5) is used for a phosphorescent light-emitting device, a light-emitting device having high emission efficiency can be potentially obtained.

Another embodiment of the present invention is an organic compound represented by General Formula (G6). Note that General Formula (G6) is different from General Formula (G4) in that the substitution sites of the naphthalene ring, which is directly bonded to the p-phenylene group, of the binaphthyl structure in General Formula (G4) are limited to 2- and 6-positions (amphi position). Thus, description of the structure, effect, and the like of the organic compound represented by General Formula (G4) can be applied to General Formula (G6).

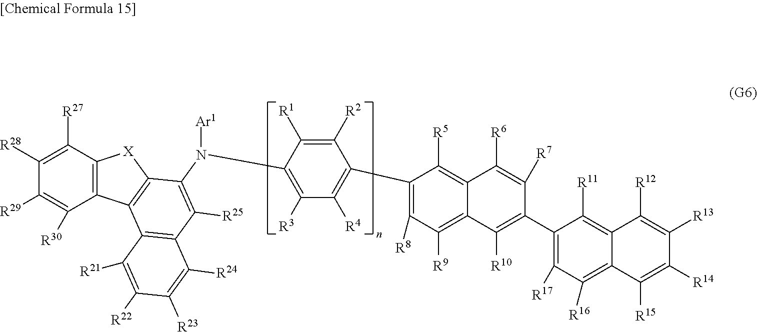

In General Formula (G6), Ar¹ represents a substituted or unsubstituted aryl group having 6 to 30 carbon atoms or a substituted or unsubstituted heteroaryl group having 2 to 30 carbon atoms; R¹ to R¹⁷ each independently represent any one of hydrogen (including deuterium), a substituted or unsubstituted alkyl group having 1 to 6 carbon atoms, and a substituted or unsubstituted cycloalkyl group having 3 to 10 carbon atoms; X represents oxygen or sulfur; R²¹ to R²⁵ and R²⁷ to R³⁰ each independently represent any one of hydrogen (including deuterium), a halogen, a cyano group, a substituted or unsubstituted alkyl group having 1 to 6 carbon atoms, a substituted or unsubstituted alkenyl group having 2 to 6 carbon atoms, a substituted or unsubstituted alkynyl group having 2 to 6 carbon atoms, a substituted or unsubstituted cycloalkyl group having 3 to 10 carbon atoms, a substituted or unsubstituted alkoxy group having 1 to 6 carbon atoms, a trialkylsilyl group having 3 to 10 carbon atoms, a substituted or unsubstituted aryl group having 6 to 30 carbon atoms, and a substituted or unsubstituted heteroaryl group having 2 to 30 carbon atoms; n represents an integer of 0 to 3; and in the case where n is 2 or more, a plurality of R¹ to R⁴ may be the same or different from each other.

Another embodiment of the present invention is an organic compound represented by General Formula (G7). Note that General Formula (G7) is different from General Formula (G5) in that the substitution sites of the naphthalene ring, which is directly bonded to the p-phenylene group, of the binaphthyl structure in General Formula (G5) are limited to 2- and 6-positions (amphi position). Thus, description of the structure, effect, and the like of the organic compound represented by General Formula (G5) can be applied to General Formula (G7).

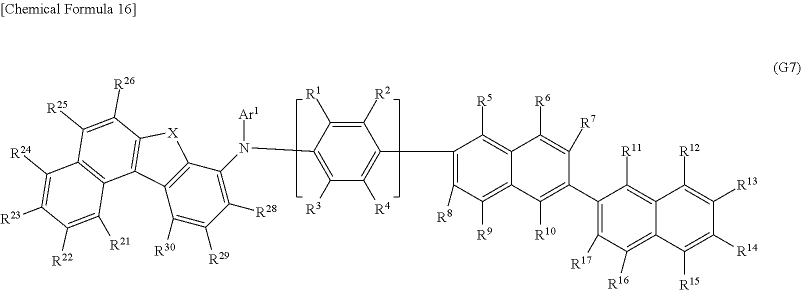

In General Formula (G7), Ar¹ represents a substituted or unsubstituted aryl group having 6 to 30 carbon atoms or a substituted or unsubstituted heteroaryl group having 2 to 30 carbon atoms; R¹ to R¹⁷ each independently represent any one of hydrogen (including deuterium), a substituted or unsubstituted alkyl group having 1 to 6 carbon atoms, and a substituted or unsubstituted cycloalkyl group having 3 to 10 carbon atoms; X represents oxygen or sulfur; R²¹ to R²⁶ and R²⁸ to R³⁰ each independently represent any one of hydrogen (including deuterium), a halogen, a cyano group, a substituted or unsubstituted alkyl group having 1 to 6 carbon atoms, a substituted or unsubstituted alkenyl group having 2 to 6 carbon atoms, a substituted or unsubstituted alkynyl group having 2 to 6 carbon atoms, a substituted or unsubstituted cycloalkyl group having 3 to 10 carbon atoms, a substituted or unsubstituted alkoxy group having 1 to 6 carbon atoms, a trialkylsilyl group having 3 to 10 carbon atoms, a substituted or unsubstituted aryl group having 6 to 30 carbon atoms, and a substituted or unsubstituted heteroaryl group having 2 to 30 carbon atoms; n represents an integer of 0 to 3; and in the case where n is 2 or more, a plurality of R¹ to R⁴ may be the same or different from each other.

As described above, in each of General Formulae (G6) and (G7), the substitution sites of the naphthalene ring, which is directly bonded to the p-phenylene group, of the binaphthyl structure in General Formulae (G4) and (G5) are limited to 2- and 6-positions (amphi position). With this structure, steric hindrance around N^{A} is reduced, whereby a hole-transport property and a hole-injection property of triarylamine can be further increased. In addition, with this structure, a conjugated system easily spreads over the binaphthyl structure, achieving highly stable distribution of the HOMO and the LUMO of the binaphthyl structure. Meanwhile, the binaphthyl structure is bonded to N^{A} not directly but through the p-phenylene group; accordingly, an organic compound having a high T₁ level can be provided. Thus, with such an organic compound, a phosphorescent light-emitting device having high efficiency can be provided. In addition, each of the naphthalene rings in General Formulae (G6) and (G7) is substituted at a β-position. With such a structure, the symmetry of expansion of π electrons is high, and repelling between hydrogen bonded to adjacent carbon and a group bonded to the naphthalene ring is weaker than that in the case of substituting each of the naphthalene rings at an α-position, whereby a structure where a planarity is increased and a π-conjugated system spreads can be obtained. Thus, when triarylamine with such a structure is used for an organic semiconductor device, the driving lifetime of the organic semiconductor device can be extended; that is, the reliability can be increased. More specifically, when triarylamine with such a structure is used for a light-emitting device or the like, the voltage change during driving can be made small, and the luminance degradation due to driving can be suppressed.

In the organic compounds represented by General Formulae (G1) to (G4), (G6), and (G7), n is further preferably an integer of 1 to 3. With this structure, steric hindrance around N^{A} is reduced, whereby a hole-transport property of triarylamine can be further increased. In addition, with this structure, the T₁ level of triarylamine can be increased. Thus, when triarylamine is used for a phosphorescent device or the like, the emission efficiency of the device can be increased.

Next, specific examples of substituents that can be used for the organic compounds represented by the above general formulae will be described. Note that groups that can be used in the above general formulae are not limited to the following specific examples. In addition, in the specific examples described below, some or all of hydrogen atoms may be deuterium.

### <<Halogen>>

Specific examples of a halogen include fluorine, chlorine, bromine, and iodine. In particular, fluorine, which is chemically stable, is preferable.

### <<Alkyl Group Having 1 to 6 Carbon Atoms>>

An alkyl group having 1 to 6 carbon atoms is a monovalent group obtained by removing one hydrogen atom from an alkane having 1 to 6 carbon atoms. Specific examples of an alkyl group having 1 to 6 carbon atoms include a methyl group, an ethyl group, a propyl group, an isopropyl group, a butyl group, a sec-butyl group, an isobutyl group, a tert-butyl group, a pentyl group, an isopentyl group, a sec-pentyl group, a tert-pentyl group, a neo-pentyl group, a hexyl group, an isohexyl group, a sec-hexyl group, a tert-hexyl group, a neo-hexyl group, a 3-methylpentyl group, a 2-methylpentyl group, a 2-ethylbutyl group, a 1,2-dimethylbutyl group, a 2,3-dimethylbutyl group, and the like. In the case where the alkyl group having 1 to 6 carbon atoms includes a substituent, specific examples of the substituent include a halogen, a cyano group, and the like.

### <<Alkenyl Group Having 2 to 6 Carbon Atoms>>

An alkenyl group having 2 to 6 carbon atoms is a monovalent group obtained by removing one hydrogen atom from an alkene having 2 to 6 carbon atoms. Specific examples of an alkenyl group having 2 to 6 carbon atoms include a vinyl group, an aryl group, and a 2,2-dimethylvinyl group. In the case where the alkenyl group having 2 to 6 carbon atoms includes a substituent, specific examples of the substituent include a halogen, a cyano group, and the like.

### <<Alkynyl Group Having 2 to 6 Carbon Atoms>>

An alkynyl group having 2 to 6 carbon atoms is a monovalent group obtained by removing one hydrogen atom from an alkyne having 2 to 6 carbon atoms. Specific examples of an alkynyl group having 2 to 6 carbon atoms include an ethinyl group and a prop-2-yn-1-yl group (also referred to as a propargyl group). In the case where the alkynyl group having 2 to 6 carbon atoms includes a substituent, specific examples of the substituent include a halogen, a cyano group, and the like.

### <<Alkoxy Group Having 1 to 6 Carbon Atoms>>

An alkoxy group having 1 to 6 carbon atoms has a structure in which an alkyl group having 1 to 6 carbon atoms is bonded to an oxygen atom. Specific examples of an alkoxy group having 1 to 6 carbon atoms include a methoxy group, an ethoxy group, an n-propoxy group, an isopropoxy group, an n-butoxy group, a sec-butoxy group, an isobutoxy group, a tert-butoxy group, an n-pentyloxy group, an isopentyloxy group, a sec-pentyloxy group, a tert-pentyloxy group, a neo-pentyloxy group, an n-hexyloxy group, an isohexyloxy group, a sec-hexyloxy group, a tert-hexyloxy group, a neo-hexyloxy group, a cyclohexyloxy group, and the like. In the case where the alkoxy group having 1 to 6 carbon atoms includes a substituent, specific examples of the substituent include a halogen, a cyano group, and the like.

### <<Trialkylsilyl Group Having 3 to 10 Carbon Atoms>>

A trialkylsilyl group having 3 to 10 carbon atoms has a structure in which three alkyl groups having 3 to 10 carbon atoms in total are bonded to a silicon atom. Specific examples of a trialkylsilyl group having 3 to 10 carbon atoms include a trimethylsilyl group, a triethylsilyl group, a tert-butyldimethylsilyl group, and the like.

### <<Cycloalkyl Group Having 3 to 10 Carbon Atoms>>

A cycloalkyl group having 3 to 10 carbon atoms is a monovalent group obtained by removing one hydrogen atom from a monocyclic or polycyclic cycloalkane having 3 to 10 carbon atoms. Specific examples of a cycloalkyl group having 3 to 10 carbon atoms include a cyclopropyl group, a cyclobutyl group, a cyclopentyl group, a cyclohexyl group, a cycloheptyl group, a cyclooctyl group, a cyclononyl group, a cyclodecyl group, a norbornyl group, a bicyclo[2,2,2]octyl group, a decahydronaphthalenyl group, an adamantyl group, and the like. In the case where the cycloalkyl group having 3 to 10 carbon atoms includes a substituent, specific examples of the substituent include a halogen, a cyano group, an alkyl group having 1 to 6 carbon atoms, an alkenyl group having 2 to 6 carbon atoms, an alkynyl group having 2 to 6 carbon atoms, an alkoxy group having 1 to 6 carbon atoms, a trialkylsilyl group having 3 to 10 carbon atoms, a phenyl group, and the like.

### <<Aryl Group Having 6 to 30 Carbon Atoms>>

An aryl group having 6 to 30 carbon atoms is a monovalent group obtained by removing one hydrogen atom from one of carbon atoms forming a ring of a monocyclic or polycyclic aromatic compound having 6 to 30 carbon atoms. Specific examples of an aryl group having 6 to 30 carbon atoms include a phenyl group, an o-tolyl group, a m-tolyl group, a p-tolyl group, a mesityl group, a biphenyl-2-yl group (o-biphenyl group), a biphenyl-3-yl group (m-biphenyl group), a biphenyl-4-yl group (p-biphenyl group), a 1-naphthyl group, a 2-naphthyl group, a phenylnaphthyl group, a naphthylphenyl group, a terphenyl group, a fluorenyl group, a 9,9-dimethylfluorenyl group, a quaterphenyl group, a spirobifluorenyl group, a phenanthrenyl group, an anthracenyl group, a binaphthylphenyl group, a fluoranthenyl group, and the like. In the case where the aryl group having 6 to 30 carbon atoms includes a substituent, specific examples of the substituent include a halogen, a cyano group, an alkyl group having 1 to 6 carbon atoms, an alkenyl group having 2 to 6 carbon atoms, an alkynyl group having 2 to 6 carbon atoms, an alkoxy group having 1 to 6 carbon atoms, a trialkylsilyl group having 3 to 10 carbon atoms, a cycloalkyl group having 3 to 10 carbon atoms, a phenyl group, and the like. In particular, in the organic compounds represented by General Formulae (G1) to (G7), Ar¹ is preferably a group having a structure in which a phenyl group bonded to N^{A} is further bonded to an aryl group (hereinafter referred to as an arylphenyl group), such as a biphenyl group, a naphthylphenyl group, or a terphenyl group, in which case a π-conjugated system can spread, electrons can be delocalized, and thus, the organic compounds can be further stabilized. Such an organic compound is preferably used for the organic semiconductor device to further increase the reliability of the organic semiconductor. Furthermore, in the arylphenyl group of the organic compound, the aryl group is preferably bonded at the ortho-orpara-position of the phenyl group bonded to N^{A}, in which case the heat resistance of the organic compound is higher than that in the case where the aryl group is bonded at the meta-position of the phenyl group. For example, when Ar¹ is a biphenyl group in the organic compounds represented by General Formulae (G1) to (G7), the biphenyl group is preferably a p-biphenyl group or an o-biphenyl group, in which case the heat resistance of the organic compound can be higher than that in the case where the biphenyl group is an m-biphenyl group; in particular, the biphenyl group is preferably a p-biphenyl group, in which case the heat resistance of the organic compound can be higher than that in the case where the biphenyl group is an o-biphenyl group or an m-biphenyl group. Note that in the case where Ar¹ is a biphenyl group in the organic compounds represented by General Formulae (G1) to (G7) and the biphenyl group is a p-biphenyl group or an o-biphenyl group, luminance degradation and voltage change due to driving are smaller than those in the case where the biphenyl group is an m-biphenyl group; accordingly, a light-emitting device having high reliability can be potentially provided.

### <<Heteroaryl Group Having 2 to 30 Carbon Atoms>>

A heteroaryl group having 2 to 30 carbon atoms is a monovalent group obtained by removing one hydrogen atom from one of carbon atoms forming a ring of a monocyclic or polycyclic heterocyclic aromatic compound having 2 to 30 carbon atoms. Specific examples of a heteroaryl group having 2 to 30 carbon atoms include a carbazolyl group, a dibenzothiophenyl group, a dibenzofuranyl group, a benzocarbazolyl group, a naphthobenzothiophenyl group, a naphthobenzofuranyl group, a dibenzocarbazolyl group, a dinaphthothiophenyl group, a dinaphthofuranyl group, a triazinyl group, a pyrimidinyl group, a pyrazinyl group, a triazolyl group, a pyridinyl group, a benzofuropyrimidinyl group, a benzothiopyrimidinyl group, a benzofuropyrazinyl group, a benzothiopyrazinyl group, a benzofuropyridinyl group, a benzothiopyridinyl group, a bicarbazolyl group, and the like. In the case where the heteroaryl group having 2 to 30 carbon atoms includes a substituent, specific examples of the substituent include a halogen, a cyano group, an alkyl group having 1 to 6 carbon atoms, an alkenyl group having 2 to 6 carbon atoms, an alkynyl group having 2 to 6 carbon atoms, an alkoxy group having 1 to 6 carbon atoms, a trialkylsilyl group having 3 to 10 carbon atoms, a cycloalkyl group having 3 to 10 carbon atoms, a phenyl group, and the like.

Note that as R²¹ to R³⁰, it is more preferable to select groups represented by any of Structural Formulae (1-1) to (1-28) below, among a hydrogen (including deuterium), a halogen, a cyano group, a substituted or unsubstituted alkyl group having 1 to 6 carbon atoms, a substituted or unsubstituted alkenyl group having 2 to 6 carbon atoms, a substituted or unsubstituted alkynyl group having 2 to 6 carbon atoms, a substituted or unsubstituted cycloalkyl group having 3 to 10 carbon atoms, a substituted or unsubstituted alkoxy group having 1 to 6 carbon atoms, a trialkylsilyl group having 3 to 10 carbon atoms, a substituted or unsubstituted aryl group having 6 to 30 carbon atoms, and a substituted or unsubstituted heteroaryl group having 2 to 30 carbon atoms. This structure can have both an effect of inhibiting a narrow gap between the HOMO and the LUMO due to a conjugated system excessively spreading over the second aryl group and an effect of improving heat resistance. Furthermore, the effect of improving heat resistance and the effect of improving a sublimation property (suppressing an excessive increase in sublimation temperature) can be achieved at the same time. In particular, in the case where any of the groups represented by Structural Formulae (1-1) to (1-28) below is introduced as R²⁶ or R²⁷, the target substance can be obtained with a higher yield than in the case where any of these groups is introduced to other substitution sites, whereby production cost can be potentially reduced.

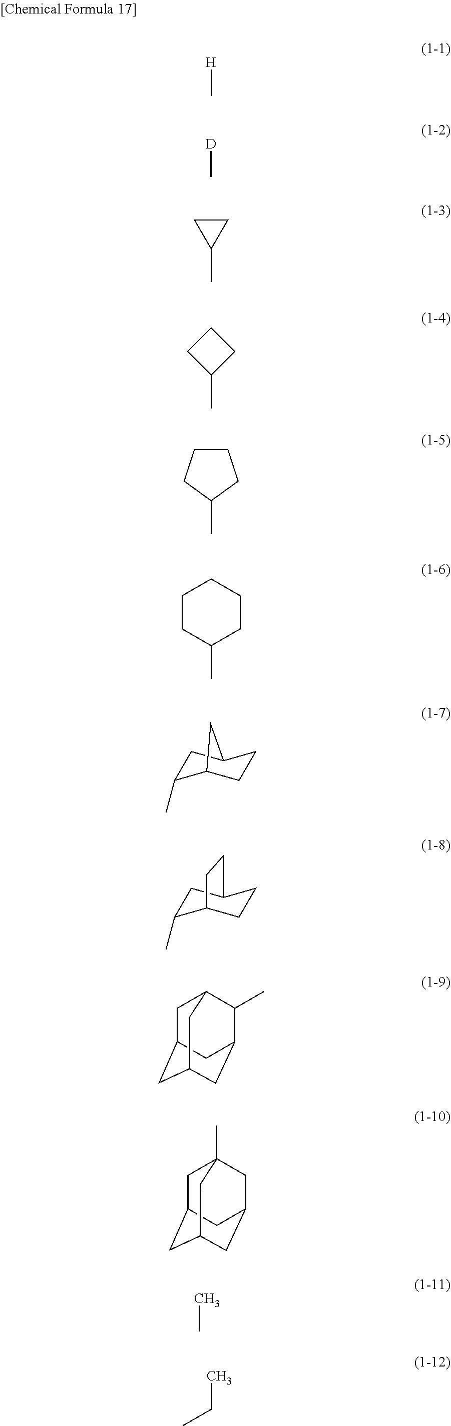

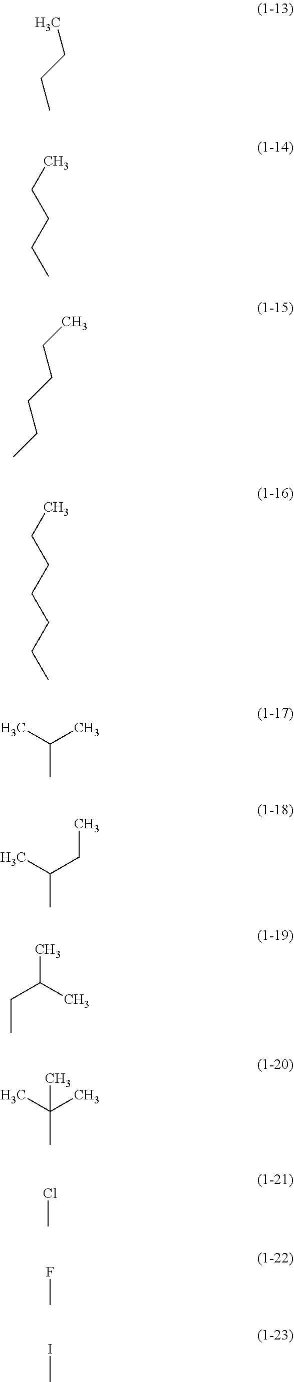

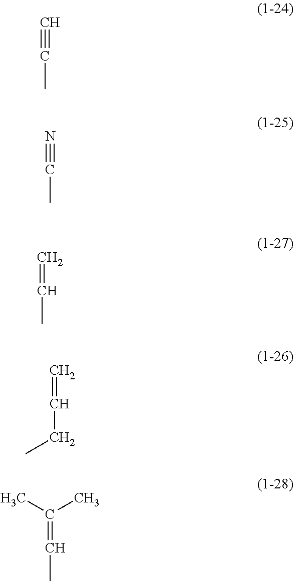

Furthermore, as Ar¹, it is preferable to select a group represented by any of Structural Formulae (2-1) to (2-55) below, among a substituted or unsubstituted aryl group having 6 to 30 carbon atoms and a substituted or unsubstituted heteroaryl group having 2 to 30 carbon atoms. Note that there is no limitation on the substitution sites in the groups represented by these structural formulae. Selecting a group represented by any of Structural Formulae (2-1) to (2-55) below can make the organic compound have a high hole mobility. Moreover, the organic compound can have a high hole-injection property. Furthermore, the organic compound that is less likely to be decomposed in the evaporation process can be obtained. In particular, selecting a group represented by any of Structural Formulae (2-1), (2-4), (2-6) to (2-9), and (2-11) to (2-55) enables the organic compound to have a high T₁ level and to be less likely to absorb light with a wavelength in the visible range. Thus, in the case where such an organic compound is used for a light-emitting device, the light-emitting device can have high emission efficiency.

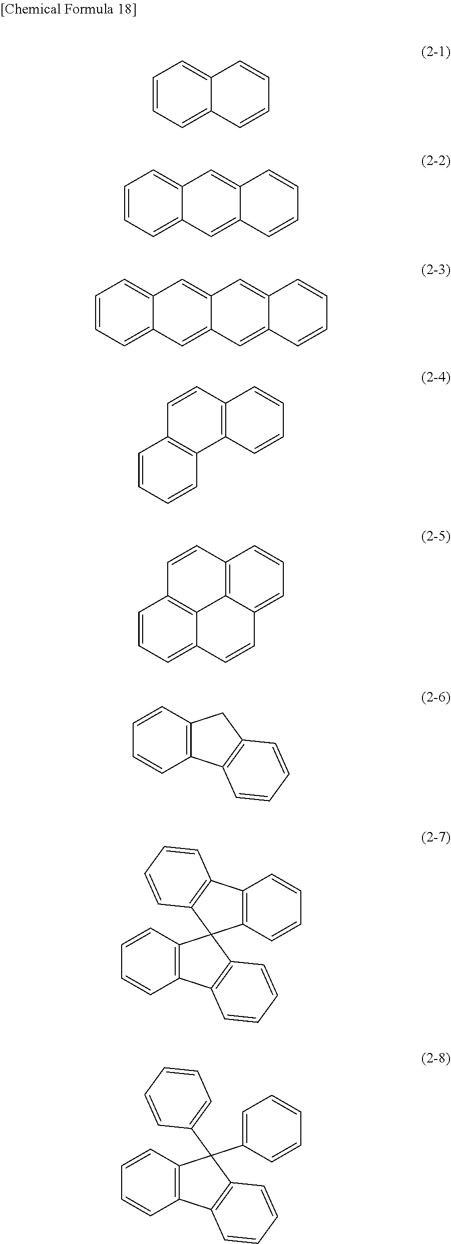

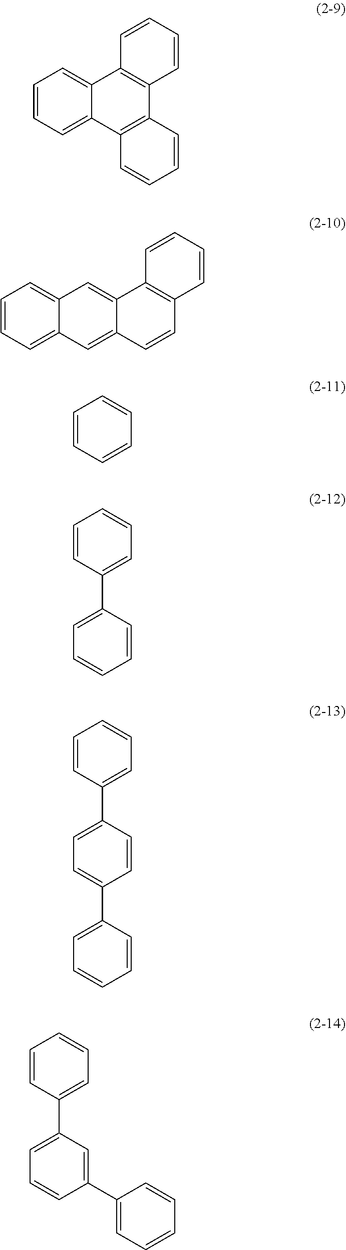

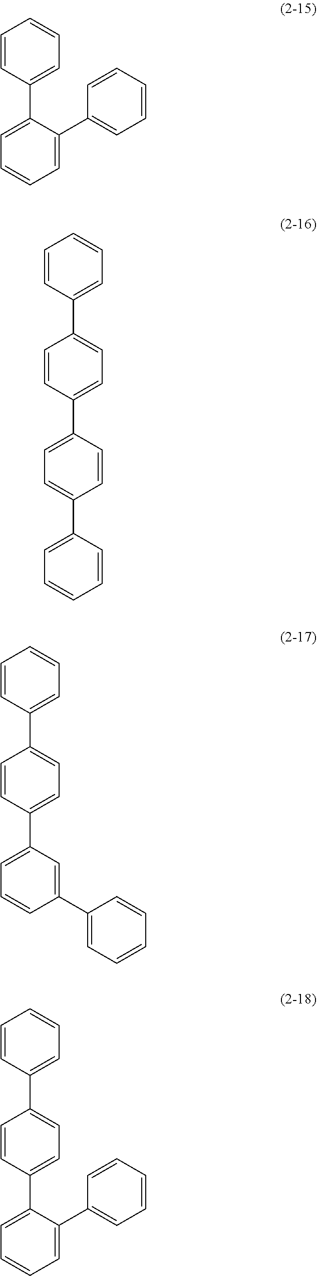

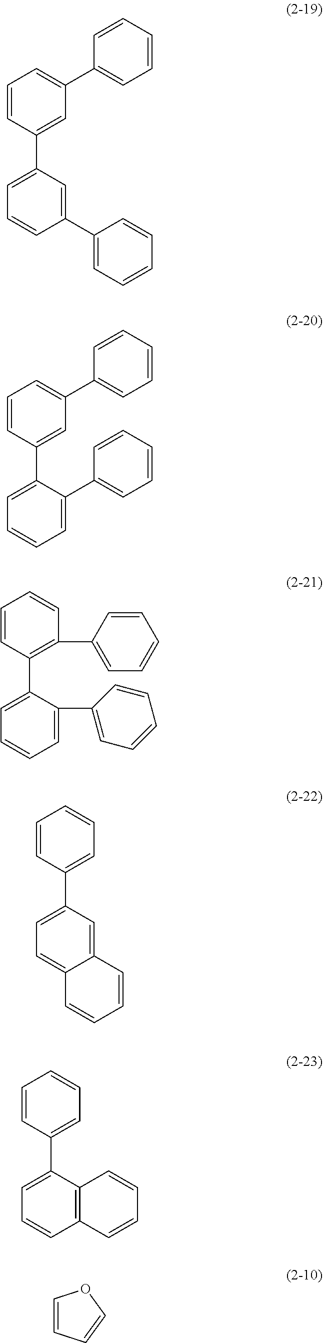

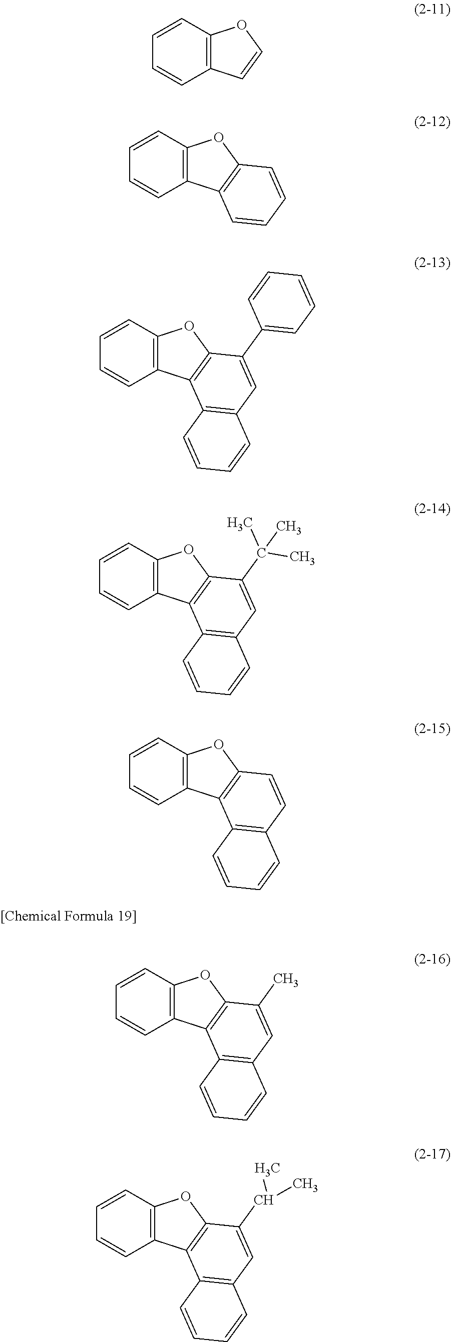

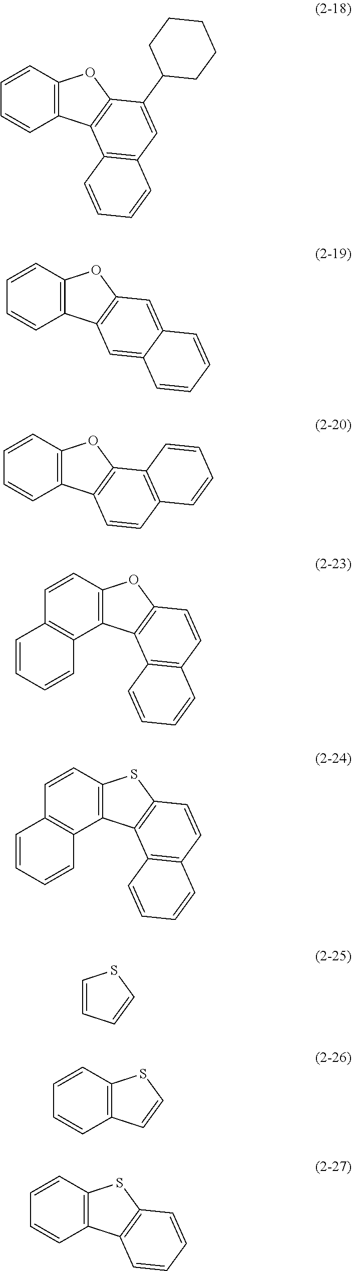

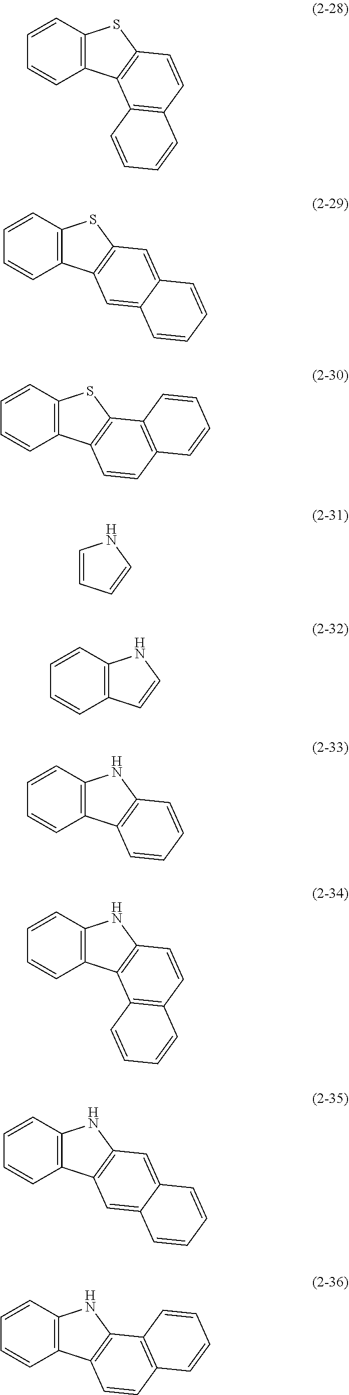

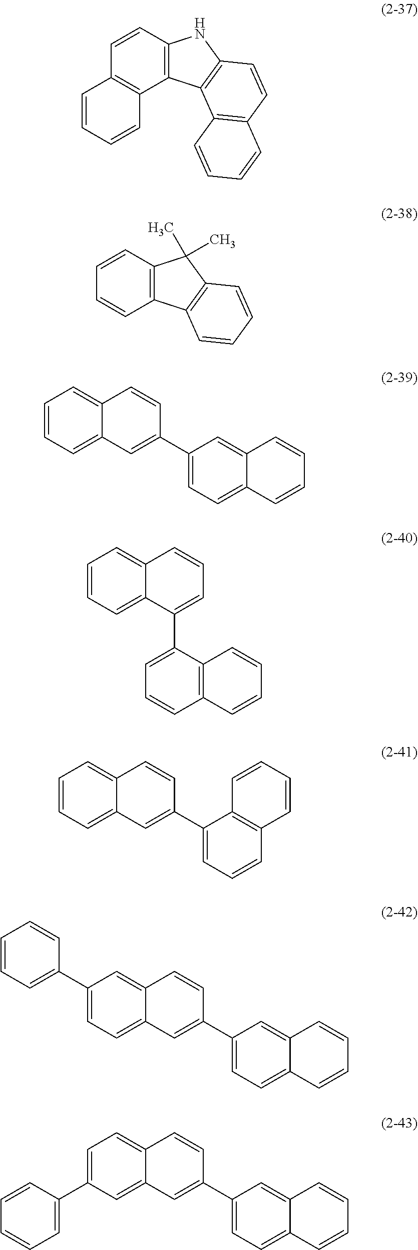

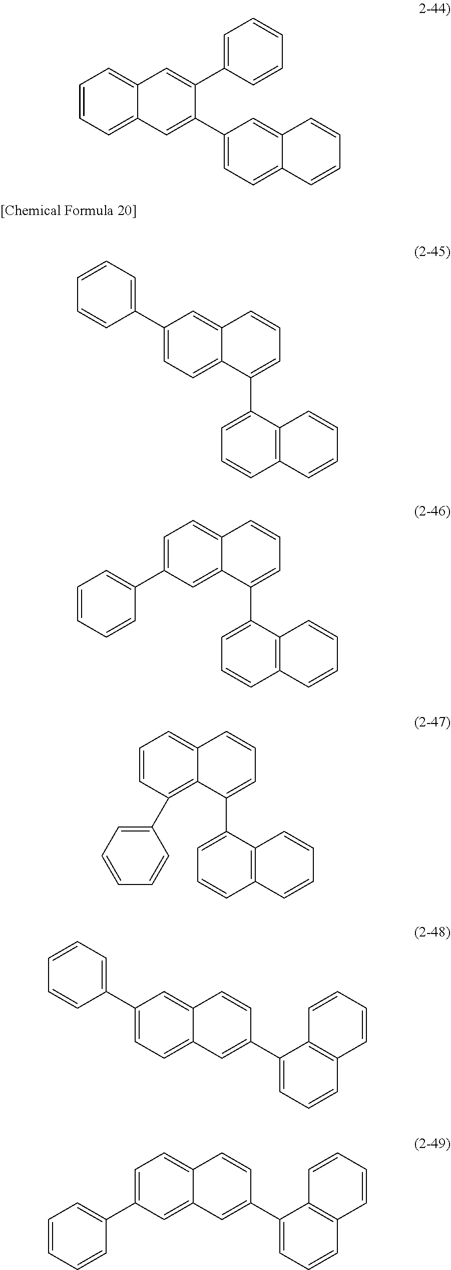

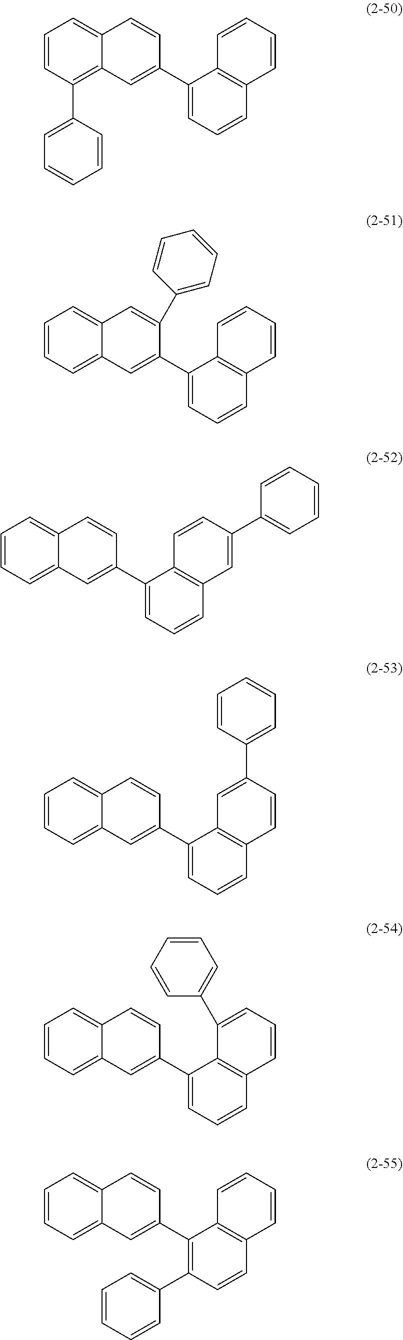

The above substituents are specific examples of the substituent that can be used for the organic compounds represented by the general formulae.

Specific examples of the organic compounds of embodiments of the present invention represented by the above general formulae include organic compounds represented by Structural Formulae (100) to (347) below. Note that the organic compound of one embodiment of the present invention is not limited to the organic compounds represented by the following structural formulae.

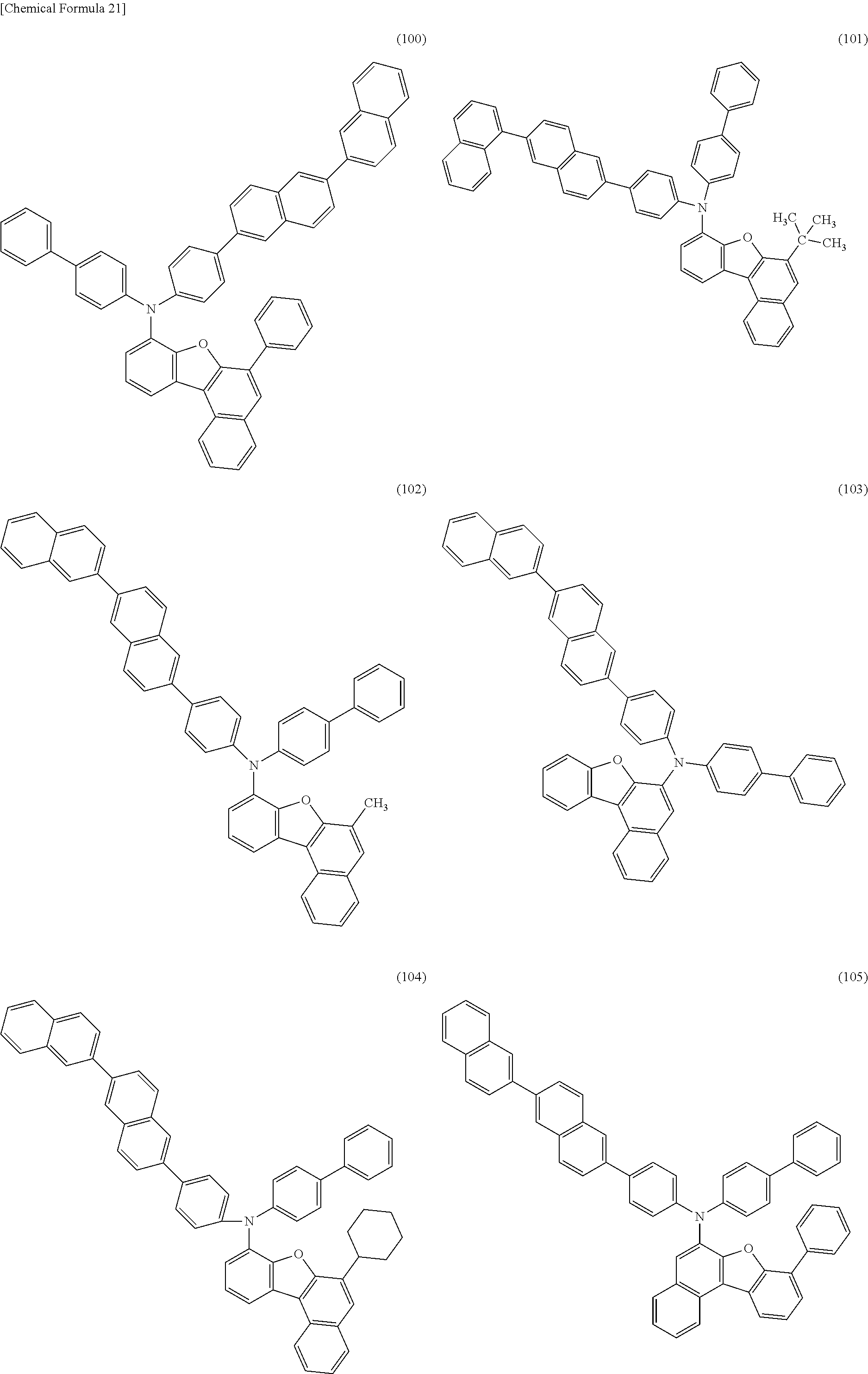

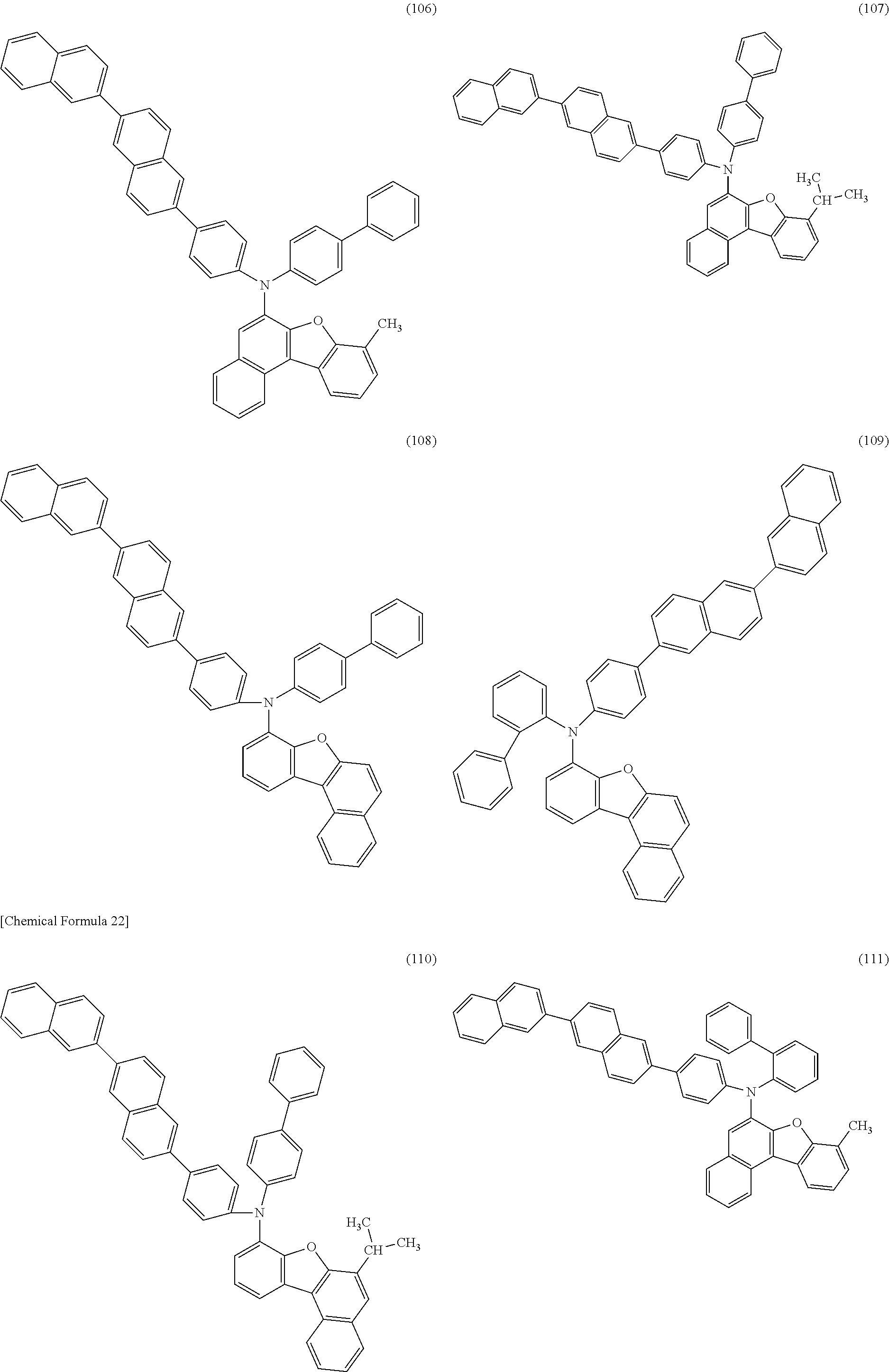

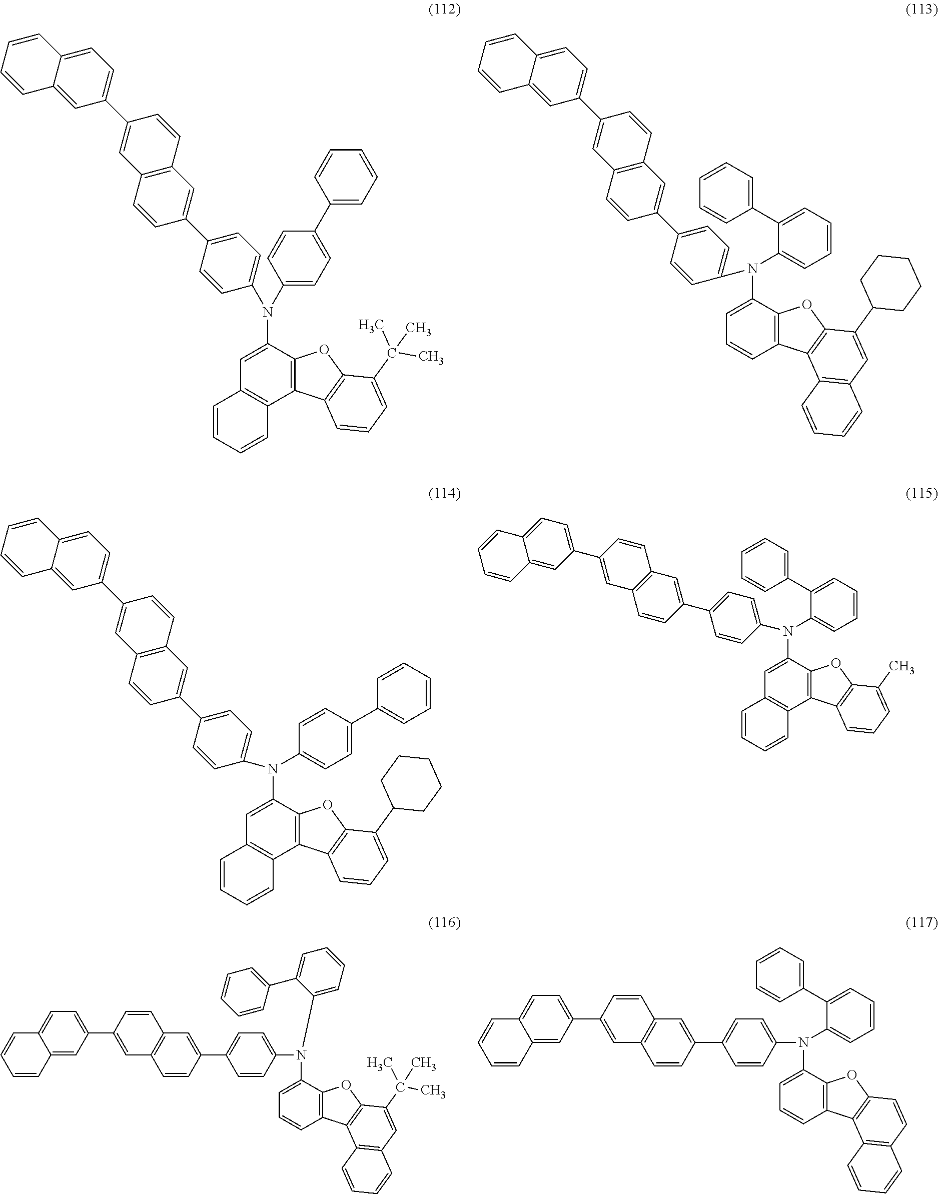

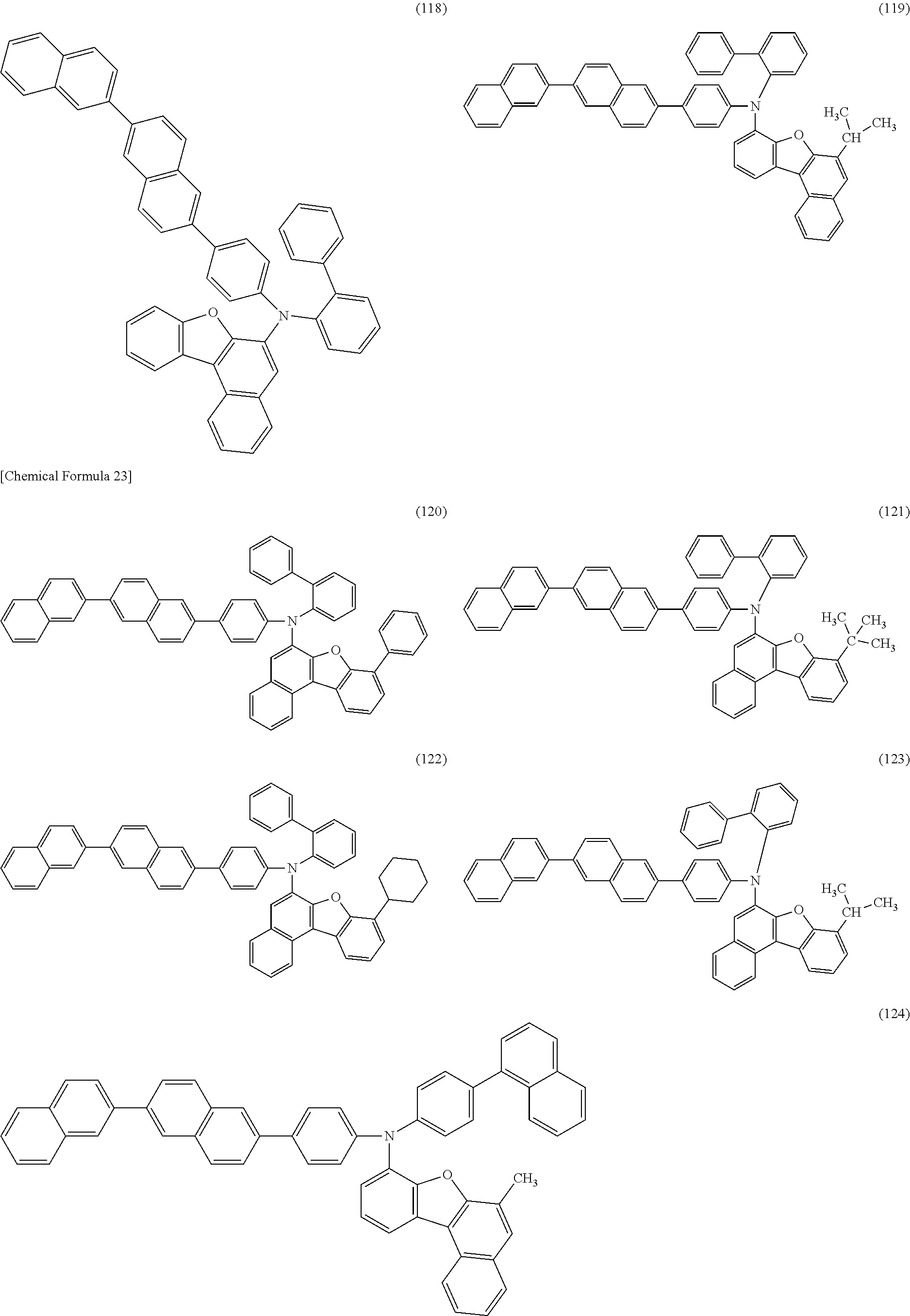

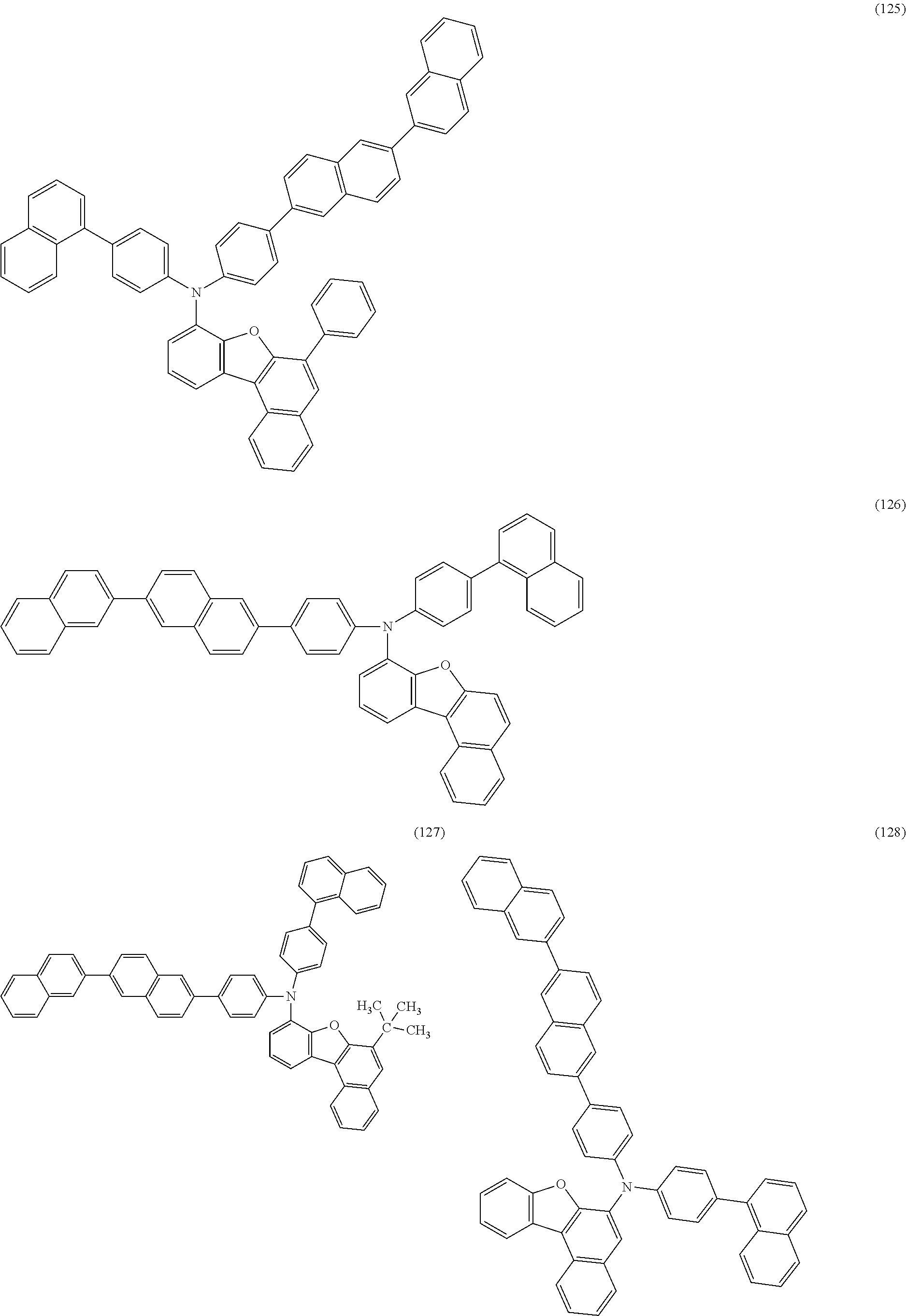

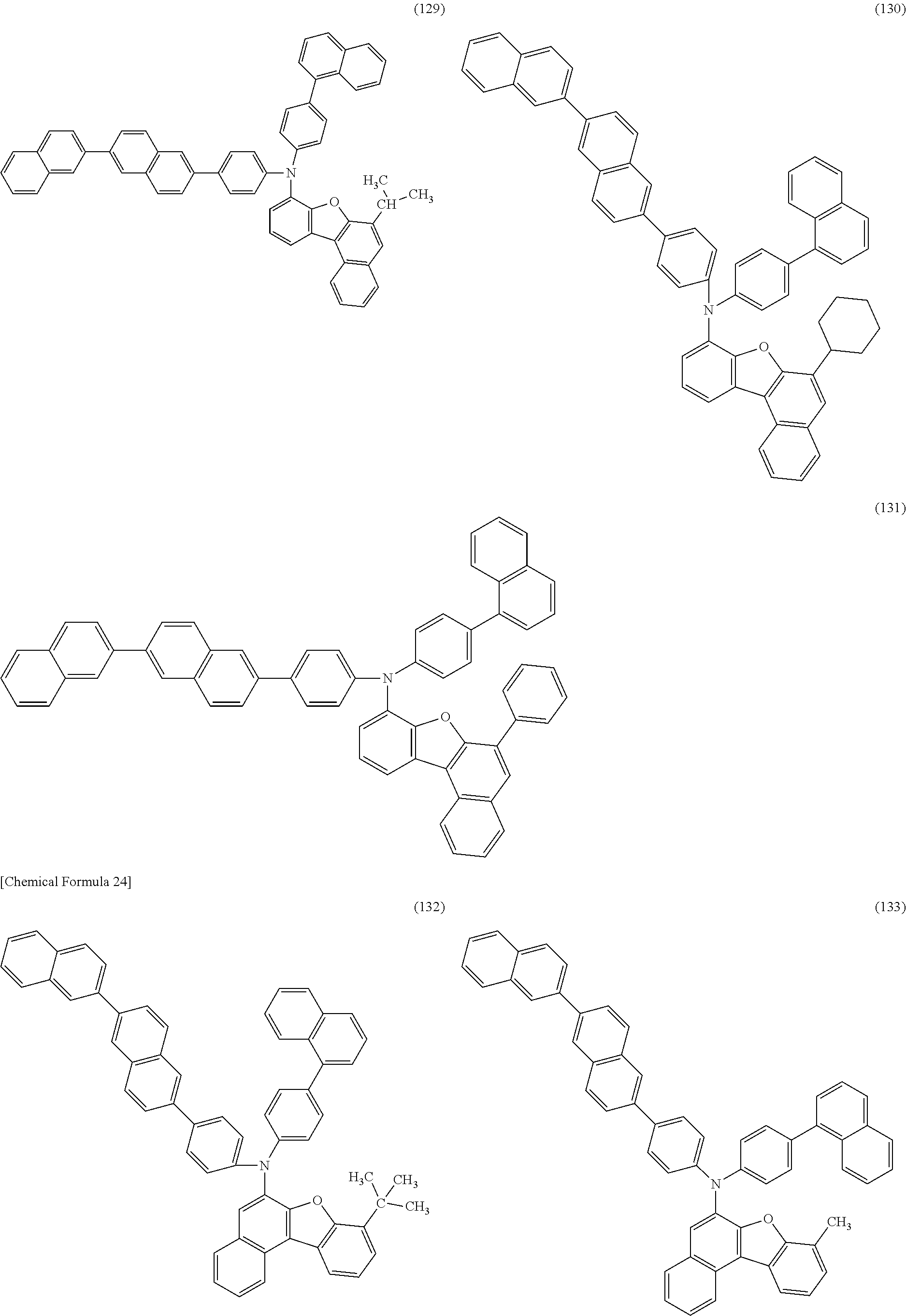

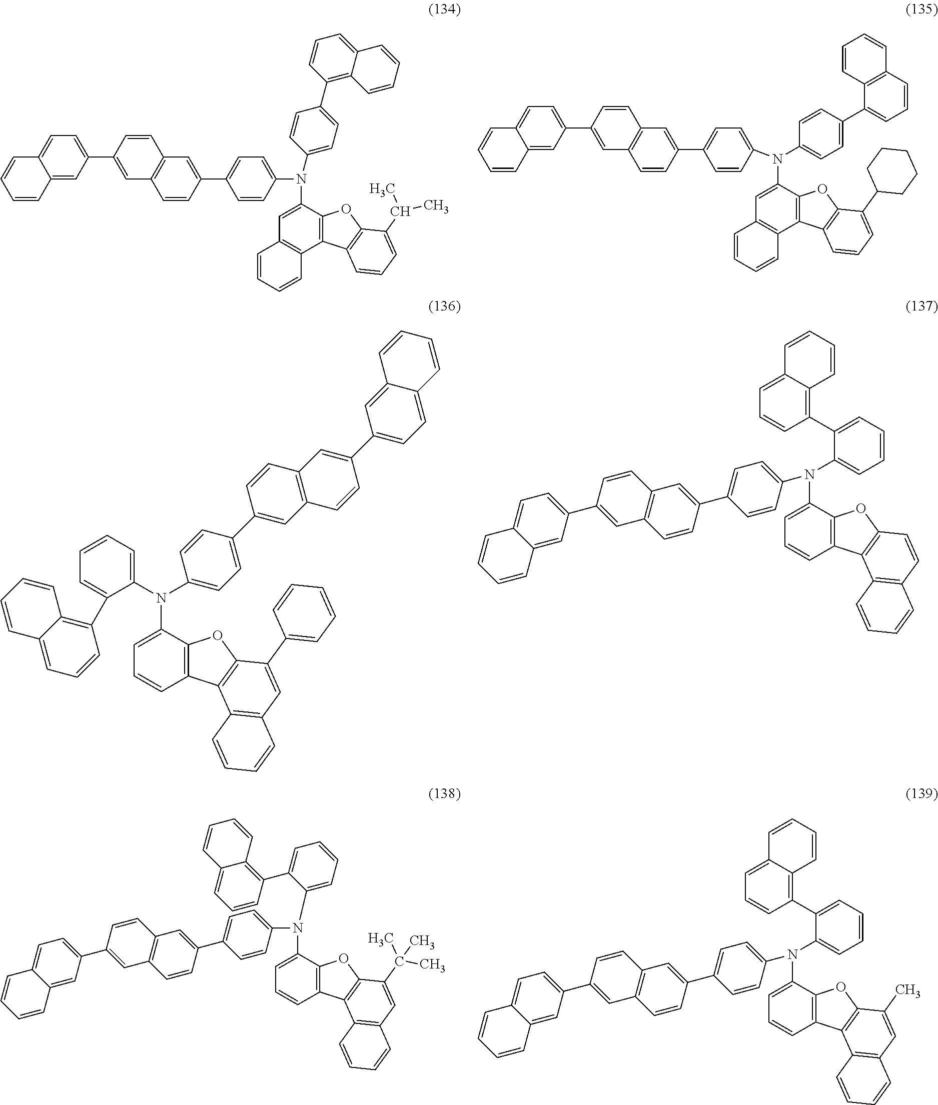

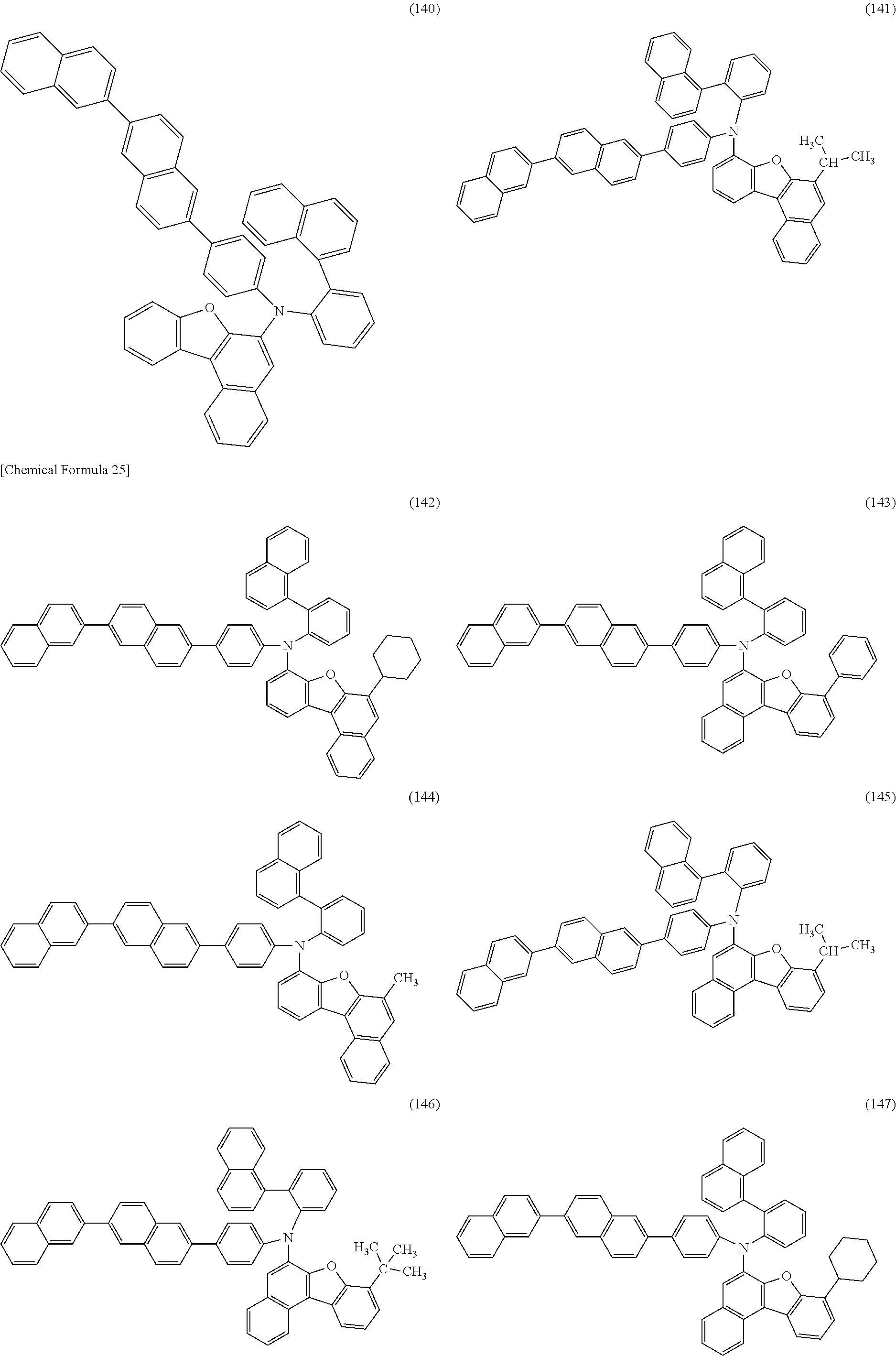

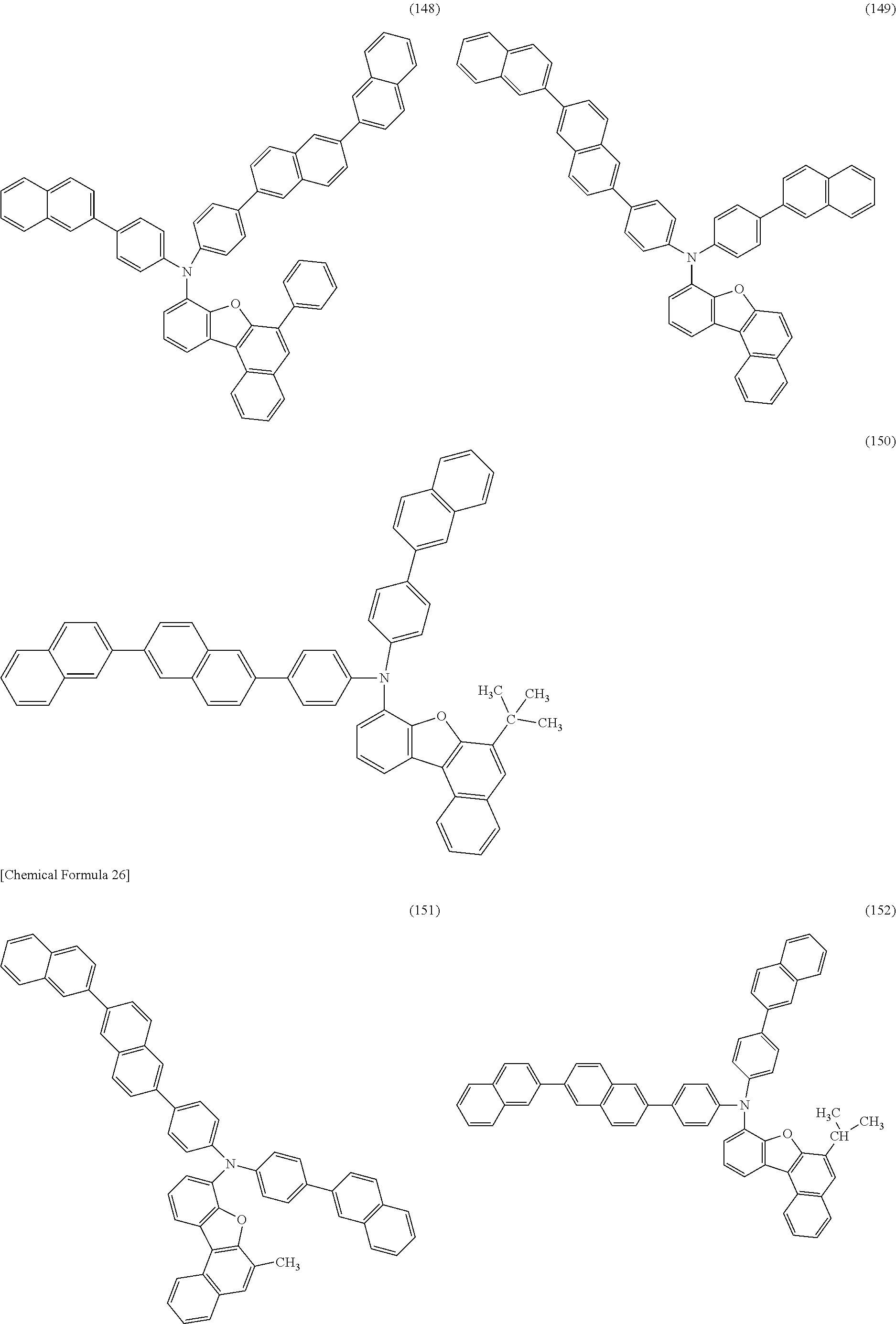

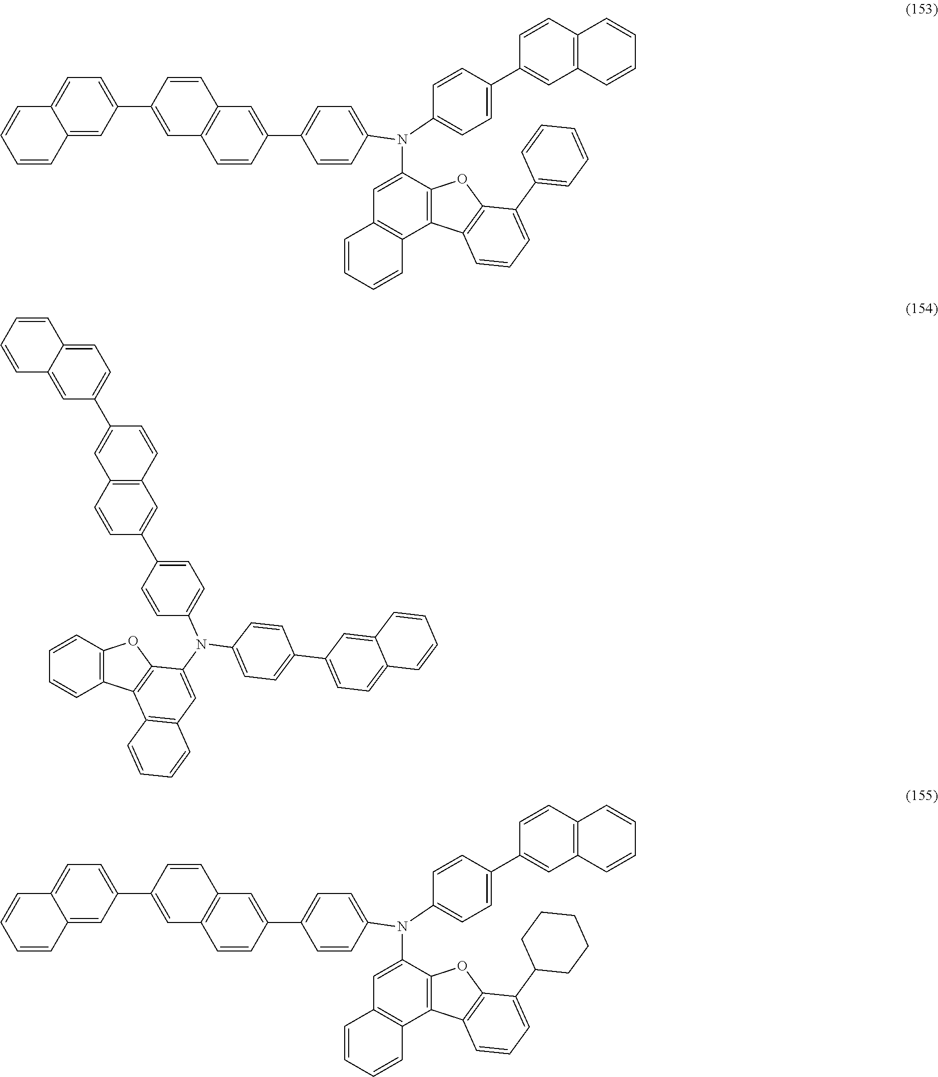

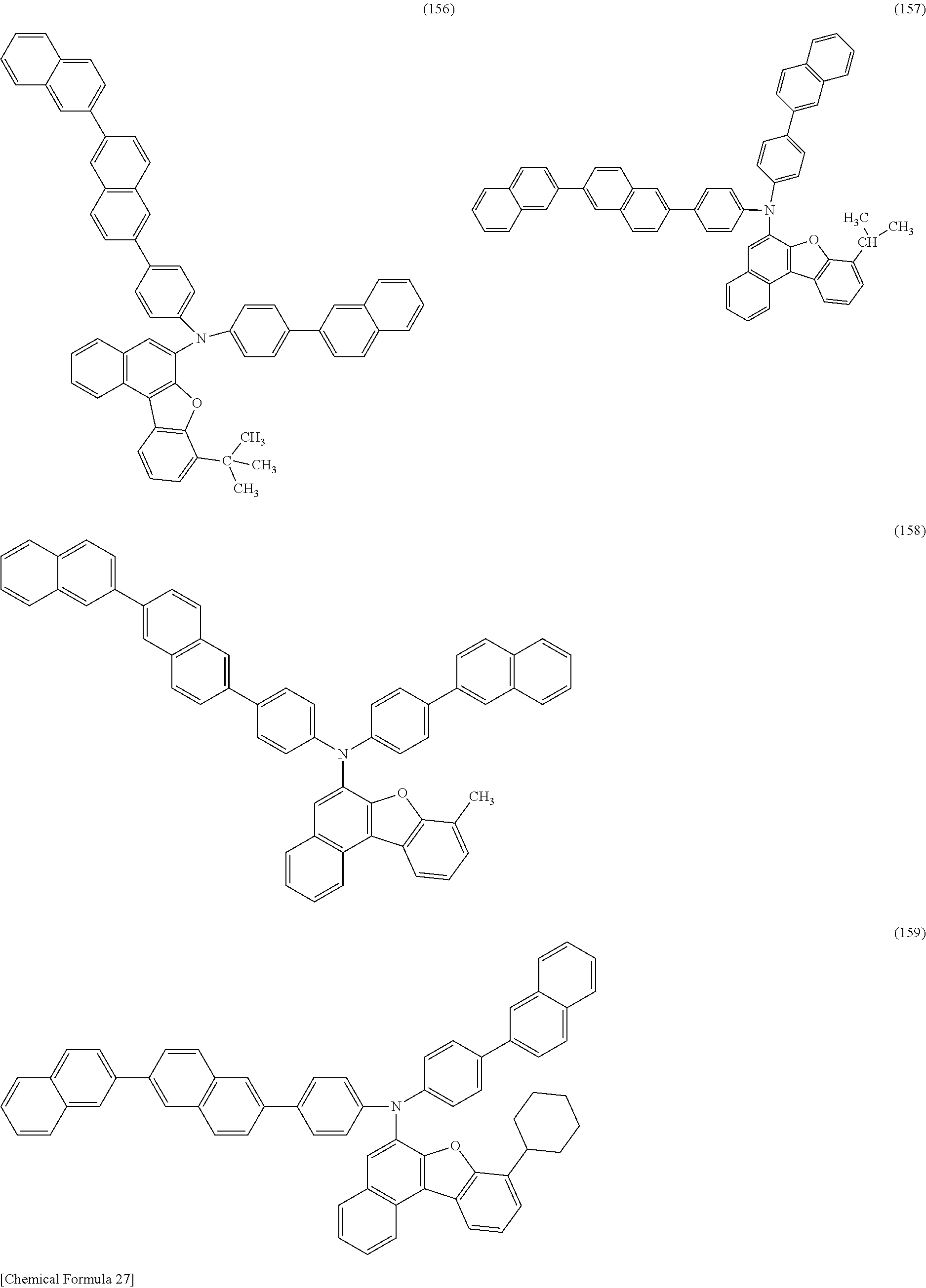

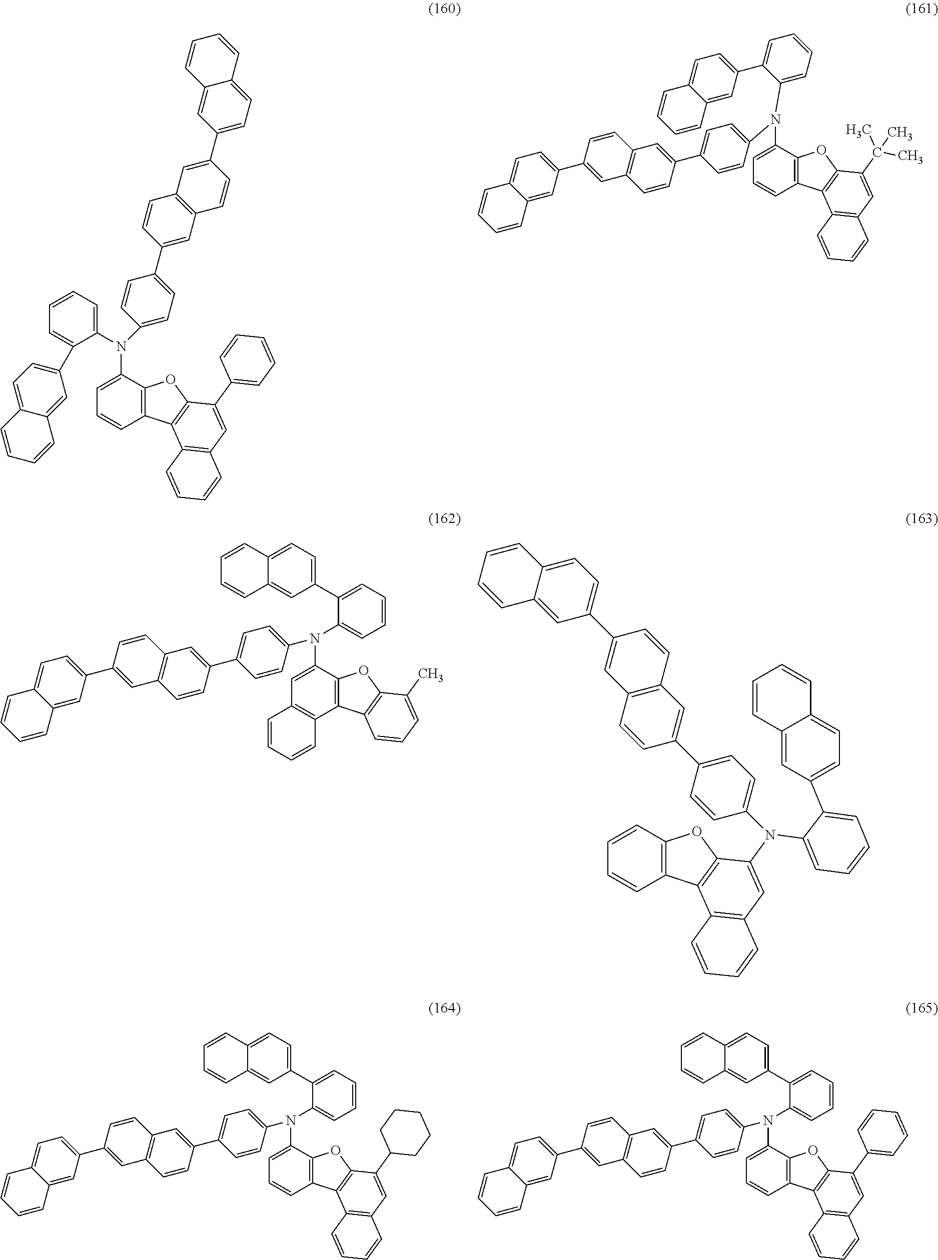

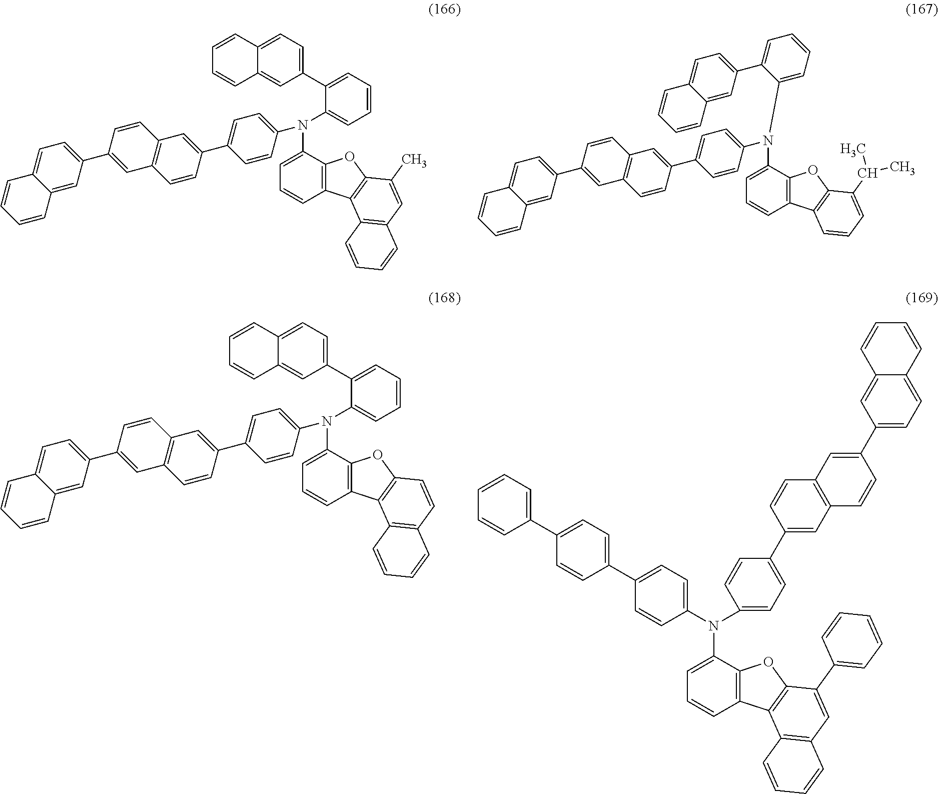

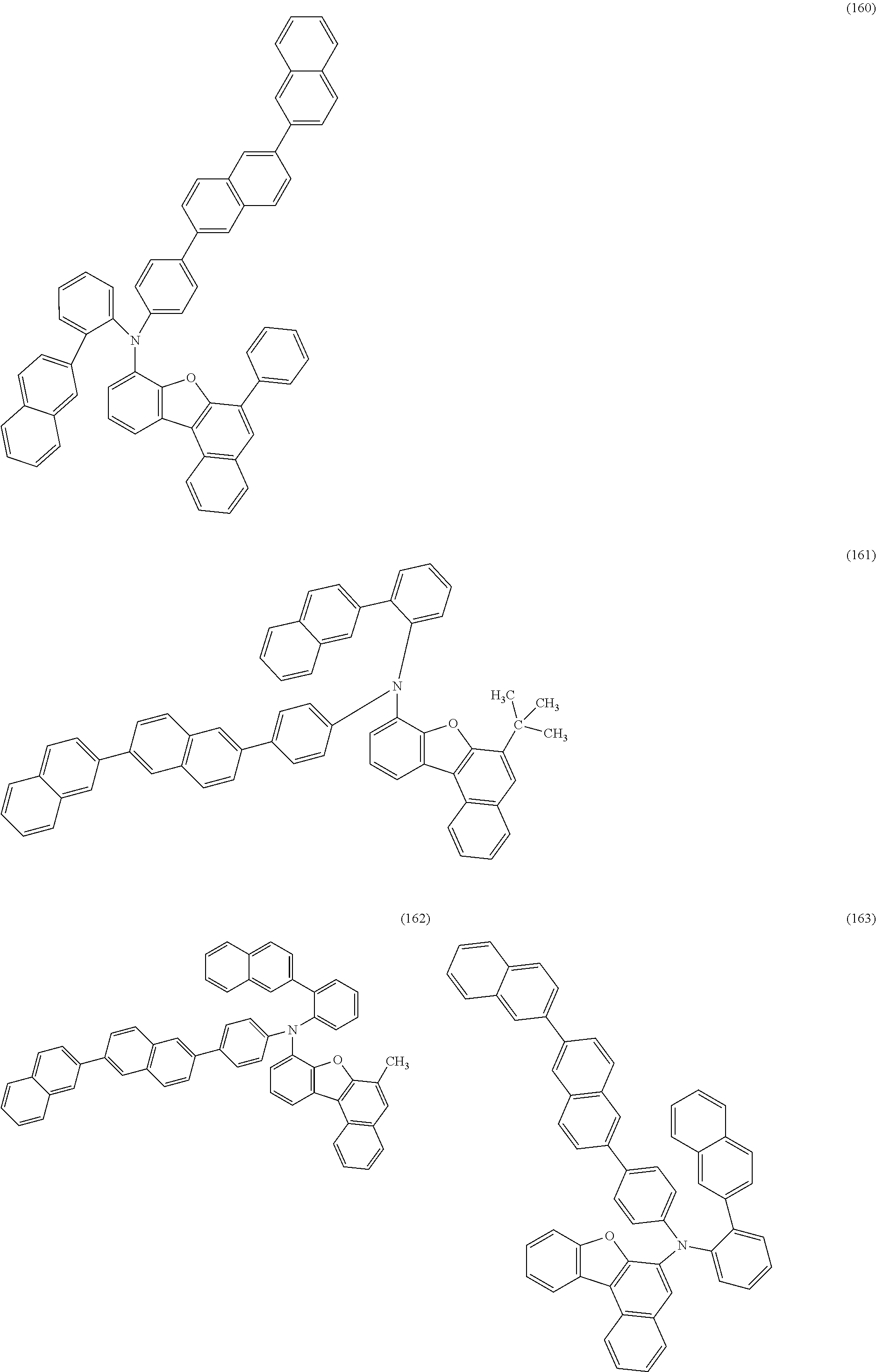

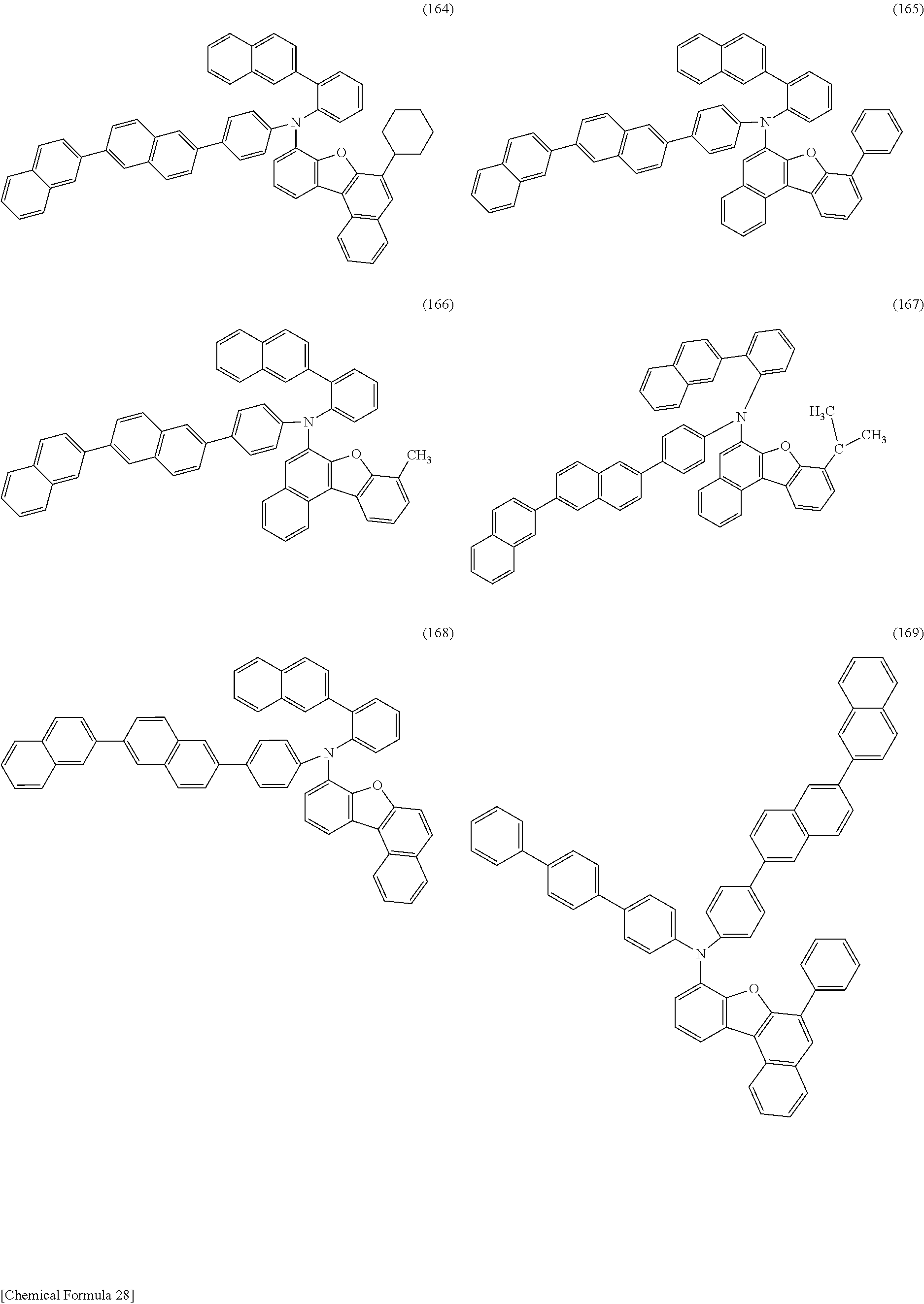

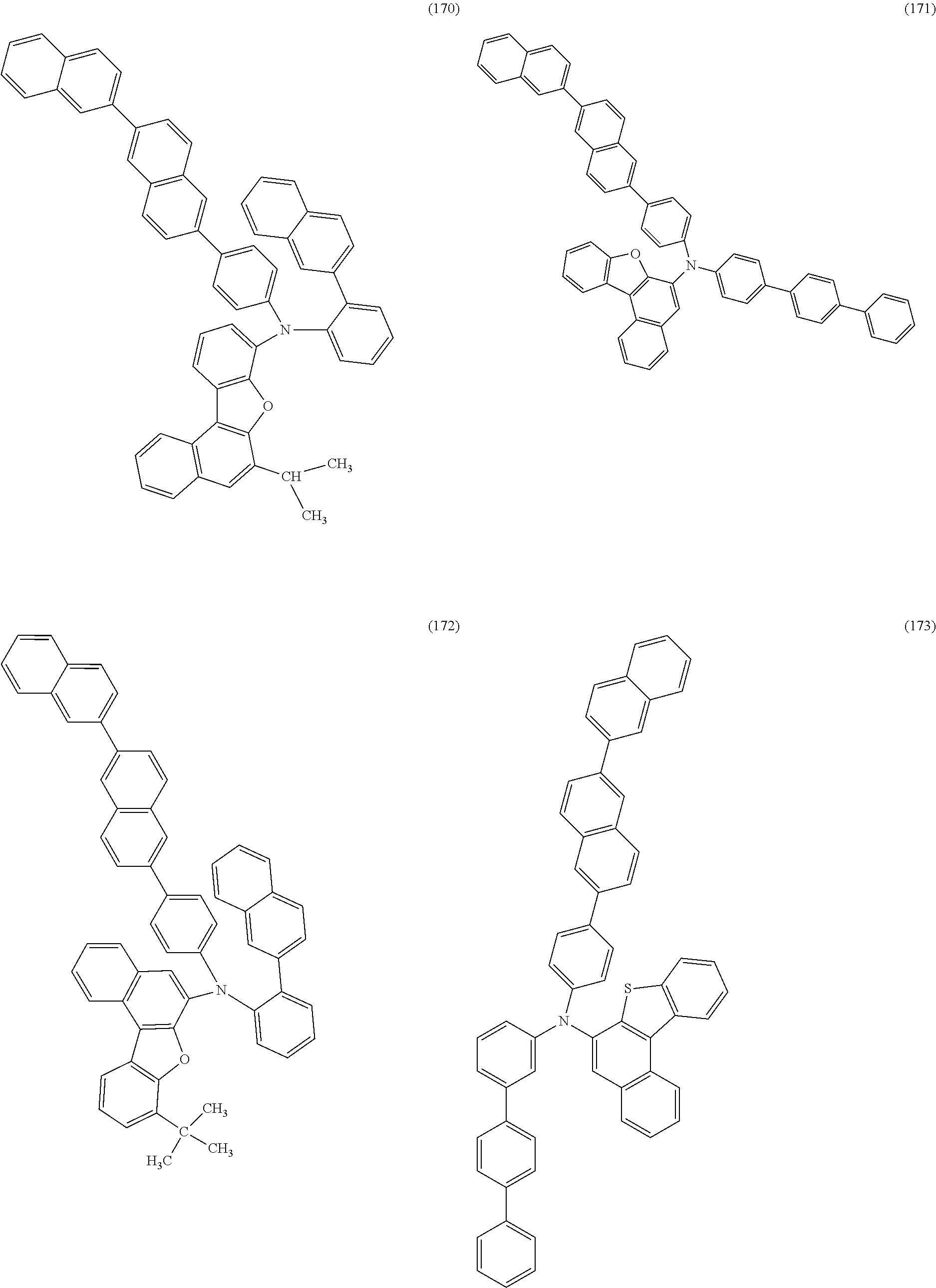

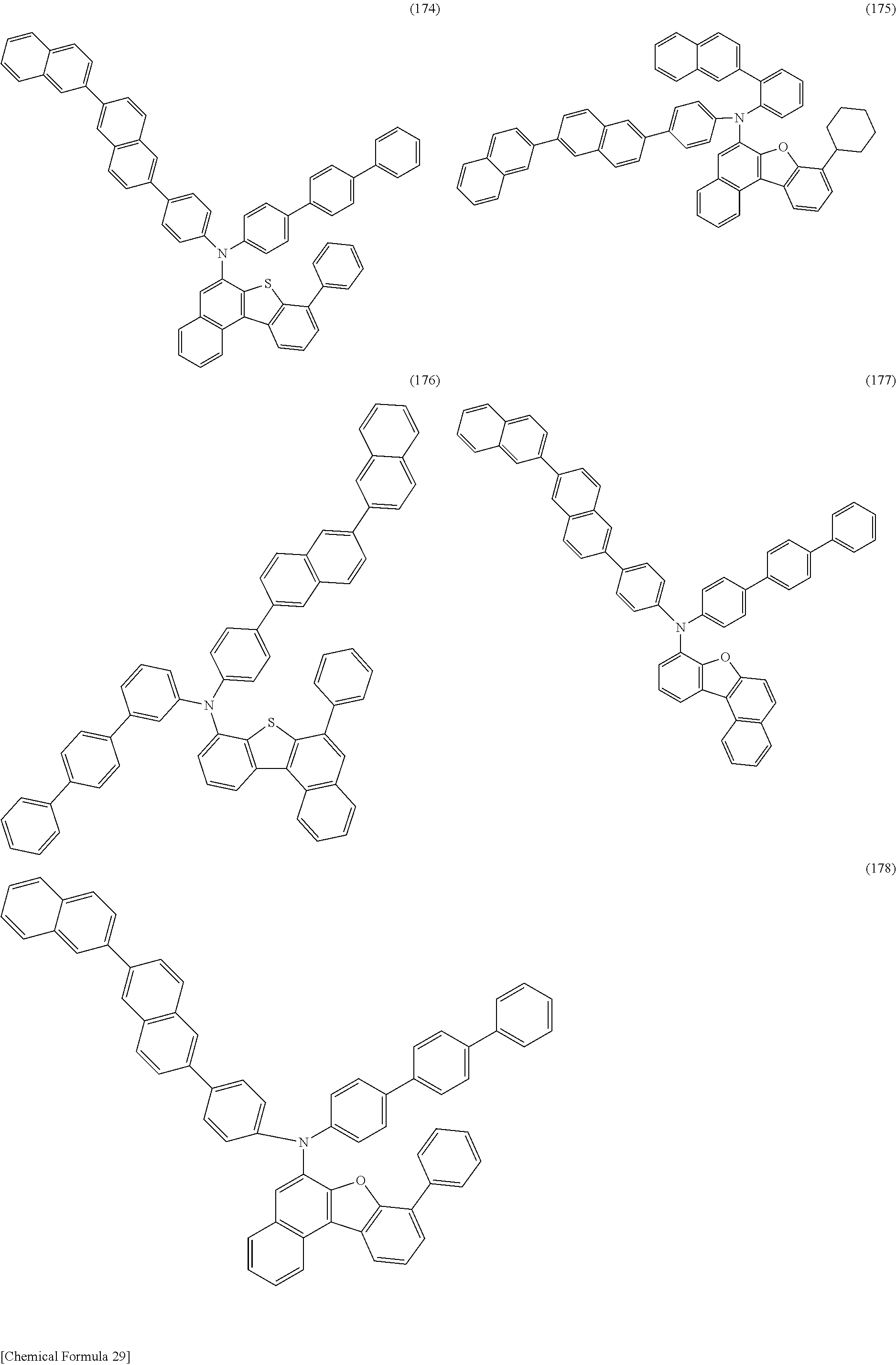

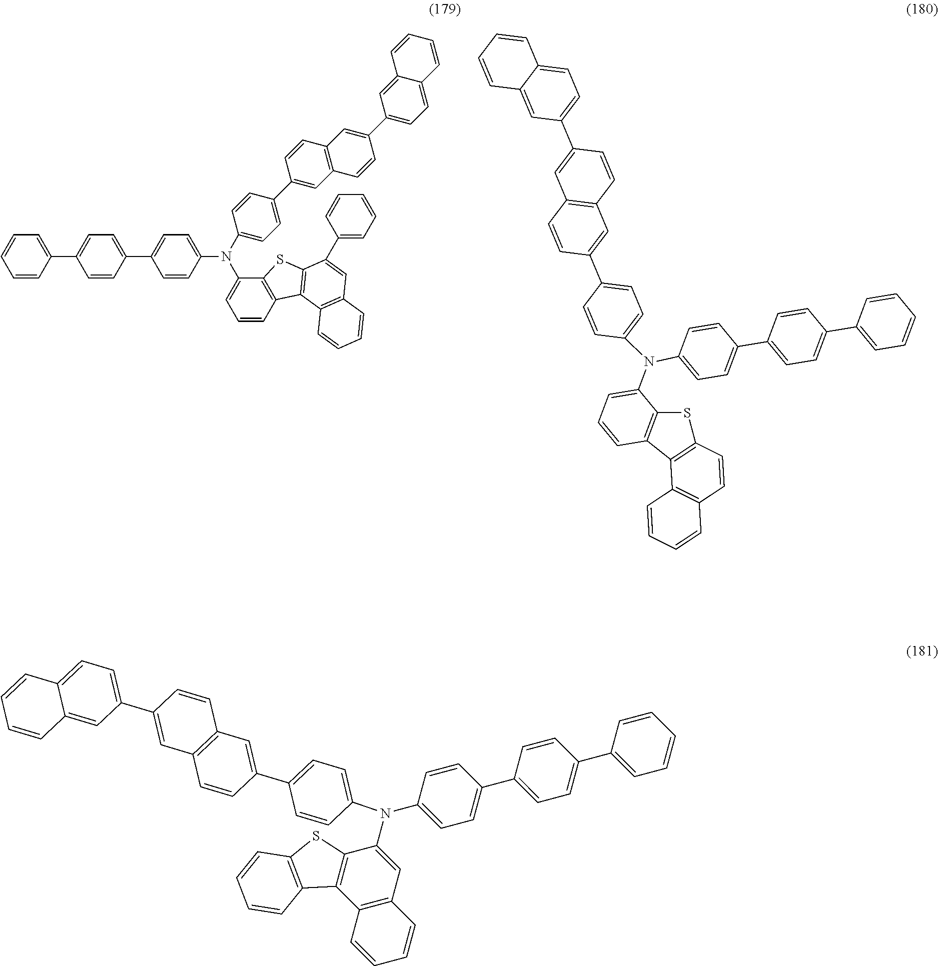

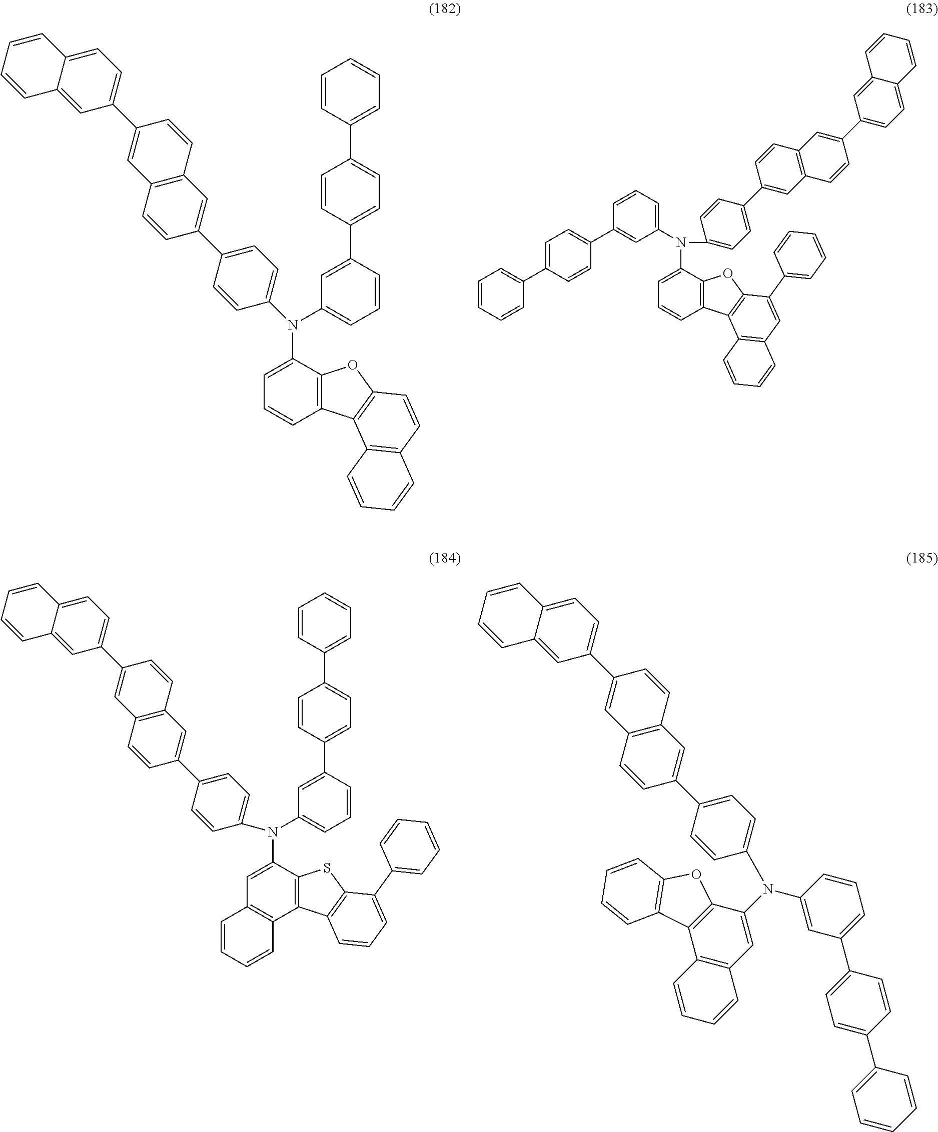

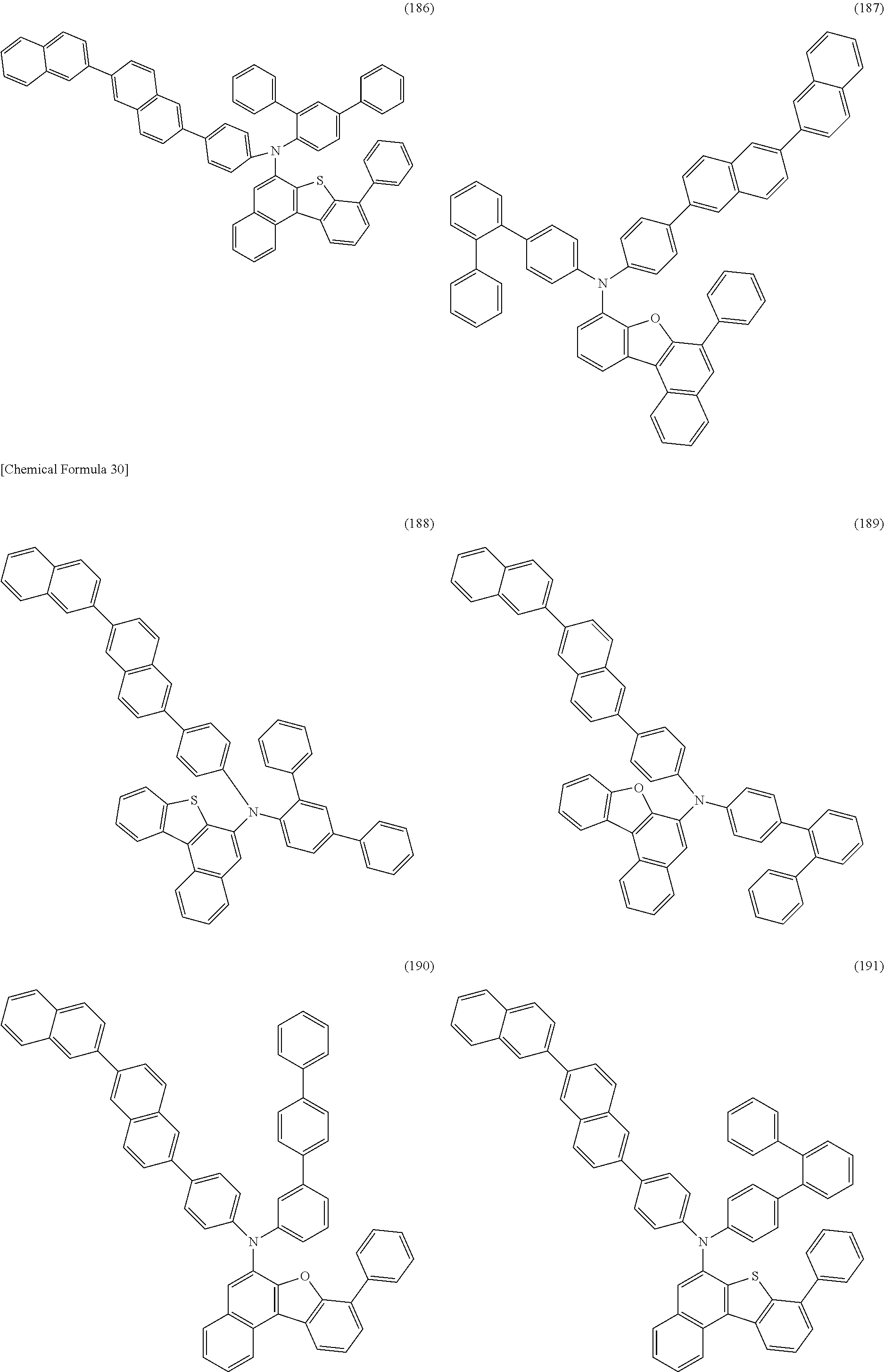

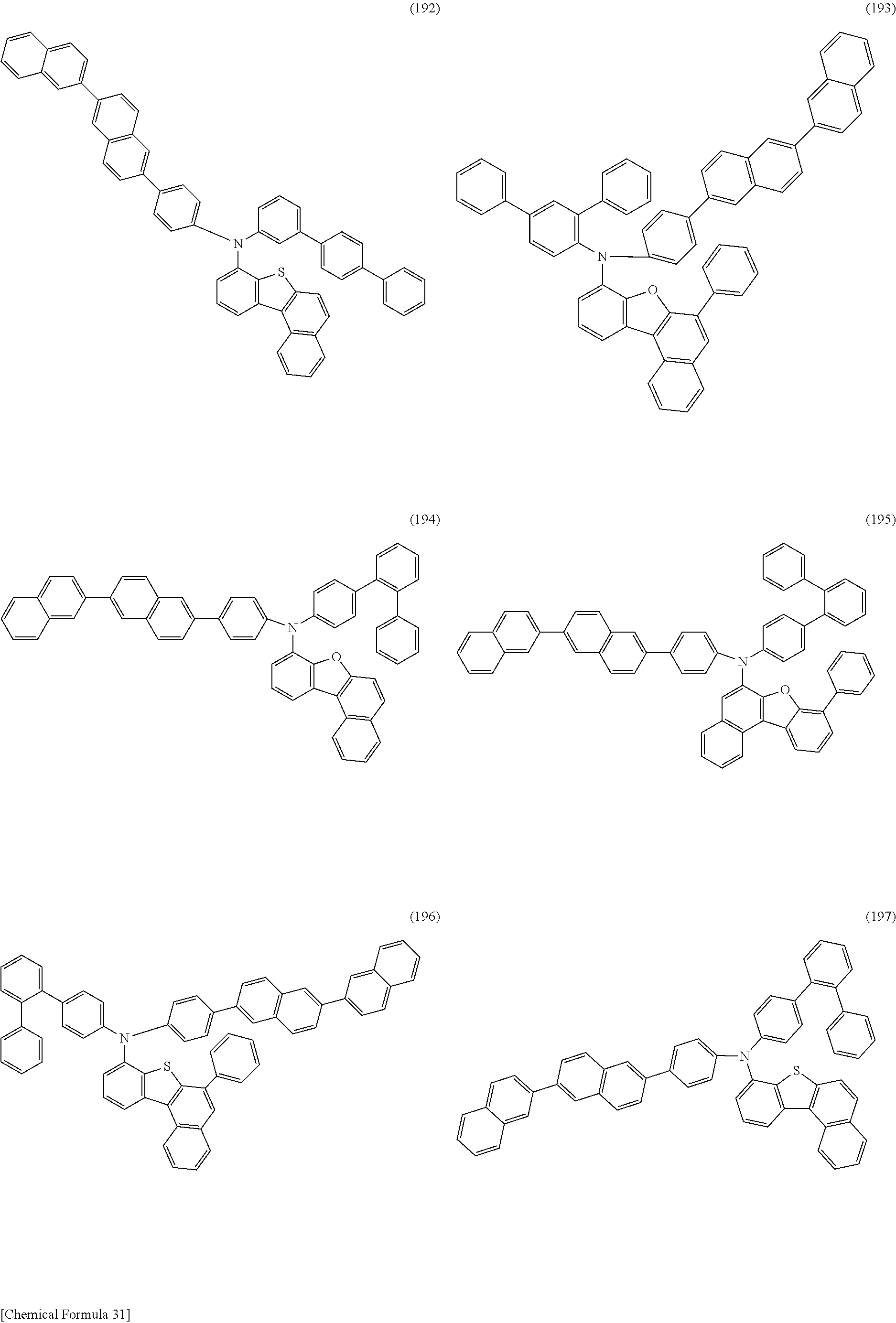

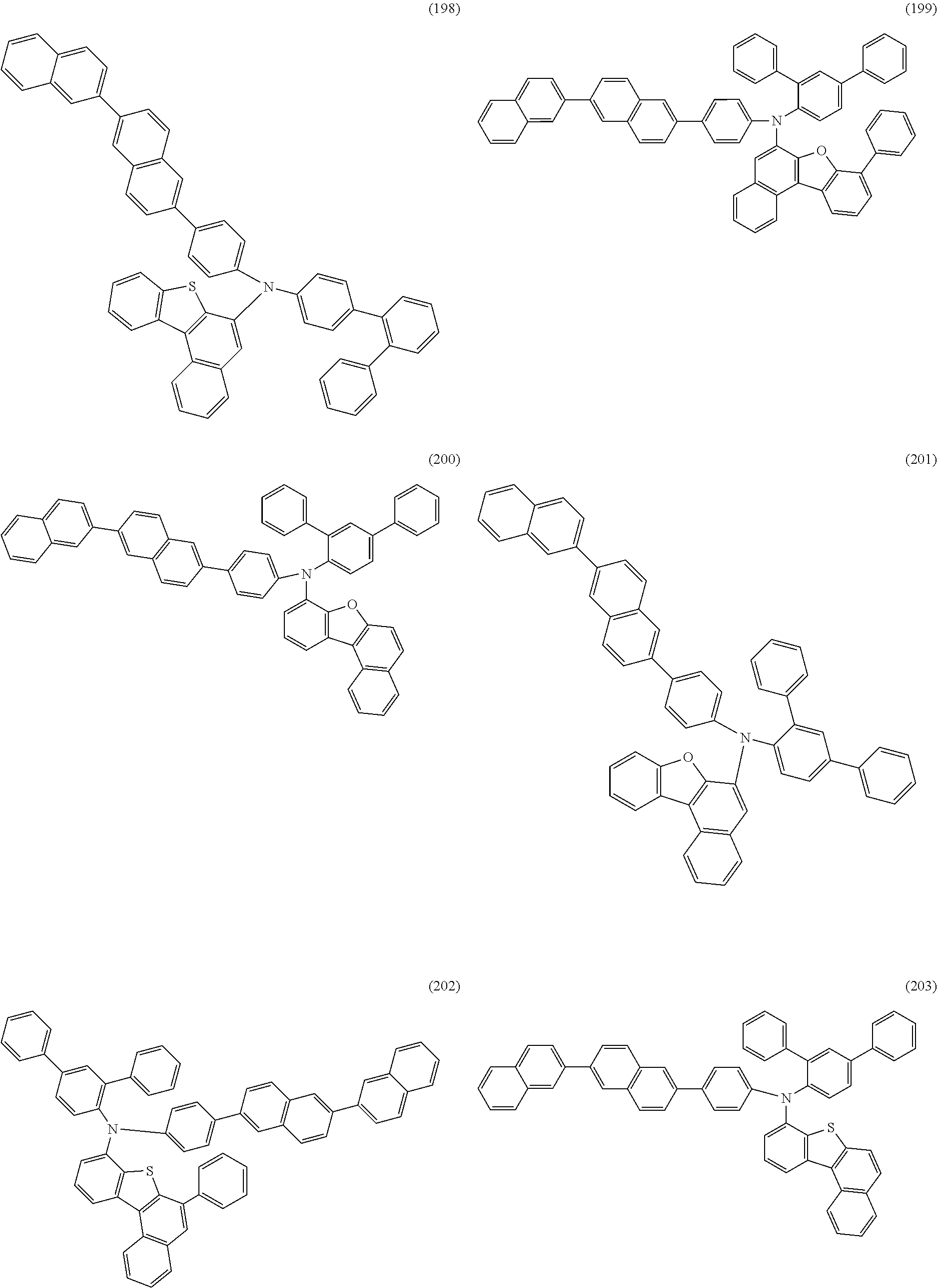

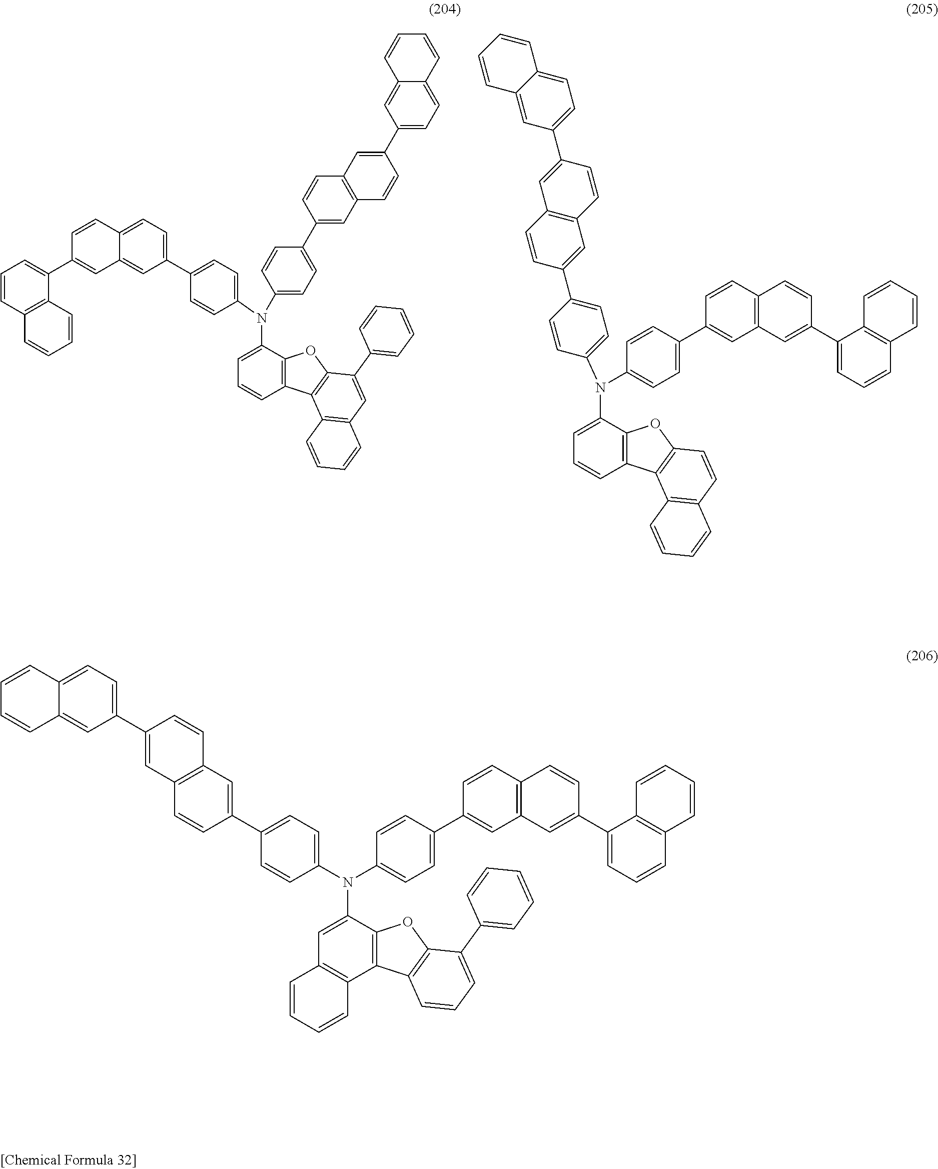

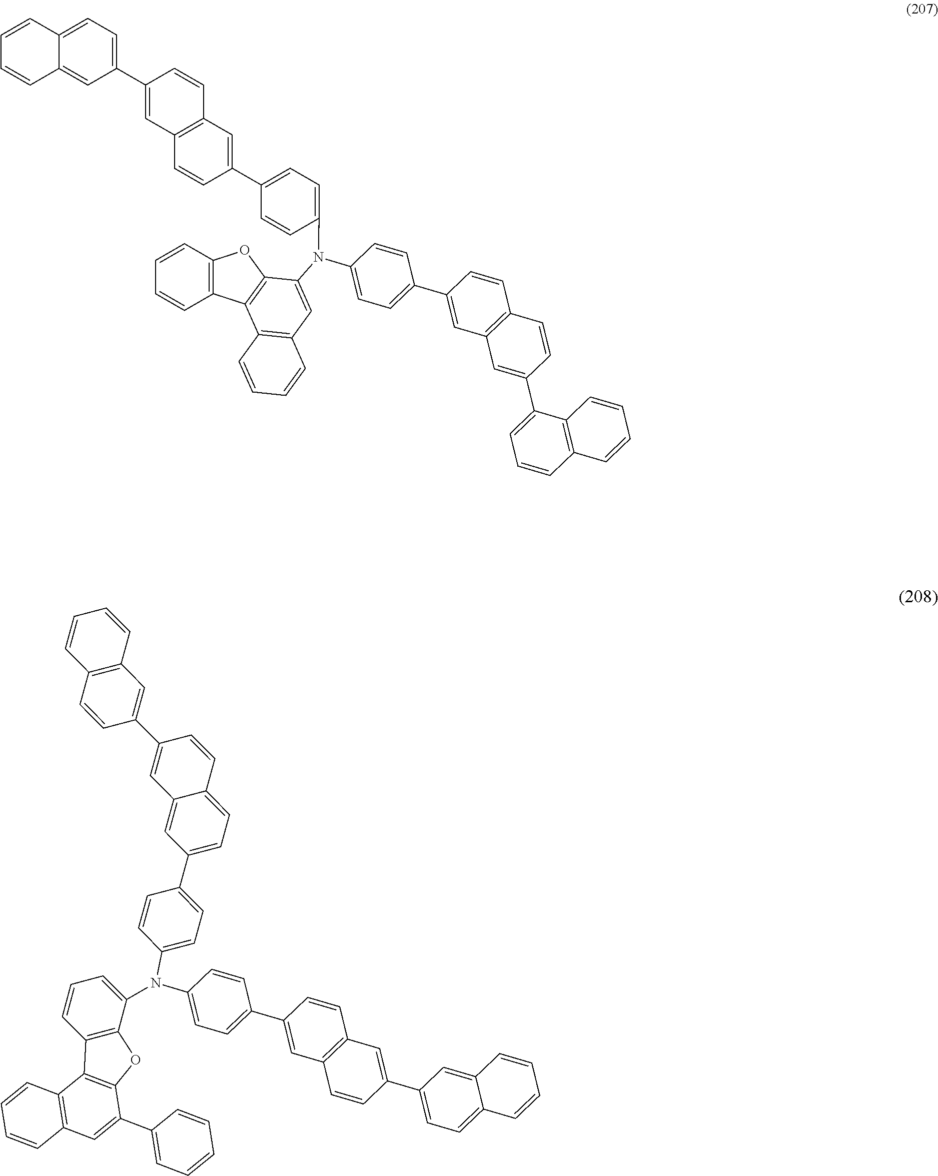

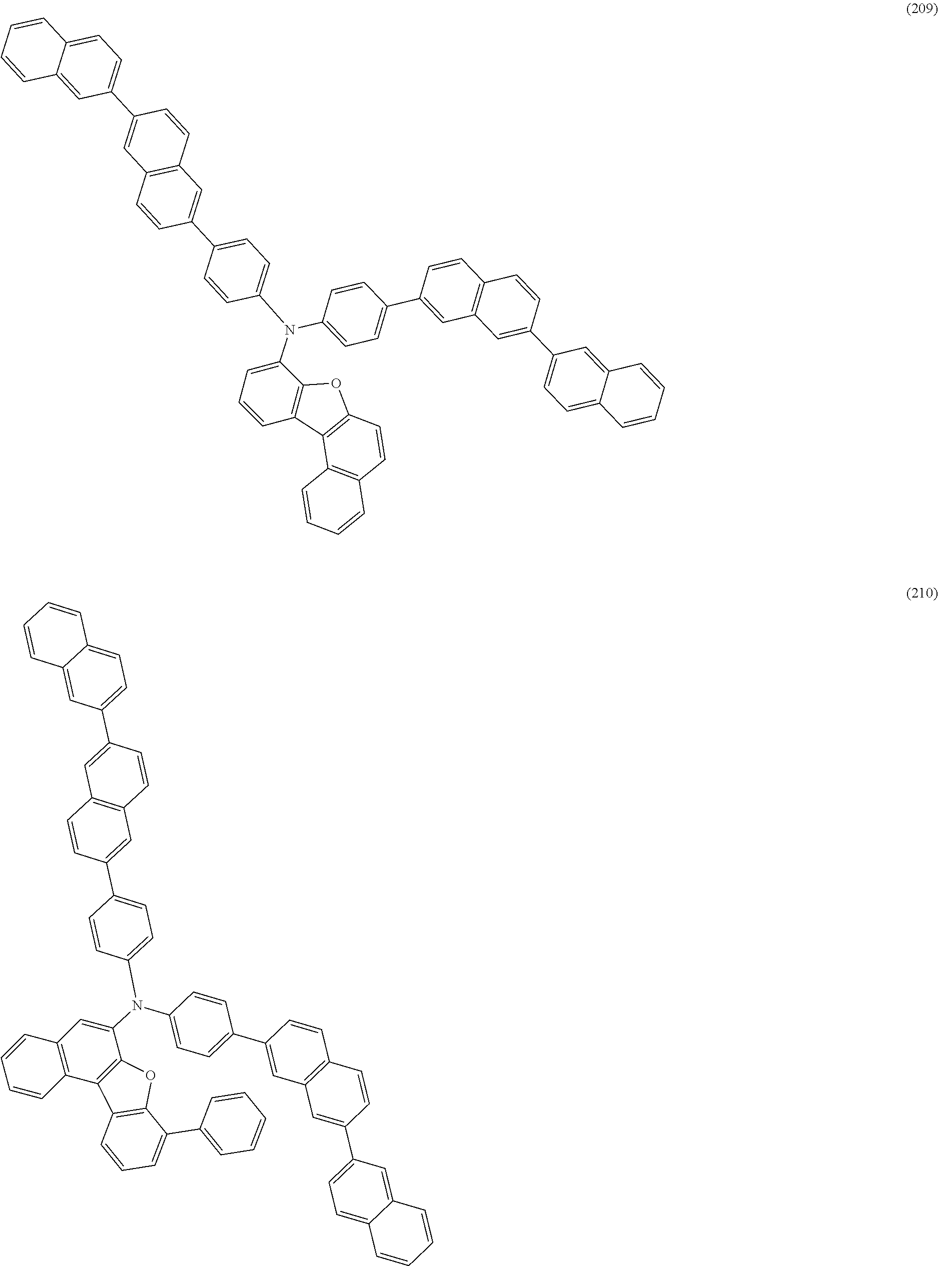

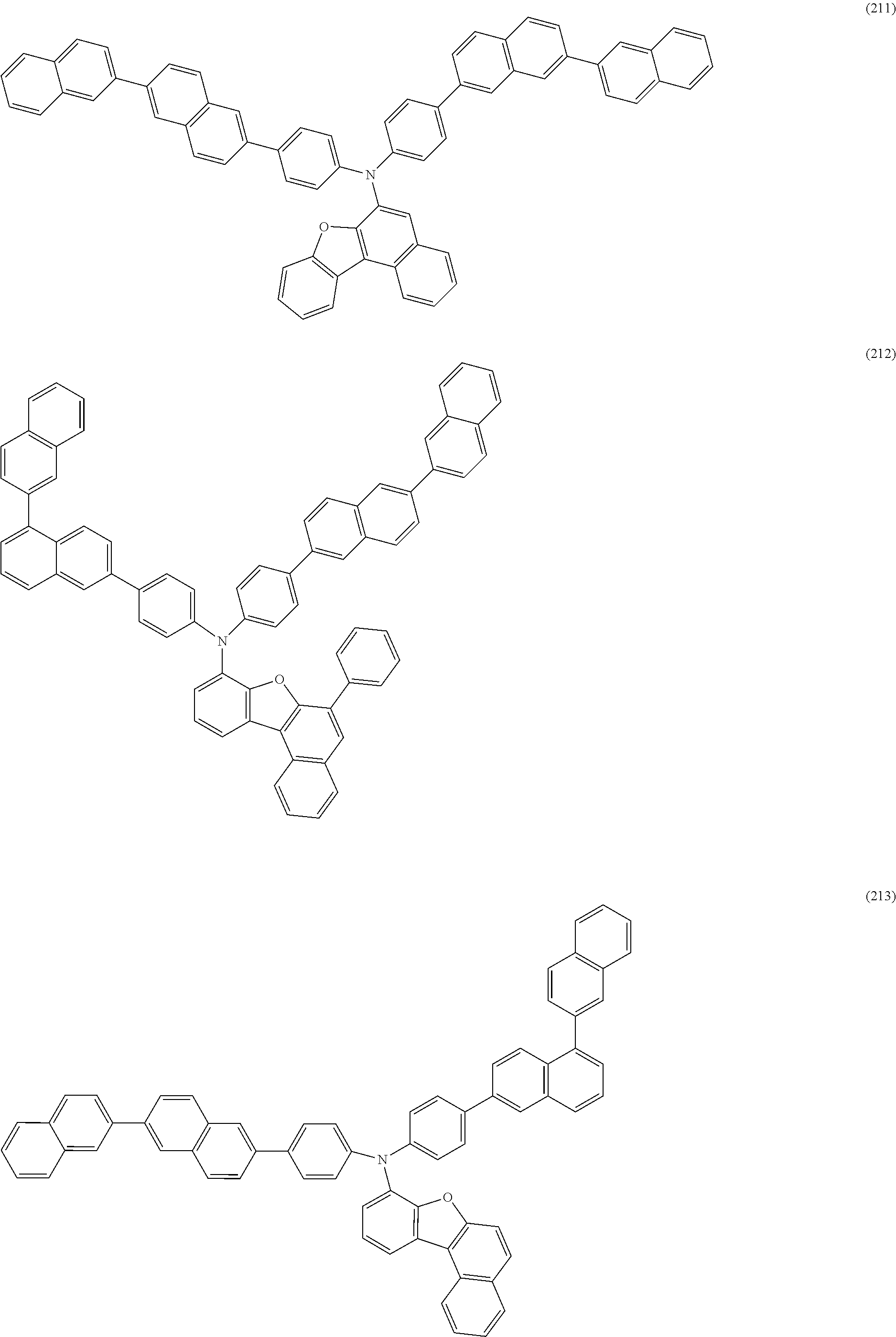

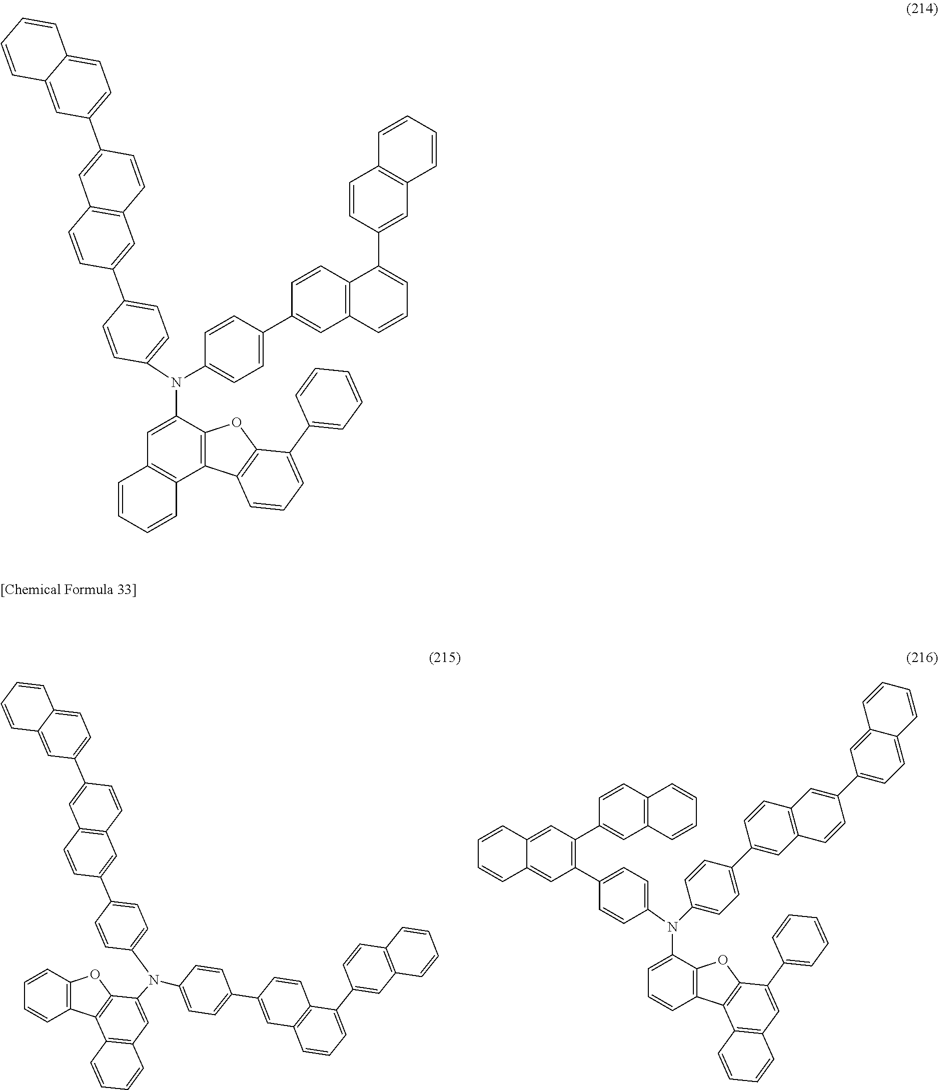

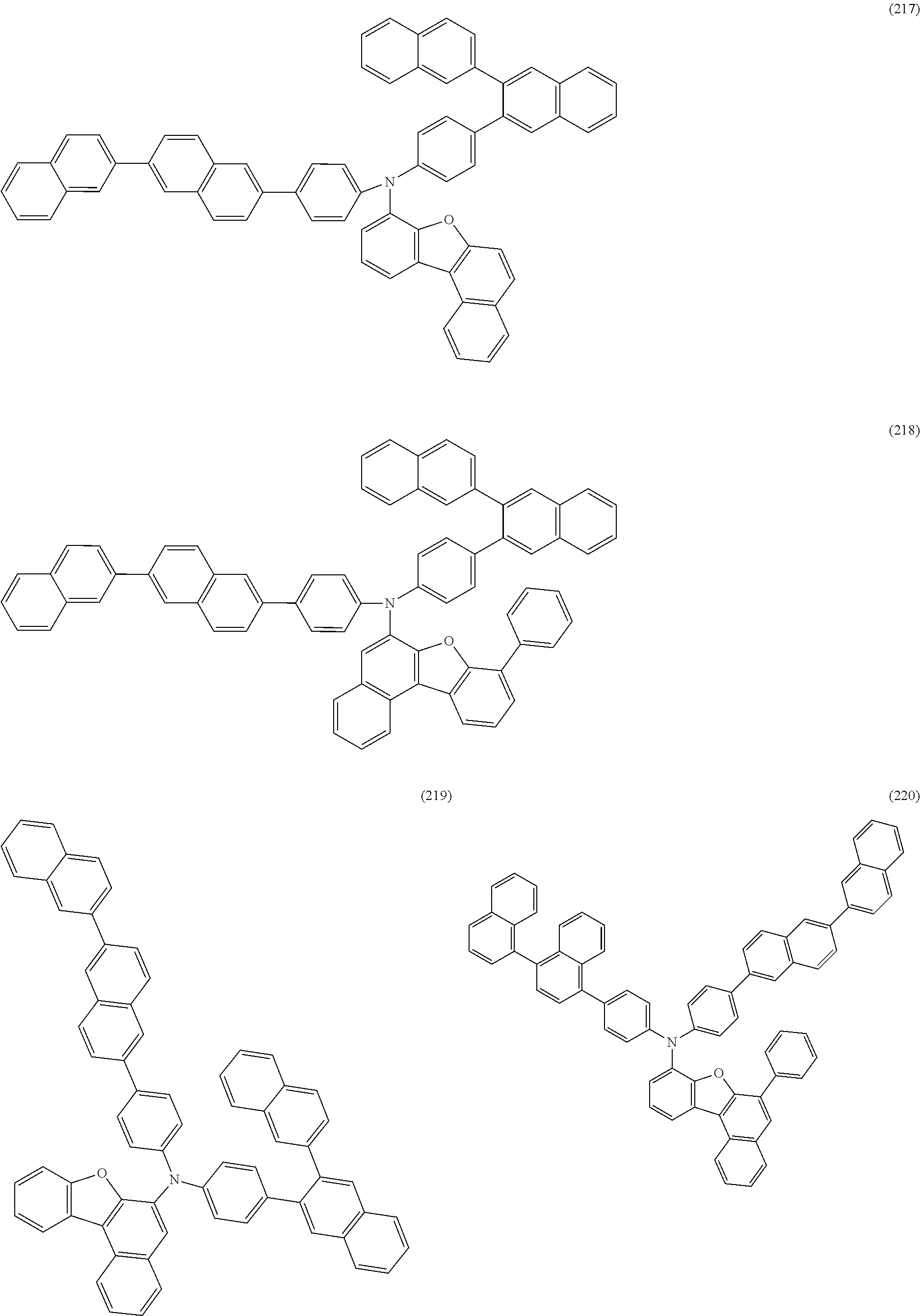

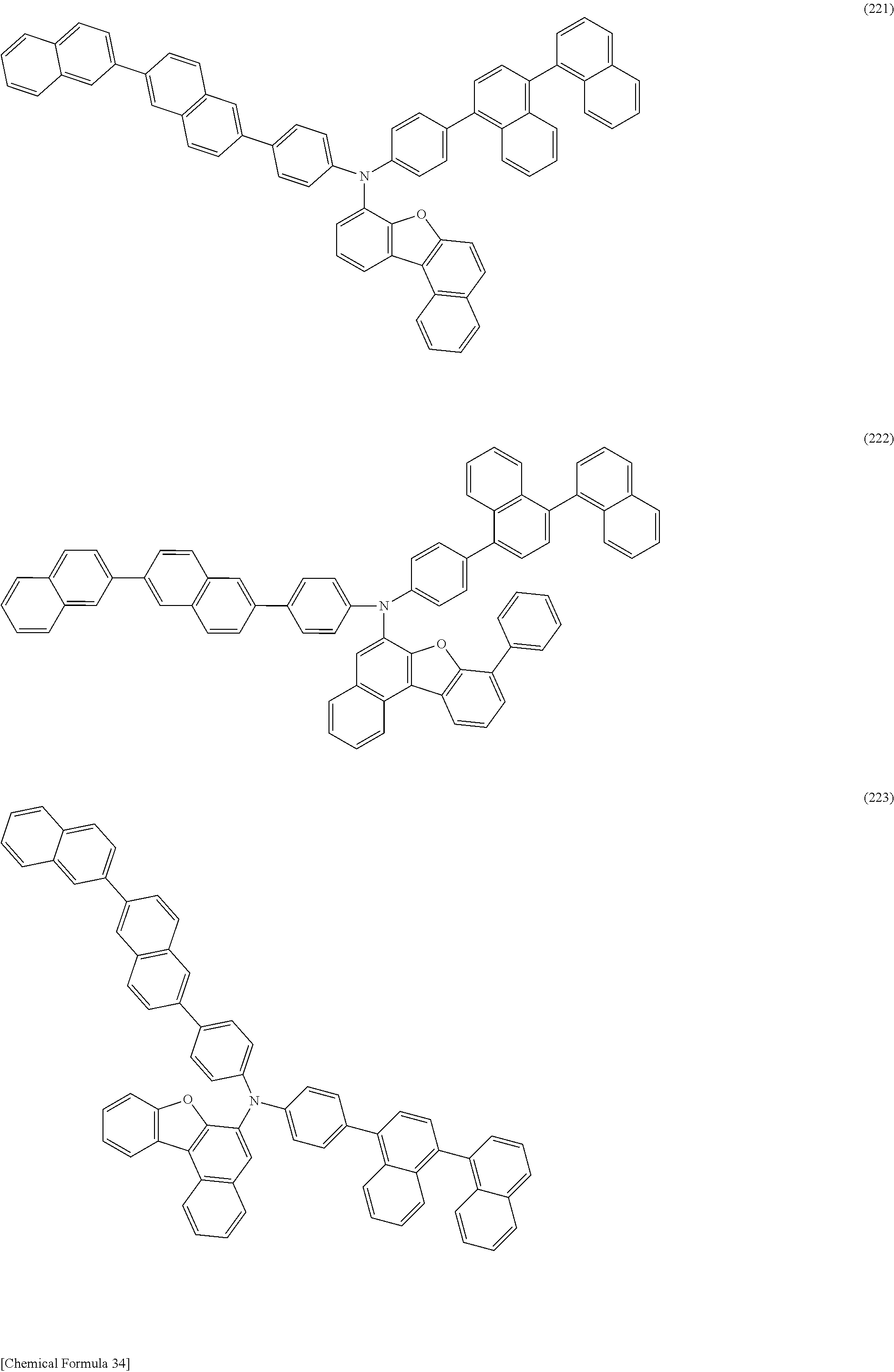

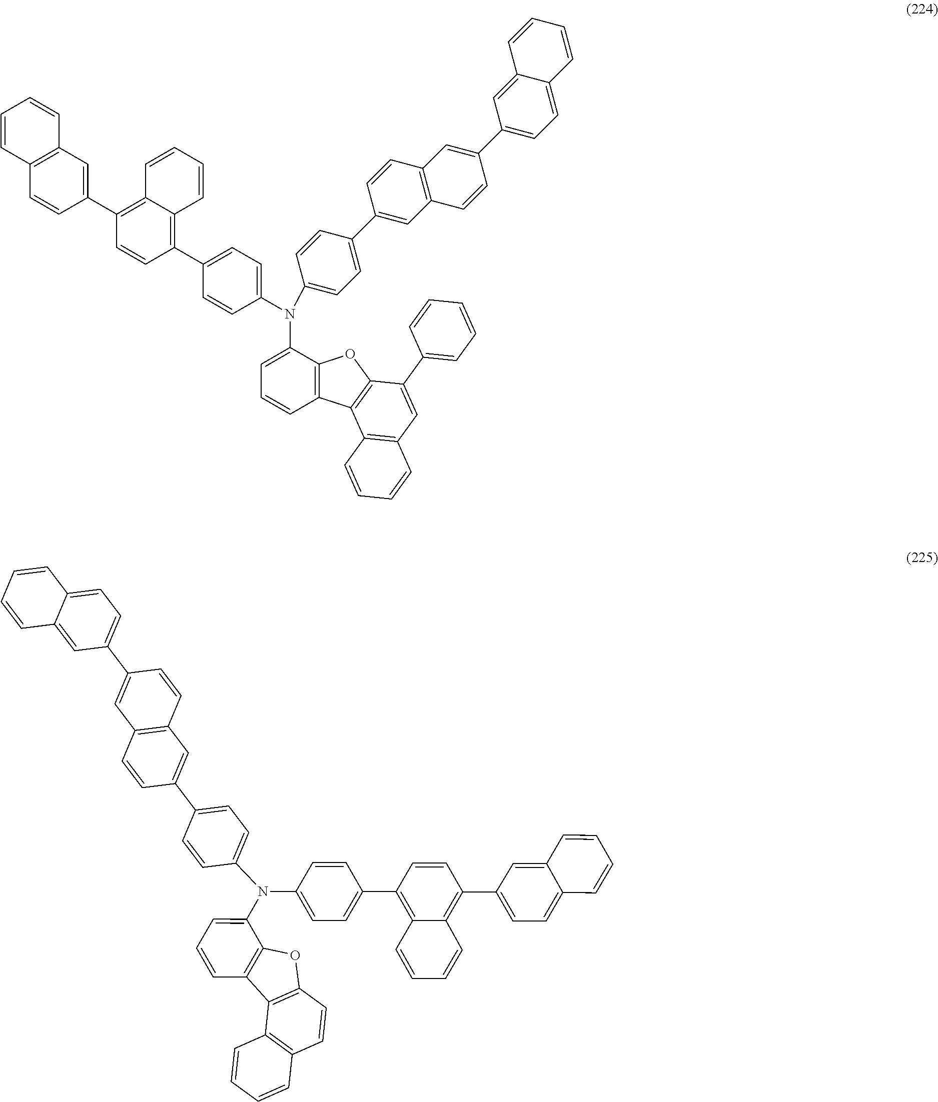

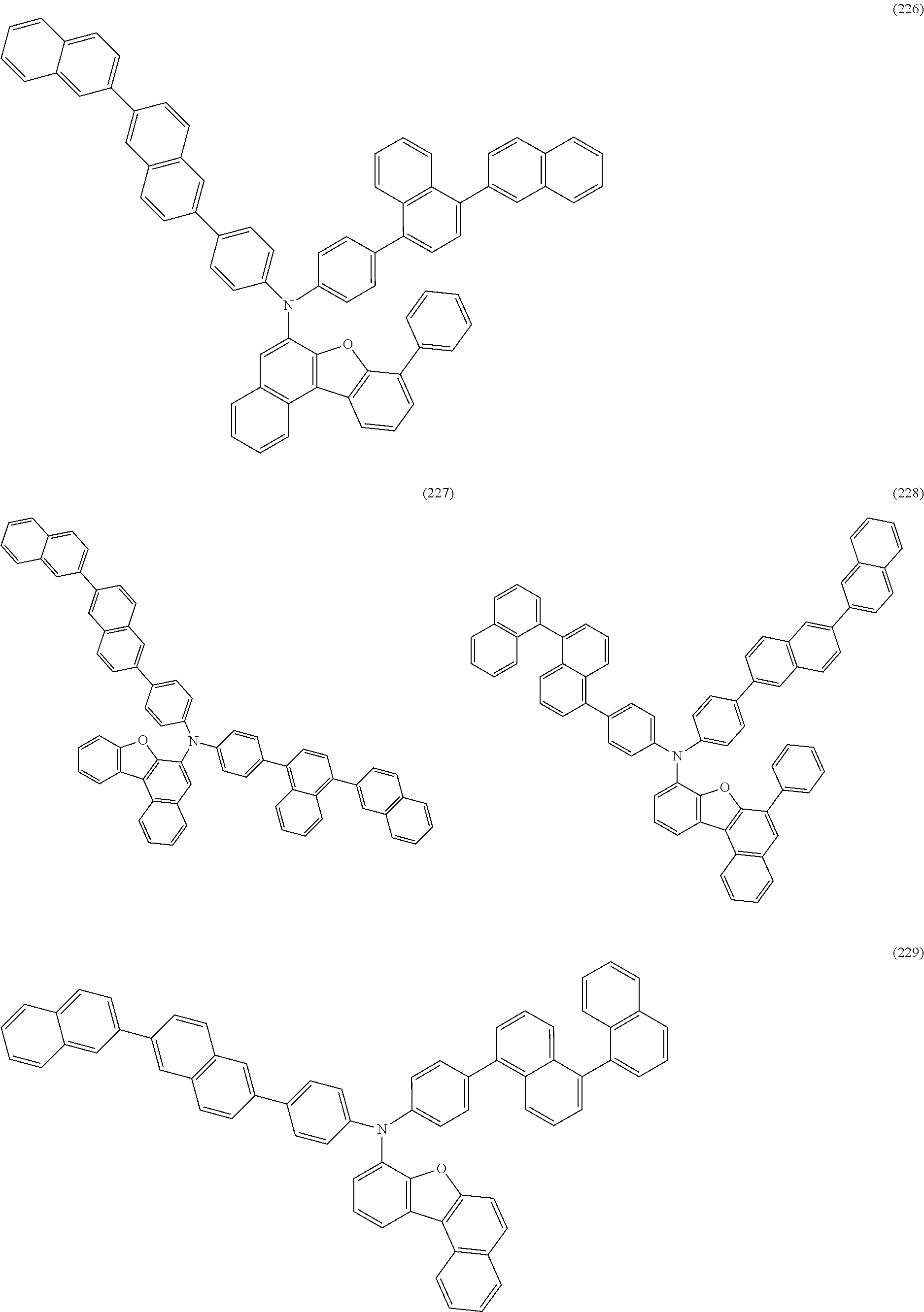

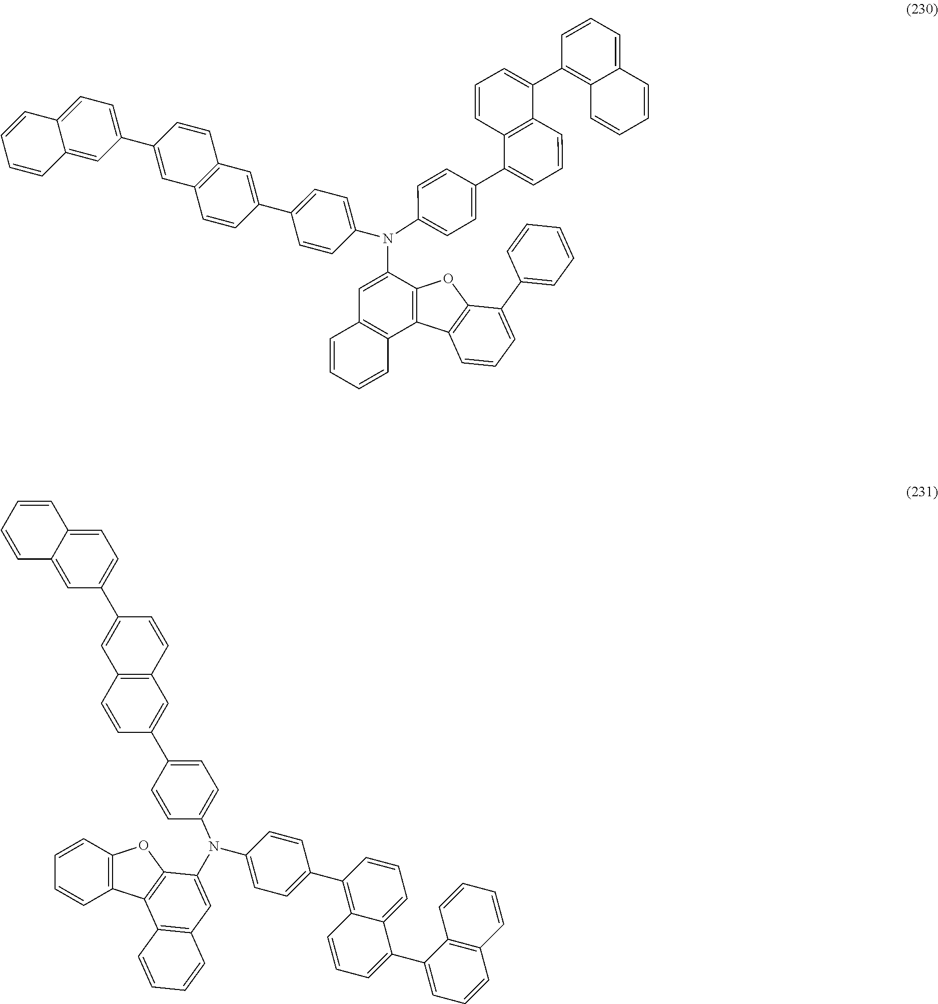

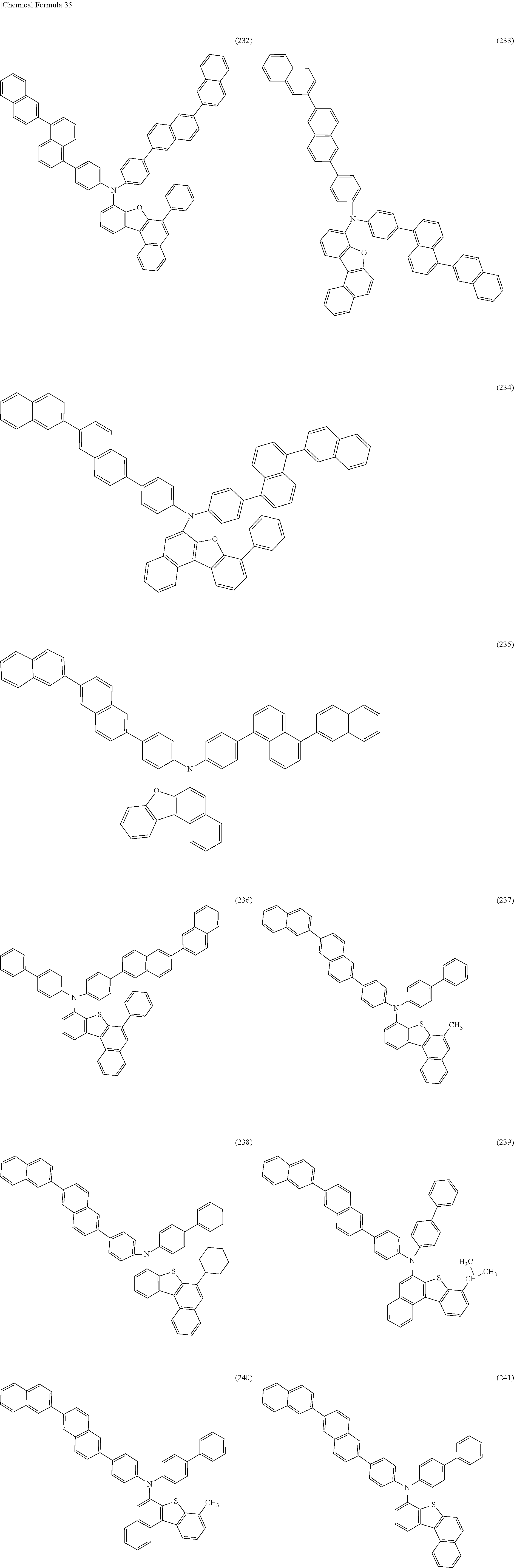

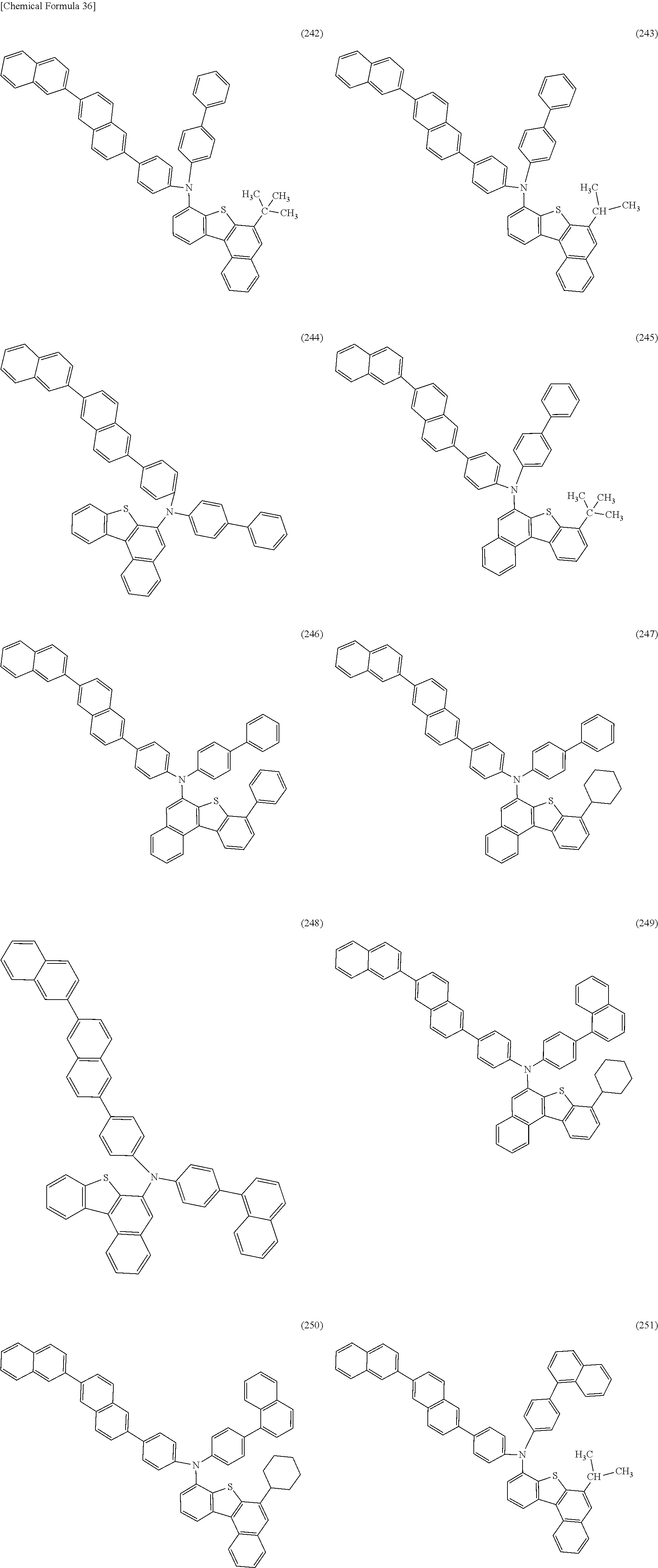

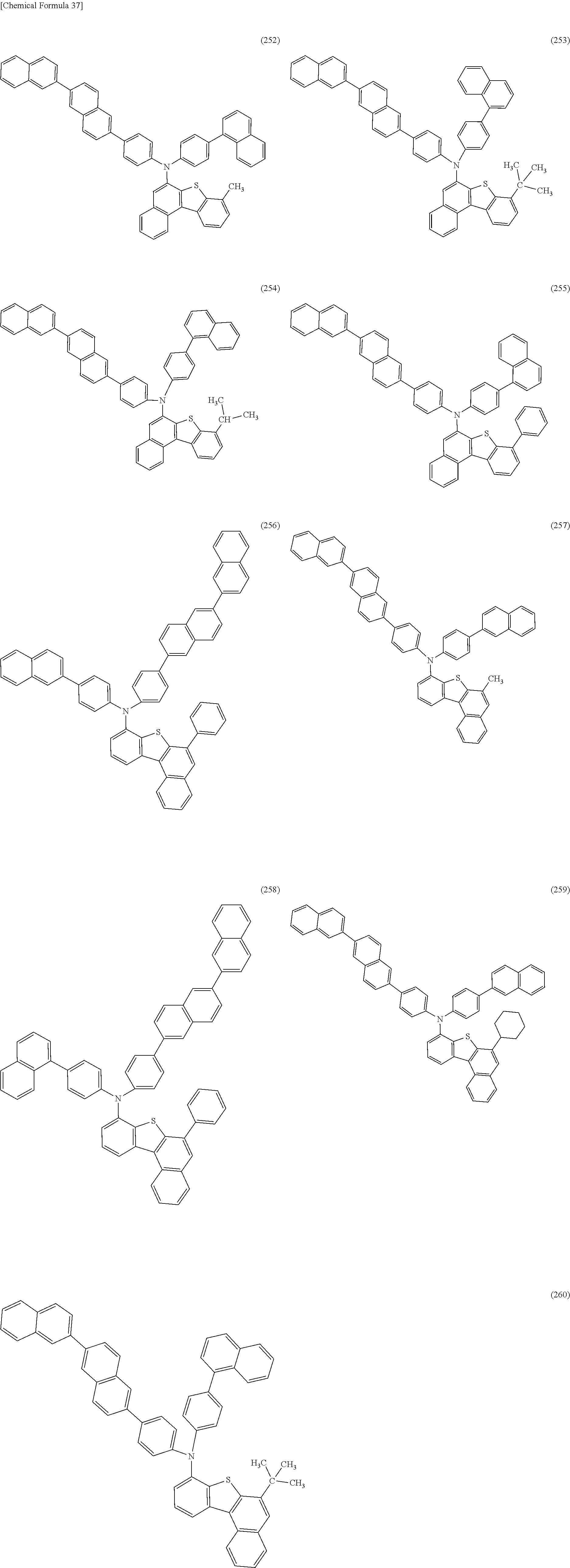

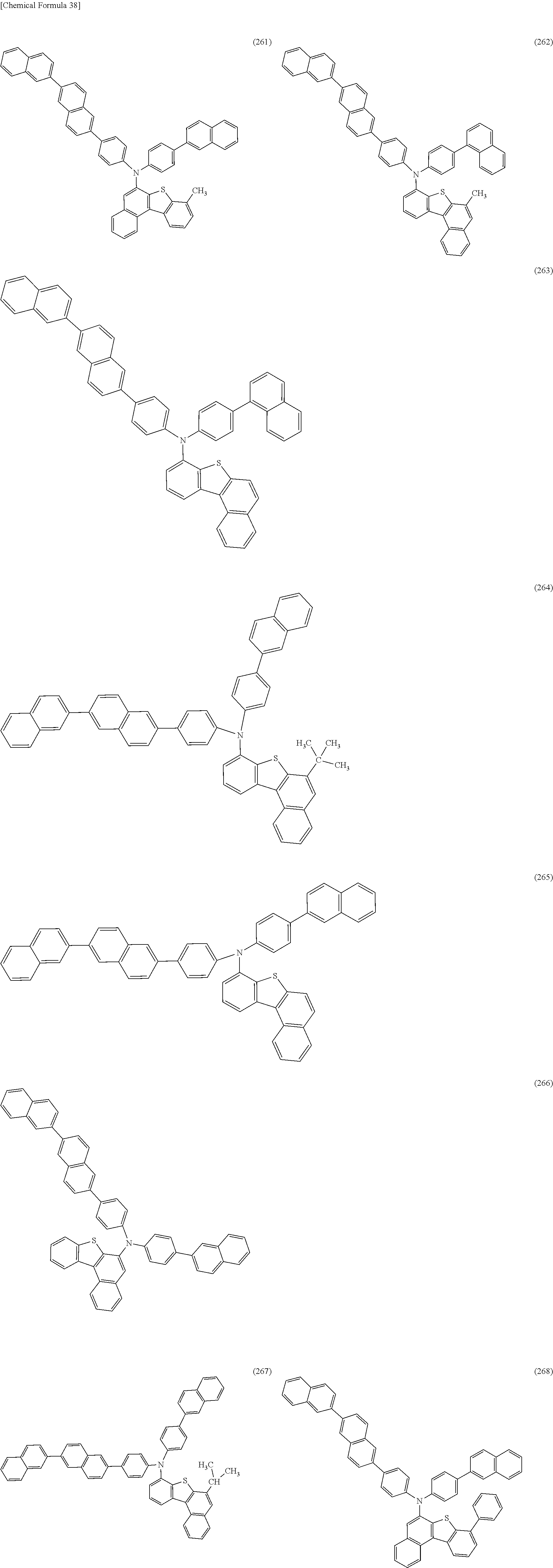

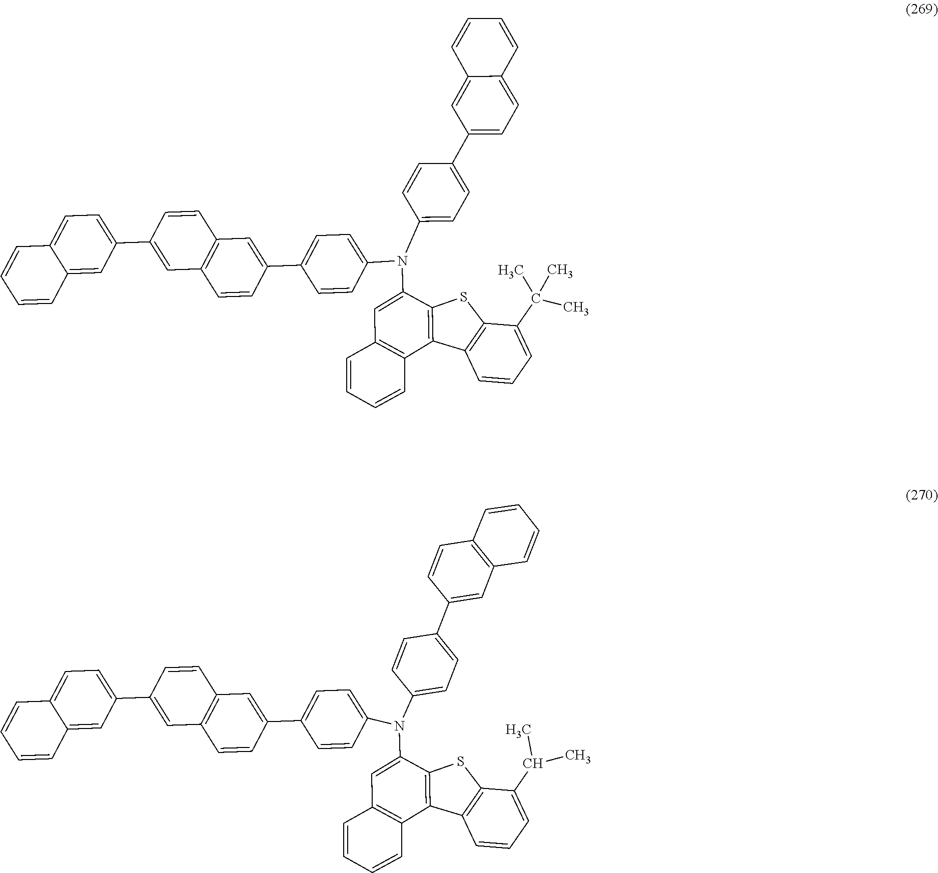

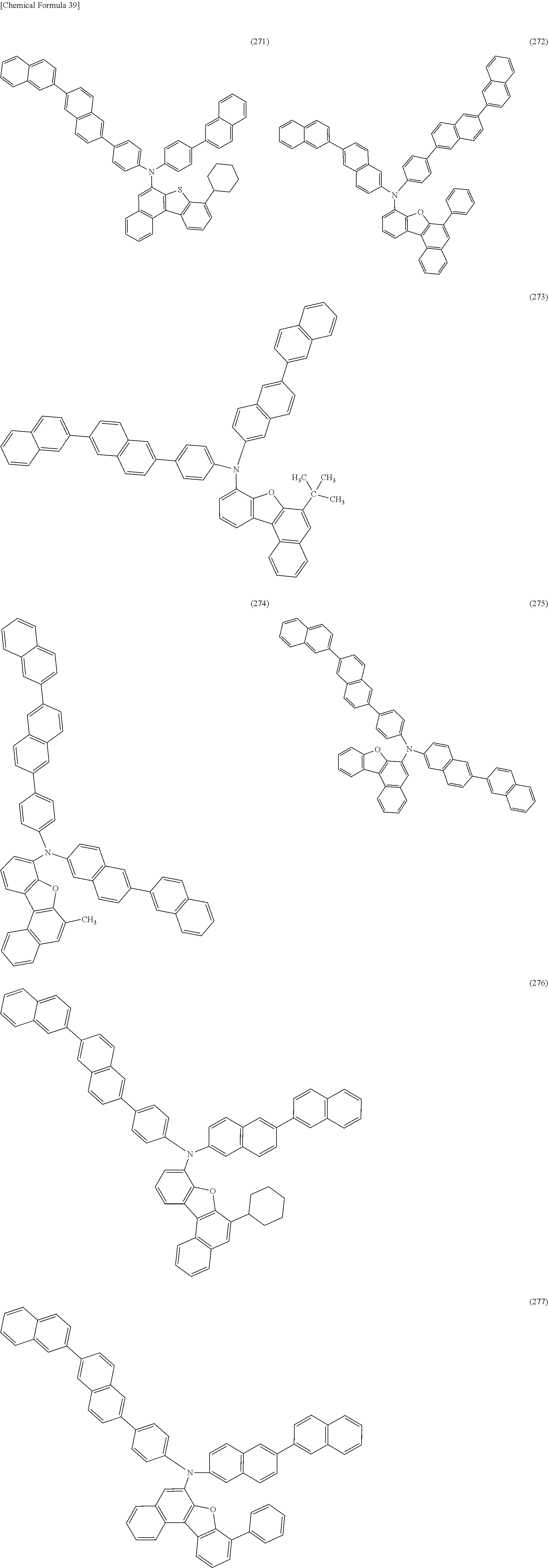

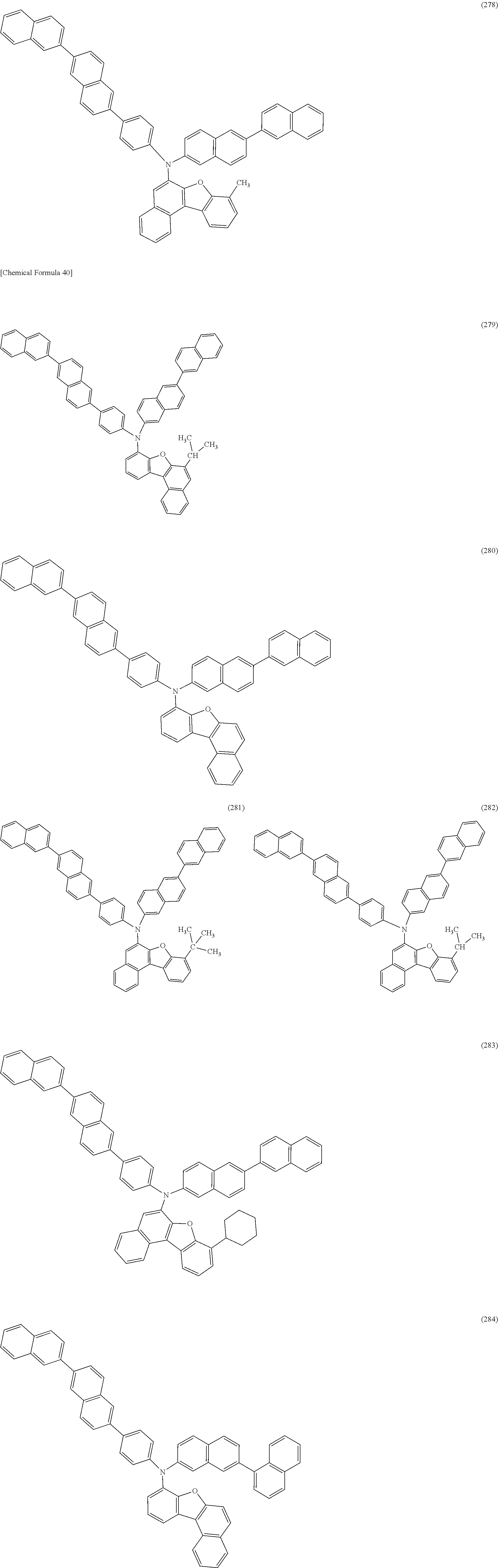

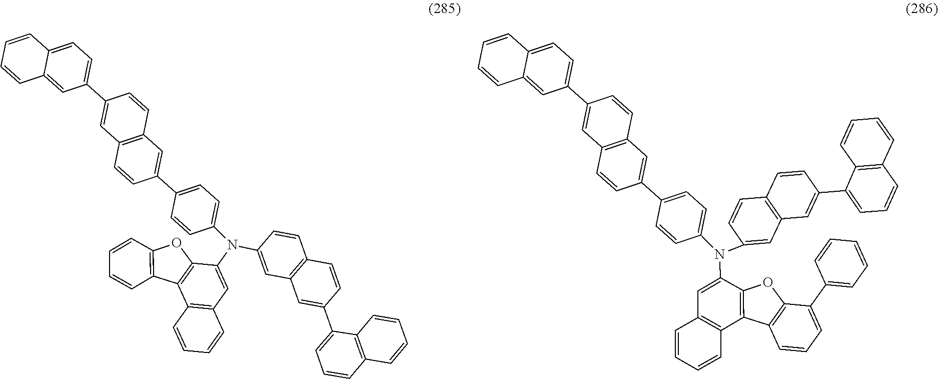

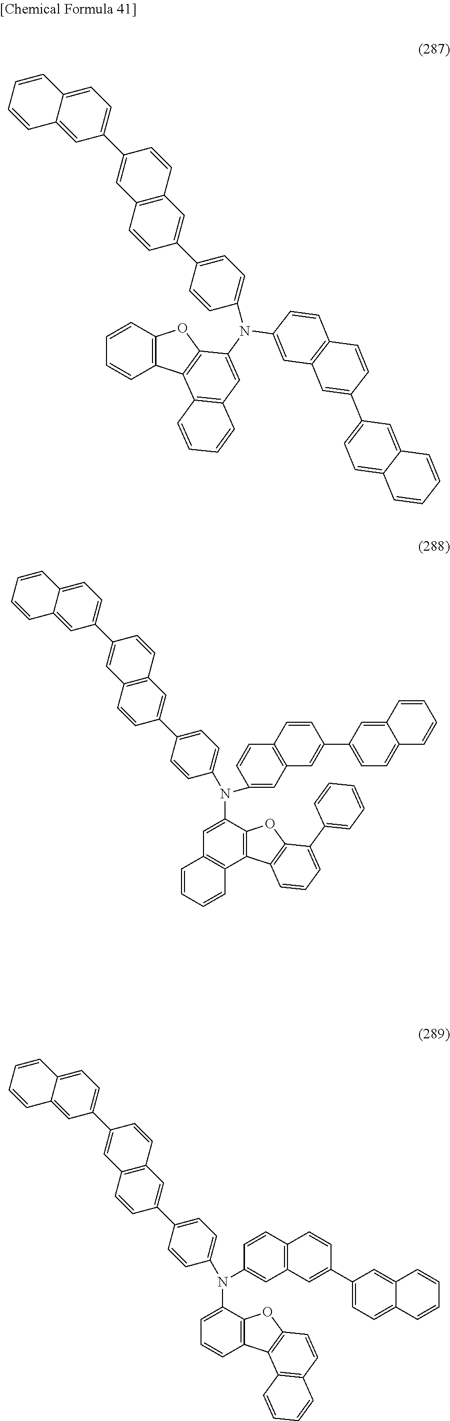

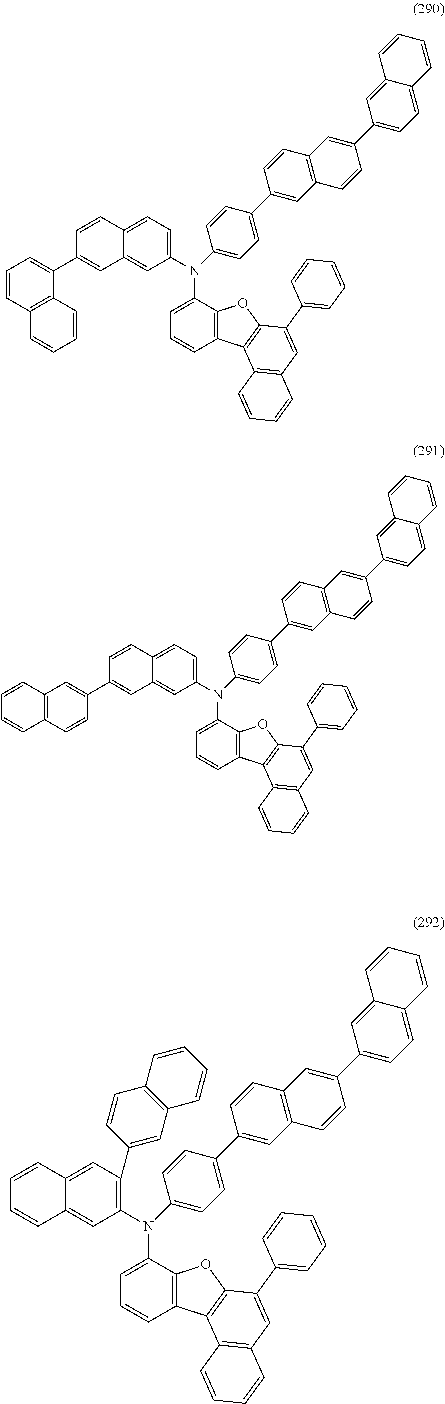

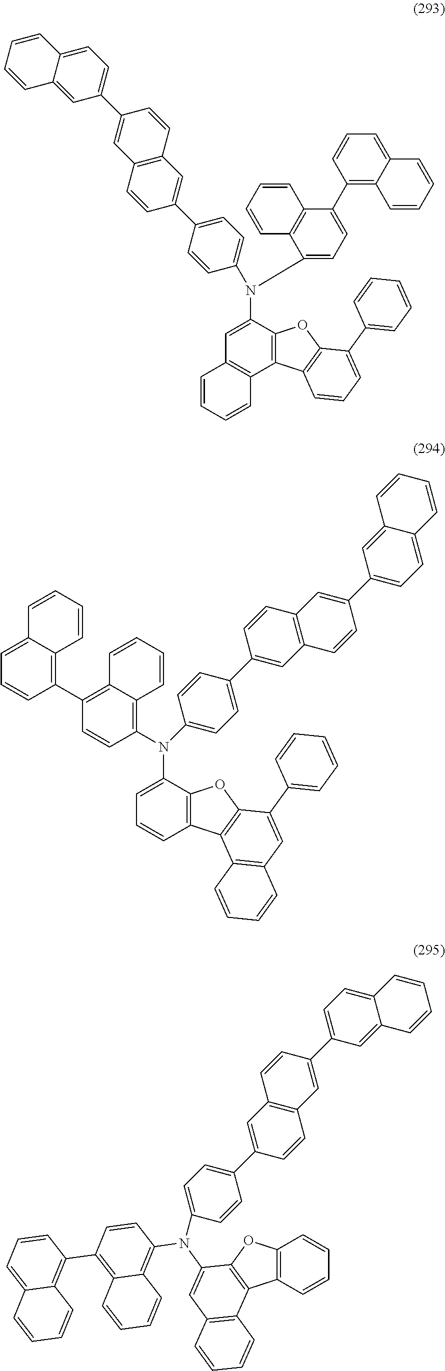

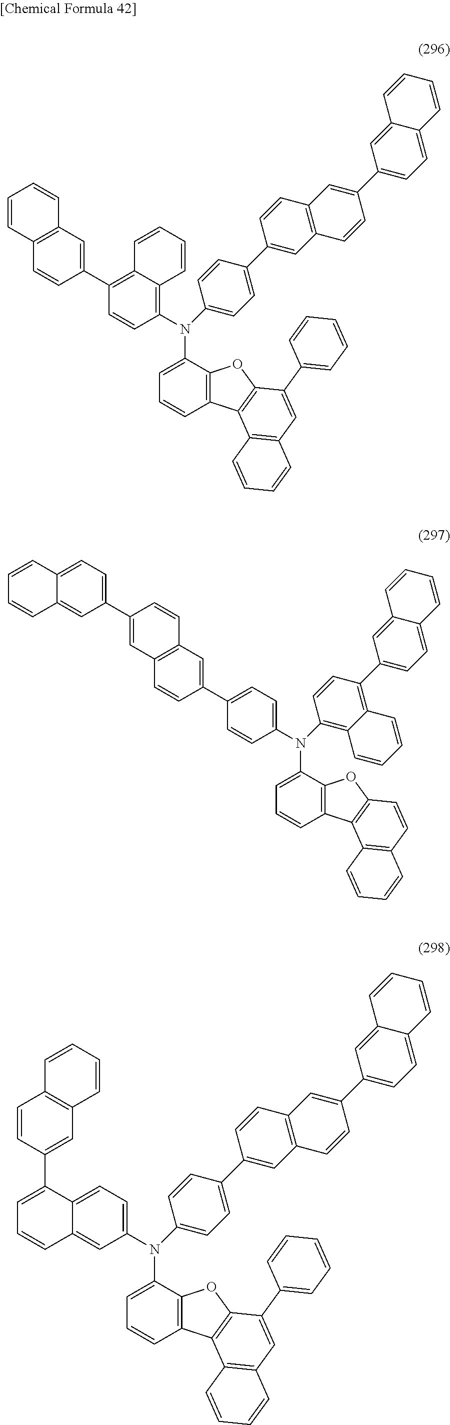

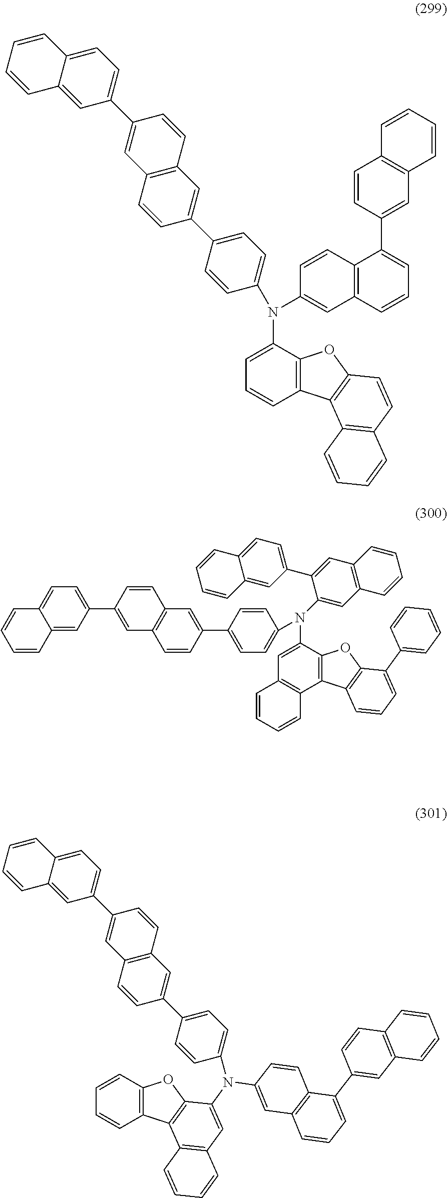

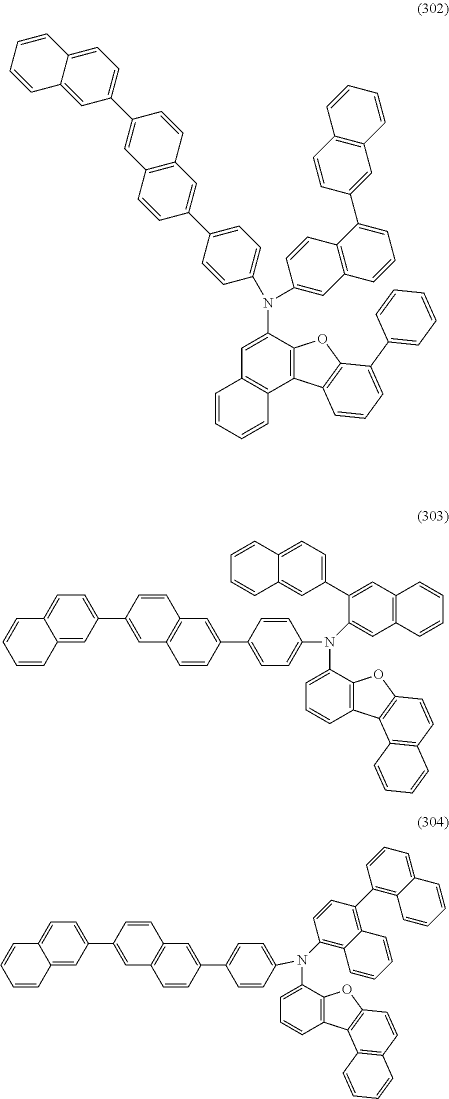

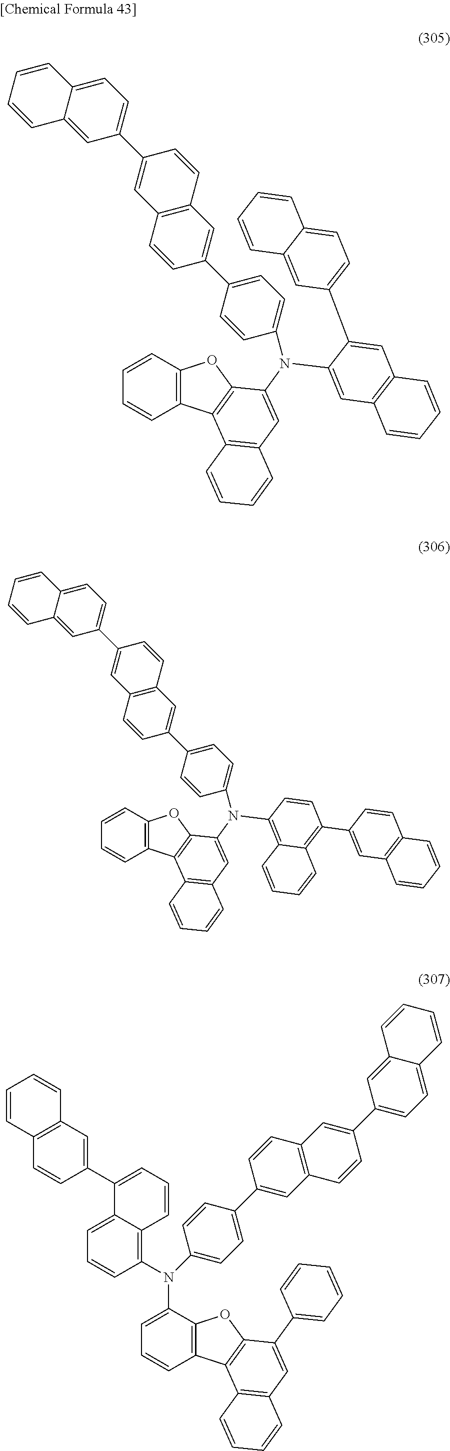

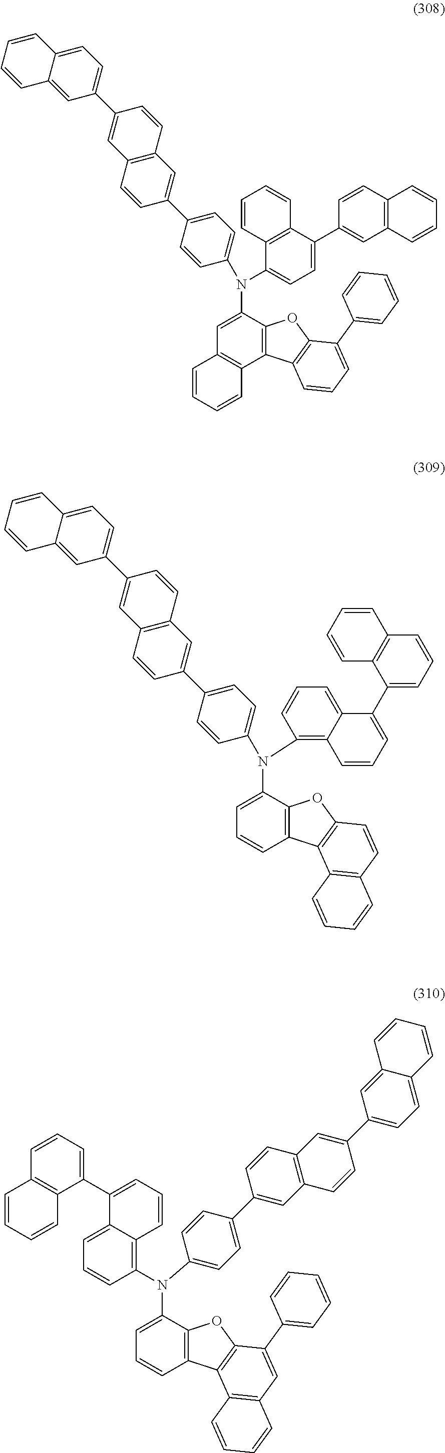

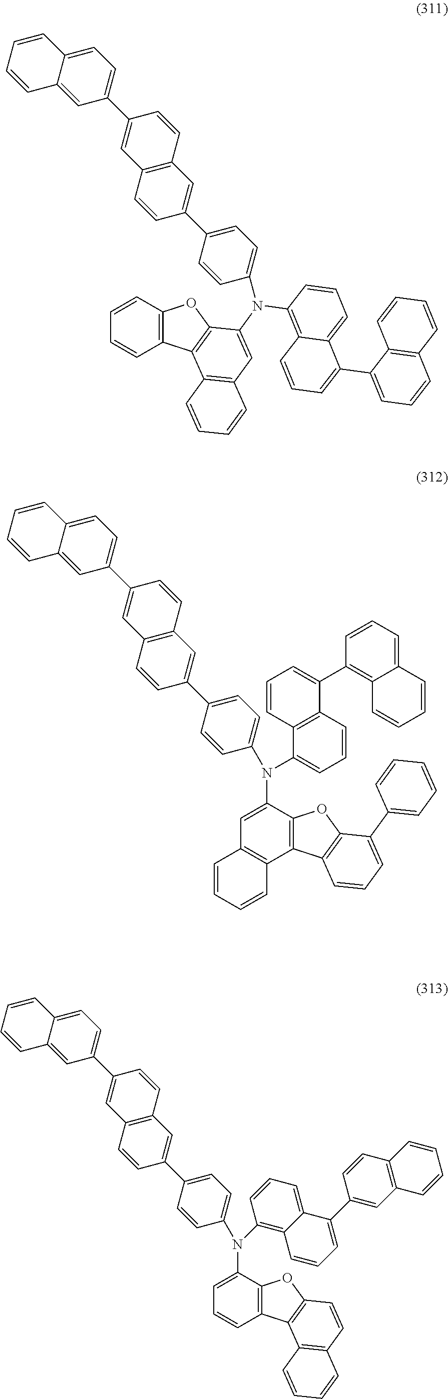

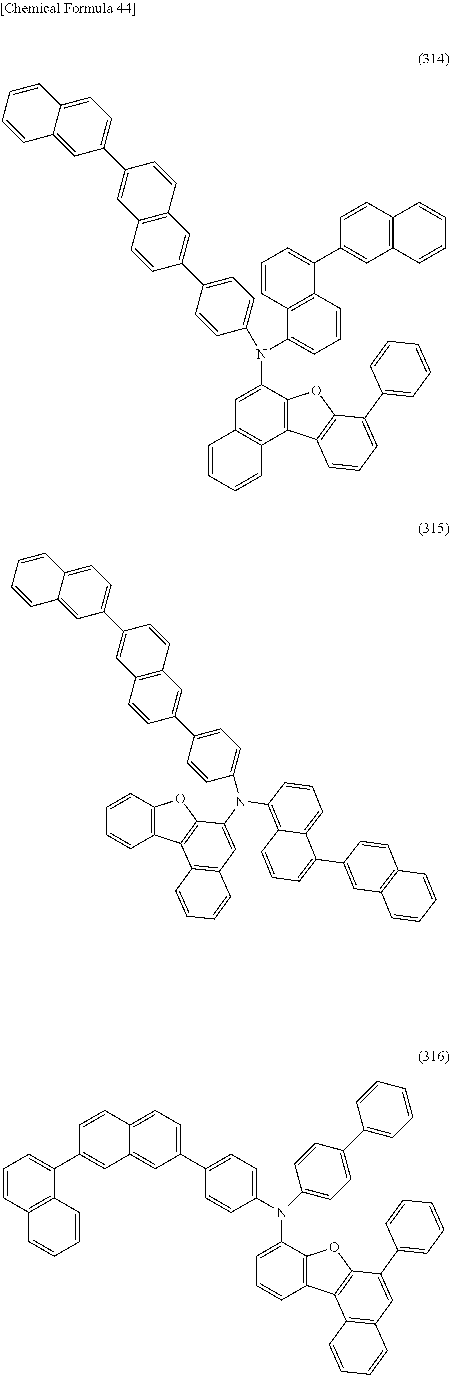

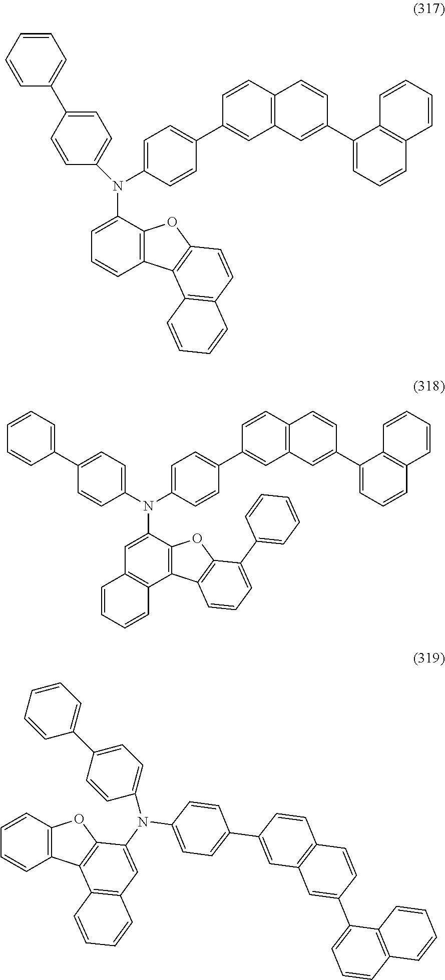

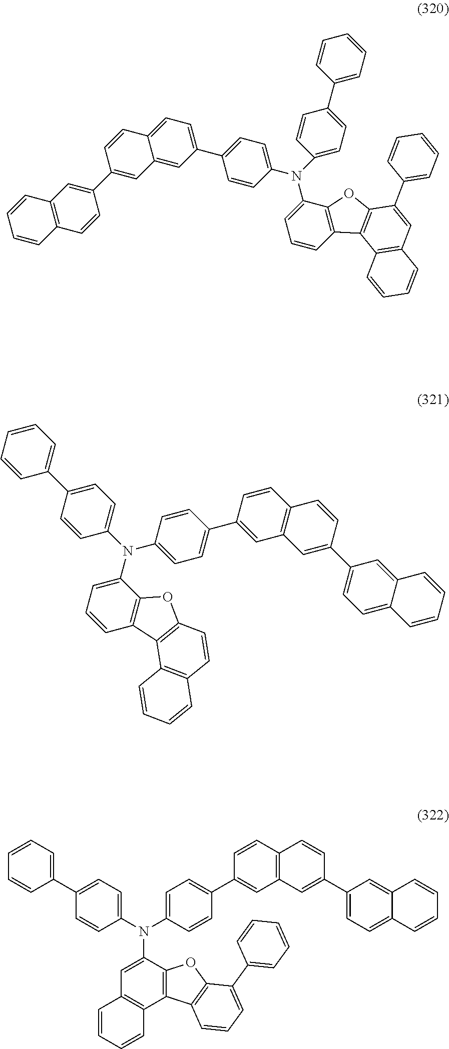

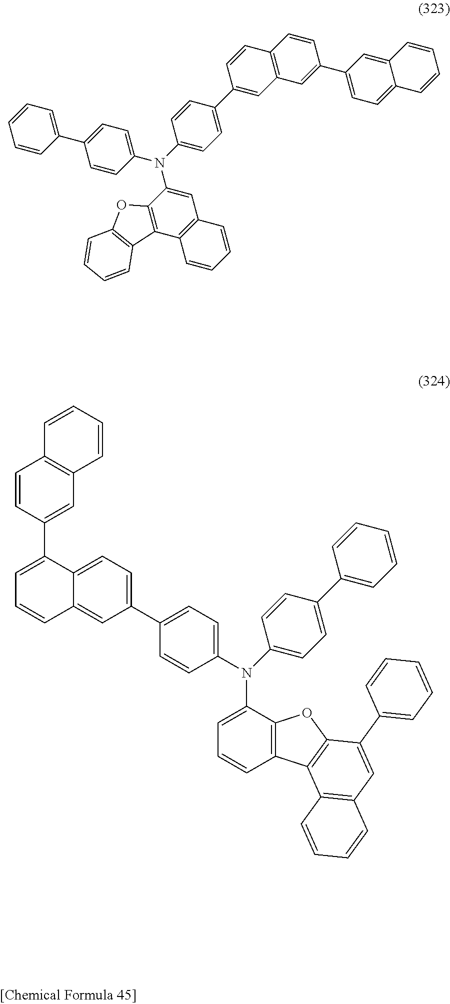

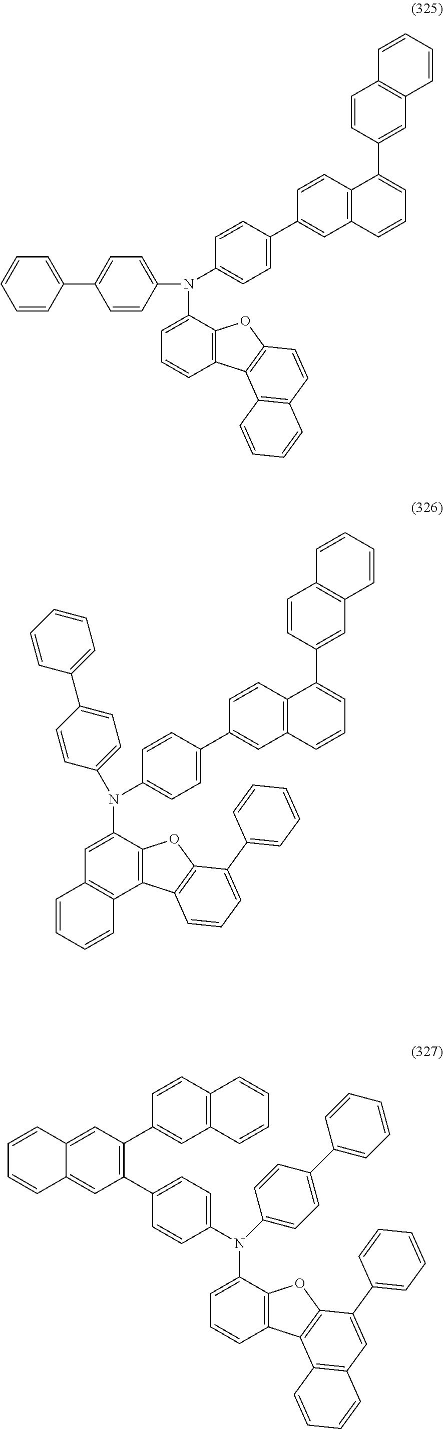

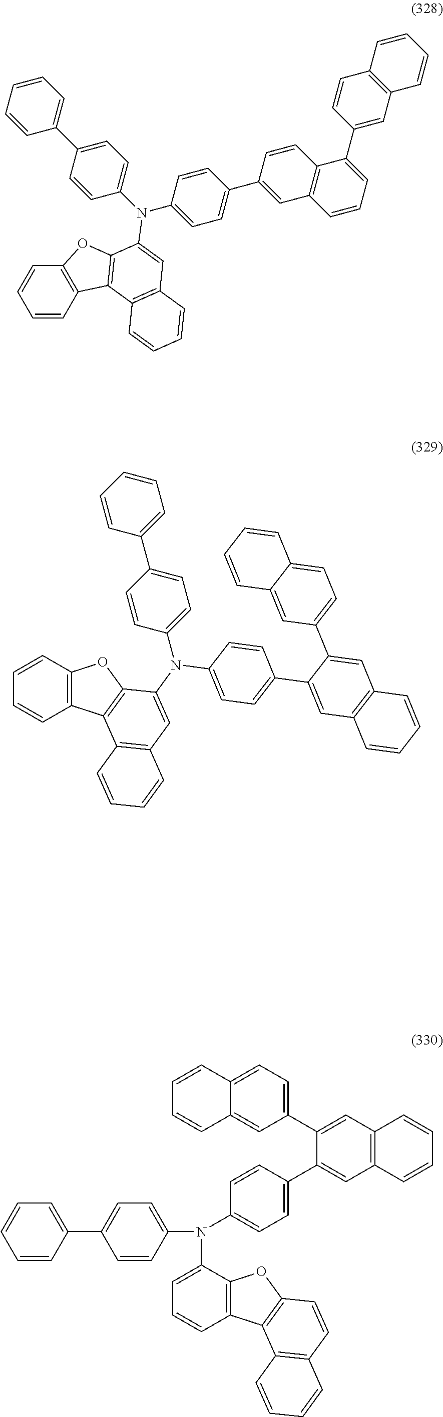

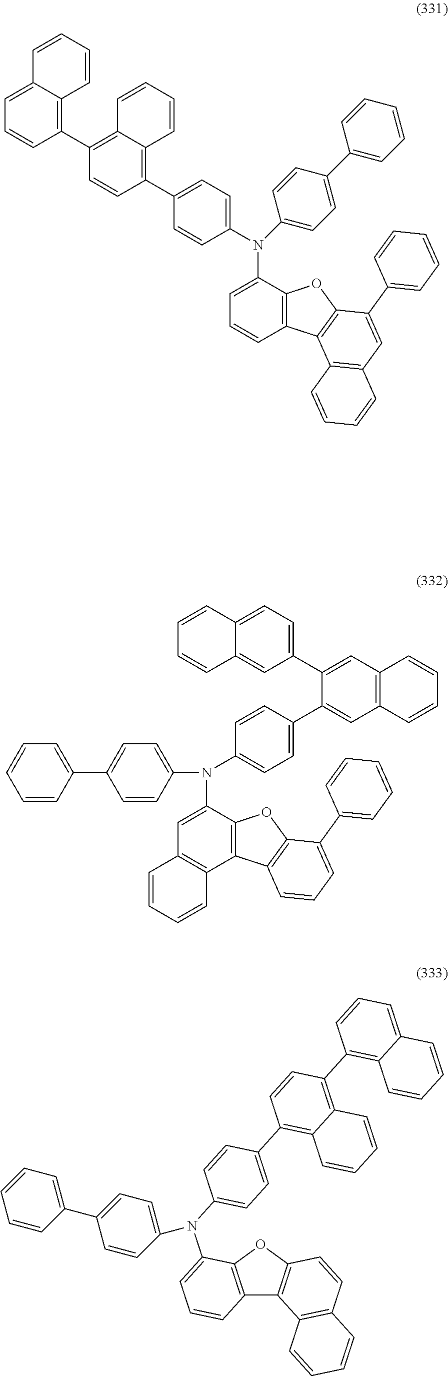

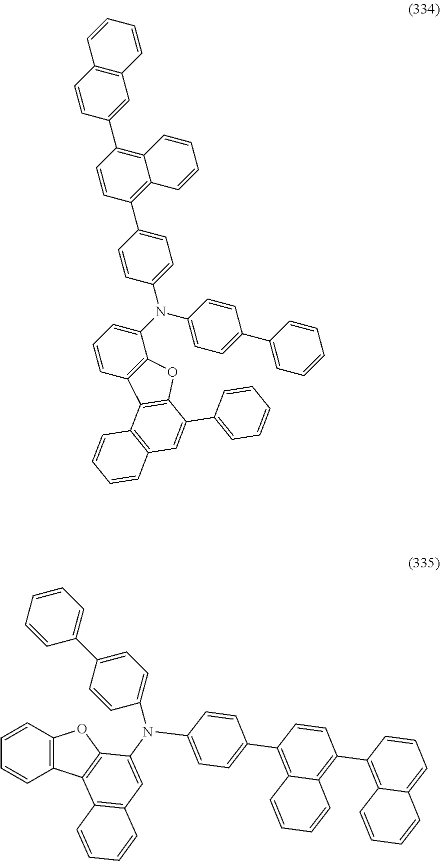

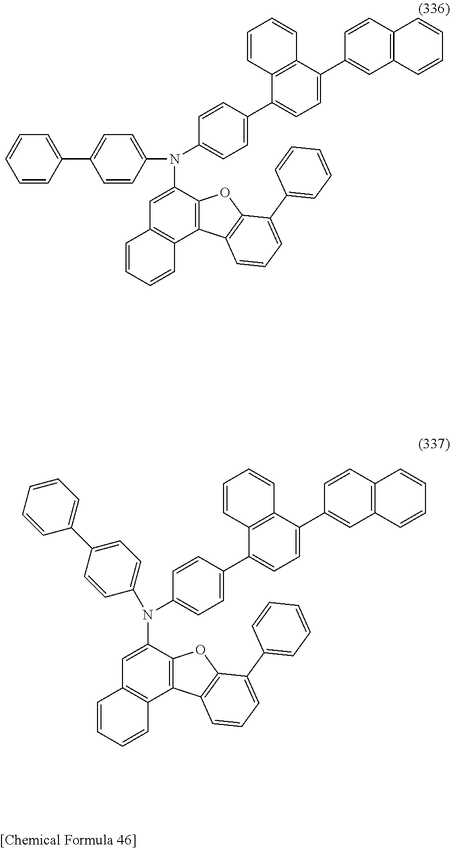

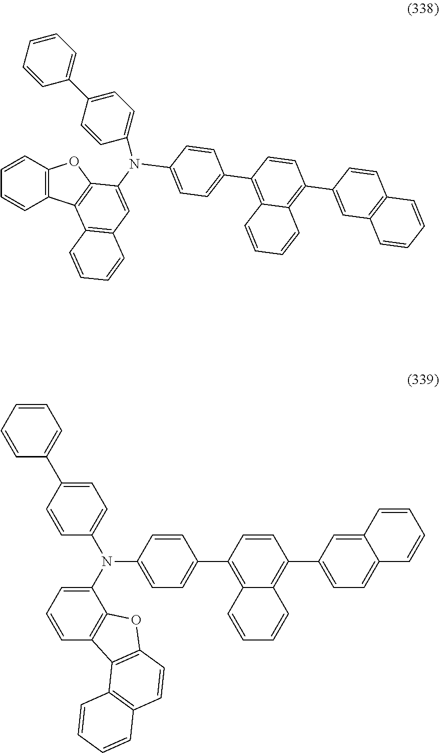

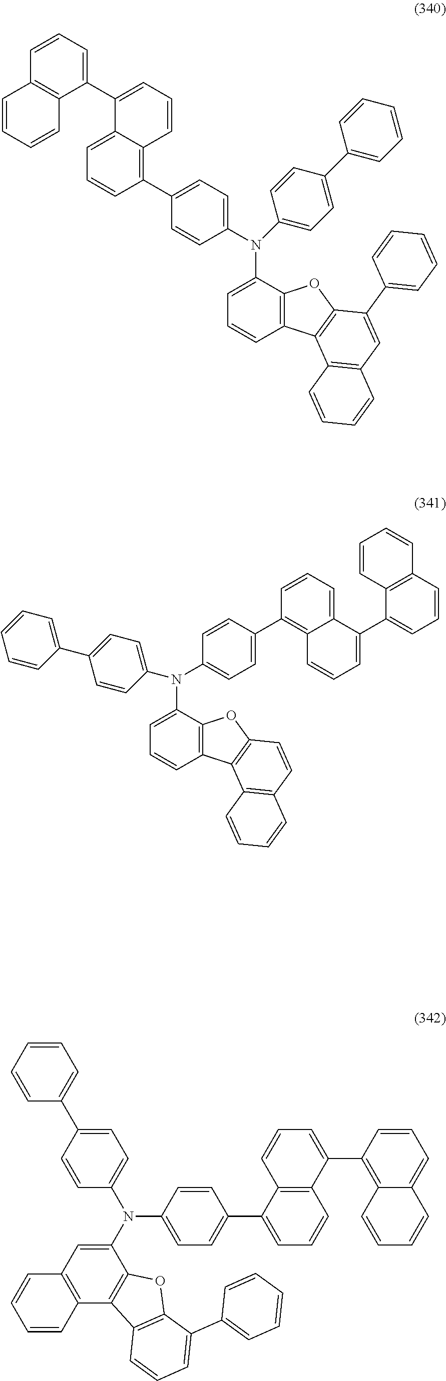

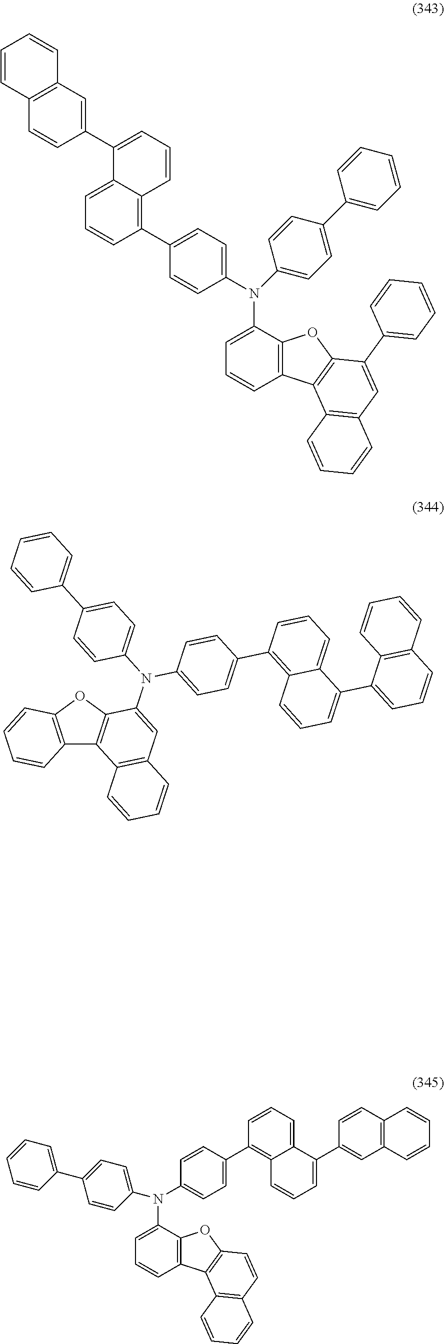

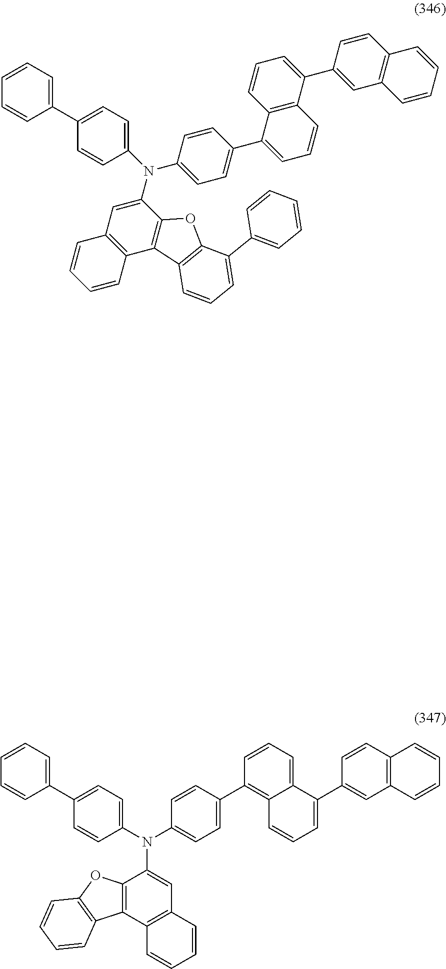

Next, as an example of a method of synthesizing the organic compound of one embodiment of the present invention, a method of synthesizing the organic compounds represented by General Formulae (G2) and (G3) will be described.

### <<Synthesis Method of Organic Compound Represented by General Formula (G2)>>

First, synthesis method of synthesizing an organic compound represented by General Formula (G2) will be described.

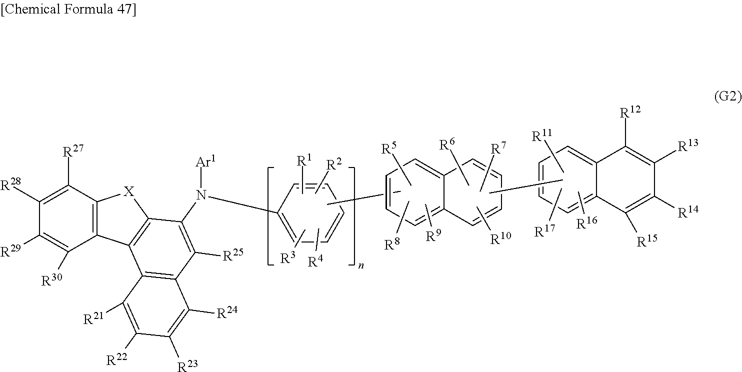

In General Formula (G2), Ar¹ represents a substituted or unsubstituted aryl group having 6 to 30 carbon atoms or a substituted or unsubstituted heteroaryl group having 2 to 30 carbon atoms; R¹ to R¹⁷ each independently represent any one of hydrogen (including deuterium), a substituted or unsubstituted alkyl group having 1 to 6 carbon atoms, and a substituted or unsubstituted cycloalkyl group having 3 to 10 carbon atoms; X represents oxygen or sulfur; R²¹ to R²⁵ and R²⁷ to R³⁰ each independently represent any one of hydrogen (including deuterium), a halogen, a cyano group, a substituted or unsubstituted alkyl group having 1 to 6 carbon atoms, a substituted or unsubstituted alkenyl group having 2 to 6 carbon atoms, a substituted or unsubstituted alkynyl group having 2 to 6 carbon atoms, a substituted or unsubstituted cycloalkyl group having 3 to 10 carbon atoms, a substituted or unsubstituted alkoxy group having 1 to 6 carbon atoms, a trialkylsilyl group having 3 to 10 carbon atoms, a substituted or unsubstituted aryl group having 6 to 30 carbon atoms, and a substituted or unsubstituted heteroaryl group having 2 to 30 carbon atoms; n represents an integer of 0 to 3; and in the case where n is 2 or more, a plurality of R¹ to R⁴ may be the same or different from each other.

### <<Synthesis Method 1 of Organic Compound Represented by General Formula (G2)>>

The organic compound represented by General Formula (G2) can be synthesized according to Synthesis Schemes (a-1-1) and (a-2) below.

First, according to Synthesis Scheme (a-1-1), an arylamine compound (Compound 1) is cross-coupled with a compound including a binaphthyl skeleton (Compound 2), whereby an amine compound including a binaphthyl skeleton (Compound 3) can be obtained.

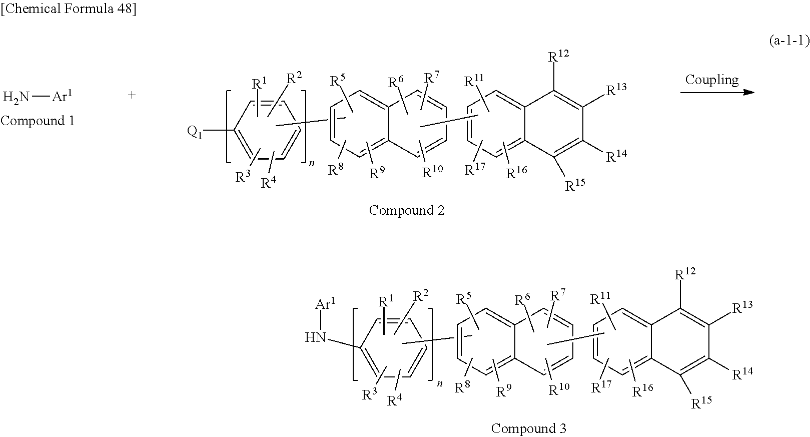

Next, according to Synthesis Scheme (a-2), the target organic compound represented by General Formula (G2) can be obtained by cross-coupling the amine compound including a binaphthyl skeleton (Compound 3) with a compound including a benzonaphthofuran skeleton or a benzonaphthothiophene skeleton (Compound 4).

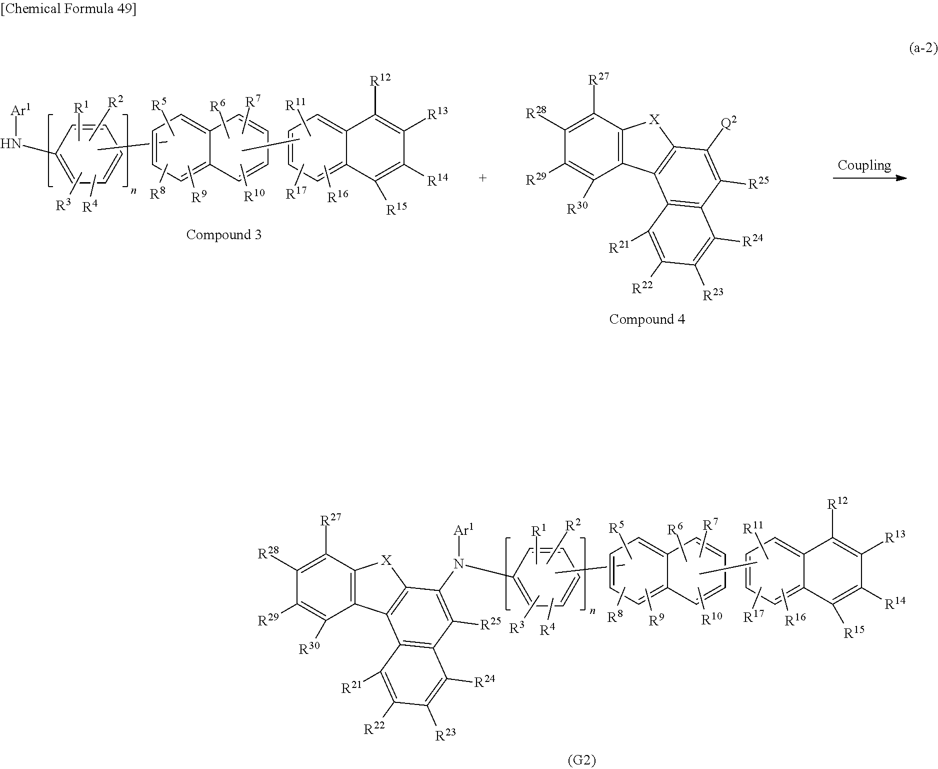

In Synthesis Schemes (a-1-1) and (a-2), Ar¹, R¹ to R⁴, R⁵ to R¹⁷, R²¹ to R²⁵, R²⁷ to R³⁰, X, and n are the same as those described above and are not described here. Each of Q¹ and Q² independently represents chlorine, bromine, iodine, or a triflate group.

In the case where the Buchwald-Hartwig reaction using a palladium catalyst is employed in Synthesis Schemes (a-1-1) and (a-2), a palladium compound such as bis(dibenzylideneacetone)palladium(0), palladium(II) acetate, [1,1-bis(diphenylphosphino)ferrocene]palladium(II) dichloride, tetrakis(triphenylphosphine)palladium(0), or allylpalladium(II) chloride (dimer) and a ligand such as tri(tert-butyl)phosphine, tri(n-hexyl)phosphine, tricyclohexylphosphine, di(1-adamantyl)-n-butylphosphine, 2-dicyclohexylphosphino-2′,6′-dimethoxybiphenyl(ortho-tolyl)phosphine, or (S)-(6,6′-dimethoxybiphenyl-2,2′-diyl)bis(diisopropylphosphine) (abbreviation: cBRIDP), can be used. In the reaction, an organic base such as sodium tert-butoxide, an inorganic base such as potassium carbonate, cesium carbonate, or sodium carbonate, or the like can be used. In the reaction, toluene, xylene, benzene, tetrahydrofuran, dioxane, or the like can be used as a solvent. Reagents that can be used in the reaction are not limited to the above-described reagents. Alternatively, a compound in which an organotin group is bonded to an amino group of Compound 1 can be used instead of Compound 1 or Compound 3.

In Synthesis Schemes (a-1-1) and (a-2), the Ullmann reaction using copper or a copper compound can also be performed. Copper or a copper compound can be used for the reaction. As an example of the base to be used in the reaction, an inorganic base such as potassium carbonate can be given. As an example of the solvent that can be used in the reaction, 1,3-dimethyl-3,4,5,6-tetrahydro-2(1H)-pyrimidinone (DMPU), toluene, xylene, benzene, and the like can be given. In the Ullmann reaction, when the reaction temperature is higher than or equal to 100° C., the target substance can be obtained in a shorter time in a higher yield; thus, it is preferable to use DMPU or xylene having a high boiling point. A higher reaction temperature such as a reaction temperature of 150° C. or higher is further preferable, and accordingly, DMPU is further preferably used. Reagents that can be used in the reaction are not limited to the above-described reagents.

Compound 3 can also be obtained by cross-coupling an aryl compound (Compound 5) with a compound including a binaphthyl skeleton (Compound 6) as shown in Synthesis Scheme (a-2-2) below.

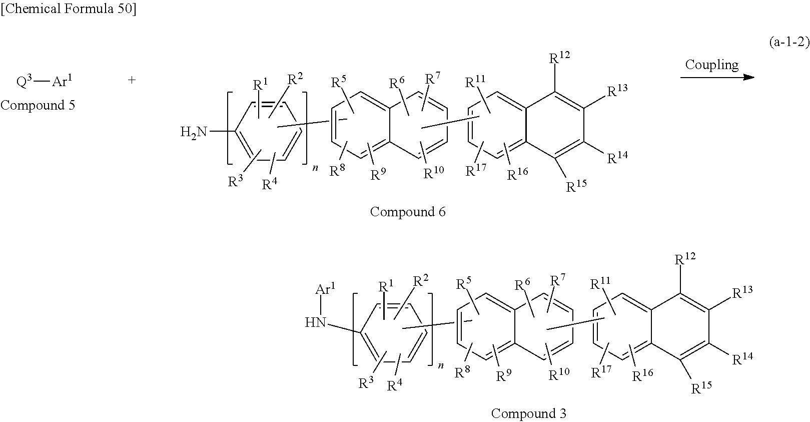

In Synthesis Scheme (a-1-2), Ar¹, R¹ to R⁴, R⁵ to R¹⁷, and n are the same as those described above and are not described here. Furthermore, Q³ represents chlorine, bromine, iodine, or a triflate group.

The same reaction conditions as those in Synthesis Schemes (a-1-1) and (a-2) can be used in Synthesis Scheme (a-1-2).

Note that in the case where n is 1 or more, Compound 3 can be obtained also by cross-coupling an arylamine compound (Compound 7) with a boron compound including a binaphthyl skeleton or a boronic acid including a binaphthyl skeleton (Compound 8) as shown in Synthesis Scheme (a-1-3) below.

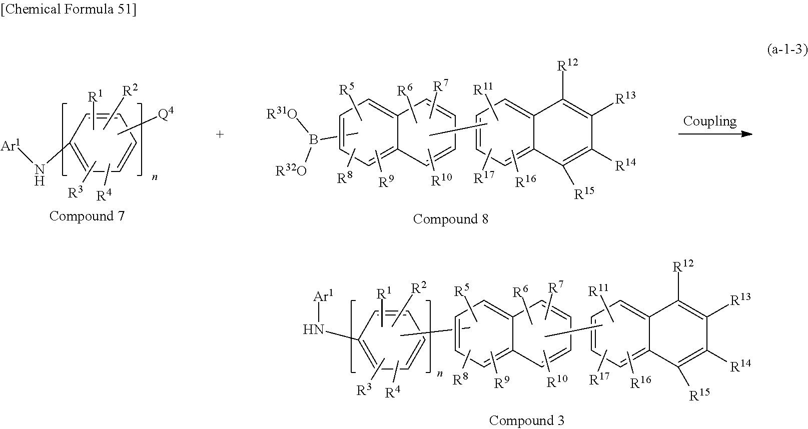

In Synthesis Scheme (a-1-3), Ar¹, R¹ to R⁴, and R⁵ to R¹⁷ are the same as those described above and are not described here. Note that n is an integer of 1 to 3. R³¹ and R³² each independently represent hydrogen or an alkyl group having 1 to 6 carbon atoms, the substituent R³¹ and the substituent R³² may be bonded to each other to form a ring, and examples of a boron compound in that case include pinacol borane. Furthermore, Q⁴ represents chlorine, bromine, iodine, or a triflate group.

In Synthesis Scheme (a-1-3), for example, palladium(II) acetate, tetrakis(triphenylphosphine)palladium(0), or bis(triphenylphosphine)palladium(II) dichloride can be used as a palladium catalyst.

In addition, for example, tri(ortho-tolyl)phosphine, triphenylphosphine, or tricyclohexylphosphine can be used as a ligand of the palladium catalyst.

As a base, an organic base such as sodium tert-butoxide, an inorganic base such as potassium carbonate or sodium carbonate, or the like can be used.

As a reaction solvent, a mixed solvent of toluene and water, a mixed solvent of xylene and water, a mixed solvent of benzene and water, a mixed solvent of water and an ether such as ethylene glycol dimethyl ether or 1,4-dioxane can be used. A boronic acid and a boron compound react at a higher rate and potentially bring about an effect of increasing the yield when dissolved in an aqueous phase; thus, it is preferable to add water. However, in the case where an ether is used for the solvent, a similar effect can be potentially brought about even when water is not added.

In addition, as the reaction solvent, a mixed solvent of toluene, water, and alcohol such as ethanol, a mixed solvent of xylene, water, and alcohol such as ethanol, a mixed solvent of benzene, water, and alcohol such as ethanol, or the like can be used. In particular, a mixed solvent of toluene and water, a mixed solvent of toluene, water, and ethanol, or a mixed solvent of water and an ether such as ethylene glycol dimethyl ether is preferable.

In the case where n is 1 or more, as shown in Synthesis Scheme (a-1-4), Compound 3 can be obtained also by cross-coupling an organoboron compound of an arylamine compound or a boronic acid of an arylamine compound (Compound 9) with a halide of a binaphthyl compound or a binaphthyl compound having a triflate group as a substituent (Compound 10) using the Suzuki-Miyaura reaction.

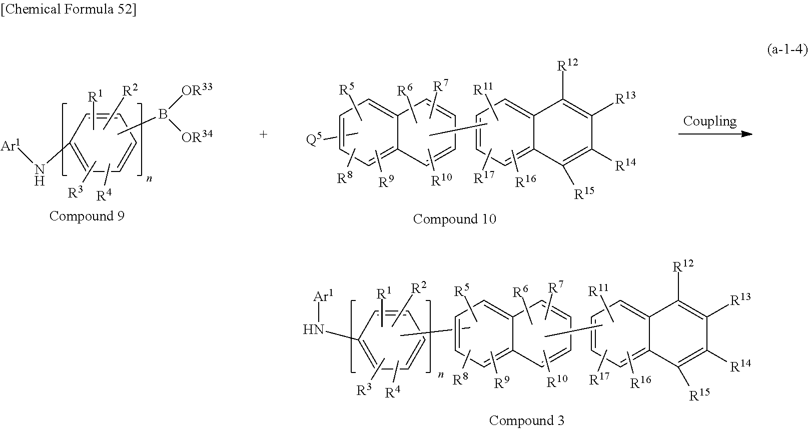

In Synthesis Scheme (a-1-4), Ar¹ and R¹ to R¹⁷ are the same as those described above and are not described here. Note that n is an integer of 1 to 3. Note that in Synthesis Scheme (a-1-4), R³³ and R³⁴ each independently represent hydrogen or an alkyl group having 1 to 6 carbon atoms, R³¹ and R³² may be bonded to each other to form a ring, and examples of a boron compound in that case include pinacol borane.

In Synthesis Scheme (a-1-4), Q⁵ represents chlorine, bromine, iodine, or a triflate group. The same reaction conditions as those in Synthesis Scheme (a-1-3) can be used in Synthesis Scheme (a-1-4).

Furthermore, instead of the organoboron compound or boronic acid, an organo aluminum compound, an organo zirconium compound, an organo zinc compound, an organo tin compound, or the like may be cross-coupled with a halide compound or a compound including a triflate group.

### <<Synthesis Method 2 of Organic Compound Represented by General Formula (G2)>>

The organic compound represented by General Formula (G2) can also be synthesized according to Synthesis Schemes (a-3-1) and (a-4) below.

First, the arylamine compound (Compound 1) is cross-coupled with the compound including a benzonaphthofuran skeleton or a benzonaphthothiophene skeleton (Compound 4), whereby Compound 11 can be obtained. The reaction formula is as shown in Synthesis Scheme (a-3-1).

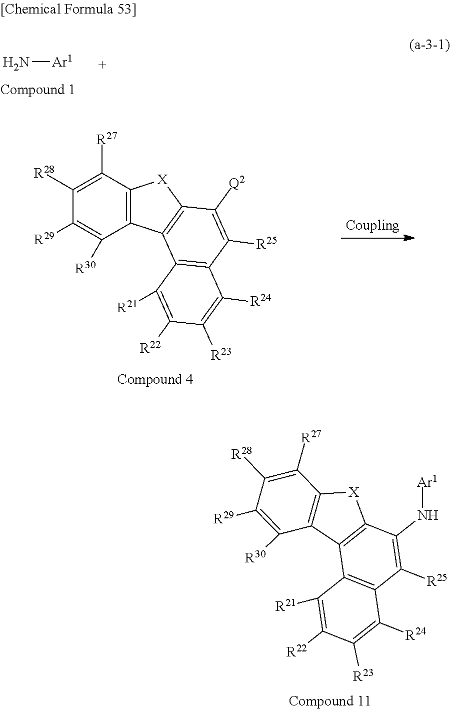

Next, Compound 11 is cross-coupled with the binaphthyl compound (Compound 2) according to Synthesis Scheme (a-4), whereby the target organic compound represented by General Formula (G2) can be obtained.

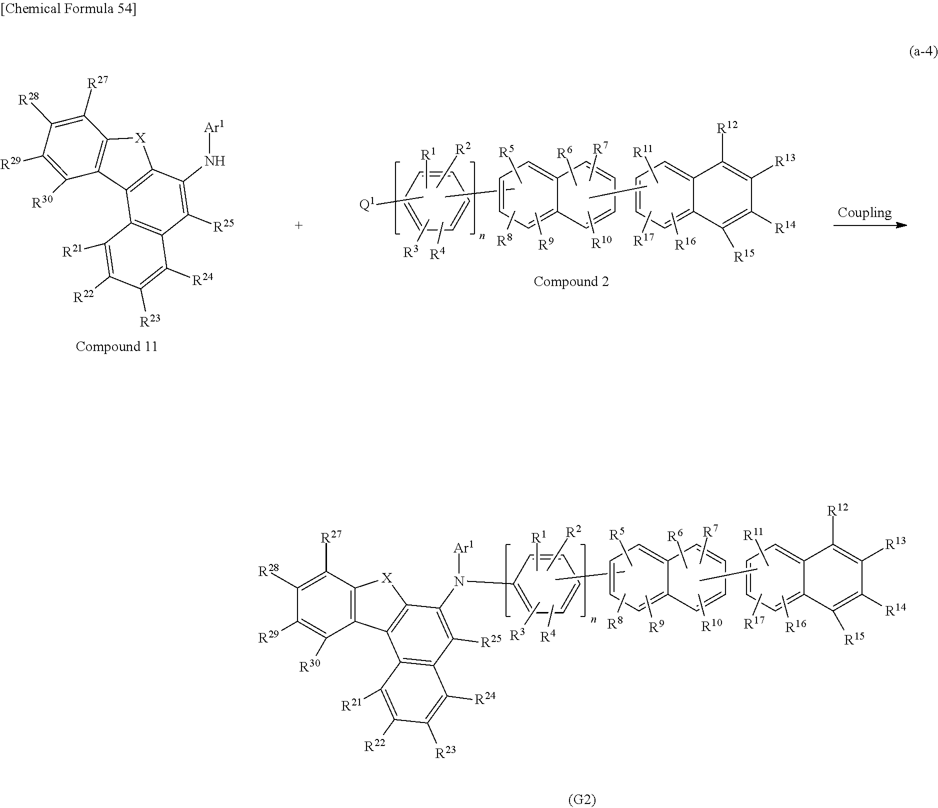

In Synthesis Schemes (a-3-1) and (a-4), Ar¹, R¹ to R⁴, R⁵ to R¹⁷, R²¹ to R²⁵, R²⁷ to R³⁰, X, and n are the same as those described above and are not described here.

In Synthesis Schemes (a-3-1) and (a-4), Q¹ and Q² each independently represent chlorine, bromine, iodine, or a triflate group.

In the case where the Buchwald-Hartwig reaction using a palladium catalyst or the Ullmann reaction using copper or copper oxide is performed in Synthesis Schemes (a-3-1) and (a-4), the same reaction conditions as those in Synthesis Schemes (a-1-1) and (a-2) can be used.

Compound 11 can be obtained also by cross-coupling the aryl compound (Compound 5) with a primary amine compound (Compound 12) as shown in Synthesis Scheme (a-3-2) below.

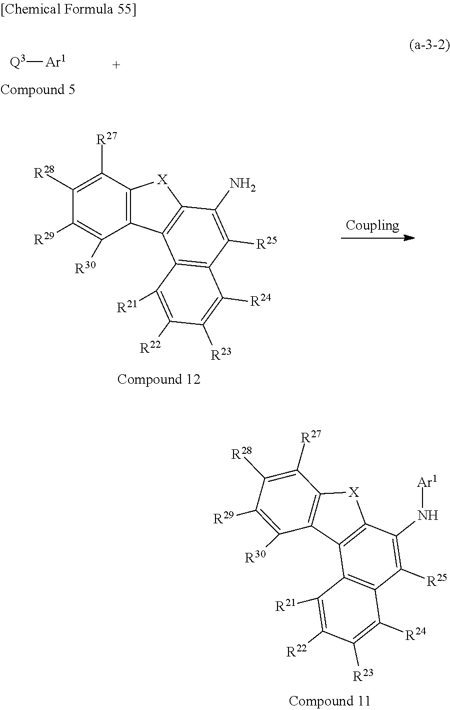

In Synthesis Scheme (a-3-2), Ar¹, R²¹ to R²⁵, and R²⁷ to R³⁰, and X are the same as those described above and are not described here.

In Synthesis Scheme (a-3-2), Q³ represents chlorine, bromine, iodine, or a triflate group. The same reaction conditions as those in Synthesis Schemes (a-1-1) and (a-2) can be used in Synthesis Scheme (a-3-2).

### <<Synthesis Method 3 of Organic Compound Represented by General Formula (G2)>>

The organic compound represented by General Formula (G2) can also be synthesized according to Synthesis Schemes (a-5-1) to (a-6) below.

First, a primary amine compound including a binaphthyl skeleton (Compound 3) is cross-coupled with the compound including a benzonaphthofuran skeleton or a benzonaphthothiophene skeleton (Compound 4) according to Reaction Formula (a-5-1), whereby Compound 13 can be obtained.

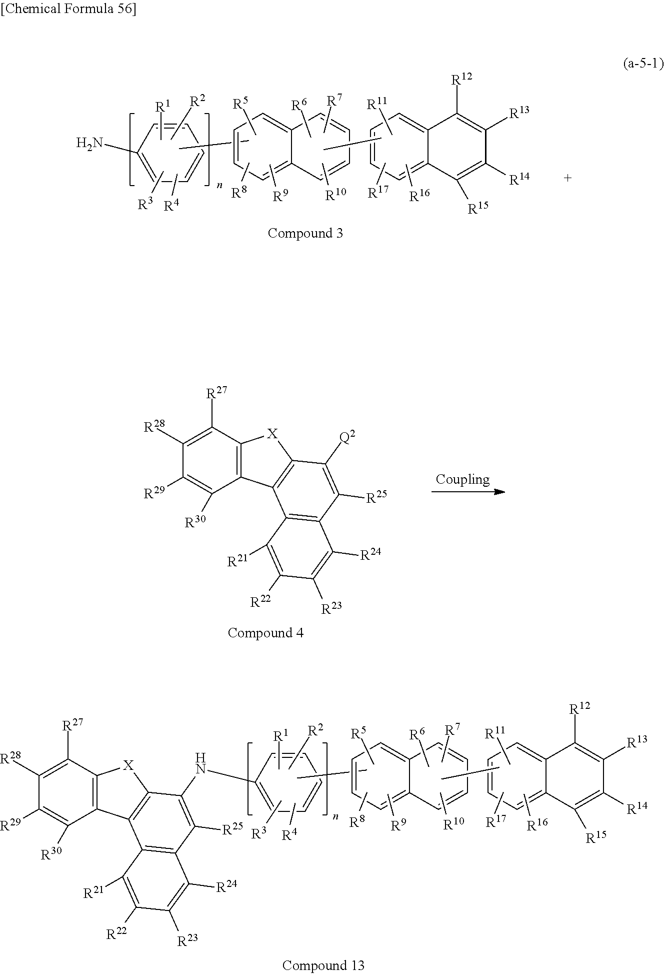

Next, the aryl compound (Compound 5) is cross-coupled with Compound 13 according to Reaction Formula (a-6), whereby the target organic compound represented by General Formula (G2) can be obtained.

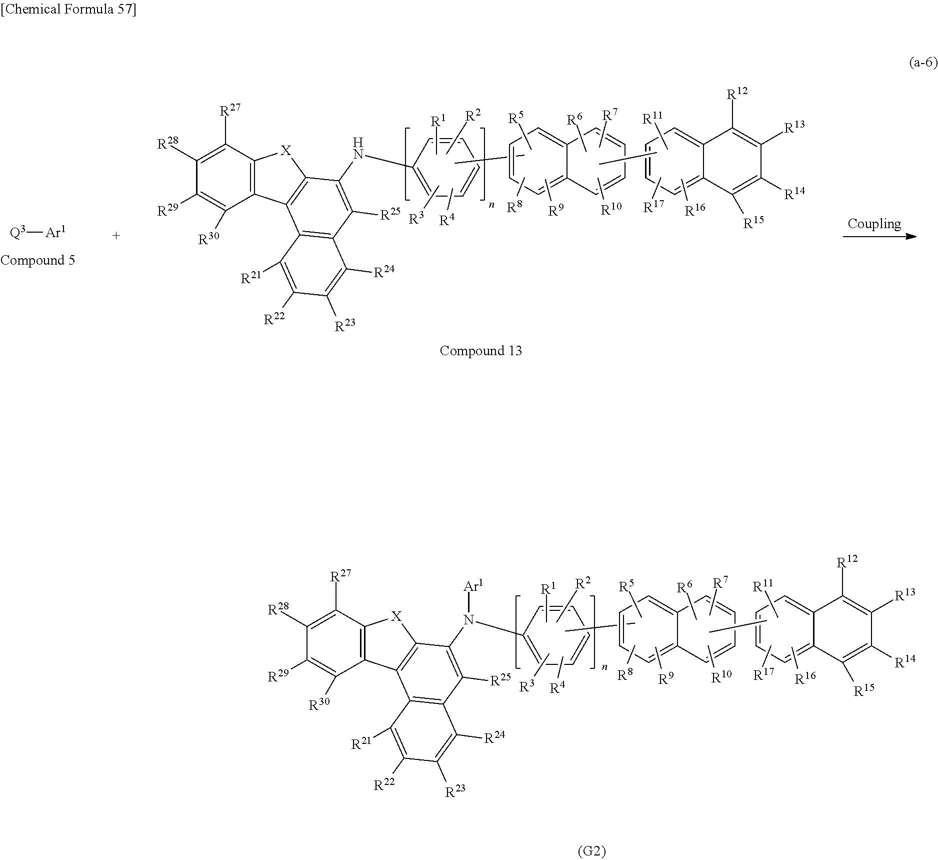

In Synthesis Schemes (a-5-1) and (a-6), Ar¹, R¹ to R⁴, R⁵ to R¹⁷, R²¹ to R²⁵, R²⁷ to R³⁰, X, and n are the same as those described above and are not described here.

In Synthesis Schemes (a-5-i) and (a-6), Q² and Q³ each independently represent chlorine, bromine, iodine, or a triflate group.

In the case where the Buchwald-Hartwig reaction using a palladium catalyst or the Ullmann reaction using copper or copper oxide is performed in Synthesis Schemes (a-5-1) and (a-6), the same reaction conditions as those in Synthesis Schemes (a-1-1) and (a-2) can be used.

Note that Compound 13 can be obtained also by cross-coupling the binaphthyl compound (Compound 2) and the primary amine compound (Compound 12) as shown in Synthesis Scheme (a-5-2) below.

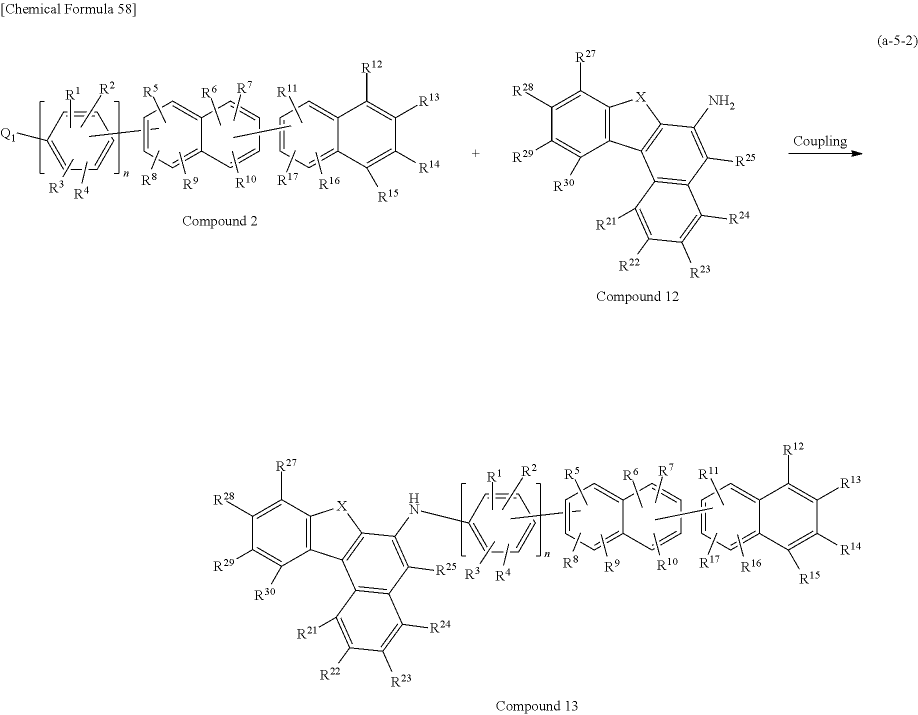

In Synthesis Scheme (a-5-2), Ar¹, R¹ to R⁴, R⁵ to R¹⁷, R²¹ to R²⁵, R²⁷ to R³⁰, X, and n are the same as those described above and are not described here.

In Synthesis Scheme (a-5-2), Q¹ represents chlorine, bromine, iodine, or a triflate group. The same reaction conditions as those in Synthesis Schemes (a-1-1) and (a-2) can be used in Synthesis Scheme (a-5-2).

Note that in the case where n is 1 or more, Compound 13 can be obtained also by cross-coupling a primary amine compound (Compound 14) with the boron compound including a binaphthyl skeleton or a boronic acid including a binaphthyl skeleton (Compound 8) as shown in Synthesis Scheme (a-5-3) below.

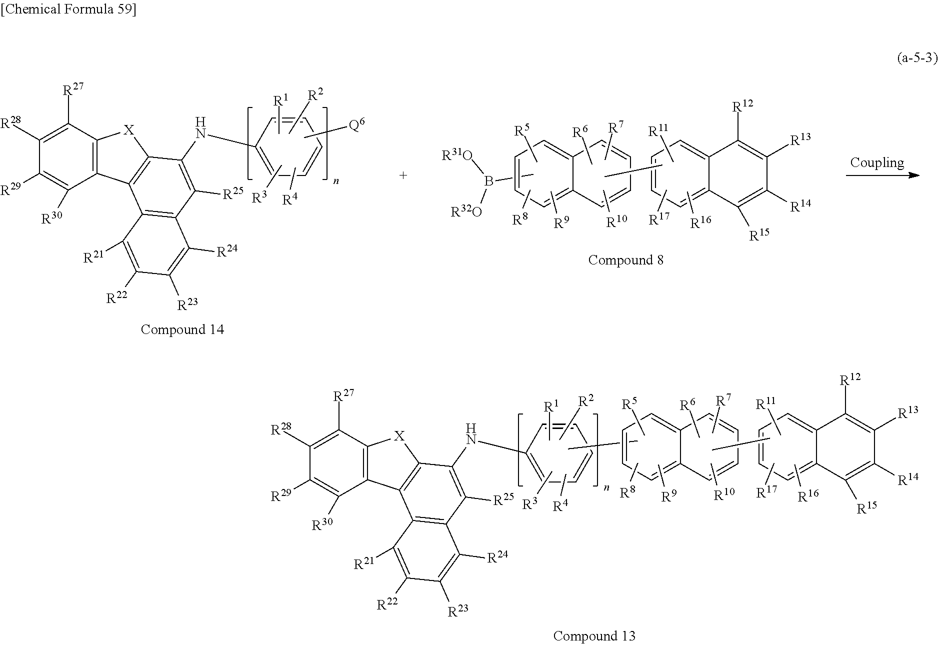

In Synthesis Scheme (a-5-3), Ar¹, R¹ to R¹⁷, R²¹ to R²⁵, R²⁷ to R³², and X are the same as those described above and are not described here. Note that n is an integer of 1 to 3. Furthermore, Q⁶ represents chlorine, bromine, iodine, or a triflate group. The same reaction conditions as those in Synthesis Scheme (a-1-3) can be used in Synthesis Scheme (a-5-3).

In the case where n is 1 or more, as shown in Synthesis Scheme (a-5-4) below, Compound 13 can be obtained also by cross-coupling an organoboron compound of a secondary amine compound or a boronic acid of a secondary amine compound (Compound 15) with the halide of a binaphthyl compound or the binaphthyl compound having a triflate group as a substituent (Compound 10) using the Suzuki-Miyaura reaction.

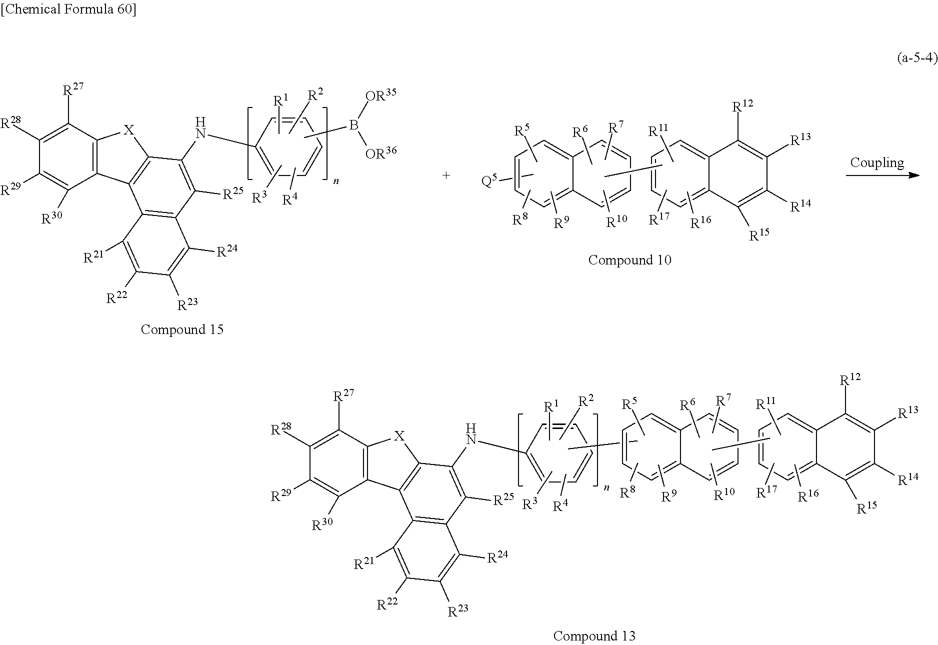

In Synthesis Scheme (a-5-4), Ar¹, R¹ to R¹⁷, R²¹ to R²⁵, R²⁷ to R³⁰, and X are the same as those described above and are not described here. Note that n is an integer of 1 to 3. R³⁵ to R³⁶ each independently represent hydrogen or an alkyl group having 1 to 6 carbon atoms, R³⁵ and R³⁶ may be bonded to each other to form a ring, and examples of a boron compound in that case include pinacol borane.

In Synthesis Scheme (a-5-4), Q⁵ represents chlorine, bromine, iodine, or a triflate group. The same reaction conditions as those in Synthesis Scheme (a-5-4) can be used in Synthesis Scheme (a-1-3).

A cross-coupling reaction using an organoaluminum, organozirconium, organozinc, or organotin compound or the like instead of the organoboron compound or boronic acid may be employed.

The above is the description on synthesis method of organic compound represented by General Formula (G2). Note that the synthesis method of the organic compound represented by the general formula (G2) is not limited to the above-described synthesis method.

### <Synthesis Method of Organic Compound Represented by General Formula (G3)>

Next, a synthesis method of the organic compound represented by General Formula (G3) will be described.

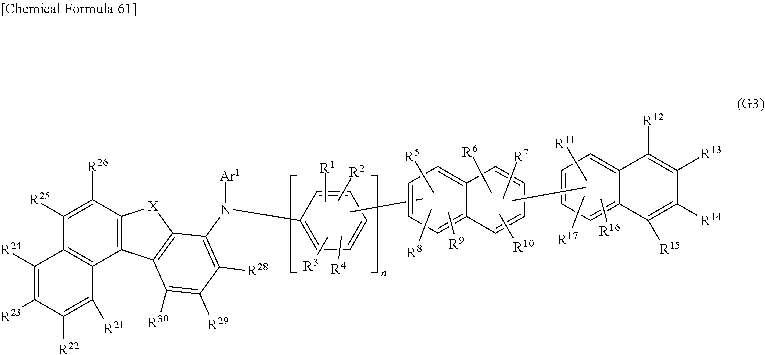

In General Formula (G3), Ar¹ represents a substituted or unsubstituted aryl group having 6 to 30 carbon atoms or a substituted or unsubstituted heteroaryl group having 2 to 30 carbon atoms; R¹ to R¹⁷ each independently represent any one of hydrogen (including deuterium), a substituted or unsubstituted alkyl group having 1 to 6 carbon atoms, and a substituted or unsubstituted cycloalkyl group having 3 to 10 carbon atoms; X represents oxygen or sulfur; R²¹ to R²⁶ and R²⁸ to R³⁰ each independently represent any one of hydrogen (including deuterium), a halogen, a cyano group, a substituted or unsubstituted alkyl group having 1 to 6 carbon atoms, a substituted or unsubstituted alkenyl group having 2 to 6 carbon atoms, a substituted or unsubstituted alkynyl group having 2 to 6 carbon atoms, a substituted or unsubstituted cycloalkyl group having 3 to 10 carbon atoms, a substituted or unsubstituted alkoxy group having 1 to 6 carbon atoms, a trialkylsilyl group having 3 to 10 carbon atoms, a substituted or unsubstituted aryl group having 6 to 30 carbon atoms, and a substituted or unsubstituted heteroaryl group having 2 to 30 carbon atoms; n represents an integer of 0 to 3; and in the case where n is 2 or more, a plurality of R¹ to R⁴ may be the same or different from each other.

### <<Synthesis Method 1 of Organic Compound Represented by General Formula (G3)>>

According to Synthesis Scheme (b-1), the target organic compound represented by General Formula (G3) can be obtained by cross-coupling the amine compound including a binaphthyl skeleton (Compound 3) with a compound including a benzonaphthofuran skeleton or a benzonaphthothiophene skeleton (Compound 16). Note that the synthesis method of Compound 3 is shown in the synthesis method of the organic compound represented by General Formula (G2) and are not described here.

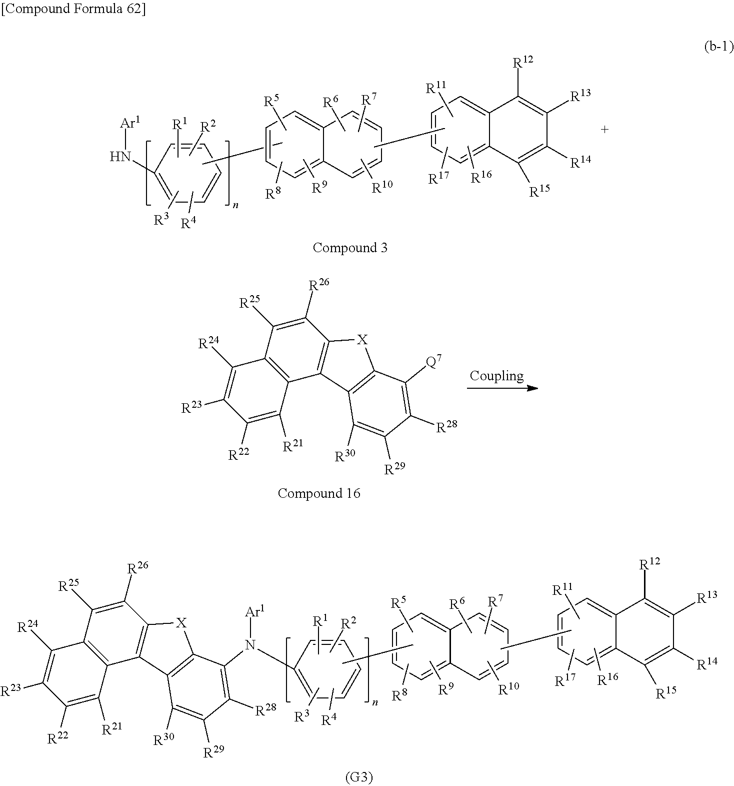

In Synthesis Scheme (b-1), Ar¹, R¹ to R⁴, R⁵ to R¹⁷, R²¹ to R²⁶, R²⁸ to R³⁰, X, and n are the same as those described above and are not described here. Reagents and conditions that can be used in Synthesis Scheme (b-1) are similar to those in Synthesis Scheme (a-2) and are not described here.

### <<Synthesis Method 2 of Organic Compound Represented by General Formula (G3)>>

The organic compound represented by General Formula (G3) can be synthesized according to Synthesis Schemes (b-2-1) and (b-3).

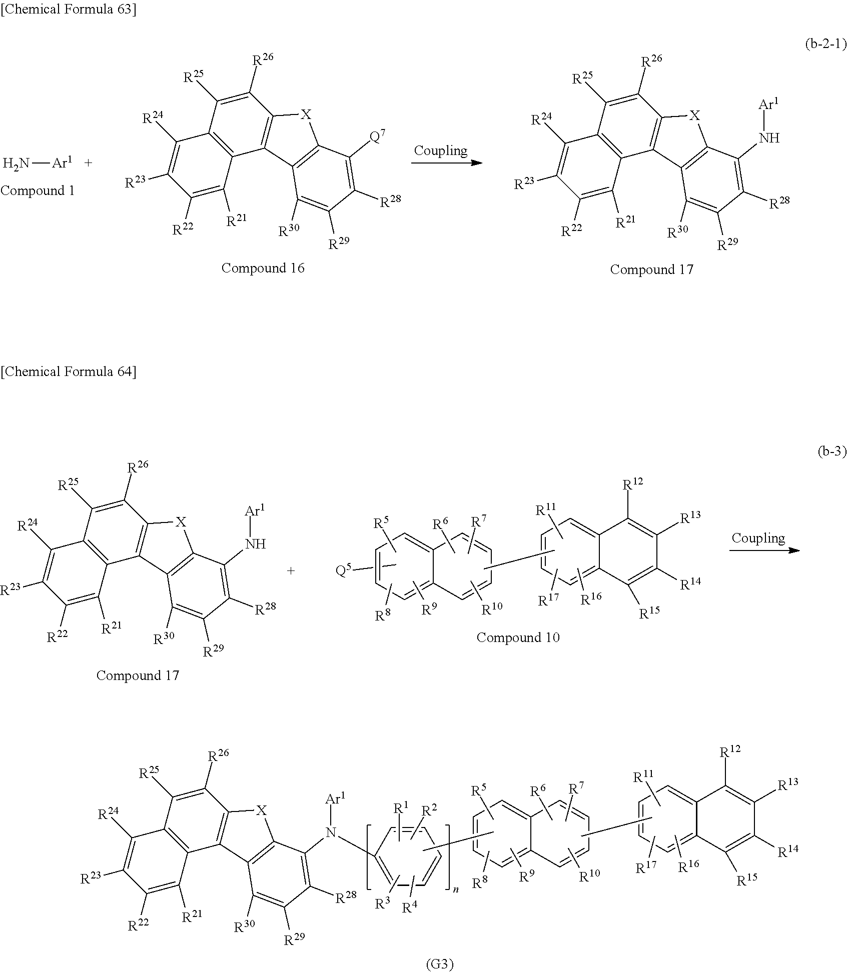

In Synthesis Scheme (b-2-1), R¹ to R⁴, R⁵ to R¹⁷, R²¹ to R²⁶, R²⁸ to R³⁰, X, and n are the same as those described above and are not described here. Reagents and conditions that can be used in Synthesis Scheme (b-2-1) are similar to those in Synthesis Scheme (a-3-1), reagents and conditions that can be used in Synthesis Scheme (b-3) are similar to those in Synthesis Scheme (a-4), and thus the description thereof is omitted here.

Compound 17 can be obtained also by cross-coupling the aryl compound (Compound 5) with a benzonaphthofuranylamine compound (Compound 18) as shown in Synthesis Scheme (b-2-2).

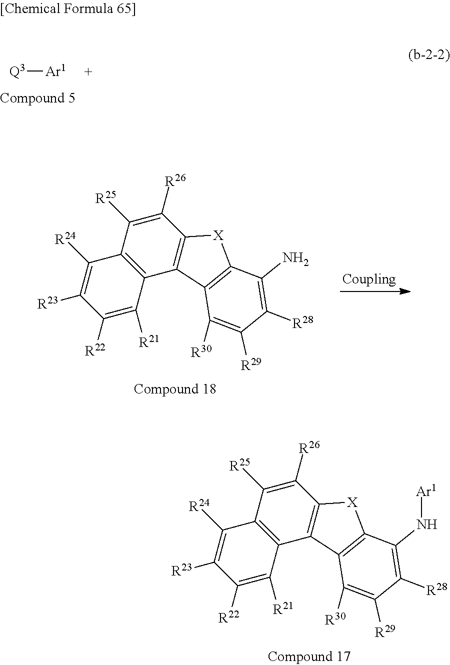

In Synthesis Scheme (b-2-2), Ar¹, R²¹ to R²⁶, and R²⁸ to R³⁰, and X are the same as those described above and are not described here. Reagents and conditions that can be used in Synthesis Scheme (b-2-2) are similar to those in Synthesis Scheme (a-3-2) and are not described here.

### <<Synthesis Method 3 of Organic Compound Represented by General Formula (G3)>>

The organic compound represented by General Formula (G3) can be synthesized according to Synthesis Schemes (b-4-1) and (b-5).

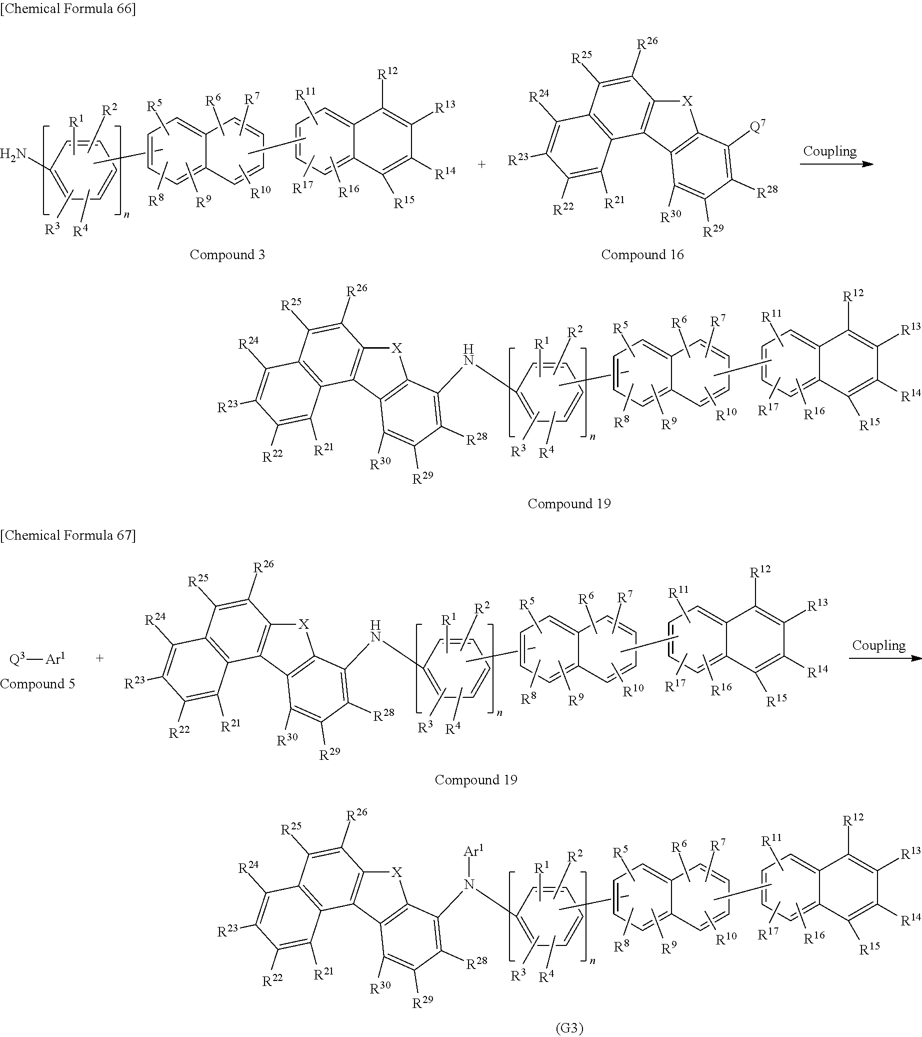

In Synthesis Schemes (b-4-1) and (b-5), Ar¹, R¹ to R⁴, R⁵ to R¹⁷, R²¹ to R²⁶, R²¹ to R³⁰, X, and n are the same as those described above and are not described here. Reagents and conditions that can be used in Synthesis Scheme (b-4-1) are similar to those in Synthesis Scheme (a-5-1), reagents and conditions that can be used in Synthesis Scheme (b-5) are similar to those in Synthesis Scheme (a-6), and thus the description thereof is omitted here.

Note that Compound 19 can be obtained also by cross-coupling a binaphthyl compound (Compound 2) with a benzonaphthofuranylamine compound (Compound 18) as shown in Synthesis Scheme (b-4-2) below.

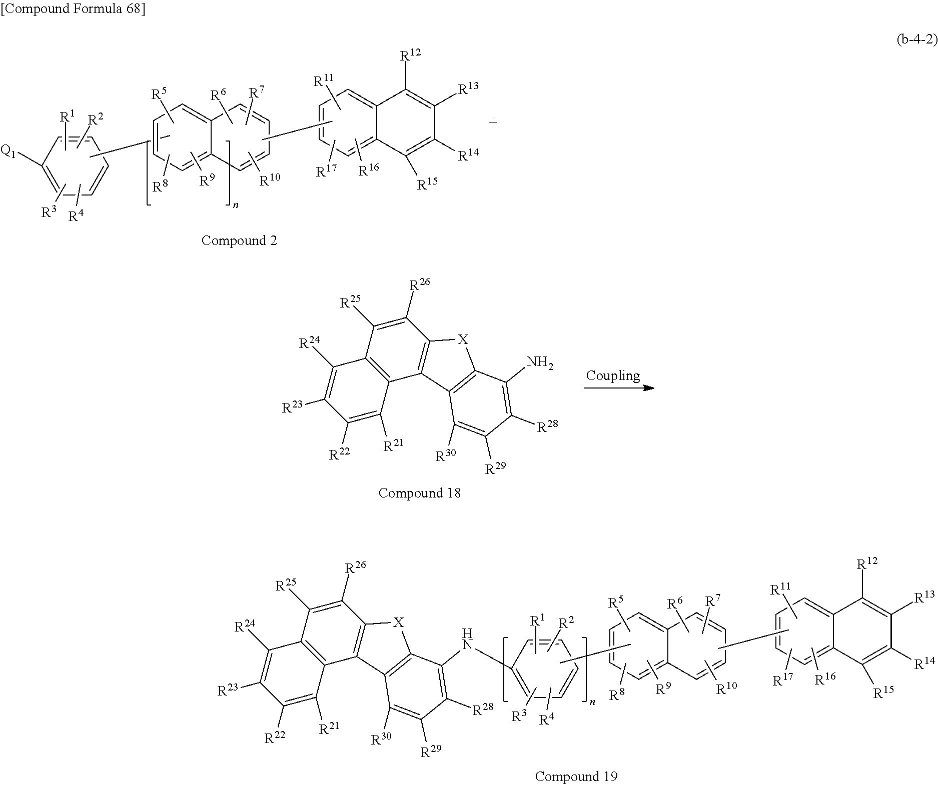

In Synthesis Scheme (b-4-2), Ar³, R¹ to R⁴, R⁵ to R¹⁷, R²¹ to R²⁶, R²⁸ to R³⁰, X, and n are the same as those described above and are not described here. Reagents and conditions that can be used in Synthesis Scheme (b-4-2) are similar to those in Synthesis Scheme (a-5-2) and are not described here.

In the case where n is 1 or more, Compound 19 can be obtained also by cross-coupling a benzonaphthofuranamine compound (Compound 20) with a boronic compound including a binaphthyl skeleton or a boronic acid including a binaphthyl skeleton (Compound 8) as shown in Synthesis Schemes (b-4-3).

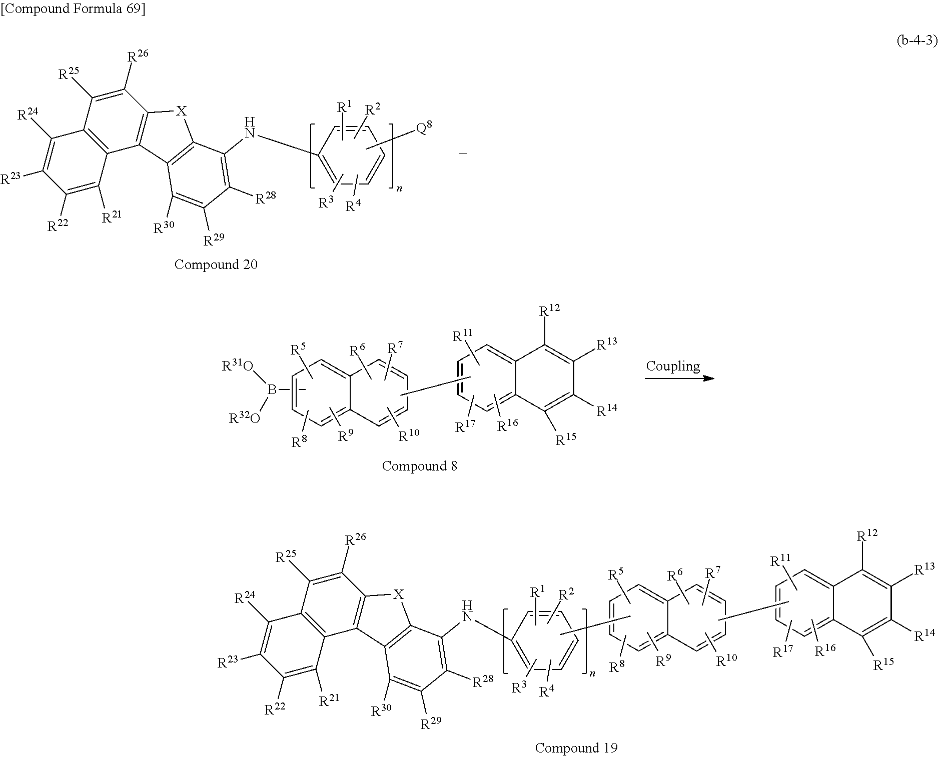

In Synthesis Scheme (b-4-3), Ar¹, R¹ to R¹⁷, R²¹ to R²⁶, R²⁸ to R³², and X are the same as those described above and are not described here. Note that n is an integer of 1 to 3. Reagents and conditions that can be used in Synthesis Scheme (b-4-3) are similar to those in Synthesis Scheme (a-5-3) and are not described here.

In the case where n is 1 or more, as shown in Synthesis Scheme (b-4-4), Compound 19 can be obtained also by cross-coupling an organoboron compound of an arylamine compound or a boronic acid of an arylamine compound (a compound 21) with a halide of a binaphthyl compound or a binaphthyl compound having a triflate group as a substituent (a compound 10) using the Suzuki-Miyaura reaction.

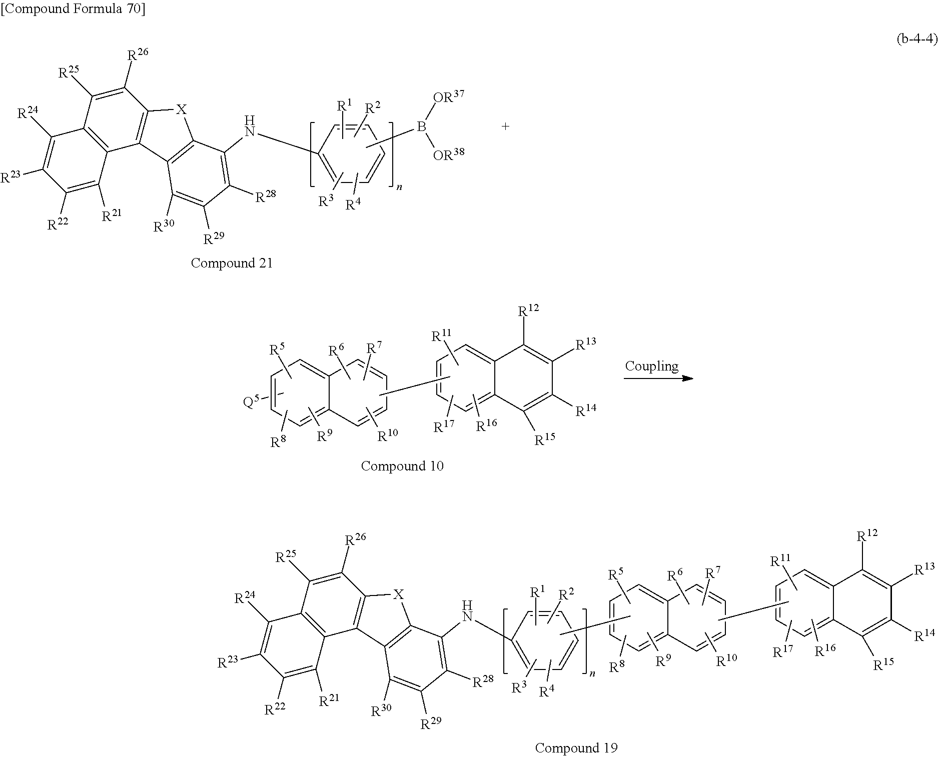

In Synthesis Scheme (b-4-4), Ar¹, R¹ to R¹⁷, R²¹ to R²⁶, R²⁸ to R³⁰, R³⁷, R³⁸, and X are the same as those described above and are not described here. Note that n is an integer of 1 to 3. Reagents and conditions that can be used in Synthesis Scheme (b-4-4) are similar to those in Synthesis Scheme (a-5-4) and are not described here.

In the above manner, the organic compound represented by General Formula (G3) can be synthesized. Note that the organic compound represented by General Formula (G3) and Ar¹, R¹ to R¹⁷, R²¹ to R²⁶, R²⁸ to R³⁴, Q¹, Q³ to Q⁵, Q⁷, Q⁸, X, and n in Synthesis Schemes (b-1) to (b-4-4) are the same as those described above and are not described here. Note that in Synthesis Scheme (b-4-4), R³⁷ and R³⁸ each independently represent hydrogen or an alkyl group having 1 to 6 carbon atoms, R³⁷ and R³¹ may be bonded to each other to form a ring, and examples of a boron compound in that case include pinacol borane.

The above is the description on the synthesis methods of the organic compounds of embodiments of the present invention. Note that the synthesis methods of the organic compounds of embodiments of the present invention are not limited thereto, and a given reaction and a given reagent other than those in the above description can be employed.

Specific examples of Compound 11, Compound 17, Compound 13, and Compound 19 in the above synthesis methods are shown in the following structural formulae. Note that specific examples of Compound 11, Compound 17, Compound 13, and Compound 19 are not limited thereto.

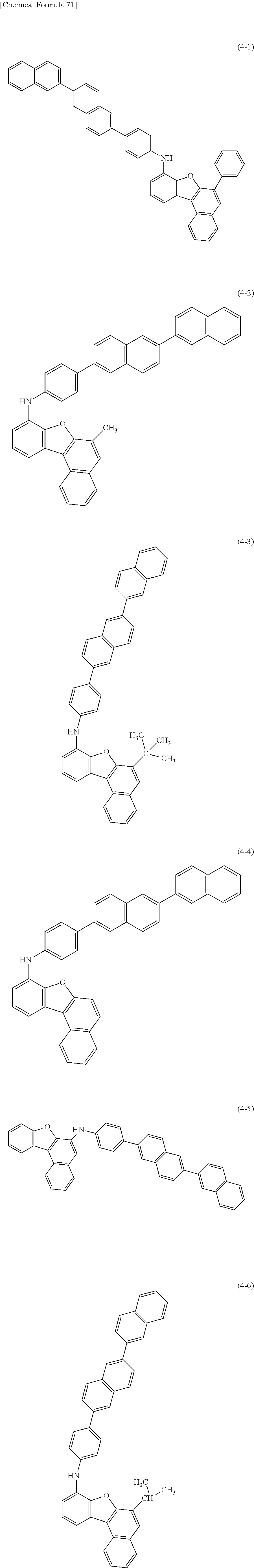 [SMILES: c1ccc(-c2cc3ccccc3c3c2oc2c(Nc4ccc(-c5ccc6cc(-c7ccc8ccccc8c7)ccc6c5)cc4)cccc23)cc1 ; Cc1cc2ccccc2c2c1oc1c(Nc3ccc(-c4ccc5cc(-c6ccc7ccccc7c6)ccc5c4)cc3)cccc12 ; CC(C)(C)c1cc2ccccc2c2c1oc1c(Nc3ccc(-c4ccc5cc(-c6ccc7ccccc7c6)ccc5c4)cc3)cccc12 ; c1ccc2cc(-c3ccc4cc(-c5ccc(Nc6cccc7c6oc6ccc8ccccc8c67)cc5)ccc4c3)ccc2c1 ; c1ccc2cc(-c3ccc4cc(-c5ccc(Nc6cc7ccccc7c7c6oc6ccccc67)cc5)ccc4c3)ccc2c1 ; CC(C)c1cc2ccccc2c2c1oc1c(Nc3ccc(-c4ccc5cc(-c6ccc7ccccc7c6)ccc5c4)cc3)cccc12]

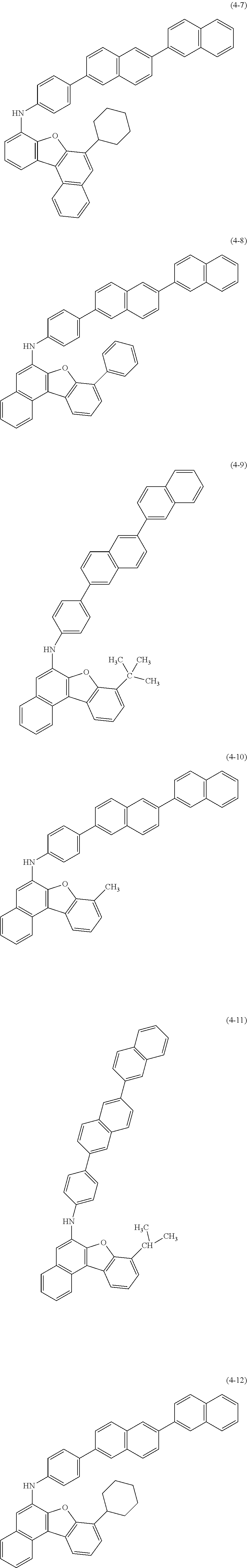 [SMILES: c1ccc2cc(-c3ccc4cc(-c5ccc(Nc6cccc7c6oc6c(C8CCCCC8)cc8ccccc8c67)cc5)ccc4c3)ccc2c1 ; c1ccc(-c2cccc3c2oc2c(Nc4ccc(-c5ccc6cc(-c7ccc8ccccc8c7)ccc6c5)cc4)cc4ccccc4c23)cc1 ; CC(C)(C)c1cccc2c1oc1c(Nc3ccc(-c4ccc5cc(-c6ccc7ccccc7c6)ccc5c4)cc3)cc3ccccc3c12 ; Cc1cccc2c1oc1c(Nc3ccc(-c4ccc5cc(-c6ccc7ccccc7c6)ccc5c4)cc3)cc3ccccc3c12 ; CC(C)c1cccc2c1oc1c(Nc3ccc(-c4ccc5cc(-c6ccc7ccccc7c6)ccc5c4)cc3)cc3ccccc3c12 ; c1ccc2cc(-c3ccc4cc(-c5ccc(Nc6cc7ccccc7c7c6oc6c(C8CCCCC8)cccc67)cc5)ccc4c3)ccc2c1]

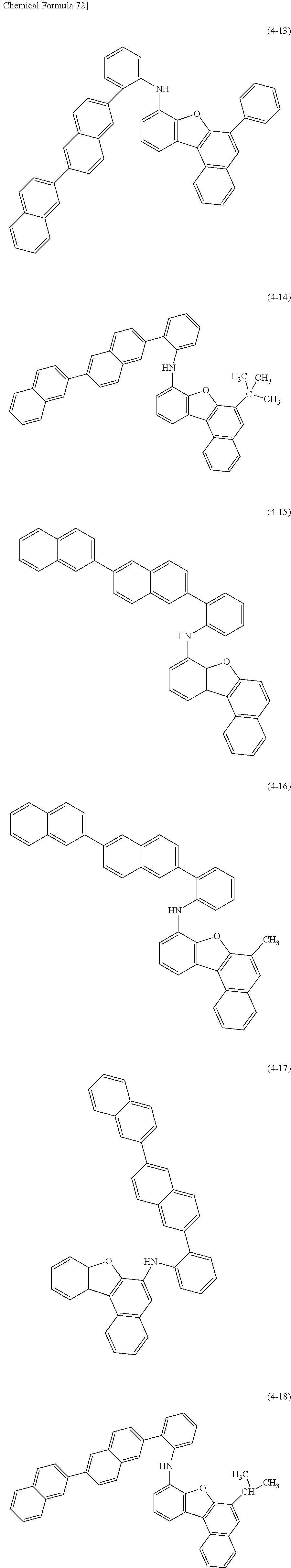 [SMILES: c1ccc(-c2cc3ccccc3c3c2oc2c(Nc4ccccc4-c4ccc5cc(-c6ccc7ccccc7c6)ccc5c4)cccc23)cc1 ; CC(C)(C)c1cc2ccccc2c2c1oc1c(Nc3ccccc3-c3ccc4cc(-c5ccc6ccccc6c5)ccc4c3)cccc12 ; c1ccc(-c2ccc3cc(-c4ccc5ccccc5c4)ccc3c2)c(Nc2cccc3c2oc2ccc4ccccc4c23)c1 ; Cc1cc2ccccc2c2c1oc1c(Nc3ccccc3-c3ccc4cc(-c5ccc6ccccc6c5)ccc4c3)cccc12 ; c1ccc(-c2ccc3cc(-c4ccc5ccccc5c4)ccc3c2)c(Nc2cc3ccccc3c3c2oc2ccccc23)c1 ; CC(C)c1cc2ccccc2c2c1oc1c(Nc3ccccc3-c3ccc4cc(-c5ccc6ccccc6c5)ccc4c3)cccc12]

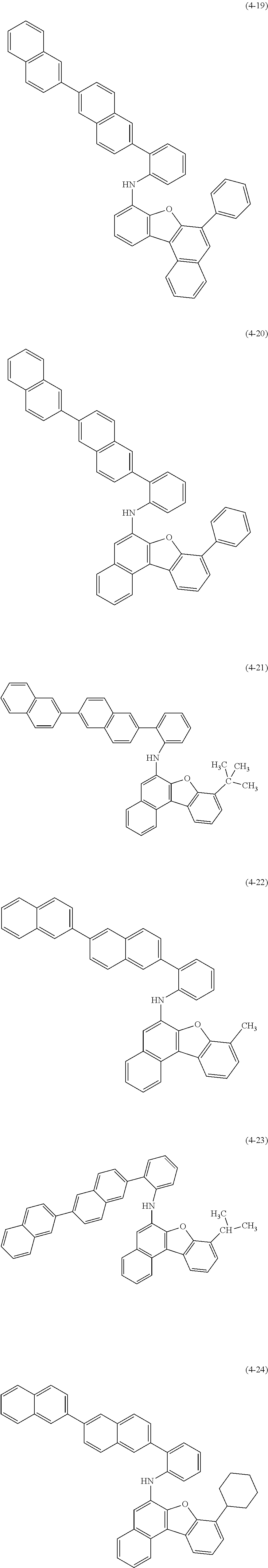 [SMILES: c1ccc(-c2cc3ccccc3c3c2oc2c(Nc4ccccc4-c4ccc5cc(-c6ccc7ccccc7c6)ccc5c4)cccc23)cc1 ; c1ccc(-c2cccc3c2oc2c(Nc4ccccc4-c4ccc5cc(-c6ccc7ccccc7c6)ccc5c4)cc4ccccc4c23)cc1 ; CC(C)(C)c1cccc2c1oc1c(Nc3ccccc3-c3ccc4cc(-c5ccc6ccccc6c5)ccc4c3)cc3ccccc3c12 ; Cc1cccc2c1oc1c(Nc3ccccc3-c3ccc4cc(-c5ccc6ccccc6c5)ccc4c3)cc3ccccc3c12 ; CC(C)c1cccc2c1oc1c(Nc3ccccc3-c3ccc4cc(-c5ccc6ccccc6c5)ccc4c3)cc3ccccc3c12 ; c1ccc(-c2ccc3cc(-c4ccc5ccccc5c4)ccc3c2)c(Nc2cc3ccccc3c3c2oc2c(C4CCCCC4)cccc23)c1]

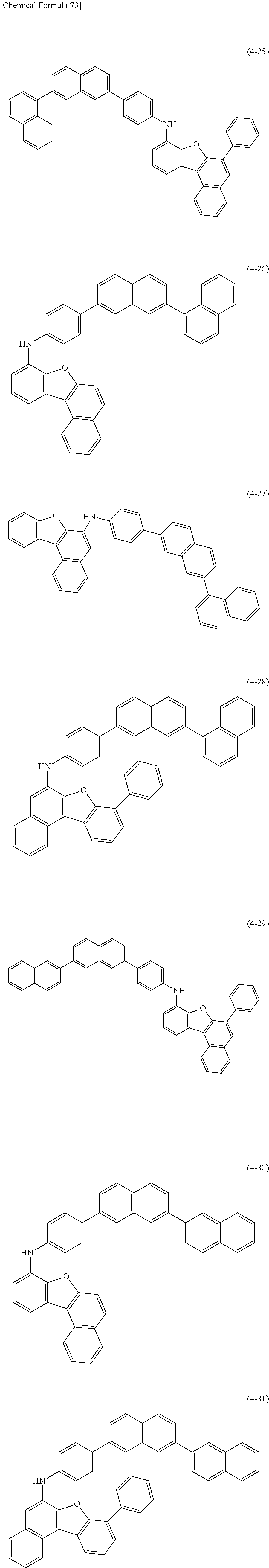

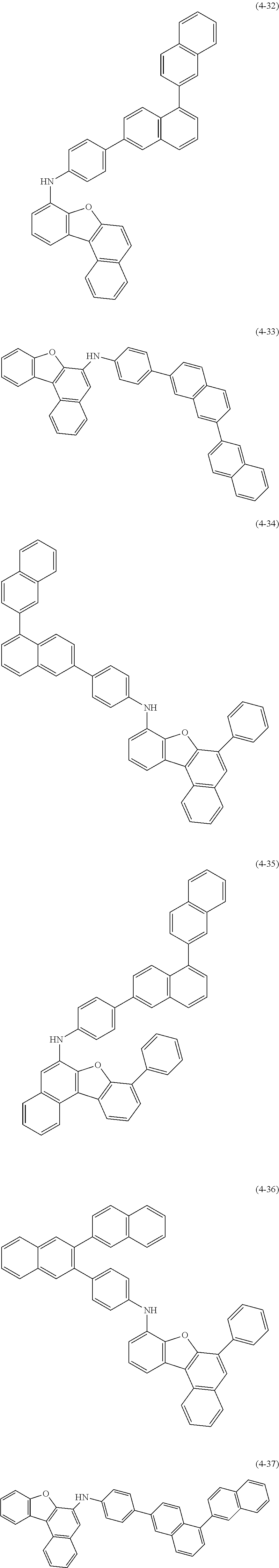 [SMILES: c1ccc2cc(-c3cccc4cc(-c5ccc(Nc6cccc7c6oc6ccc8ccccc8c67)cc5)ccc34)ccc2c1 ; c1ccc2cc(-c3ccc4ccc(-c5ccc(Nc6cc7ccccc7c7c6oc6ccccc67)cc5)cc4c3)ccc2c1 ; c1ccc(-c2cc3ccccc3c3c2oc2c(Nc4ccc(-c5ccc6c(-c7ccc8ccccc8c7)cccc6c5)cc4)cccc23)cc1 ; c1ccc(-c2cccc3c2oc2c(Nc4ccc(-c5ccc6c(-c7ccc8ccccc8c7)cccc6c5)cc4)cc4ccccc4c23)cc1 ; c1ccc(-c2cc3ccccc3c3c2oc2c(Nc4ccc(-c5cc6ccccc6cc5-c5ccc6ccccc6c5)cc4)cccc23)cc1 ; c1ccc2cc(-c3cccc4cc(-c5ccc(Nc6cc7ccccc7c7c6oc6ccccc67)cc5)ccc34)ccc2c1]

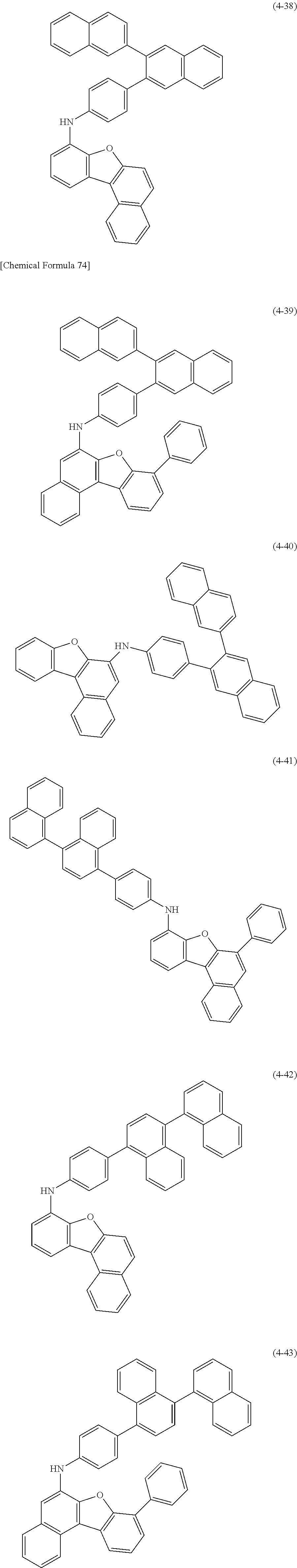 [SMILES: c1ccc2cc(-c3cc4ccccc4cc3-c3ccc(Nc4cccc5c4oc4ccc6ccccc6c45)cc3)ccc2c1 ; c1ccc(-c2cccc3c2oc2c(Nc4ccc(-c5cc6ccccc6cc5-c5ccc6ccccc6c5)cc4)cc4ccccc4c23)cc1 ; c1ccc2cc(-c3cc4ccccc4cc3-c3ccc(Nc4cc5ccccc5c5c4oc4ccccc45)cc3)ccc2c1 ; c1ccc(-c2cc3ccccc3c3c2oc2c(Nc4ccc(-c5ccc(-c6cccc7ccccc67)c6ccccc56)cc4)cccc23)cc1 ; c1ccc2c(-c3ccc(-c4ccc(Nc5cccc6c5oc5ccc7ccccc7c56)cc4)c4ccccc34)cccc2c1 ; c1ccc(-c2cccc3c2oc2c(Nc4ccc(-c5ccc(-c6cccc7ccccc67)c6ccccc56)cc4)cc4ccccc4c23)cc1]

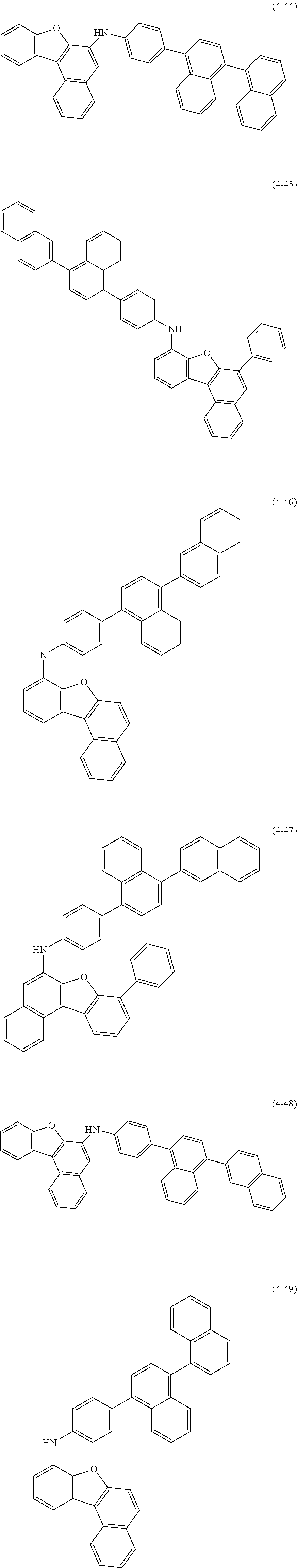 [SMILES: c1ccc2c(-c3ccc(-c4ccc(Nc5cc6ccccc6c6c5oc5ccccc56)cc4)c4ccccc34)cccc2c1 ; c1ccc(-c2cc3ccccc3c3c2oc2c(Nc4ccc(-c5ccc(-c6ccc7ccccc7c6)c6ccccc56)cc4)cccc23)cc1 ; c1ccc2cc(-c3ccc(-c4ccc(Nc5cccc6c5oc5ccc7ccccc7c56)cc4)c4ccccc34)ccc2c1 ; c1ccc(-c2cccc3c2oc2c(Nc4ccc(-c5ccc(-c6ccc7ccccc7c6)c6ccccc56)cc4)cc4ccccc4c23)cc1 ; c1ccc2cc(-c3ccc(-c4ccc(Nc5cc6ccccc6c6c5oc5ccccc56)cc4)c4ccccc34)ccc2c1 ; c1ccc2c(-c3ccc(-c4ccc(Nc5cccc6c5oc5ccc7ccccc7c56)cc4)c4ccccc34)cccc2c1]

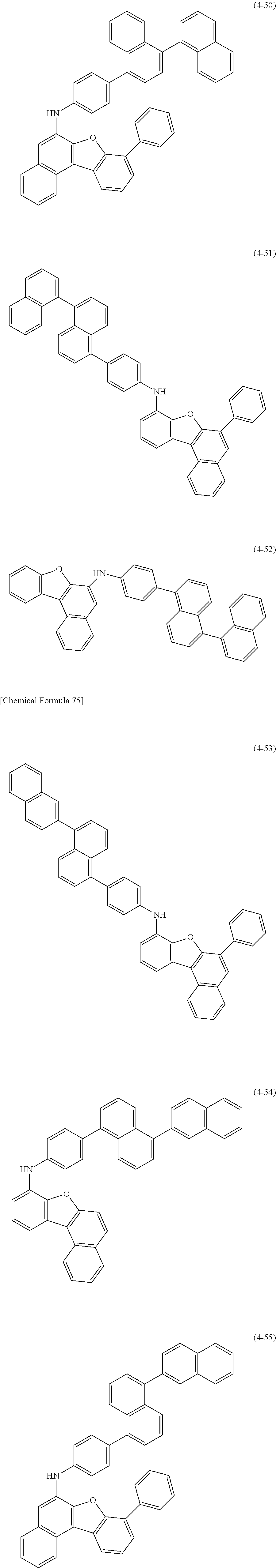 [SMILES: c1ccc(-c2cccc3c2oc2c(Nc4ccc(-c5ccc(-c6cccc7ccccc67)c6ccccc56)cc4)cc4ccccc4c23)cc1 ; c1ccc(-c2cc3ccccc3c3c2oc2c(Nc4ccc(-c5cccc6c(-c7cccc8ccccc78)cccc56)cc4)cccc23)cc1 ; c1ccc2c(-c3cccc4c(-c5ccc(Nc6cc7ccccc7c7c6oc6ccccc67)cc5)cccc34)cccc2c1 ; c1ccc(-c2cc3ccccc3c3c2oc2c(Nc4ccc(-c5cccc6c(-c7ccc8ccccc8c7)cccc56)cc4)cccc23)cc1 ; c1ccc2cc(-c3cccc4c(-c5ccc(Nc6cccc7c6oc6ccc8ccccc8c67)cc5)cccc34)ccc2c1 ; c1ccc(-c2cccc3c2oc2c(Nc4ccc(-c5cccc6c(-c7ccc8ccccc8c7)cccc56)cc4)cc4ccccc4c23)cc1]

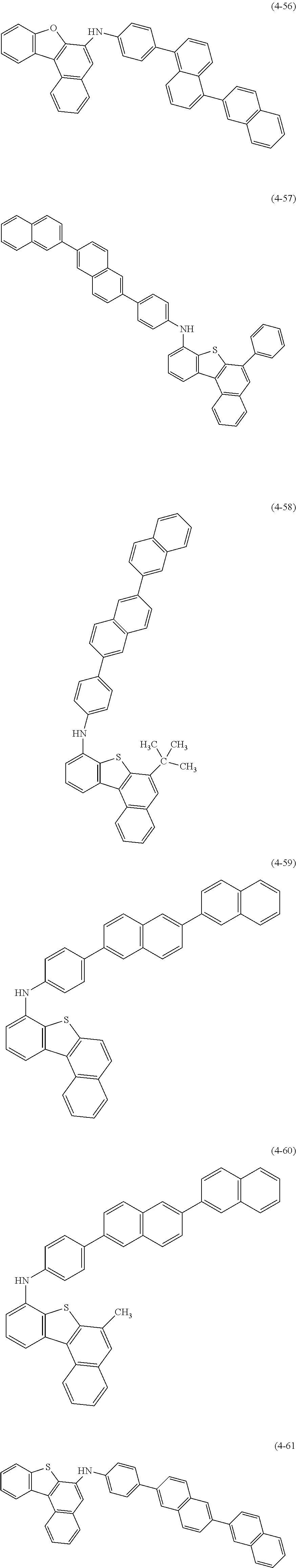 [SMILES: c1ccc2cc(-c3cccc4c(-c5ccc(Nc6cc7ccccc7c7c6oc6ccccc67)cc5)cccc34)ccc2c1 ; c1ccc(-c2cc3ccccc3c3c2sc2c(Nc4ccc(-c5ccc6cc(-c7ccc8ccccc8c7)ccc6c5)cc4)cccc23)cc1 ; CC(C)(C)c1cc2ccccc2c2c1sc1c(Nc3ccc(-c4ccc5cc(-c6ccc7ccccc7c6)ccc5c4)cc3)cccc12 ; c1ccc2cc(-c3ccc4cc(-c5ccc(Nc6cccc7c6sc6ccc8ccccc8c67)cc5)ccc4c3)ccc2c1 ; Cc1cc2ccccc2c2c1sc1c(Nc3ccc(-c4ccc5cc(-c6ccc7ccccc7c6)ccc5c4)cc3)cccc12 ; c1ccc2cc(-c3ccc4cc(-c5ccc(Nc6cc7ccccc7c7c6sc6ccccc67)cc5)ccc4c3)ccc2c1]

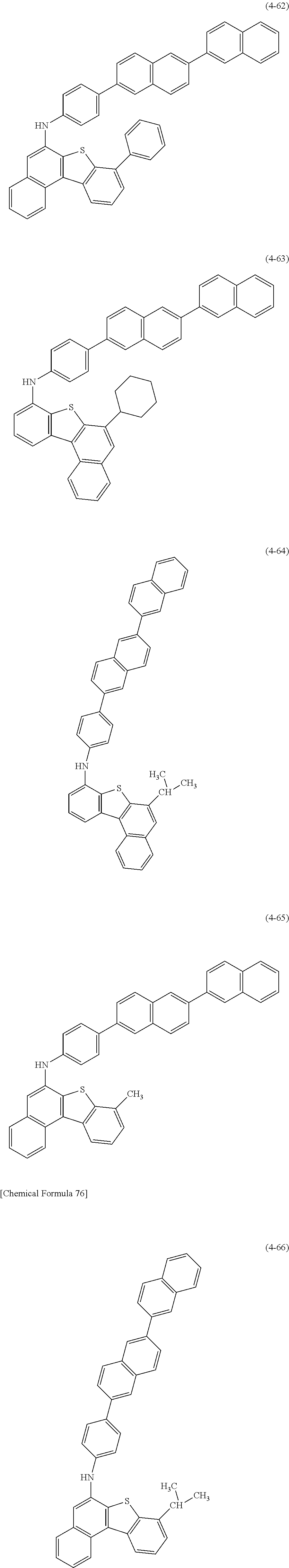

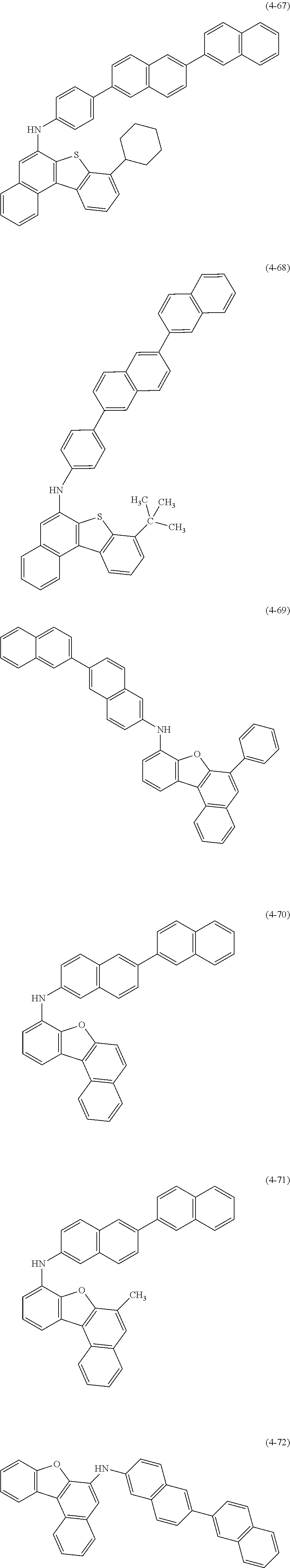 [SMILES: c1ccc2cc(-c3ccc4cc(-c5ccc(Nc6cc7ccccc7c7c6sc6c(C8CCCCC8)cccc67)cc5)ccc4c3)ccc2c1 ; CC(C)(C)c1cccc2c1sc1c(Nc3ccc(-c4ccc5cc(-c6ccc7ccccc7c6)ccc5c4)cc3)cc3ccccc3c12 ; c1ccc(-c2cc3ccccc3c3c2oc2c(Nc4ccc5cc(-c6ccc7ccccc7c6)ccc5c4)cccc23)cc1 ; c1ccc2cc(-c3ccc4cc(Nc5cccc6c5oc5ccc7ccccc7c56)ccc4c3)ccc2c1 ; Cc1cc2ccccc2c2c1oc1c(Nc3ccc4cc(-c5ccc6ccccc6c5)ccc4c3)cccc12 ; c1ccc2cc(-c3ccc4cc(Nc5cc6ccccc6c6c5oc5ccccc56)ccc4c3)ccc2c1]

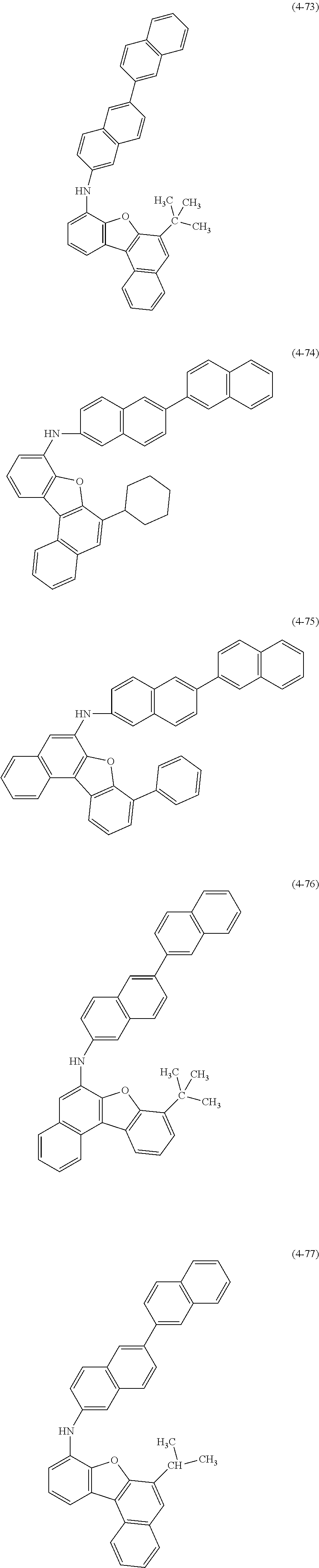 [SMILES: CC(C)(C)c1cc2ccccc2c2c1oc1c(Nc3ccc4cc(-c5ccc6ccccc6c5)ccc4c3)cccc12 ; c1ccc2cc(-c3ccc4cc(Nc5cccc6c5oc5c(C7CCCCC7)cc7ccccc7c56)ccc4c3)ccc2c1 ; c1ccc(-c2cccc3c2oc2c(Nc4ccc5cc(-c6ccc7ccccc7c6)ccc5c4)cc4ccccc4c23)cc1 ; CC(C)(C)c1cccc2c1oc1c(Nc3ccc4cc(-c5ccc6ccccc6c5)ccc4c3)cc3ccccc3c12 ; CC(C)c1cc2ccccc2c2c1oc1c(Nc3ccc4cc(-c5ccc6ccccc6c5)ccc4c3)cccc12]

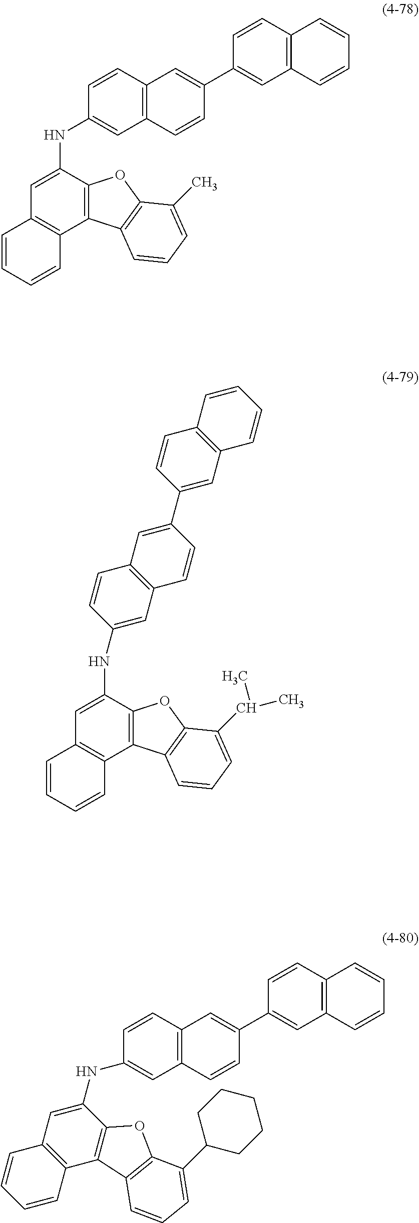 [SMILES: Cc1cccc2c1oc1c(Nc3ccc4cc(-c5ccc6ccccc6c5)ccc4c3)cc3ccccc3c12 ; CC(C)c1cccc2c1oc1c(Nc3ccc4cc(-c5ccc6ccccc6c5)ccc4c3)cc3ccccc3c12 ; c1ccc2cc(-c3ccc4cc(Nc5cc6ccccc6c6c5oc5c(C7CCCCC7)cccc56)ccc4c3)ccc2c1]

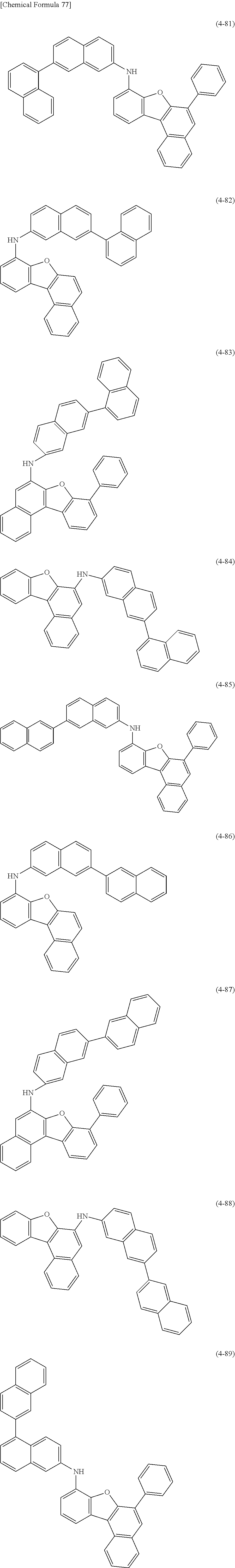

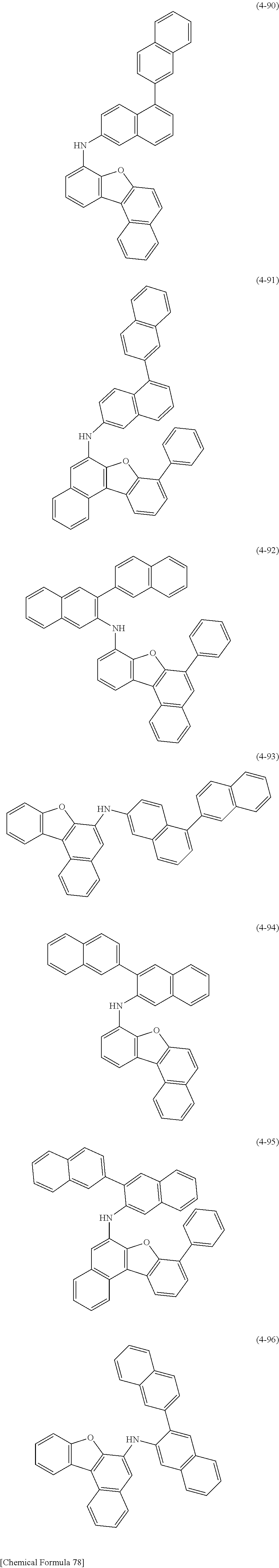

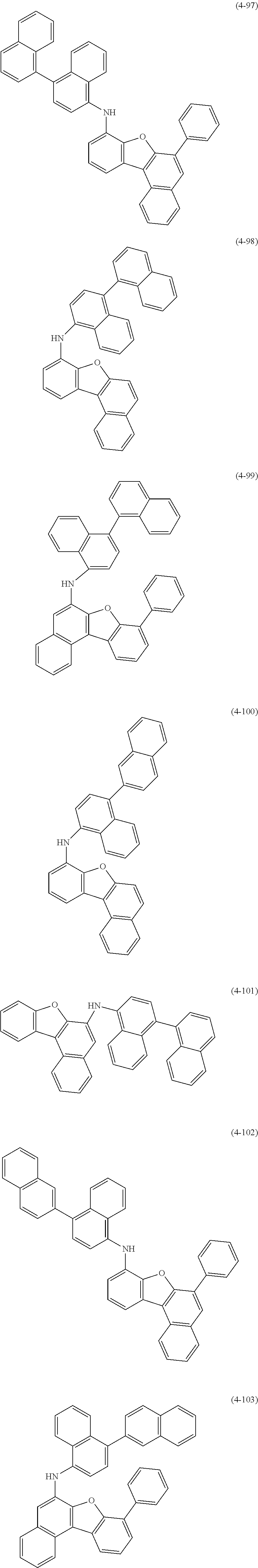

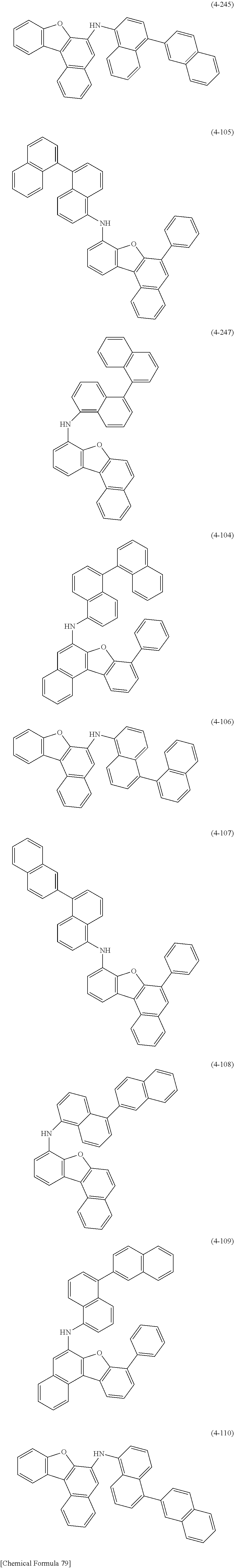

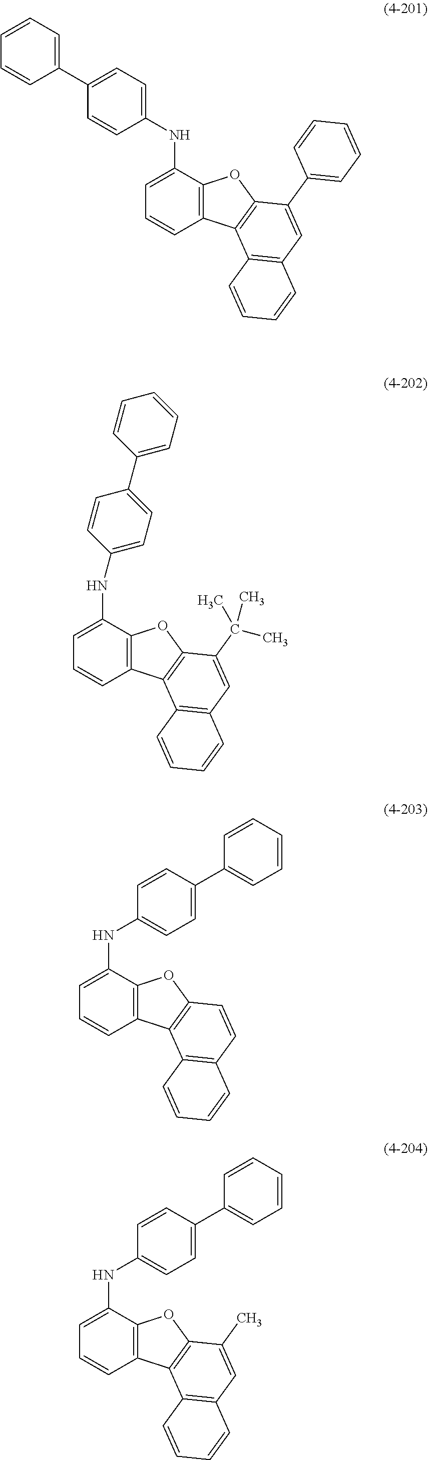 [SMILES: c1ccc(-c2ccc(Nc3cccc4c3oc3c(-c5ccccc5)cc5ccccc5c34)cc2)cc1 ; CC(C)(C)c1cc2ccccc2c2c1oc1c(Nc3ccc(-c4ccccc4)cc3)cccc12 ; c1ccc(-c2ccc(Nc3cccc4c3oc3ccc5ccccc5c34)cc2)cc1 ; Cc1cc2ccccc2c2c1oc1c(Nc3ccc(-c4ccccc4)cc3)cccc12]

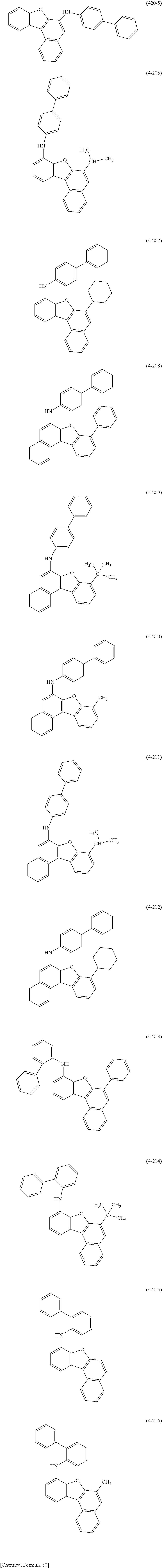

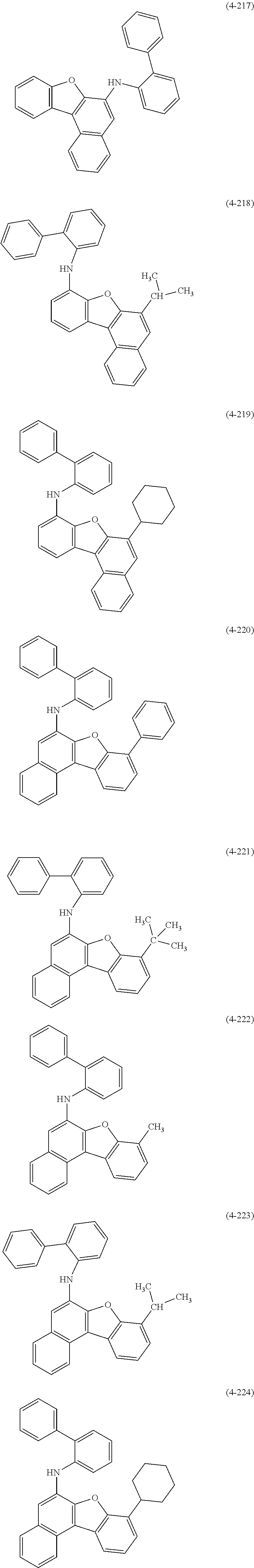

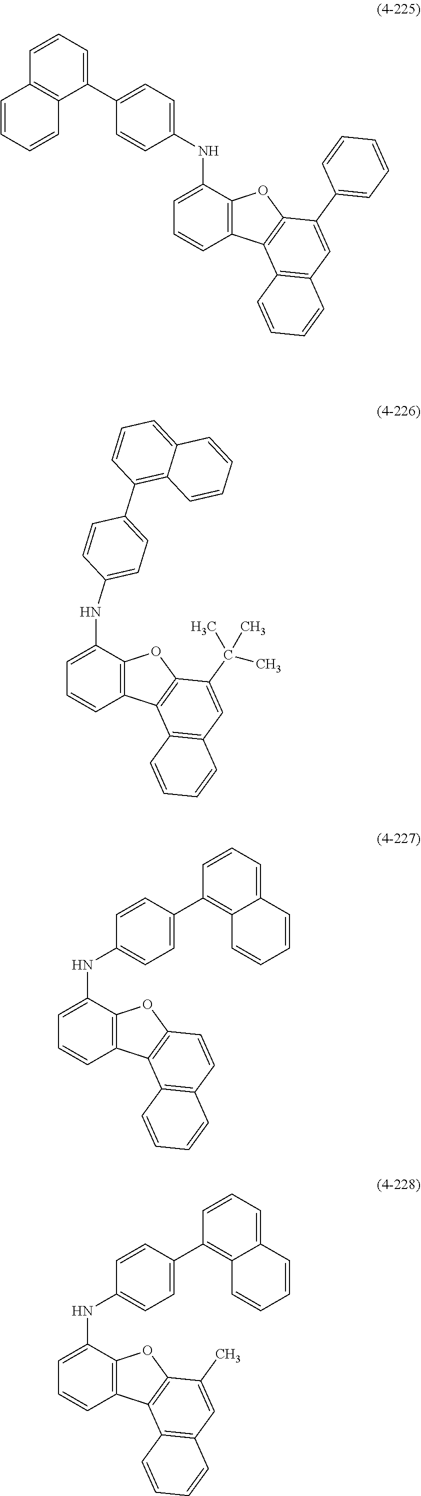 [SMILES: c1ccc(-c2cc3ccccc3c3c2oc2c(Nc4ccc(-c5cccc6ccccc56)cc4)cccc23)cc1 ; CC(C)(C)c1cc2ccccc2c2c1oc1c(Nc3ccc(-c4cccc5ccccc45)cc3)cccc12 ; c1ccc2c(-c3ccc(Nc4cccc5c4oc4ccc6ccccc6c45)cc3)cccc2c1 ; Cc1cc2ccccc2c2c1oc1c(Nc3ccc(-c4cccc5ccccc45)cc3)cccc12]

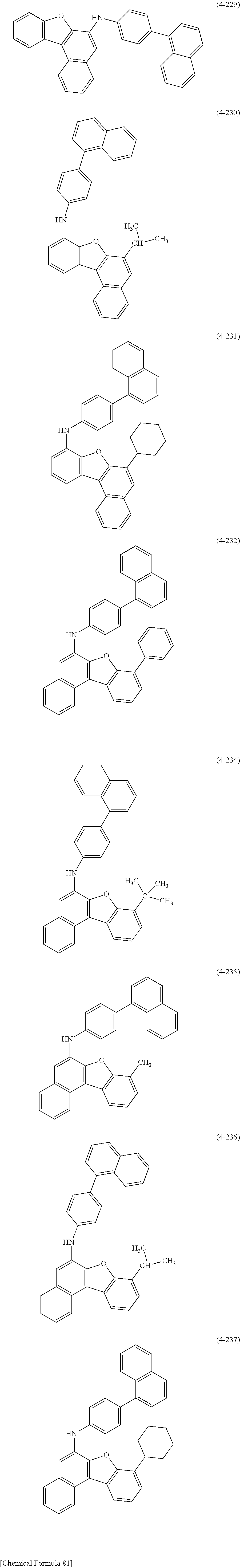

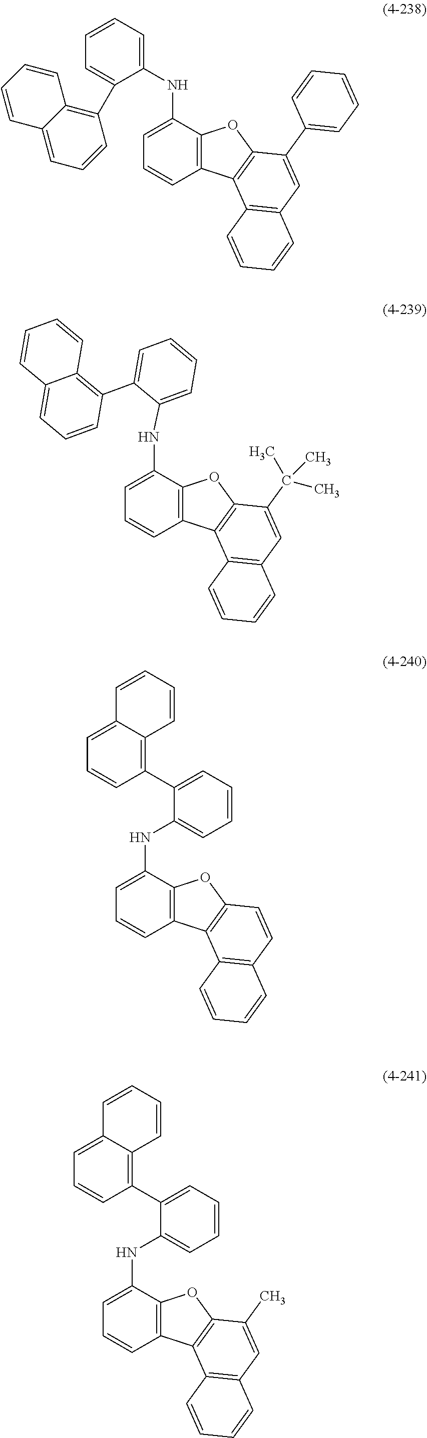 [SMILES: c1ccc(-c2cc3ccccc3c3c2oc2c(Nc4ccccc4-c4cccc5ccccc45)cccc23)cc1 ; CC(C)(C)c1cc2ccccc2c2c1oc1c(Nc3ccccc3-c3cccc4ccccc34)cccc12 ; c1ccc(-c2cccc3ccccc23)c(Nc2cccc3c2oc2ccc4ccccc4c23)c1 ; Cc1cc2ccccc2c2c1oc1c(Nc3ccccc3-c3cccc4ccccc34)cccc12]

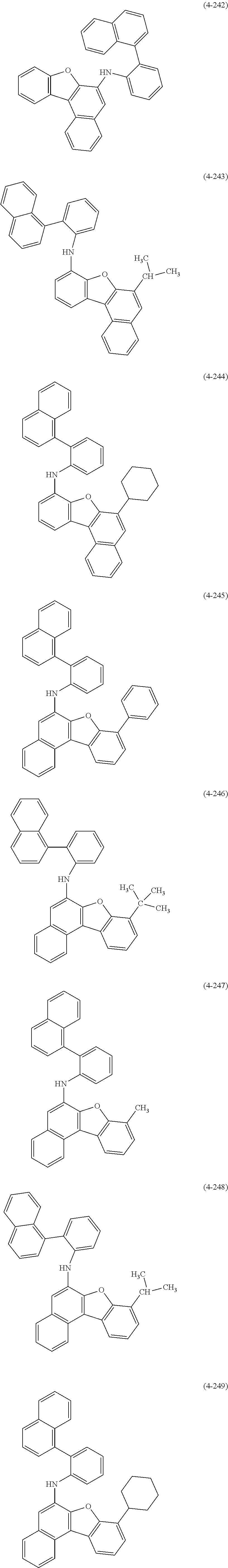

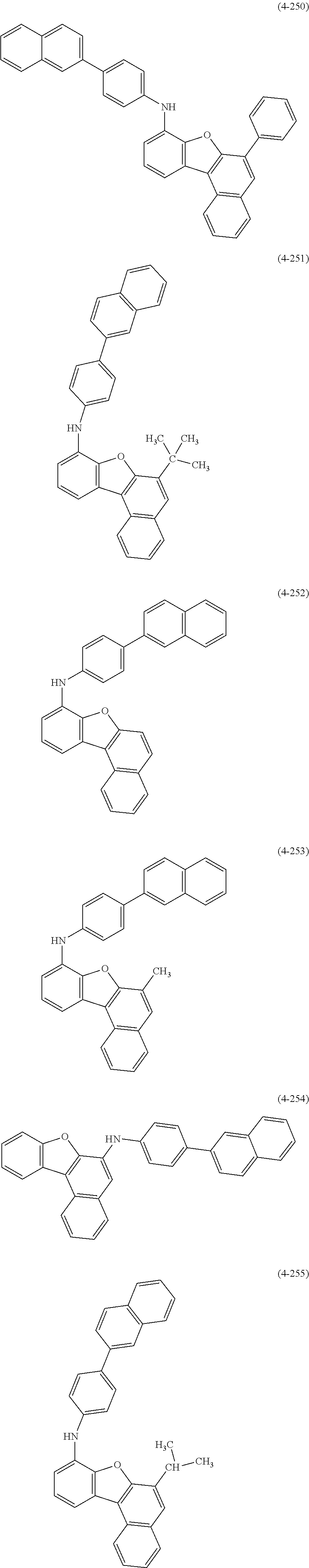 [SMILES: c1ccc(-c2cc3ccccc3c3c2oc2c(Nc4ccc(-c5ccc6ccccc6c5)cc4)cccc23)cc1 ; CC(C)(C)c1cc2ccccc2c2c1oc1c(Nc3ccc(-c4ccc5ccccc5c4)cc3)cccc12 ; c1ccc2cc(-c3ccc(Nc4cccc5c4oc4ccc6ccccc6c45)cc3)ccc2c1 ; Cc1cc2ccccc2c2c1oc1c(Nc3ccc(-c4ccc5ccccc5c4)cc3)cccc12 ; c1ccc2cc(-c3ccc(Nc4cc5ccccc5c5c4oc4ccccc45)cc3)ccc2c1 ; CC(C)c1cc2ccccc2c2c1oc1c(Nc3ccc(-c4ccc5ccccc5c4)cc3)cccc12]

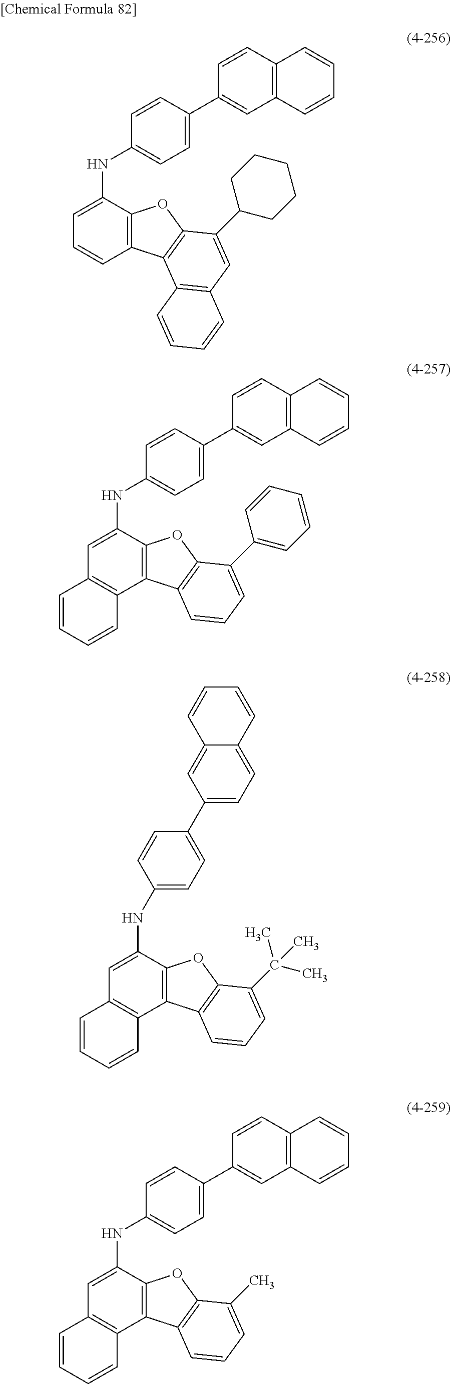 [SMILES: c1ccc2cc(-c3ccc(Nc4cccc5c4oc4c(C6CCCCC6)cc6ccccc6c45)cc3)ccc2c1 ; c1ccc(-c2cccc3c2oc2c(Nc4ccc(-c5ccc6ccccc6c5)cc4)cc4ccccc4c23)cc1 ; CC(C)(C)c1cccc2c1oc1c(Nc3ccc(-c4ccc5ccccc5c4)cc3)cc3ccccc3c12 ; Cc1cccc2c1oc1c(Nc3ccc(-c4ccc5ccccc5c4)cc3)cc3ccccc3c12]

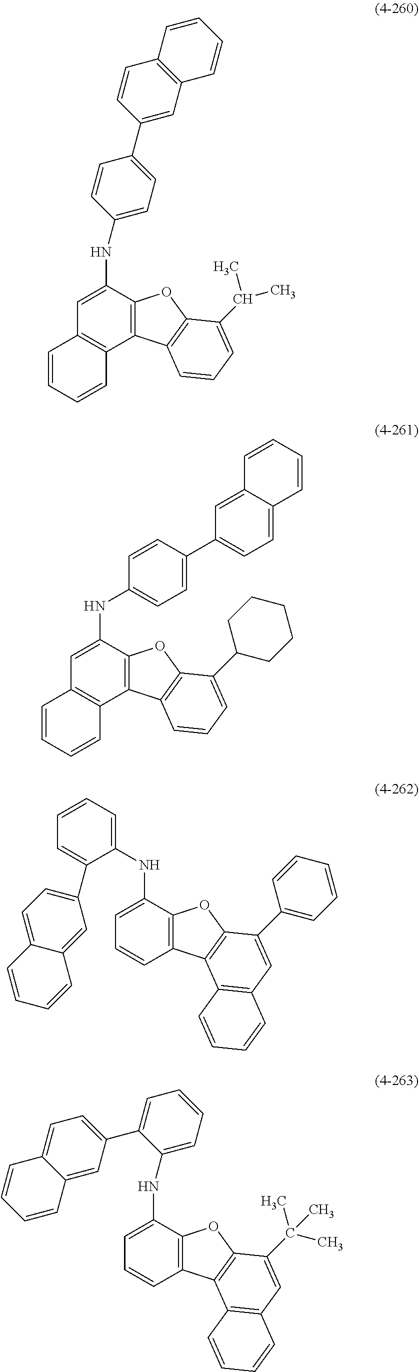

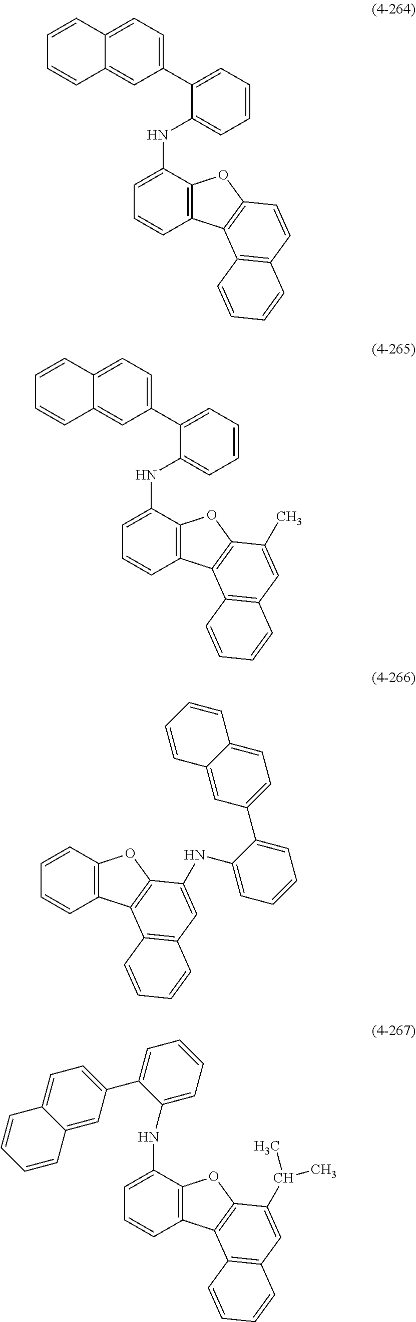

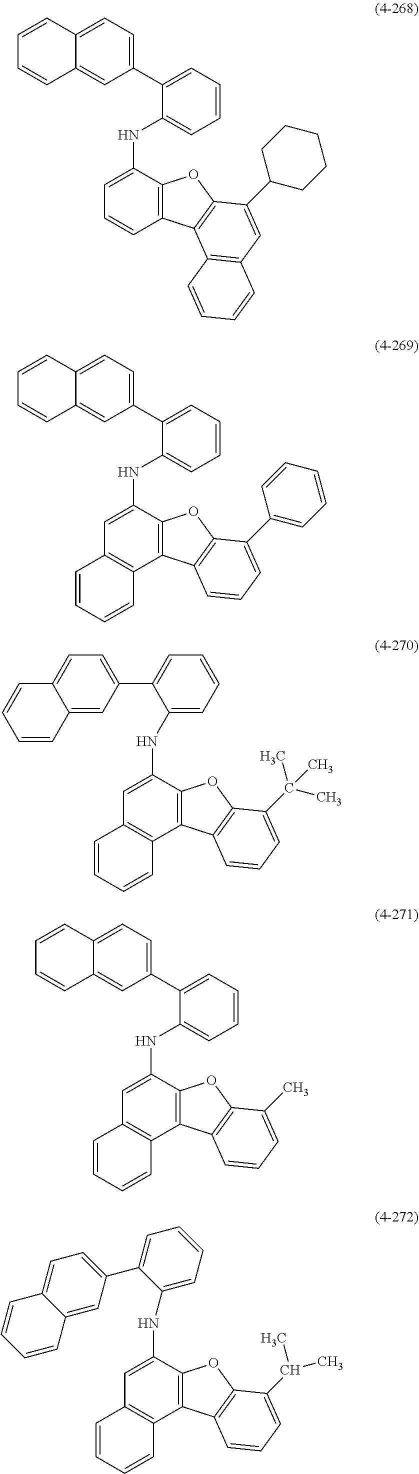

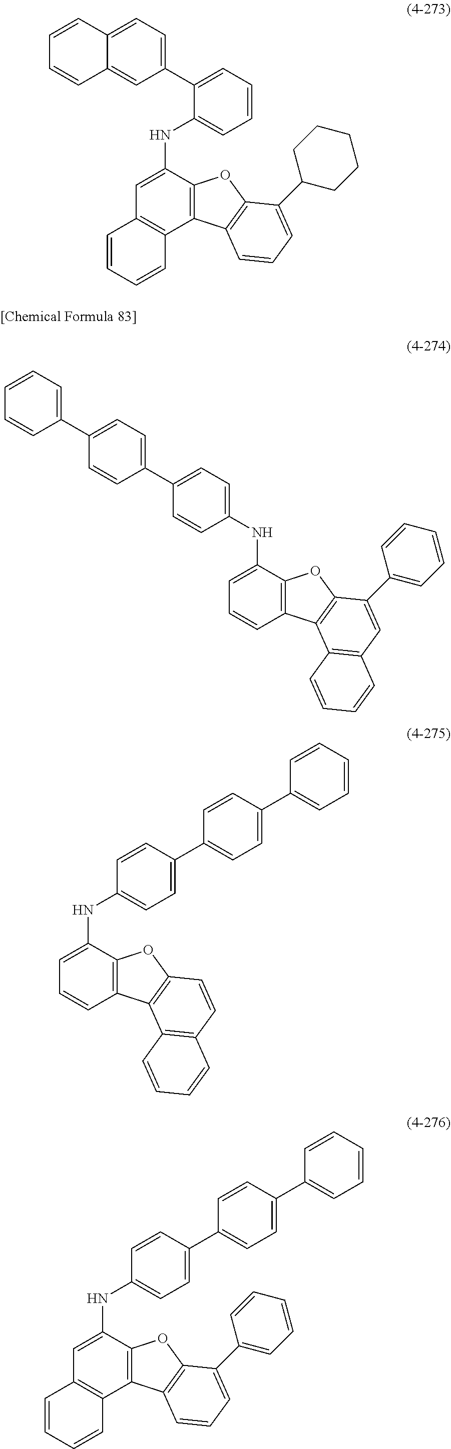

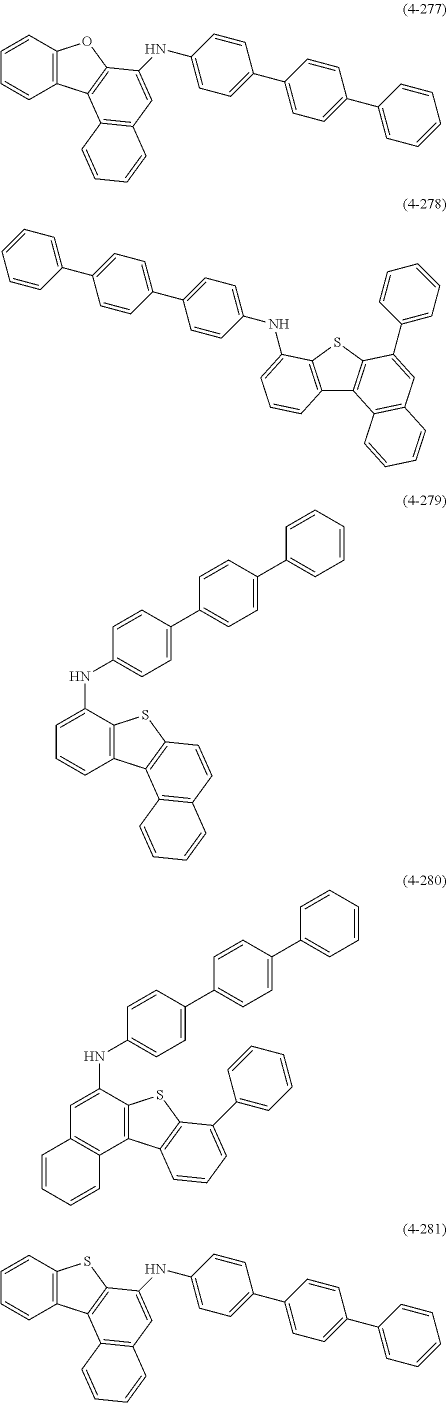 [SMILES: c1ccc(-c2ccc(-c3ccc(Nc4cc5ccccc5c5c4oc4ccccc45)cc3)cc2)cc1 ; c1ccc(-c2ccc(-c3ccc(Nc4cccc5c4sc4c(-c6ccccc6)cc6ccccc6c45)cc3)cc2)cc1 ; c1ccc(-c2ccc(-c3ccc(Nc4cccc5c4sc4ccc6ccccc6c45)cc3)cc2)cc1 ; c1ccc(-c2ccc(-c3ccc(Nc4cc5ccccc5c5c4sc4c(-c6ccccc6)cccc45)cc3)cc2)cc1 ; c1ccc(-c2ccc(-c3ccc(Nc4cc5ccccc5c5c4sc4ccccc45)cc3)cc2)cc1]

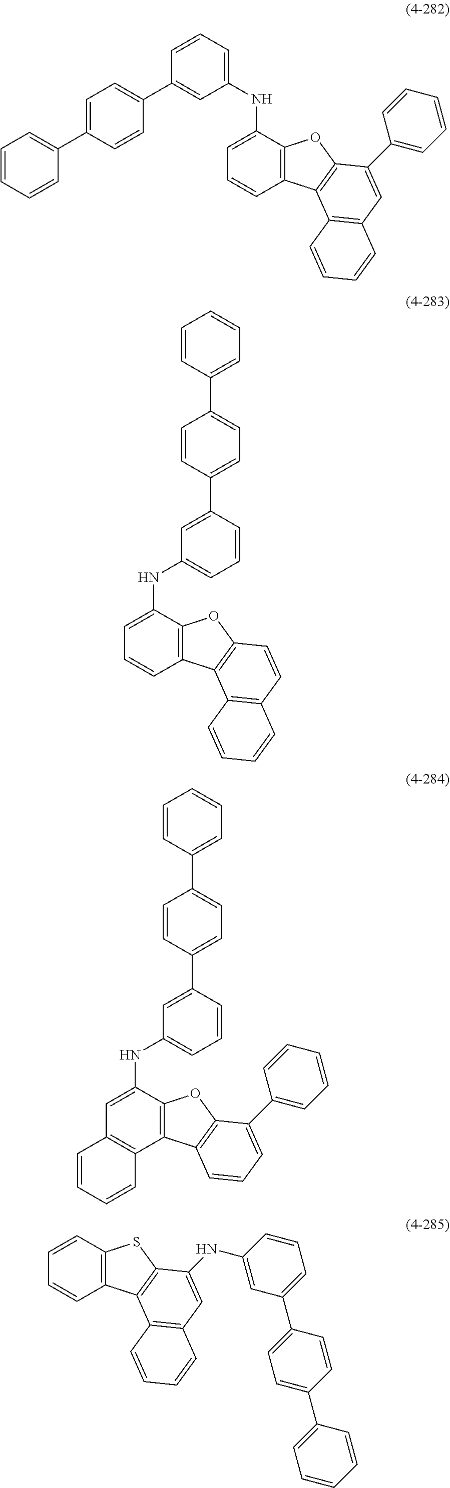

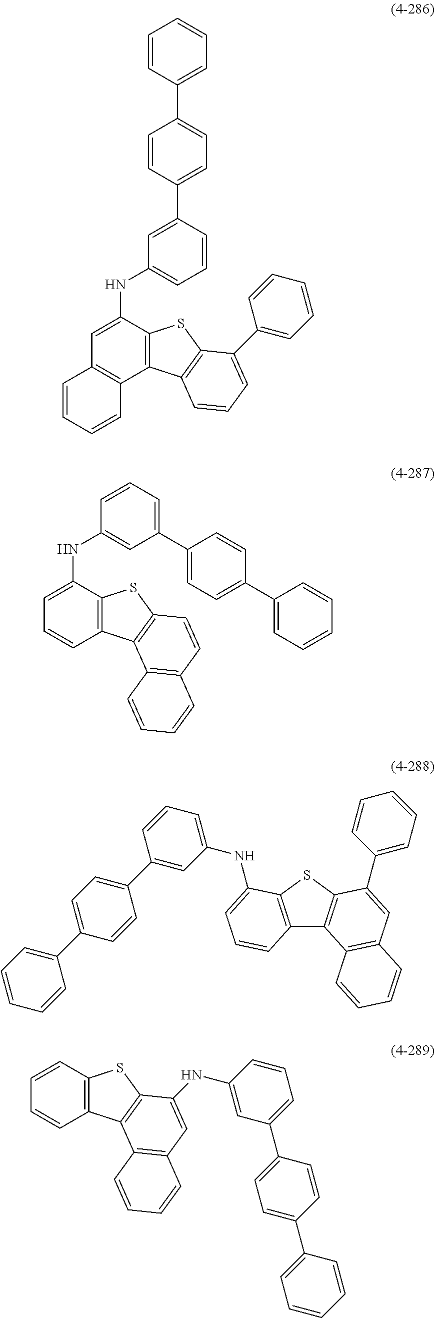 [SMILES: c1ccc(-c2ccc(-c3cccc(Nc4cc5ccccc5c5c4sc4c(-c6ccccc6)cccc45)c3)cc2)cc1 ; c1ccc(-c2ccc(-c3cccc(Nc4cccc5c4sc4ccc6ccccc6c45)c3)cc2)cc1 ; c1ccc(-c2ccc(-c3cccc(Nc4cccc5c4sc4c(-c6ccccc6)cc6ccccc6c45)c3)cc2)cc1 ; c1ccc(-c2ccc(-c3cccc(Nc4cc5ccccc5c5c4sc4ccccc45)c3)cc2)cc1]

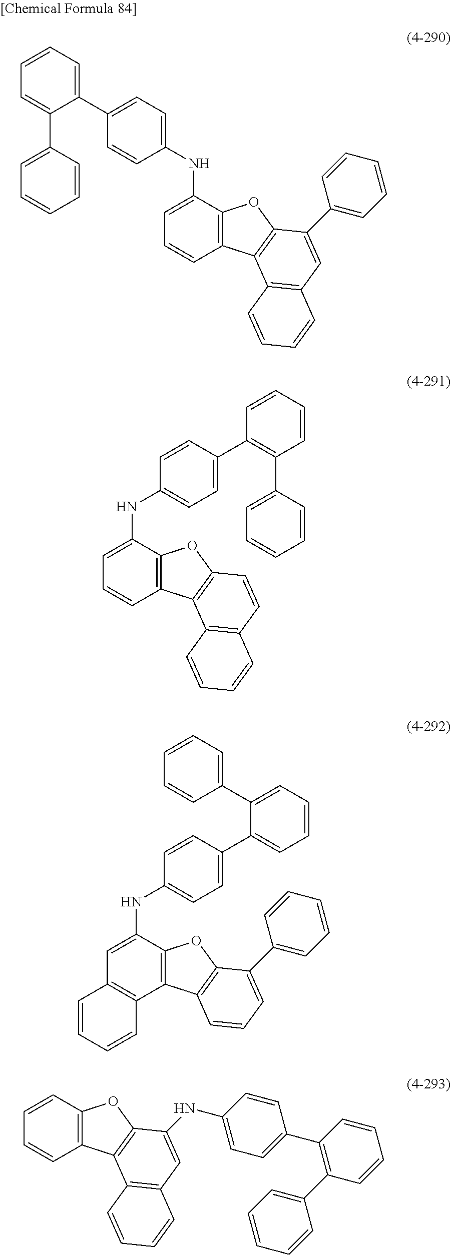 [SMILES: c1ccc(-c2ccccc2-c2ccc(Nc3cccc4c3oc3c(-c5ccccc5)cc5ccccc5c34)cc2)cc1 ; c1ccc(-c2ccccc2-c2ccc(Nc3cccc4c3oc3ccc5ccccc5c34)cc2)cc1 ; c1ccc(-c2ccccc2-c2ccc(Nc3cc4ccccc4c4c3oc3c(-c5ccccc5)cccc34)cc2)cc1 ; c1ccc(-c2ccccc2-c2ccc(Nc3cc4ccccc4c4c3oc3ccccc34)cc2)cc1]

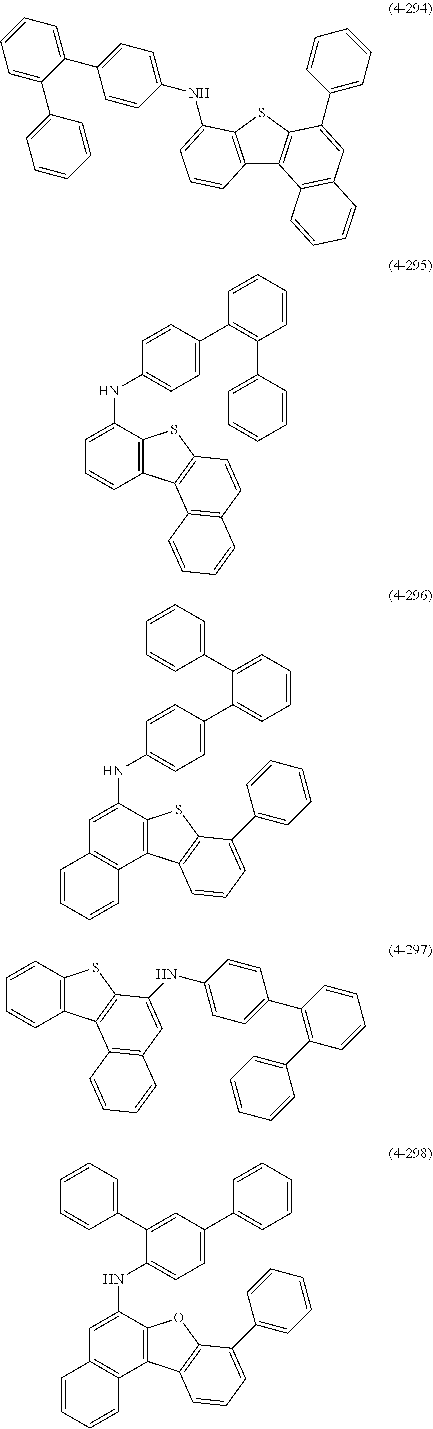

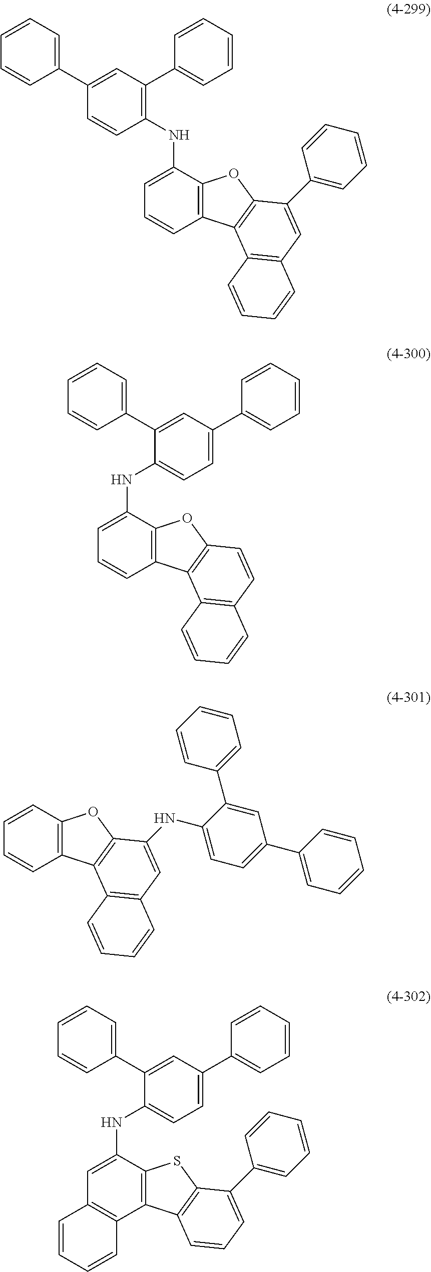

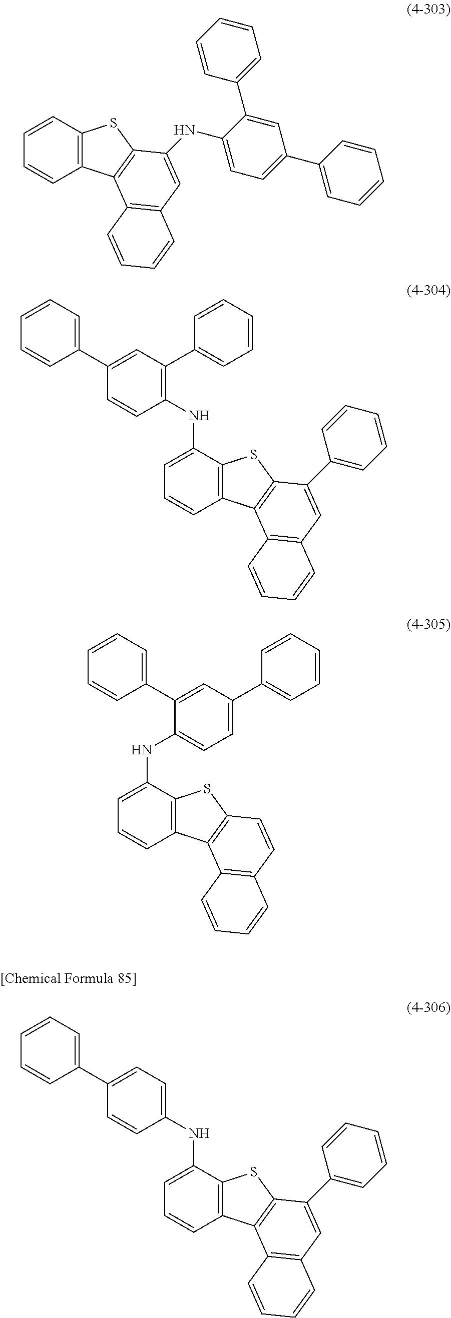

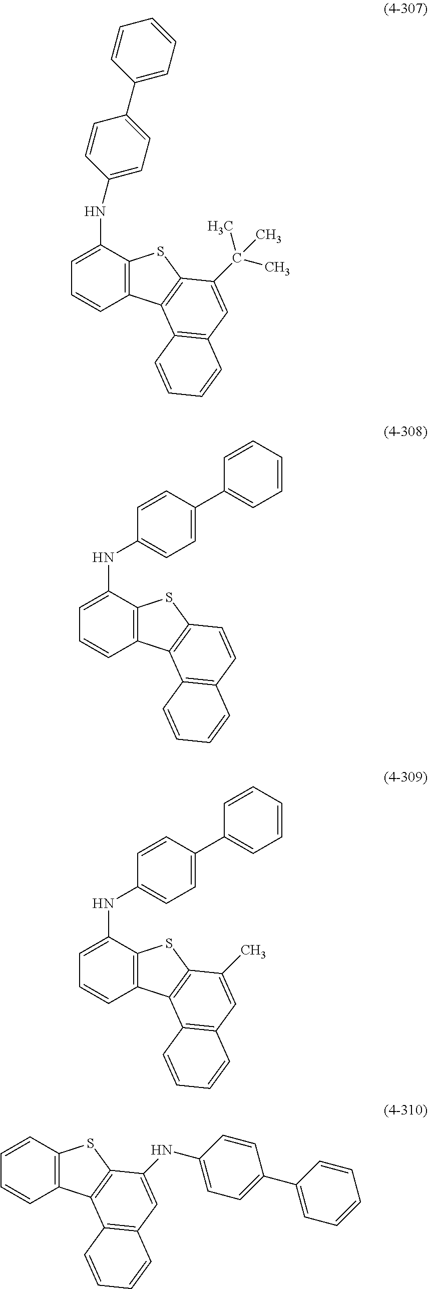 [SMILES: CC(C)(C)c1cc2ccccc2c2c1sc1c(Nc3ccc(-c4ccccc4)cc3)cccc12 ; c1ccc(-c2ccc(Nc3cccc4c3sc3ccc5ccccc5c34)cc2)cc1 ; Cc1cc2ccccc2c2c1sc1c(Nc3ccc(-c4ccccc4)cc3)cccc12 ; c1ccc(-c2ccc(Nc3cc4ccccc4c4c3sc3ccccc34)cc2)cc1]

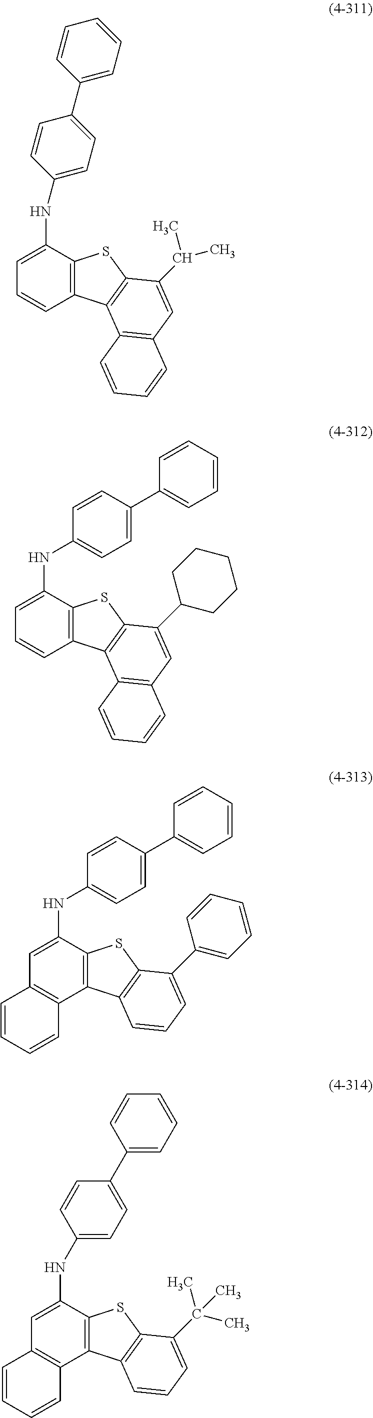 [SMILES: CC(C)c1cc2ccccc2c2c1sc1c(Nc3ccc(-c4ccccc4)cc3)cccc12 ; c1ccc(-c2ccc(Nc3cccc4c3sc3c(C5CCCCC5)cc5ccccc5c34)cc2)cc1 ; c1ccc(-c2ccc(Nc3cc4ccccc4c4c3sc3c(-c5ccccc5)cccc34)cc2)cc1 ; CC(C)(C)c1cccc2c1sc1c(Nc3ccc(-c4ccccc4)cc3)cc3ccccc3c12]

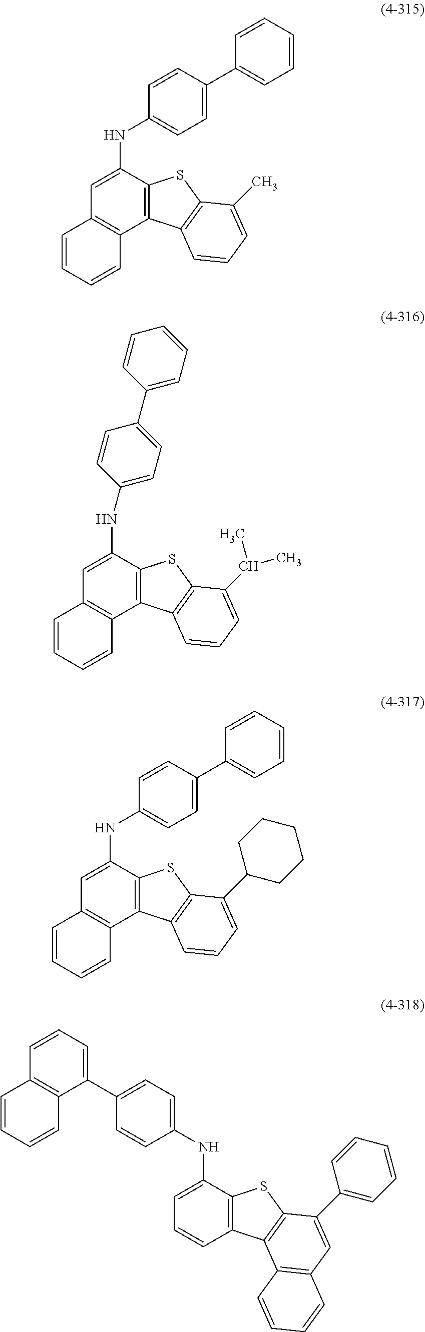 [SMILES: Cc1cccc2c1sc1c(Nc3ccc(-c4ccccc4)cc3)cc3ccccc3c12 ; CC(C)c1cccc2c1sc1c(Nc3ccc(-c4ccccc4)cc3)cc3ccccc3c12 ; c1ccc(-c2ccc(Nc3cc4ccccc4c4c3sc3c(C5CCCCC5)cccc34)cc2)cc1 ; c1ccc(-c2cc3ccccc3c3c2sc2c(Nc4ccc(-c5cccc6ccccc56)cc4)cccc23)cc1]

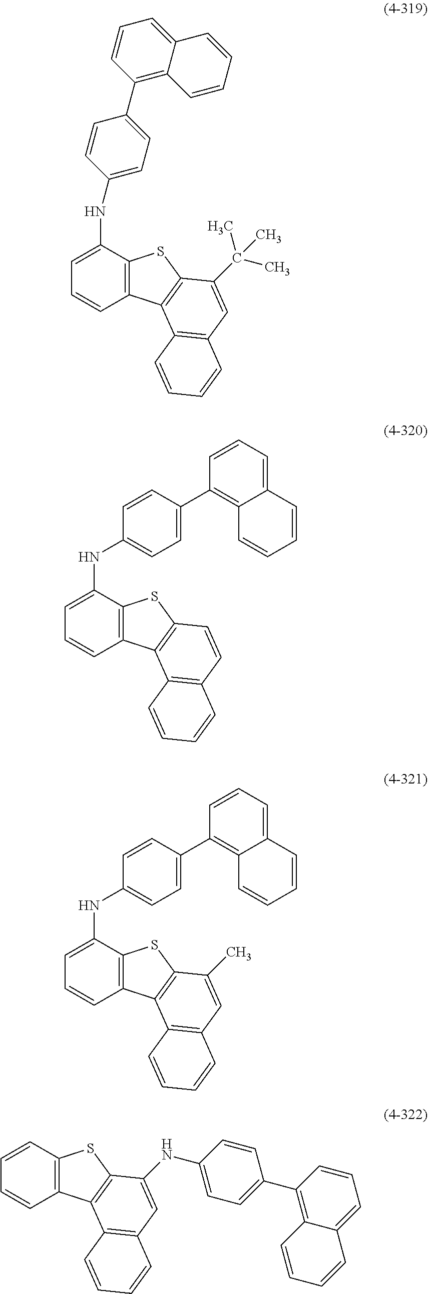 [SMILES: CC(C)(C)c1cc2ccccc2c2c1sc1c(Nc3ccc(-c4cccc5ccccc45)cc3)cccc12 ; c1ccc2c(-c3ccc(Nc4cccc5c4sc4ccc6ccccc6c45)cc3)cccc2c1 ; Cc1cc2ccccc2c2c1sc1c(Nc3ccc(-c4cccc5ccccc45)cc3)cccc12 ; c1ccc2c(-c3ccc(Nc4cc5ccccc5c5c4sc4ccccc45)cc3)cccc2c1]

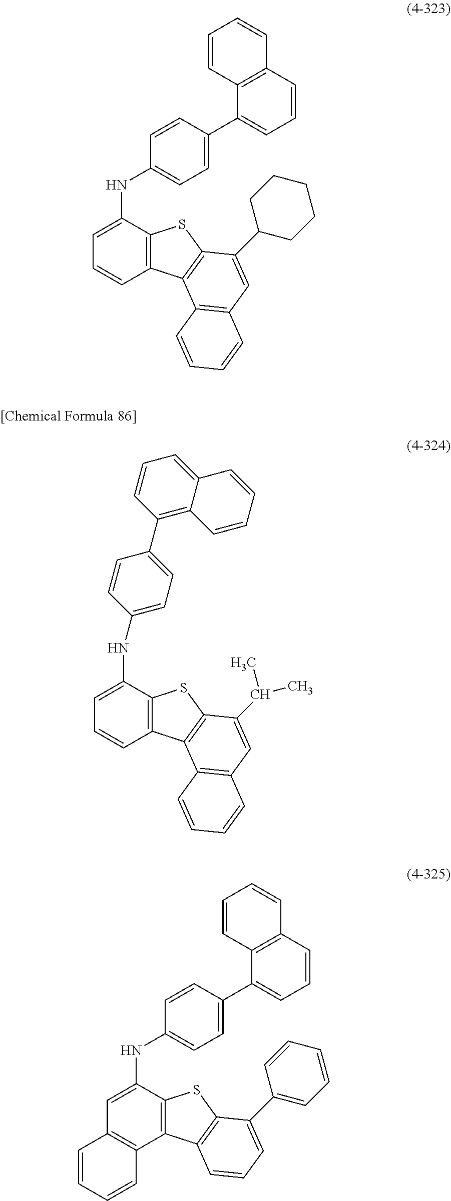 [SMILES: c1ccc2c(-c3ccc(Nc4cccc5c4sc4c(C6CCCCC6)cc6ccccc6c45)cc3)cccc2c1 ; CC(C)c1cc2ccccc2c2c1sc1c(Nc3ccc(-c4cccc5ccccc45)cc3)cccc12 ; c1ccc(-c2cccc3c2sc2c(Nc4ccc(-c5cccc6ccccc56)cc4)cc4ccccc4c23)cc1]

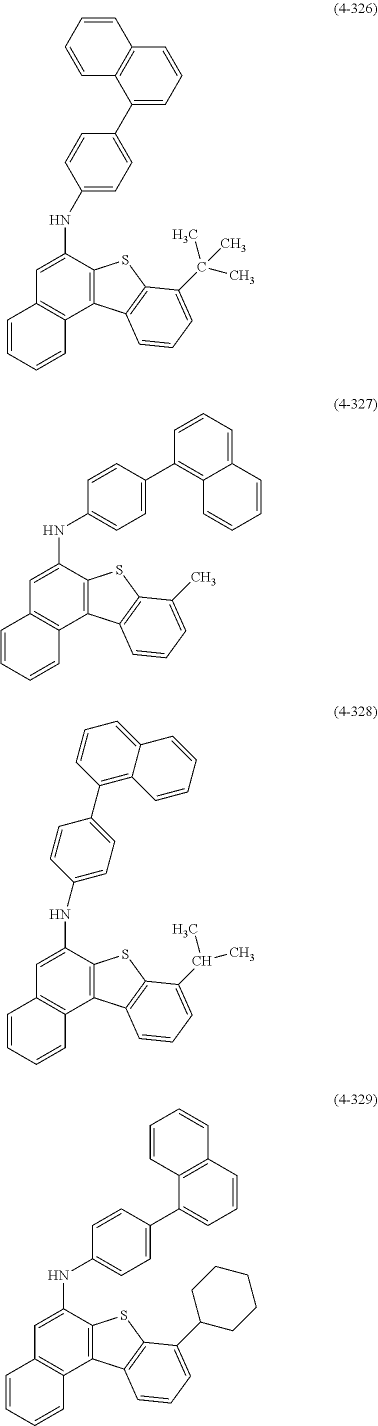 [SMILES: CC(C)(C)c1cccc2c1sc1c(Nc3ccc(-c4cccc5ccccc45)cc3)cc3ccccc3c12 ; Cc1cccc2c1sc1c(Nc3ccc(-c4cccc5ccccc45)cc3)cc3ccccc3c12 ; CC(C)c1cccc2c1sc1c(Nc3ccc(-c4cccc5ccccc45)cc3)cc3ccccc3c12 ; c1ccc2c(-c3ccc(Nc4cc5ccccc5c5c4sc4c(C6CCCCC6)cccc45)cc3)cccc2c1]

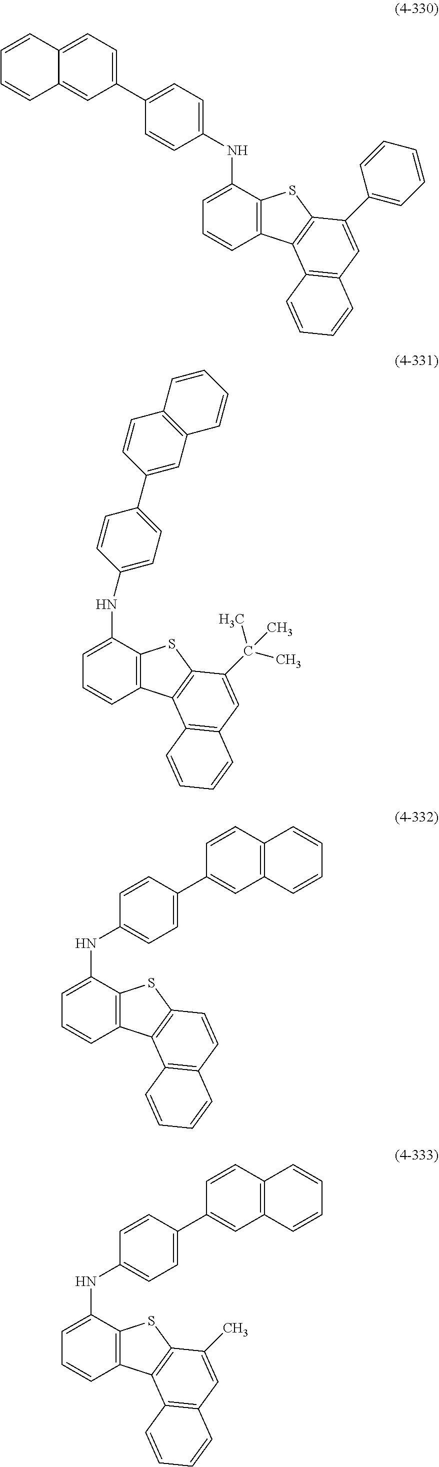

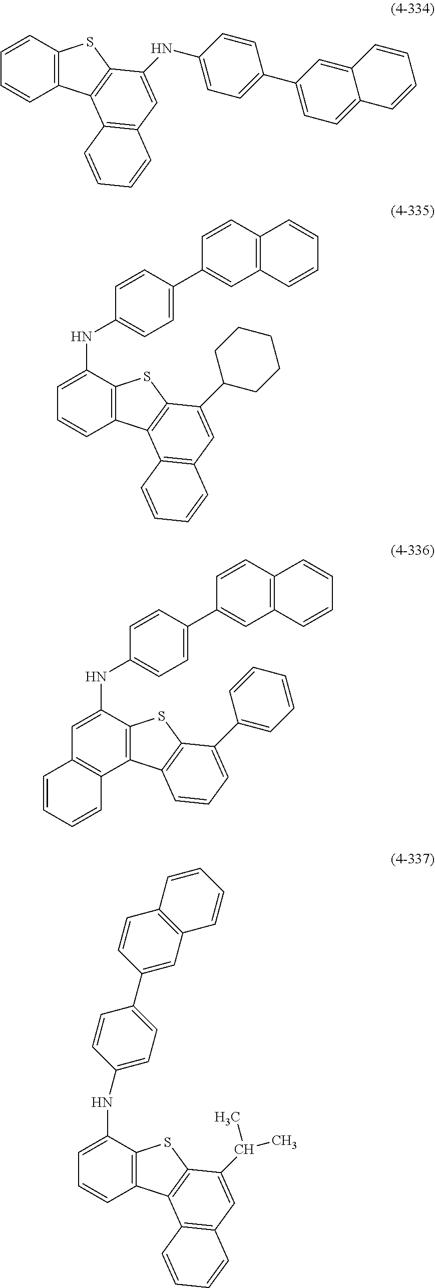 [SMILES: c1ccc2cc(-c3ccc(Nc4cc5ccccc5c5c4sc4ccccc45)cc3)ccc2c1 ; c1ccc2cc(-c3ccc(Nc4cccc5c4sc4c(C6CCCCC6)cc6ccccc6c45)cc3)ccc2c1 ; c1ccc(-c2cccc3c2sc2c(Nc4ccc(-c5ccc6ccccc6c5)cc4)cc4ccccc4c23)cc1 ; CC(C)c1cc2ccccc2c2c1sc1c(Nc3ccc(-c4ccc5ccccc5c4)cc3)cccc12]

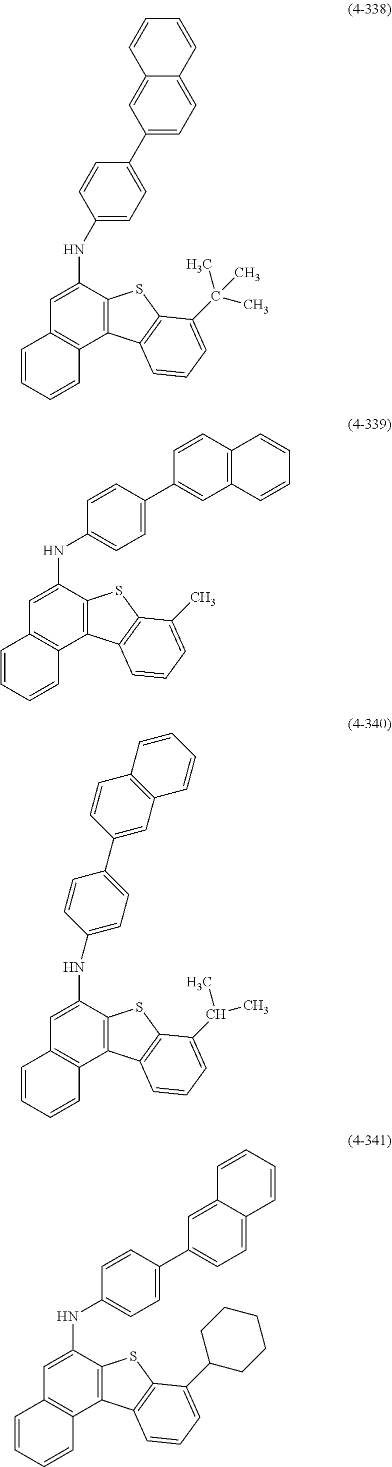 [SMILES: CC(C)(C)c1cccc2c1sc1c(Nc3ccc(-c4ccc5ccccc5c4)cc3)cc3ccccc3c12 ; Cc1cccc2c1sc1c(Nc3ccc(-c4ccc5ccccc5c4)cc3)cc3ccccc3c12 ; CC(C)c1cccc2c1sc1c(Nc3ccc(-c4ccc5ccccc5c4)cc3)cc3ccccc3c12 ; c1ccc2cc(-c3ccc(Nc4cc5ccccc5c5c4sc4c(C6CCCCC6)cccc45)cc3)ccc2c1]

The organic compounds of embodiments of the present invention can be synthesized by the above methods, but the present invention is not limited thereto and other synthesis methods may be employed.

The structures described in this embodiment can be used in combination with any of the structures described in the other embodiments as appropriate.

### Embodiment 2

In this embodiment, the organic semiconductor device of one embodiment of the present invention where the organic compound of one embodiment of the present invention can be used will be described.

The organic compound of one embodiment of the present invention is suitable for a light-emitting device of organic semiconductor devices, such as an organic light-emitting diode (OLED) and can also be used for other organic semiconductor devices. Examples of other applications include photoelectric conversion devices such as an organic optical sensor and an organic thin film solar cell, an organic field-effect transistor, a semiconductor gas sensor, a diode, an inverter, and a storage device.

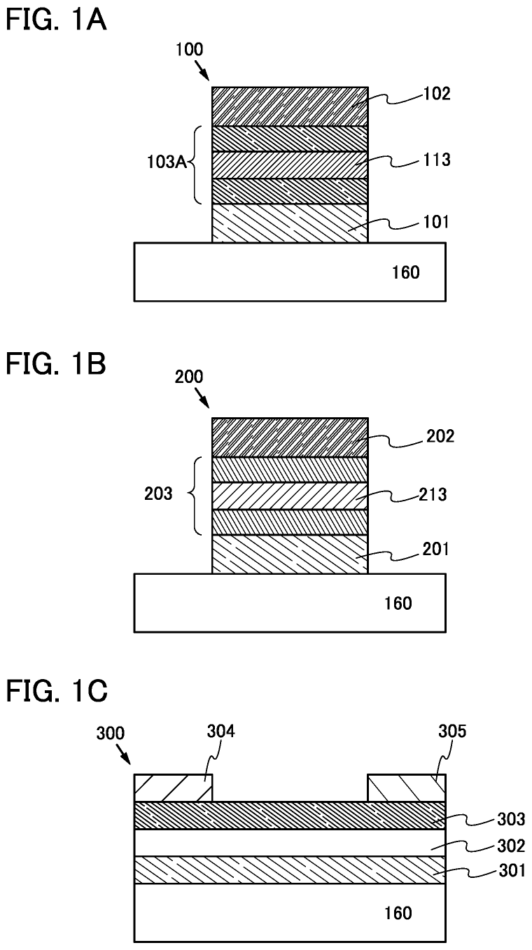

FIGS. 1A to 1C are cross-sectional views illustrating a light-emitting device, a photoelectric conversion device, and an organic field-effect transistor, each of which is an example of the organic semiconductor device of one embodiment of the present invention.

FIG. 1A is a cross-sectional view of a light-emitting device 100. The light-emitting device 100 includes a first electrode 101 and a second electrode 102 which are provided over a substrate 160, and an organic compound layer 103A held between the first electrode 101 and the second electrode 102. The organic compound layer 103A includes a light-emitting layer 113, and the light-emitting layer 113 contains a light-emitting material. When voltage is applied between the first electrode 101 and the second electrode 102, light is emitted from the organic compound layer 103A; accordingly, the light-emitting device 100 can be used as an organic light-emitting diode. Although not illustrated, the organic compound layer 103A may include, in addition to the light-emitting layer 113, a variety of layers such as a hole-injection layer, a hole-transport layer, an electron-transport layer, an electron-injection layer, a hole-blocking layer, an electron-blocking layer, a charge-generation layer, and a cap layer. The organic compound layer 103A may include a plurality of the light-emitting layers 113. In this specification, the organic compound layer 103A included in the light-emitting device 100 is referred to as an EL layer in some cases. The organic compound of one embodiment of the present invention can be used for, among the layers included in the organic compound layer 103A, the light-emitting layer 113, the hole-injection layer, the hole-transport layer, the electron-blocking layer, the cap layer, and the like.

FIG. 1B is a cross-sectional view of a photoelectric conversion device 200. The photoelectric conversion device 200 includes a first electrode 201 and a second electrode 202 which are provided over the substrate 160, and an organic compound layer 203 held between the first electrode 201 and the second electrode 202. The organic compound layer 203 contains a photoelectric conversion layer 213, and the photoelectric conversion layer 213 contains a photoelectric conversion material. Examples of the photoelectric conversion material include inorganic semiconductors such as silicon and organic semiconductors such as organic compounds. The photoelectric conversion device 200 can generate electric charge from light entering the organic compound layer 203 and extract the electric charge as current; thus, the photoelectric conversion device 200 can be utilized for an organic optical sensor, an organic solar cell, or the like. Note that voltage may be applied between the first electrode 201 and the second electrode 202. Although not illustrated, the organic compound layer 203 may include, in addition to the photoelectric conversion layer 213, a variety of layers such as a hole-injection layer, a hole-transport layer, an electron-transport layer, an electron-injection layer, a hole-blocking layer, an electron-blocking layer, and a charge-generation layer. The organic compound layer 203 may include a plurality of the photoelectric conversion layers 213. The organic compound of one embodiment of the present invention can be used for, among the layers included in the organic compound layer 203, the photoelectric conversion layer 213, the hole-injection layer, the hole-transport layer, the electron-blocking layer, and the like.

FIG. 1C is a cross-sectional view of an organic field-effect transistor 300. The organic field-effect transistor 300 includes a gate electrode 301 provided over the substrate 160, a gate insulating layer 302 over the gate electrode, an organic compound layer 303 over the gate insulating layer 302, and an electrode 304 and an electrode 305 over the organic compound layer 303. The electrode 304 functions as one of a source electrode and a drain electrode, and the electrode 305 functions as the other of the source electrode and the drain electrode. For the organic compound layer 303, the organic compound of one embodiment of the present invention can be used. Although FIG. 1C illustrates the organic field-effect transistor 300 which is of a top-contact bottom-gate type, the present invention is not limited thereto. The organic field-effect transistor 300 may have a bottom-contact bottom-gate type structure, a bottom-contact top-gate type structure, a top-contact top-gate type structure, a top-bottom contact structure, a vertical metal-base structure, or a floating metal vertical structure, for example.

When the organic compound of one embodiment of the present invention is used for the organic compound layer 103A of the light-emitting device 100, the organic compound layer 203 of the photoelectric conversion device 200, and the organic compound layer 303 of the organic field-effect transistor 300, holes can be transferred smoothly in the organic compound layers. In addition, the highly reliable organic semiconductor devices having high quality can be manufactured. Moreover, the driving lifetime of the organic semiconductor devices can be extended, that is, the reliability thereof can be increased. Furthermore, power consumption of the organic semiconductor devices can be reduced.

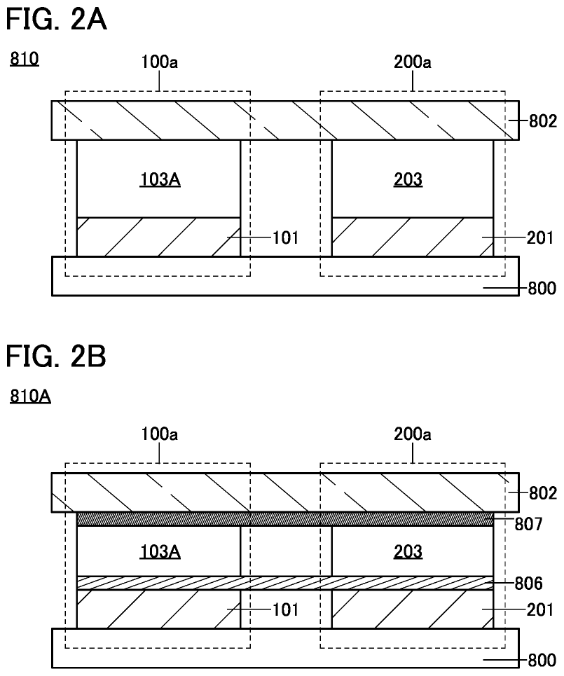

Next, a device 810 in which the light-emitting device 100 and the photoelectric conversion device 200 are provided over the same plane will be described. FIG. 2A illustrates the device 810 including a light-emitting device 100a and a photoelectric conversion device 200a over a substrate 800. Although not illustrated, in the device 810, a partition may be provided in a region surrounded by the substrate 800, the first electrode 101, the organic compound layer 103A, a second electrode 802, the organic compound layer 203, and the first electrode 201. Providing the partition can prevent a short circuit between the devices. It can also inhibit unevenness in the second electrode 802 from being generated and causing problems such as disconnection.

Note that in the device 810, the light-emitting device 100a is used as an organic light-emitting diode, and the photoelectric conversion device 200a is used as an organic optical sensor. The light-emitting device 100a and the photoelectric conversion device 200a are formed over the same substrate. Thus, the device 810 can have a structure in which an organic optical sensor is incorporated in a display device including an organic light-emitting diode, whereby the device 810 can have a function of displaying an image using the light-emitting device 100a and performing image capturing and sensing using the photoelectric conversion device 200a. The organic semiconductor device, which is easily made thin, lightweight, and large in area and has a high degree of freedom for shape and design, can be used in a variety of display devices.

Specific examples of light detected by the photoelectric conversion device 200a include visible light and infrared light. In this specification and the like, a blue (B) wavelength range is greater than or equal to 400 nm and less than 490 nm, and blue (B) light has at least one emission spectrum peak in the wavelength range. A green (G) wavelength range is greater than or equal to 490 nm and less than 580 nm, and green (G) light has at least one emission spectrum peak in the wavelength range. A red (R) wavelength range is greater than or equal to 580 nm and less than 700 nm, and red (R) light has at least one emission spectrum peak in the wavelength range. In this specification and the like, a visible wavelength range is greater than or equal to 400 nm and less than 700 nm, and visible light has at least one emission spectrum peak in the wavelength range. An infrared (IR) wavelength range is greater than or equal to 700 nm and less than 900 nm, and infrared (IR) light has at least one emission spectrum peak in the wavelength range.

In the device 810, the first electrode 101 and the first electrode 201 are provided over the same plane. In FIG. 2A, the first electrodes 101 and 201 are provided over the substrate 800. The first electrodes 101 and 201 can be formed by processing a conductive film formed over the substrate 800 into island shapes, for example. In other words, the first electrodes 101 and 201 can be formed through the same process.

As the substrate 800, a substrate having heat resistance high enough to withstand the formation of the light-emitting device 100a and the photoelectric conversion device 200a can be used. When an insulating substrate is used, a glass substrate, a quartz substrate, a sapphire substrate, a ceramic substrate, an organic resin substrate, or the like can be used as the substrate 800. Alternatively, a semiconductor substrate such as a single crystal semiconductor substrate or a polycrystalline semiconductor substrate of silicon, silicon carbide, or the like; a compound semiconductor substrate of silicon germanium or the like; an SOI substrate; or the like can be used.

In particular, it is preferable to use, as the substrate 800, the insulating substrate or the semiconductor substrate where a semiconductor circuit including a semiconductor element such as a transistor is formed. The semiconductor circuit preferably forms a pixel circuit, a gate line driver circuit (a gate driver), a source line driver circuit (a source driver), or the like. In addition to the above, an arithmetic circuit, a memory circuit, or the like may be formed.

A conductive film that transmits visible light and infrared light is used as the electrode through which light is emitted or enters, among the electrodes included in the light-emitting device 100a and the photoelectric conversion device 200a. As the electrode through which light is not emitted and does not enter, a conductive film that reflects visible light and infrared light is preferably used.

In the device 810, the second electrode 802 functions as the second electrode of each of the light-emitting device 100a and the photoelectric conversion device 200a.

The relation between the potentials of the electrodes in the case where the first electrode 101 of the light-emitting device 100a has a potential higher than that of the second electrode 802 is described. In this case, the first electrode 101 functions as an anode of the light-emitting device 100a, and the second electrode 802 functions as a cathode of the light-emitting device 100a. The first electrode 201 of the photoelectric conversion device 200a has a potential lower than that of the second electrode 802. That is, when a first potential, a second potential, and a third potential are supplied to the first electrode 101, the second electrode 802, and the first electrode 201, respectively, the first potential is higher than the second potential, and the second potential is higher than the third potential.

Next, the case where the first electrode 101 of the light-emitting device 100a has a potential lower than that of the second electrode 802 is described. In this case, the first electrode 101 functions as a cathode of the light-emitting device 100a, and the second electrode 802 functions as an anode of the light-emitting device 100a. The first electrode 201 of the photoelectric conversion device 200a has a potential lower than that of the second electrode 802 and a potential higher than that of the first electrode 101. That is, when the first potential, the second potential, and the third potential are supplied to the first electrode 101, the second electrode 802, and the first electrode 201, respectively, the second potential is higher than the third potential, and the third potential is higher than the first potential.

In the device 810, the organic compound of one embodiment of the present invention is preferably used for one or both of the organic compound layer 103A and the organic compound layer 203. In that case, holes can be transferred smoothly in the organic compound layer 103A and the organic compound layer 203. In addition, the light-emitting device 100a and the photoelectric conversion device 200a can be highly reliable organic semiconductor devices having high quality. Moreover, the driving lifetime of the light-emitting device 100a and the photoelectric conversion device 200a can be extended, that is, the reliability thereof can be improved. The process in which a hole-injection layer of the organic compound layer 103A and a hole-injection layer of the organic compound layer 203 are formed collectively and the process in which a hole-transport layer of the organic compound layer 103A and a hole-transport layer of the organic compound layer 203 are formed collectively can simplify a process and reduce production cost, which is preferable in mass production.

FIG. 2B illustrates a device 810A that is a variation example of the device 810. The device 810A is different from the device 810 in that the organic compound layer 103A and the organic compound layer 203 include a common layer 806 and a common layer 807. In the light-emitting device 100a, the common layers 806 and 807 function as part of the organic compound layer 103A. In the photoelectric conversion device 200a, the common layers 806 and 807 function as part of the organic compound layer 203. The common layer 806 includes a hole-injection layer and a hole-transport layer, for example. The common layer 807 includes an electron-transport layer and an electron-injection layer, for example.

With the common layers 806 and 807, a photoelectric conversion device can be incorporated in the device 810 without a significant increase in the number of times of separate formation of devices, whereby the device 810A can be manufactured with a high throughput.

In the device 810A, a first organic compound is preferably used for the common layer 806. Accordingly, holes can be transferred smoothly in the organic compound layer 103A and the organic compound layer 203. In addition, the light-emitting device 100a and the photoelectric conversion device 200a can be highly reliable organic semiconductor devices having high quality. Furthermore, the driving lifetime of the light-emitting device 100a and the photoelectric conversion device 200a can be extended, that is, the reliability thereof can be improved.

The resolution of the photoelectric conversion device 200a described in this embodiment can be higher than or equal to 100 ppi, preferably higher than or equal to 200 ppi, further preferably higher than or equal to 300 ppi, still further preferably higher than or equal to 400 ppi, and still further preferably higher than or equal to 500 ppi, and lower than or equal to 2000 ppi, lower than or equal to 1000 ppi, or lower than or equal to 600 ppi, for example. In particular, when the light-receiving devices 200a are arranged at a resolution higher than or equal to 200 ppi and lower than or equal to 600 ppi, preferably higher than or equal to 300 ppi and lower than or equal to 600 ppi, the photoelectric conversion device 200a can be suitably used for image capturing of a fingerprint. In fingerprint authentication with a light-emitting and light-receiving apparatus of one embodiment of the present invention, the increased resolution of the photoelectric conversion device 200a enables, for example, highly accurate extraction of the minutiae of fingerprints; thus, the accuracy of the fingerprint authentication can be increased. The resolution is preferably higher than or equal to 500 ppi, in which case the authentication conforms to the standard by the National Institute of Standards and Technology (NIST) or the like. On the assumption that the resolution at which the light-receiving devices are arranged is 500 ppi, the size of each pixel is 50.8 μm, which is adequate for image capturing of a fingerprint ridge distance (typically, greater than or equal to 300 μm and less than or equal to 500 μm).

The structures described in this embodiment can be used in appropriate combination with any of the structures described in the other embodiments.

### Embodiment 3

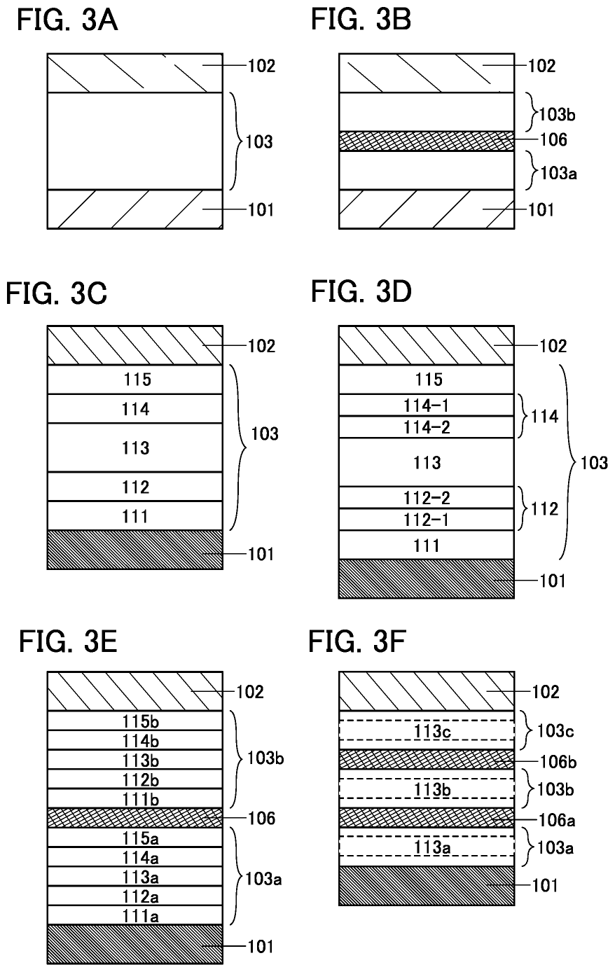

In this embodiment, structures of the organic semiconductor devices of one embodiment of the present invention are described with reference to FIGS. 3A to 3F.

### <<Basic Structure of Light-Emitting Device>>

A basic structure of a light-emitting device is described. FIG. 3A illustrates a light-emitting device including, between a pair of electrodes, an EL layer including a light-emitting layer. Specifically, an EL layer 103 is positioned between a first electrode 101 and a second electrode 102.

FIG. 3B illustrates a light-emitting device that has a stacked-layer structure (tandem structure) in which a plurality of EL layers (two EL layers 103a and 103b in FIG. 3B) are provided between a pair of electrodes and a charge-generation layer 106 is provided between the EL layers. A light-emitting device having a tandem structure enables manufacturing of a light-emitting apparatus that has high efficiency without changing the amount of current.

The charge-generation layer 106 has a function of injecting electrons into one of the EL layers 103a and 103b and injecting holes into the other of the EL layers 103a and 103b when a potential difference is caused between the first electrode 101 and the second electrode 102. Thus, when voltage is applied in FIG. 3B such that the potential of the first electrode 101 is higher than that of the second electrode 102, the charge-generation layer 106 injects electrons into the EL layer 103a and injects holes into the EL layer 103b.

Note that in terms of light extraction efficiency, the charge-generation layer 106 preferably has a property of transmitting visible light (specifically, the charge-generation layer 106 preferably has a visible light transmittance of 40% or more). The charge-generation layer 106 functions even if it has lower conductivity than the first electrode 101 or the second electrode 102.

FIG. 3C illustrates a stacked-layer structure of the EL layer 103 in the light-emitting device of one embodiment of the present invention. In this case, the first electrode 101 is regarded as functioning as an anode, and the second electrode 102 is regarded as functioning as a cathode. The EL layer 103 has a structure in which a hole-injection layer 111, a hole-transport layer 112, the light-emitting layer 113, an electron-transport layer 114, and an electron-injection layer 115 are stacked in this order over the first electrode 101. Note that the light-emitting layer 113 may have a stacked-layer structure of a plurality of light-emitting layers that emit light of different colors. For example, a light-emitting layer containing a light-emitting substance that emits red light, a light-emitting layer containing a light-emitting substance that emits green light, and a light-emitting layer containing a light-emitting substance that emits blue light may be stacked with or without a layer containing a carrier-transport material therebetween. Alternatively, a light-emitting layer containing a light-emitting substance that emits yellow light and a light-emitting layer containing a light-emitting substance that emits blue light may be used in combination. Note that the stacked-layer structure of the light-emitting layer 113 is not limited to the above. For example, the light-emitting layer 113 may have a stacked-layer structure of a plurality of light-emitting layers that emit light of the same color. For example, a first light-emitting layer containing a light-emitting substance that emits blue light and a second light-emitting layer containing a light-emitting substance that emits blue light may be stacked with or without a layer containing a carrier-transport material therebetween. The structure in which a plurality of light-emitting layers that emit light of the same color are stacked can extend driving lifetime; in other words, the structure can achieve higher reliability than a single-layer structure in some cases. In the case where a plurality of EL layers are provided as in the tandem structure illustrated in FIG. 3B, the layers in each EL layer are sequentially stacked from the anode side as described above. When the first electrode 101 is the cathode and the second electrode 102 is the anode, the stacking order of the layers in the EL layer 103 is reversed. Specifically, the layer 111 over the first electrode 101 serving as the cathode is an electron-injection layer; the layer 112 is an electron-transport layer; the layer 113 is a light-emitting layer; the layer 114 is a hole-transport layer; and the layer 115 is a hole-injection layer.

The light-emitting layer 113 included in the EL layers (103, 103a, and 103b) contains an appropriate combination of a light-emitting substance and a plurality of substances, so that fluorescent light of a desired color or phosphorescent light of a desired color can be obtained. The plurality of EL layers (103a and 103b) in FIG. 3B may exhibit their respective emission colors. In that case, the light-emitting substances and other substances can be different between the light-emitting layers.

The light-emitting device of one embodiment of the present invention can have a micro optical resonator (microcavity) structure when, for example, the first electrode 101 is a reflective electrode and the second electrode 102 is a transflective electrode in FIG. 3C. Thus, light from the light-emitting layer 113 in the EL layer 103 can be resonated between the electrodes and light emitted through the second electrode 102 can be intensified.

Note that when the first electrode 101 of the light-emitting device is a reflective electrode having a stacked-layer structure of a reflective conductive material and a light-transmitting conductive material (transparent conductive film), optical adjustment can be performed by adjusting the thickness of the transparent conductive film. Specifically, when the wavelength of light obtained from the light-emitting layer 113 is k, the optical path length between the first electrode 101 and the second electrode 102 (the product of the thickness and the refractive index) is preferably adjusted to be mλ/2 (m is an integer of 1 or more) or close to mλ/2.

To amplify desired light (wavelength: k) obtained from the light-emitting layer 113, it is preferable to adjust each of the optical path length from the first electrode 101 to a region where the desired light is obtained in the light-emitting layer 113 (light-emitting region) and the optical path length from the second electrode 102 to the region where the desired light is obtained in the light-emitting layer 113 (light-emitting region) to be (2m′+1)λ/4 (m′ is an integer of 1 or more) or close to (2m′+1)λ/4. Here, the light-emitting region means a region where holes and electrons are recombined in the light-emitting layer 113.

By such optical adjustment, the spectrum of specific monochromatic light obtained from the light-emitting layer 113 can be narrowed and light emission with high color purity can be obtained.

In the above case, the optical path length between the first electrode 101 and the second electrode 102 is, to be exact, the total thickness from a reflective region in the first electrode 101 to a reflective region in the second electrode 102. However, it is difficult to precisely determine the reflective regions in the first electrode 101 and the second electrode 102; thus, it is assumed that the above effect can be sufficiently obtained wherever the reflective regions may be set in the first electrode 101 and the second electrode 102. Furthermore, the optical path length between the first electrode 101 and the light-emitting layer that emits the desired light is, to be exact, the optical path length between the reflective region in the first electrode 101 and the light-emitting region in the light-emitting layer that emits the desired light. However, it is difficult to precisely determine the reflective region in the first electrode 101 and the light-emitting region in the light-emitting layer that emits the desired light; thus, it is assumed that the above effect can be sufficiently obtained wherever the reflective region and the light-emitting region may be set in the first electrode 101 and the light-emitting layer that emits the desired light, respectively.

FIG. 3D illustrates a modification example of the stacked-layer structure illustrated in FIG. 3C. Also in this case, the first electrode 101 is regarded as functioning as an anode, and the second electrode 102 is regarded as functioning as a cathode. In this modification example, the hole-transport layer 112 and the electron-transport layer 114 each have a stacked-layer structure of two layers. In other words, the EL layer 103 has a structure in which a hole-injection layer 111, a first hole-transport layer 112-1, a second hole-transport layer 112-2, a light-emitting layer 113, a second electron-transport layer 114-2, a first electron-transport layer 114-1, and an electron-injection layer 115 are stacked in this order over the first electrode 101. Note that the light-emitting layer 113 is positioned between the first electrode 101 and the second electrode 102. The first hole-transport layer 112-1 is positioned between the first electrode 101 and the light-emitting layer 113. The first electron-transport layer 114-1 is positioned between the light-emitting layer 113 and the second electrode 102. The hole-injection layer 111 is positioned between the first electrode 101 and the hole-transport layer 112. The electron-injection layer 115 is positioned between the electron-transport layer 114 and the second electrode 102. The second hole-transport layer 112-2 is positioned between the first hole-transport layer 112-1 and the light-emitting layer 113. In other words, the second electron-transport layer 114-2 is positioned between the light-emitting layer 113 and the first electron-transport layer 114-1. In the case where the EL layer 103 has such a stacked-layer structure, one or more of the hole-injection layer 111, the hole-transport layer 112, and the light-emitting layer 113 preferably contains the first organic compound.

The second hole-transport layer 112-2 is provided to prevent passing of electrons from the light-emitting layer 113 to the first electrode 101 side, for example. Thus, the second hole-transport layer 112-2 can also be referred to as an electron-blocking layer. The second electron-transport layer 114-2 is provided to prevent passing of holes from the light-emitting layer 113 to the second electrode 102 side, for example. Thus, the second electron-transport layer 114-2 can also be referred to as a hole-blocking layer.

The light-emitting device illustrated in FIG. 3E is a light-emitting device having a tandem structure. Owing to a microcavity structure of the light-emitting device, light (monochromatic light) with different wavelengths from the EL layers (103a and 103b) can be extracted. Thus, it is unnecessary to separately form EL layers for obtaining a plurality of emission colors (e.g., R, G, and B). Thus, high resolution can be easily achieved. A combination with coloring layers (color filters) is also possible. Furthermore, the emission intensity of light with a specific wavelength in the front direction can be increased, whereby power consumption can be reduced.

The light-emitting device illustrated in FIG. 3F is an example of the light-emitting device having the tandem structure illustrated in FIG. 3B, and includes three EL layers (103a, 103b, and 103c) stacked with charge-generation layers (106a and 106b) positioned therebetween, as illustrated in FIG. 3F. The three EL layers (103a, 103b, and 103c) include respective light-emitting layers (113a, 113b, and 113c), and the emission colors of the light-emitting layers can be selected freely. For example, the light-emitting layer 113a can emit blue light, the light-emitting layer 113b can emit red light, green light, or yellow light, and the light-emitting layer 113c can emit blue light, or the light-emitting layer 113a can emit red light, the light-emitting layer 113b can emit blue light, green light, or yellow light, and the light-emitting layer 113c can emit red light.

In the light-emitting device of one embodiment of the present invention, at least one of the first electrode 101 and the second electrode 102 is a light-transmitting electrode (e.g., a transparent electrode or a transflective electrode). In the case where the light-transmitting electrode is a transparent electrode, the transparent electrode has a visible light transmittance higher than or equal to 40%. In the case where the light-transmitting electrode is a transflective electrode, the transflective electrode has a visible light reflectance higher than or equal to 20% and lower than or equal to 80%, preferably higher than or equal to 40% and lower than or equal to 70%. These electrodes preferably have a resistivity of 1×10⁻² Ωcm or less.

When one of the first electrode 101 and the second electrode 102 is a reflective electrode in the light-emitting device of one embodiment of the present invention, the visible light reflectance of the reflective electrode is higher than or equal to 40% and lower than or equal to 100%, preferably higher than or equal to 70% and lower than or equal to 100%. This electrode preferably has a resistivity of 1×10⁻² Ωcm or less.

### <<Specific Structure of Light-Emitting Device>>

Next, a specific structure of the light-emitting device of one embodiment of the present invention will be described. Here, the description is made using FIG. 3E illustrating the tandem structure. Note that the structure of the EL layer applies also to the structure of the light-emitting devices having a single structure in FIGS. 3A and 3C. When the light-emitting device in FIG. 3E has a microcavity structure, the first electrode 101 is formed as a reflective electrode and the second electrode 102 is formed as a transflective electrode. Thus, a single-layer structure or a stacked-layer structure can be formed using one or more kinds of desired electrode materials. Note that the second electrode 102 is formed after formation of the EL layer 103b, with the use of a material selected as appropriate.

### <First Electrode and Second Electrode>

As materials for the first electrode 101 and the second electrode 102, any of the following materials can be used in an appropriate combination as long as the above functions of the electrodes can be fulfilled. For example, a metal, an alloy, an electrically conductive compound, a mixture of these, and the like can be used as appropriate. Specifically, an In—Sn oxide (also referred to as ITO), an In—Si—Sn oxide (also referred to as ITSO), an In—Zn oxide, or an In—W—Zn oxide can be used. In addition, it is possible to use a metal such as aluminum (Al), titanium (Ti), chromium (Cr), manganese (Mn), iron (Fe), cobalt (Co), nickel (Ni), copper (Cu), gallium (Ga), zinc (Zn), indium (In), tin (Sn), molybdenum (Mo), tantalum (Ta), tungsten (W), palladium (Pd), gold (Au), platinum (Pt), silver (Ag), yttrium (Y), or neodymium (Nd) or an alloy containing an appropriate combination of any of these metals. It is also possible to use a Group 1 element or a Group 2 element in the periodic table that is not described above (e.g., lithium (Li), cesium (Cs), calcium (Ca), or strontium (Sr)), a rare earth metal such as europium (Eu) or ytterbium (Yb), an alloy containing an appropriate combination of any of these elements, graphene, or the like.

In the light-emitting device in FIG. 3E, when the first electrode 101 is the anode, a hole-injection layer 111a and a hole-transport layer 112a of the EL layer 103a are sequentially stacked over the first electrode 101 by a vacuum evaporation method. After the EL layer 103a and the charge-generation layer 106 are formed, a hole-injection layer 111b and a hole-transport layer 112b of the EL layer 103b are sequentially stacked over the charge-generation layer 106 in a similar manner.

The light-emitting device illustrated in FIG. 3E can have a micro optical resonator (microcavity) structure when the first electrode 101 is a reflective electrode and the second electrode 102 is a transflective electrode. Thus, light from the light-emitting layer 113 in the EL layer 103 can be resonated between the electrodes and light emitted through the second electrode 102 can be intensified.

Note that when the first electrode 101 of the light-emitting device is a reflective electrode having a stacked-layer structure of a reflective conductive material and a light-transmitting conductive material (transparent conductive film), optical adjustment can be performed by adjusting the thickness of the transparent conductive film. Specifically, when the wavelength of light obtained from the light-emitting layer 113 is k, the optical path length between the first electrode 101 and the second electrode 102 (the product of the thickness and the refractive index) is preferably adjusted to be mλ/2 (m is an integer of 1 or more) or close to mλ/2.

To amplify desired light (wavelength: k) obtained from the light-emitting layer 113, it is preferable to adjust each of the optical path length from the first electrode 101 to a region where the desired light is obtained in the light-emitting layer 113 (light-emitting region) and the optical path length from the second electrode 102 to the region where the desired light is obtained in the light-emitting layer 113 (light-emitting region) to be (2m′+1)λ/4 (m′ is an integer of 1 or more) or close to (2m′+1)λ/4. Here, the light-emitting region means a region where holes and electrons are recombined in the light-emitting layer 113.

By such optical adjustment, the spectrum of specific monochromatic light obtained from the light-emitting layer 113 can be narrowed and light emission with high color purity can be obtained.

In the above case, the optical path length between the first electrode 101 and the second electrode 102 is, to be exact, the total thickness from a reflective region in the first electrode 101 to a reflective region in the second electrode 102. However, it is difficult to precisely determine the reflective regions in the first electrode 101 and the second electrode 102; thus, it is assumed that the above effect can be sufficiently obtained wherever the reflective regions may be set in the first electrode 101 and the second electrode 102. Furthermore, the optical path length between the first electrode 101 and the light-emitting layer that emits the desired light is, to be exact, the optical path length between the reflective region in the first electrode 101 and the light-emitting region in the light-emitting layer that emits the desired light. However, it is difficult to precisely determine the reflective region in the first electrode 101 and the light-emitting region in the light-emitting layer that emits the desired light; thus, it is assumed that the above effect can be sufficiently obtained wherever the reflective region and the light-emitting region may be set in the first electrode 101 and the light-emitting layer that emits the desired light, respectively.

In the light-emitting device of one embodiment of the present invention, at least one of the first electrode 101 and the second electrode 102 is a light-transmitting electrode (e.g., a transparent electrode or a transflective electrode). In the case where the light-transmitting electrode is a transparent electrode, the transparent electrode has a visible light transmittance higher than or equal to 40%. In the case where the light-transmitting electrode is a transflective electrode, the transflective electrode has a visible light reflectance higher than or equal to 20% and lower than or equal to 80%, preferably higher than or equal to 40% and lower than or equal to 70%. These electrodes preferably have a resistivity of 1×10⁻² Ωcm or less.

When one of the first electrode 101 and the second electrode 102 is a reflective electrode in the light-emitting device of one embodiment of the present invention, the visible light reflectance of the reflective electrode is higher than or equal to 40% and lower than or equal to 100%, preferably higher than or equal to 70% and lower than or equal to 100%. This electrode preferably has a resistivity of 1×10⁻² Ωcm or less.

### <Hole-Injection Layer>

The hole-injection layers (111, 111a, and 111b) inject holes from the first electrode 101 serving as the anode and the charge-generation layers (106, 106a, and 106b) to the EL layers (103, 103a, and 103b) and contain an organic acceptor material or a material having a high hole-injection property.

The organic acceptor material allows holes to be generated in another organic compound whose HOMO level is close to the LUMO level of the organic acceptor material when charge separation is caused between the organic acceptor material and the organic compound. Thus, as the organic acceptor material, a compound having an electron-withdrawing group (e.g., a halogen group or a cyano group), such as a quinodimethane derivative, a chloranil derivative, and a hexaazatriphenylene derivative, can be used. Examples of the organic acceptor material include 7,7,8,8-tetracyano-2,3,5,6-tetrafluoroquinodimethane (abbreviation: F₄-TCNQ), 3,6-difluoro-2,5,7,7,8,8-hexacyanoquinodimethane, chloranil, 2,3,6,7,10,11-hexacyano-1,4,5,8,9,12-hexaazatriphenylene (abbreviation: HAT-CN), 1,3,4,5,7,8-hexafluorotetracyano-naphthoquinodimethane (abbreviation: F6-TCNNQ), and 2-(7-dicyanomethylen-1,3,4,5,6,8,9,10-octafluoro-7H-pyren-2-ylidene)malononitrile. Note that among organic acceptor materials, a compound in which electron-withdrawing groups are bonded to fused aromatic rings each having a plurality of heteroatoms, such as HAT-CN, is particularly preferable because it has a high acceptor property and stable film quality against heat. Besides, a [3]radialene derivative having an electron-withdrawing group (particularly a cyano group or a halogen group such as a fluoro group), which has a very high electron-accepting property, is preferred; specific examples include α,α′,α″-1,2,3-cyclopropanetriylidenetris[4-cyano-2,3,5,6-tetrafluorobenzeneacetonitrile], α,α′,α″-1,2,3-cyclopropanetriylidenetris[2,6-dichloro-3,5-difluoro-4-(trifluoromethyl)benzeneacetonitrile], and α,α′,α″-1,2,3-cyclopropanetriylidenetris[2,3,4,5,6-pentafluorobenzeneacetonitrile].

As the material having a high hole-injection property, an oxide of a metal belonging to Group 4 to Group 8 of the periodic table (e.g., a transition metal oxide such as molybdenum oxide, vanadium oxide, ruthenium oxide, tungsten oxide, or manganese oxide) can be used. Specific examples include molybdenum oxide, vanadium oxide, niobium oxide, tantalum oxide, chromium oxide, tungsten oxide, manganese oxide, and rhenium oxide. Among the above oxides, molybdenum oxide is preferable because it is stable in the air, has a low hygroscopic property, and is easily handled. Other examples include a perylenetetracarboxylic acid derivative such as diquinoxalino[2,3-a:2′,3′-c]phenazine (abbreviation: HATNA), 2,3,8,9,14,15-hexafluorodiquinoxalino[2,3-a:2′,3′-c]phenazine (abbreviation: HATNA-F6), 3,4,9,10-perylenetetracarboxylic diimide (abbreviation: PTCDI), or 3,4,9,10-perylenetetracarboxyl-bis-benzimidazole (abbreviation: PTCBI); (C₆₀-Iₕ) [5,6]fullerene (abbreviation: C₆₀); (C₇₀-D₅ₕ) [5,6]fullerene (abbreviation: C₇₀); an organic compound such as phthalocyanine (abbreviation: H₂Pc); and a metal phthalocyanine containing copper, zinc, cobalt, iron, chromium, nickel, or the like or a derivative thereof, such as copper phthalocyanine (abbreviation: CuPc), zinc phthalocyanine (abbreviation: ZnPc), cobalt phthalocyanine (abbreviation: CoPc), iron phthalocyanine (abbreviation: FePc), tin phthalocyanine (abbreviation: SnPc), tin oxide phthalocyanine (abbreviation: SnOPc), titanium oxide phthalocyanine (abbreviation: TiOPc), or vanadium oxide phthalocyanine (abbreviation: VOPc). A phthalocyanine-based metal complex such as CuPc or ZnPc and 2,3,8,9,14,15-hexafluorodiquinoxalino[2,3-a:2′,3′-c]phenazine are especially preferable. Among these materials, CuPc and ZnPc are preferable because they are inexpensive and have favorable characteristics. Using ZnPc, which has a low diffusion coefficient with respect to silicon, reduces the probability that metal diffusion to a semiconductor adversely affects the semiconductor characteristics; accordingly, ZnPc is particularly suitable for a display device manufactured using a silicon semiconductor.

Other examples are aromatic amine compounds, which are low-molecular compounds, such as 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), 4,4′-bis[N-(4-diphenylaminophenyl)-N-phenylamino]biphenyl (abbreviation: DPAB), N,N-bis[4-bis(3-methylphenyl)aminophenyl]-N,N-diphenyl-4,4′-diaminobiphenyl (abbreviation: DNTPD), 1,3,5-tris[N-(4-diphenylaminophenyl)-N-phenylamino]benzene (abbreviation: DPA3B), 3-[N-(9-phenylcarbazol-3-yl)-N-phenylamino]-9-phenylcarbazole (abbreviation: PCzPCA1), 3,6-bis[N-(9-phenylcarbazol-3-yl)-N-phenylamino]-9-phenylcarbazole (abbreviation: PCzPCA2), and 3-[N-(1-naphthyl)-N-(9-phenylcarbazol-3-yl)amino]-9-phenylcarbazole (abbreviation: PCzPCN1).

Other examples are high-molecular compounds (e.g., oligomers, dendrimers, and polymers) such as poly(N-vinylcarbazole) (abbreviation: PVK), poly(4-vinyltriphenylamine) (abbreviation: PVTPA), poly[N-(4-{N¹-[4-(4-diphenylamino)phenyl]phenyl-N-phenylamino}phenyl)methacrylamide] (abbreviation: PTPDMA), and poly[N,N-bis(4-butylphenyl)-N,N-bis(phenyl)benzidine] (abbreviation: Poly-TPD). Alternatively, it is possible to use a high-molecular compound to which acid is added, such as poly(3,4-ethylenedioxythiophene)/polystyrenesulfonic acid (abbreviation: PEDOT/PSS) or polyaniline/polystyrenesulfonic acid (abbreviation: PAni/PSS), for example.

As the material having a high hole-injection property, a mixed material containing a hole-transport material and the above-described organic acceptor material (electron-accepting material) can be used. In that case, the organic acceptor material extracts electrons from the hole-transport material, so that holes are generated in the hole-injection layer 111 and the holes are injected into the light-emitting layer 113 through the hole-transport layer 112. Note that the hole-injection layer 111 may be formed to have a single-layer structure using a mixed material containing a hole-transport material and an organic acceptor material (electron-accepting material), or a stacked-layer structure of a layer containing a hole-transport material and a layer containing an organic acceptor material (electron-accepting material).

The hole-transport material preferably has a hole mobility higher than or equal to 1×10⁻⁶ cm²/Vs in the case where the square root of the electric field strength [V/cm] is 600. Note that other substances can also be used as long as the substances have hole-transport properties higher than electron-transport properties.

As the hole-transport material, materials having a high hole-transport property, such as a compound having a π-electron rich heteroaromatic ring (e.g., a carbazole derivative, a furan derivative, and a thiophene derivative) and an aromatic amine (an organic compound having an aromatic amine skeleton), are preferable. The organic compound described in Embodiment 1 has a hole-transport property and thus can be used as a hole-transport material.

Examples of the carbazole derivative (an organic compound having a carbazole ring) include a bicarbazole derivative (e.g., a 3,3′-bicarbazole derivative) and an aromatic amine having a carbazolyl group.

Specific examples of the bicarbazole derivative (e.g., a 3,3′-bicarbazole derivative) include 3,3′-bis(9-phenyl-9H-carbazole) (abbreviation: PCCP), 9,9′-bis(biphenyl-4-yl)-3,3′-bi-9H-carbazole (abbreviation: BisBPCz), 9,9′-bis(biphenyl-3-yl)-3,3′-bi(9H-carbazole) (abbreviation: BismBPCz), 9-(biphenyl-3-yl)-9′-(biphenyl-4-yl)-9H,9′H-3,3′-bicarbazole (abbreviation: mBPCCBP), and 9-(2-naphthyl)-9′-phenyl-9H,9′H-3,3′-bicarbazole (abbreviation: PNCCP).

Specific examples of the aromatic amine having a carbazolyl group include 4-phenyl-4′-(9-phenyl-9H-carbazol-3-yl)triphenylamine (abbreviation: PCBA1BP), N-(4-biphenyl)-N-(9,9-dimethyl-9H-fluoren-2-yl)-9-phenyl-9H-carbazol-3-amine (abbreviation: PCBiF), N-(biphenyl-4-yl)-N-[4-(9-phenyl-9H-carbazol-3-yl)phenyl]-9,9-dimethyl-9H-fluoren-2-amine (abbreviation: PCBBiF), N-[4-(9-phenyl-9H-carbazol-3-yl)phenyl]bis(9,9-dimethyl-9H-fluoren-2-yl)amine (abbreviation: PCBFF), N-(1,1′-biphenyl-4-yl)-N-[4-(9-phenyl-9H-carbazol-3-yl)phenyl]-9,9-dimethyl-9H-fluoren-4-amine, N-[4-(9-phenyl-9H-carbazol-3-yl)phenyl]-(9,9-dimethyl-9H-fluoren-2-yl)-9,9-dimethyl-9H-fluoren-4-amine, N-(1,1′-biphenyl-4-yl)-N-[4-(9-phenyl-9H-carbazol-3-yl)phenyl]-9,9-diphenyl-9H-fluoren-2-amine, N-(1,1′-biphenyl-4-yl)-N-[4-(9-phenyl-9H-carbazol-3-yl)phenyl]-9,9-diphenyl-9H-fluoren-4-amine, N-(1,1′-biphenyl-4-yl)-N-[4-(9-phenyl-9H-carbazol-3-yl)phenyl]-9,9′-spirobi(9H-fluoren)-2-amine, N-(1,1′-biphenyl-4-yl)-N-[4-(9-phenyl-9H-carbazol-3-yl)phenyl]-9,9′-spirobi(9H-fluoren)-4-amine, N-[4-(9-phenyl-9H-carbazol-3-yl)phenyl]-N-[1,1′: 3′,1″-terphenyl-4-yl]-9,9-dimethyl-9H-fluoren-2-amine, N-[4-(9-phenyl-9H-carbazol-3-yl)phenyl]-N-[1,1′: 4′,1″-terphenyl-4-yl]-9,9-dimethyl-9H-fluoren-2-amine, N-[4-(9-phenyl-9H-carbazol-3-yl)phenyl]-N-[1,1′: 3′,1″-terphenyl-4-yl]-9,9-dimethyl-9H-fluoren-4-amine, N-[4-(9-phenyl-9H-carbazol-3-yl)phenyl]-N-[1,1′: 4′,1″-terphenyl-4-yl]-9,9-dimethyl-9H-fluoren-4-amine, 4,4′-diphenyl-4″-(9-phenyl-9H-carbazol-3-yl)triphenylamine (abbreviation: PCBBi1BP), 4-(1-naphthyl)-4′-(9-phenyl-9H-carbazol-3-yl)triphenylamine (abbreviation: PCBANB), 4,4′-di(1-naphthyl)-4″-(9-phenyl-9H-carbazol-3-yl)triphenylamine (abbreviation: PCBNBB), 4-phenyldiphenyl-(9-phenyl-9H-carbazol-3-yl)amine (abbreviation: PCA1BP), N,N-bis(9-phenylcarbazol-3-yl)-N,N-diphenylbenzene-1,3-diamine (abbreviation: PCA2B), N,N,N′-triphenyl-N,N,N′-tris(9-phenylcarbazol-3-yl)benzene-1,3,5-triamine (abbreviation: PCA3B), 9,9-dimethyl-N-phenyl-N-[4-(9-phenyl-9H-carbazol-3-yl)phenyl]fluoren-2-amine (abbreviation: PCBAF), N-phenyl-N-[4-(9-phenyl-9H-carbazol-3-yl)phenyl]-9,9′-spirobi[9H-fluoren]-2-amine (abbreviation: PCBASF), 3-[N-(9-phenylcarbazol-3-yl)-N-phenylamino]-9-phenylcarbazole (abbreviation: PCzPCA1), 3,6-bis[N-(9-phenylcarbazol-3-yl)-N-phenylamino]-9-phenylcarbazole (abbreviation: PCzPCA2), 3-[N-(1-naphthyl)-N-(9-phenylcarbazol-3-yl)amino]-9-phenylcarbazole (abbreviation: PCzPCN1), 3-[N-(4-diphenylaminophenyl)-N-phenylamino]-9-phenylcarbazole (abbreviation: PCzDPA1), 3,6-bis[N-(4-diphenylaminophenyl)-N-phenylamino]-9-phenylcarbazole (abbreviation: PCzDPA2), 3,6-bis[N-(4-diphenylaminophenyl)-N-(1-naphthyl)amino]-9-phenylcarbazole (abbreviation: PCzTPN2), N-(9,9-spirobi[9H-fluoren]-2-yl)-N,9-diphenylcarbazol-3-amine (abbreviation: PCASF), N-[4-(9H-carbazol-9-yl)phenyl]-N-(4-phenyl)phenylaniline (abbreviation: YGA1BP), N,N-bis[4-(carbazol-9-yl)phenyl]-N,N-diphenyl-9,9-dimethylfluorene-2,7-diamine (abbreviation: YGA2F), and 4,4′,4″-tris(carbazol-9-yl)triphenylamine (abbreviation: TCTA).

Other examples of the carbazole derivative include 9-[4-(9-phenyl-9H-carbazol-3-yl)phenyl]phenanthrene (abbreviation: PCPPn), 3-[4-(1-naphthyl)-phenyl]-9-phenyl-9H-carbazole (abbreviation: PCPN), 1,3-bis(N-carbazolyl)benzene (abbreviation: mCP), 4,4′-di(N-carbazolyl)biphenyl (abbreviation: CBP), 3,6-bis(3,5-diphenylphenyl)-9-phenylcarbazole (abbreviation: CzTP), 1,3,5-tris[4-(N-carbazolyl)phenyl]benzene (abbreviation: TCPB), and 9-[4-(10-phenyl-9-anthracenyl)phenyl]-9H-carbazole (abbreviation: CzPA).

Specific examples of the furan derivative (an organic compound having a furan ring) include 4,4′,4″-(benzene-1,3,5-triyl)tri(dibenzofuran) (abbreviation: DBF3P-II) and 4-{3-[3-(9-phenyl-9H-fluoren-9-yl)phenyl]phenyl}dibenzofuran (abbreviation: mmDBFFLBi-II).

Specific examples of the thiophene derivative (an organic compound having a thiophene ring) include 4,4′,4″-(benzene-1,3,5-triyl)tri(dibenzothiophene) (abbreviation: DBT3P-II), 2,8-diphenyl-4-[4-(9-phenyl-9H-fluoren-9-yl)phenyl]dibenzothiophene (abbreviation: DBTFLP-III), and 4-[4-(9-phenyl-9H-fluoren-9-yl)phenyl]-6-phenyldibenzothiophene (abbreviation: DBTFLP-IV).

Specific examples of the aromatic amine include 4,4′-bis[N-(1-naphthyl)-N-phenylamino]biphenyl (abbreviation: NPB or α-NPD), N,N-diphenyl-N,N-bis(3-methylphenyl)-4,4′-diaminobiphenyl (abbreviation: TPD), N,N-bis(9,9′-spirobi[9H-fluoren]-2-yl)-N,N-diphenyl-4-4′-diaminobiphenyl (abbreviation: BSPB), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), N-(9,9-dimethyl-9H-fluoren-2-yl)-N-{9,9-dimethyl-2-[N-phenyl-N-(9,9-dimethyl-9H-fluoren-2-yl)amino]-9H-fluoren-7-yl}phenylamine (abbreviation: DFLADFL), N-(9,9-dimethyl-2-diphenylamino-9H-fluoren-7-yl)diphenylamine (abbreviation: DPNF), N-(9,9-spirobi[9H-fluoren]-2-yl)-N,N,N-triphenyl-1,4-phenyldiamine (abbreviation: DPASF), N,N-diphenyl-N,N-bis(4-diphenylaminophenyl)spirobi[9H-fluorene]-2,7-diamine (abbreviation: DPA2SF), 4,4′,4″-tris[N-(1-naphthyl)-N-phenylamino]triphenylamine (abbreviation: 1′-TNATA), 4,4′,4″-tris(N,N-diphenylamino)triphenylamine (abbreviation: TDATA), 4,4′,4″-tris[N-(3-methylphenyl)-N-phenylamino]triphenylamine (abbreviation: m-MTDATA), N,N-di(p-tolyl)-N,N-diphenyl-p-phenylenediamine (abbreviation: DTDPPA), 4,4′-bis[N-(4-diphenylaminophenyl)-N-phenylamino]biphenyl (abbreviation: DPAB), DNTPD, 1,3,5-tris[N-(4-diphenylaminophenyl)-N-phenylamino]benzene (abbreviation: DPA3B), N-(4-biphenyl)-6,N-diphenylbenzo[b]naphtho[1,2-d]furan-8-amine (abbreviation: BnfABP), N,N-bis(4-biphenyl)-6-phenylbenzo[b]naphtho[1,2-d]furan-8-amine (abbreviation: BBABnf), 4,4′-bis(6-phenylbenzo[b]naphtho[1,2-d]furan-8-yl)-4″-phenyltriphenylamine (abbreviation: BnfBB1BP), N,N-bis(4-biphenyl)benzo[b]naphtho[1,2-d]furan-6-amine (abbreviation: BBABnf(6)), N,N-bis(4-biphenyl)benzo[b]naphtho[1,2-d]furan-8-amine (abbreviation: BBABnf(8)), N,N-bis(4-biphenyl)benzo[b]naphtho[2,3-d]furan-4-amine (abbreviation: BBABnf(II)(4)), N,N-bis[4-(dibenzofuran-4-yl)phenyl]-4-amino-p-terphenyl (abbreviation: DBfBB1TP), N-[4-(dibenzothiophen-4-yl)phenyl]-N-phenyl-4-biphenylamine (abbreviation: ThBA1BP), 4-(2-naphthyl)-4′,4″-diphenyltriphenylamine (abbreviation: BBAβNB), 4-[4-(2-naphthyl)phenyl]-4′,4″-diphenyltriphenylamine (abbreviation: BBAβNBi), 4,4′-diphenyl-4″-(6;1′-binaphthyl-2-yl)triphenylamine (abbreviation: BBAαNβNB), 4,4′-diphenyl-4″-(7;1′-binaphthyl-2-yl)triphenylamine (abbreviation: BBAαNβNB-03), 4,4′-diphenyl-4″-(7-phenyl)naphthyl-2-yltriphenylamine (abbreviation: BBAPPNB-03), 4,4′-diphenyl-4″-(6;2′-binaphthyl-2-yl)triphenylamine (abbreviation: BBA(βN2)B), 4,4′-diphenyl-4″-(7;2′-binaphthyl-2-yl)triphenylamine (abbreviation: BBA(βN2)B-03), 4,4′-diphenyl-4″-(4;2′-binaphthyl-1-yl)triphenylamine (abbreviation: BBAβNαNB), 4,4′-diphenyl-4″-(5;2′-binaphthyl-1-yl)triphenylamine (abbreviation: BBAβNαNB-02), 4-(4-biphenylyl)-4′-(2-naphthyl)-4″-phenyltriphenylamine (abbreviation: TPBiAβNB), 4-(3-biphenylyl)-4′-[4-(2-naphthyl)phenyl]-4″-phenyltriphenylamine (abbreviation: mTPBiAβNBi), 4-(4-biphenylyl)-4′-[4-(2-naphthyl)phenyl]-4″-phenyltriphenylamine (abbreviation: TPBiAPNBi), 4-phenyl-4′-(1-naphthyl)-triphenylamine (abbreviation: αNBA1BP), 4,4′-bis(1-naphthyl)triphenylamine (abbreviation: αNBB1BP), 4,4′-diphenyl-4″-[4′-(carbazol-9-yl)biphenyl-4-yl]triphenylamine (abbreviation: YGTBi1BP), 4′-[4-(3-phenyl-9H-carbazol-9-yl)phenyl]tris(biphenyl-4-yl)amine (abbreviation: YGTBi1BP-02), 4-[4′-(carbazol-9-yl)biphenyl-4-yl]-4′-(2-naphthyl)-4″-phenyltriphenylamine (abbreviation: YGTBiPNB), N-[4-(9-phenyl-9H-carbazol-3-yl)phenyl]-N-[4-(1-naphthyl)phenyl]-9,9′-spirobi[9H-fluoren]-2-amine (abbreviation: PCBNBSF), N,N-bis(biphenyl-4-yl)-9,9′-spirobi[9H-fluoren]-2-amine (abbreviation: BBASF), N,N-bis(biphenyl-4-yl)-9,9′-spirobi[9H-fluoren]-4-amine (abbreviation: BBASF(4)), N-(biphenyl-2-yl)-N-(9,9-dimethyl-9H-fluoren-2-yl)-9,9′-spirobi[9H-fluoren]-4-amine (abbreviation: oFBiSF), N-(biphenyl-4-yl)-N-(9,9-dimethyl-9H-fluoren-2-yl)dibenzofuran-4-amine (abbreviation: FrBiF), N-[4-(1-naphthyl)phenyl]-N-[3-(6-phenyldibenzofuran-4-yl)phenyl]-1-naphthylamine (abbreviation: mPDBfBNBN), 4-phenyl-4′-[4-(9-phenylfluoren-9-yl)phenyl]triphenylamine (abbreviation: BPAFLBi), N,N-bis(9,9-dimethyl-9H-fluoren-2-yl)-9,9′-spirobi-9H-fluoren-4-amine, N,N-bis(9,9-dimethyl-9H-fluoren-2-yl)-9,9′-spirobi-9H-fluoren-3-amine, N,N-bis(9,9-dimethyl-9H-fluoren-2-yl)-9,9′-spirobi-9H-fluoren-2-amine, and N,N-bis(9,9-dimethyl-9H-fluoren-2-yl)-9,9′-spirobi-9H-fluoren-1-amine.

Other examples of the hole-transport material include high-molecular compounds (e.g., oligomers, dendrimers, and polymers) such as poly(N-vinylcarbazole) (abbreviation: PVK), poly(4-vinyltriphenylamine) (abbreviation: PVTPA), poly[N-(4-{N-[4-(4-diphenylamino)phenyl]phenyl-N-phenylamino}phenyl)methacrylamide](abbreviation: PTPDMA), and poly[N,N-bis(4-butylphenyl)-N,N-bis(phenyl)benzidine](abbreviation: Poly-TPD). Alternatively, it is possible to use a high-molecular compound to which acid is added, such as poly(3,4-ethylenedioxythiophene)/polystyrenesulfonic acid (abbreviation: PEDOT/PSS) or polyaniline/polystyrenesulfonic acid (abbreviation: PAni/PSS), for example.

Note that the hole-transport material is not limited to the above examples, and any of a variety of known materials may be used alone or in combination as the hole-transport material.

The hole-injection layers (111, 111a, and 111b) can be formed by any of known film formation methods such as a vacuum evaporation method.

### <Hole-Transport Layer>

The hole-transport layers (112, 112a, and 112b) transport the holes, which are injected from the first electrodes 101 by the hole-injection layers (111, 111a, and 111b), to the light-emitting layers (113, 113a, and 113b). Note that the hole-transport layers (112, 112a, and 112b) each contain a hole-transport material. Thus, the hole-transport layers (112, 112a, and 112b) can be formed using hole-transport materials that can be used for the hole-injection layers (111, 111a, and 111b).

Note that in the light-emitting device of one embodiment of the present invention, the organic compound used for the hole-transport layers (112, 112a, and 112b) can also be used for the light-emitting layers (113, 113a, and 113b). The use of the same organic compound for the hole-transport layers (112, 112a, and 112b) and the light-emitting layers (113, 113a, and 113b) is preferable, in which case holes can be efficiently transported from the hole-transport layers (112, 112a, and 112b) to the light-emitting layers (113, 113a, and 113b).

### <Light-Emitting Layer>

The light-emitting layers (113, 113a, and 113b) contain a light-emitting substance. Note that as a light-emitting substance that can be used in the light-emitting layers (113, 113a, and 113b), a substance whose emission color is blue, violet, bluish violet, green, yellowish green, yellow, orange, red, or the like can be used as appropriate. When a plurality of light-emitting layers are provided, the use of different light-emitting substances for the light-emitting layers enables a structure that exhibits different emission colors (e.g., white light emission obtained by a combination of complementary emission colors). When a plurality of light-emitting layers are provided, the light-emitting layers can exhibit the same color. The structure in which a plurality of light-emitting layers that emit light of the same color are stacked can sometimes achieve higher reliability than a single-layer structure. Furthermore, one light-emitting layer may have a stacked-layer structure including different light-emitting substances.

The light-emitting layers (113, 113a, and 113b) may each contain one or more kinds of organic compounds (e.g., a host material) in addition to a light-emitting substance (a guest material).

In the case where a plurality of host materials are used in the light-emitting layers (113, 113a, and 113b), a second host material that is additionally used is preferably a substance having a larger energy gap than those of a known guest material and a first host material. Preferably, the lowest singlet excitation energy level (S1 level) of the second host material is higher than that of the first host material, and the lowest triplet excitation energy level (T1 level) of the second host material is higher than that of the guest material. Preferably, the lowest triplet excitation energy level (T1 level) of the second host material is higher than that of the first host material. With such a structure, an exciplex can be formed by the two kinds of host materials. To form an exciplex efficiently, it is particularly preferable to combine a compound that easily accepts holes (hole-transport material) and a compound that easily accepts electrons (electron-transport material). With the above structure, high efficiency, low voltage, and a long lifetime can be achieved at the same time.

As an organic compound used as the host material (including the first host material and the second host material), organic compounds such as the hole-transport materials usable for the hole-transport layers (112, 112a, and 112b) described above and electron-transport materials usable for electron-transport layers (114, 114a, and 114b) described later can be used as long as they satisfy requirements for the host material used in the light-emitting layer. Another example is an exciplex formed by two or more kinds of organic compounds (the first host material and the second host material). An exciplex whose excited state is formed by two or more kinds of organic compounds has an extremely small difference between the S1 level and the T1 level and functions as a thermally activated delayed fluorescent (TADF) material capable of converting triplet excitation energy into singlet excitation energy. In an example of a preferable combination of two or more kinds of organic compounds forming an exciplex, one compound of the two or more kinds of organic compounds has a π-electron deficient heteroaromatic ring and the other compound has a π-electron rich heteroaromatic ring. A phosphorescent substance such as an iridium-, rhodium-, or platinum-based organometallic complex or a metal complex may be used as one compound of the combination for forming an exciplex. The organic compound described in Embodiment 1 has a hole-transport property and thus can be used as the host material.

There is no particular limitation on the light-emitting substances that can be used for the light-emitting layers (113, 113a, and 113b), and a light-emitting substance that converts singlet excitation energy into light in the visible light range or a light-emitting substance that converts triplet excitation energy into light in the visible light range can be used.

<<Light-Emitting Substance that Converts Singlet Excitation Energy into Light>>

The following substances that emit fluorescent light (fluorescent substances) can be given as examples of the light-emitting substance that converts singlet excitation energy into light and can be used in the light-emitting layers (113, 113a, and 113b): a pyrene derivative, an anthracene derivative, a triphenylene derivative, a fluorene derivative, a carbazole derivative, a dibenzothiophene derivative, a dibenzofuran derivative, a dibenzoquinoxaline derivative, a quinoxaline derivative, a pyridine derivative, a pyrimidine derivative, a phenanthrene derivative, and a naphthalene derivative. A pyrene derivative is particularly preferable because it has a high emission quantum yield. Specific examples of the pyrene derivative include N,N-bis(3-methylphenyl)-N,N-bis[3-(9-phenyl-9H-fluoren-9-yl)phenyl]pyrene-1,6-diamine (abbreviation: 1,6mMemFLPAPrn), N,N′-diphenyl-N,N-bis[4-(9-phenyl-9H-fluoren-9-yl)phenyl]pyrene-1,6-diamine (abbreviation: 1,6FLPAPrn), N,N′-bis(dibenzofuran-2-yl)-N,N′-diphenylpyrene-1,6-diamine (abbreviation: 1,6FrAPrn), N,N′-bis(dibenzothiophen-2-yl)-N,N′-diphenylpyrene-1,6-diamine (abbreviation: 1,6ThAPrn), N,N′-(pyrene-1,6-diyl)bis[(N-phenylbenzo[b]naphtho[1,2-d]furan)-6-amine] (abbreviation: 1,6BnfAPrn), N,N′-(pyrene-1,6-diyl)bis[(N-phenylbenzo[b]naphtho[1,2-d]furan)-8-amine](abbreviation: 1,6BnfAPrn-02), and N,N′-(pyrene-1,6-diyl)bis[(6,N-diphenylbenzo[b]naphtho[1,2-d]furan)-8-amine] (abbreviation: 1,6BnfAPrn-03).

In addition, it is possible to use, for example, 5,6-bis[4-(10-phenyl-9-anthryl)phenyl]-2,2′-bipyridine (abbreviation: PAP2BPy), 5,6-bis[4′-(10-phenyl-9-anthryl)biphenyl-4-yl]-2,2′-bipyridine (abbreviation: PAPP2BPy), N,N-bis[4-(9H-carbazol-9-yl)phenyl]-N,N-diphenylstilbene-4,4′-diamine (abbreviation: YGA2S), 4-(9H-carbazol-9-yl)-4′-(10-phenyl-9-anthryl)triphenylamine (abbreviation: YGAPA), 4-(9H-carbazol-9-yl)-4′-(9,10-diphenyl-2-anthryl)triphenylamine (abbreviation: 2YGAPPA), N,9-diphenyl-N-[4-(10-phenyl-9-anthryl)phenyl]-9H-carbazol-3-amine (abbreviation: PCAPA), 4-(10-phenyl-9-anthryl)-4′-(9-phenyl-9H-carbazol-3-yl)triphenylamine (abbreviation: PCBAPA), 4-[4-(10-phenyl-9-anthryl)phenyl]-4′-(9-phenyl-9H-carbazol-3-yl)triphenylamine (abbreviation: PCBAPBA), perylene, 2,5,8,11-tetra-tert-butylperylene (abbreviation: TBP), N,N′-(2-tert-butylanthracene-9,10-diyldi-4,1-phenylene)bis(N,N,N-triphenyl-1,4-phenylenediamine) (abbreviation: DPABPA), N,9-diphenyl-N-[4-(9,10-diphenyl-2-anthryl)phenyl]-9H-carbazol-3-amine (abbreviation: 2PCAPPA), and N-[4-(9,10-diphenyl-2-anthryl)phenyl]-N,N,N-triphenyl-1,4-phenylenediamine (abbreviation: 2DPAPPA).

It is also possible to use, for example, N-[9,10-bis(biphenyl-2-yl)-2-anthryl]-N,9-diphenyl-9H-carbazol-3-amine (abbreviation: 2PCABPhA), N-(9,10-diphenyl-2-anthryl)-N,N,N-triphenyl-1,4-phenylenediamine (abbreviation: 2DPAPA), N-[9,10-bis(biphenyl-2-yl)-2-anthryl]-N,N,N-triphenyl-1,4-phenylenediamine (abbreviation: 2DPABPhA), 9,10-bis(biphenyl-2-yl)-N-[4-(9H-carbazol-9-yl)phenyl]-N-phenylanthracen-2-amine (abbreviation: 2YGABPhA), N,N,9-triphenylanthracen-9-amine (abbreviation: DPhAPhA), coumarin 545T, N,N-diphenylquinacridone (abbreviation: DPQd), rubrene, 5,12-bis(biphenyl-4-yl)-6,11-diphenyltetracene (abbreviation: BPT), 2-(2-{2-[4-(dimethylamino)phenyl]ethenyl}-6-methyl-4H-pyran-4-ylidene)propanedinitrile (abbreviation: DCM1), 2-{2-methyl-6-[2-(2,3,6,7-tetrahydro-1H,5H-benzo[ij]quinolizin-9-yl)ethenyl]-4H-pyran-4-ylidene}propanedinitrile (abbreviation: DCM2), N,N,N,N-tetrakis(4-methylphenyl)tetracene-5,11-diamine (abbreviation: p-mPhTD), 7,14-diphenyl-N,N,N,N-tetrakis(4-methylphenyl)acenaphtho[1,2-a]fluoranthene-3,10-diamine (abbreviation: p-mPhAFD), 2-{2-isopropyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTI), 2-{2-tert-butyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTB), 2-(2,6-bis{2-[4-(dimethylamino)phenyl]ethenyl}-4H-pyran-4-ylidene)propanedinitrile (abbreviation: BisDCM), 2-{2,6-bis[2-(8-methoxy-1,1,7,7-tetramethyl-2,3,6,7-tetrahydro-1H,5H-benzo[ij]quinolizin-9-yl)ethenyl]-4H-pyran-4-ylidene}propanedinitrile (abbreviation: BisDCJTM), 1,6BnfAPrn-03, 3,10-bis[N-(9-phenyl-9H-carbazol-2-yl)-N-phenylamino]naphtho[2,3-b;6,7-b′]bisbenzofuran (abbreviation: 3,10PCA2Nbf(IV)-02), and 3,10-bis[N-(dibenzofuran-3-yl)-N-phenylamino]naphtho[2,3-b;6,7-b′]bisbenzofuran (abbreviation: 3,10FrA2Nbf(IV)-02). In particular, pyrenediamine compounds such as 1,6FLPAPrn, 1,6mMemFLPAPrn, and 1,6BnfAPrn-03 can be used, for example.

<<Light-Emitting Substance that Converts Triplet Excitation Energy into Light>>

Examples of the light-emitting substance that converts triplet excitation energy into light and can be used in the light-emitting layer 113 include substances that emit phosphorescent light (phosphorescent substances) and TADF materials that exhibit thermally activated delayed fluorescence.

A phosphorescent substance is a compound that emits phosphorescent light but does not emit fluorescent light at a temperature higher than or equal to a low temperature (e.g., 77 K) and lower than or equal to room temperature (i.e., higher than or equal to 77 K and lower than or equal to 313 K). The phosphorescent substance preferably contains a metal element with large spin-orbit interaction, and can be an organometallic complex, a metal complex (platinum complex), or a rare earth metal complex, for example. Specifically, the phosphorescent substance preferably contains a transition metal element. It is preferable that the phosphorescent substance contain a platinum group element (ruthenium (Ru), rhodium (Rh), palladium (Pd), osmium (Os), iridium (Ir), or platinum (Pt)), especially iridium, in which case the probability of direct transition between the singlet ground state and the triplet excited state can be increased.

<<Phosphorescent Substance (from 450 nm to 570 nm: Blue or Green)>>

As examples of a phosphorescent substance which emits blue or green light and whose emission spectrum has a peak wavelength of greater than or equal to 450 nm and less than or equal to 570 nm, the following substances can be given.

Examples include organometallic complexes having a 4H-triazole ring, such as tris{2-[5-(2-methylphenyl)-4-(2,6-dimethylphenyl)-4H-1,2,4-triazol-3-yl-κN²]phenyl-κC}iridium(III) (abbreviation: [Ir(mpptz-dmp)₃]), tris(5-methyl-3,4-diphenyl-4H-1,2,4-triazolato)iridium(III) (abbreviation: [Ir(Mptz)₃]), tris[4-(3-biphenyl)-5-isopropyl-3-phenyl-4H-1,2,4-triazolato]iridium(III) (abbreviation: [Ir(iPrptz-3b)₃]), and tris[3-(5-biphenyl)-5-isopropyl-4-phenyl-4H-1,2,4-triazolato]iridium(III) (abbreviation: [Ir(iPr5btz)₃]); organometallic complexes having a 1H-triazole ring, such as tris[3-methyl-1-(2-methylphenyl)-5-phenyl-1H-1,2,4-triazolato]iridium(III) (abbreviation: [Ir(Mptz1-mp)₃]) and tris(1-methyl-5-phenyl-3-propyl-1H-1,2,4-triazolato)iridium(III) (abbreviation: [Ir(Prptz1-Me)₃]); organometallic complexes having an imidazole ring, such as fac-tris[1-(2,6-diisopropylphenyl)-2-phenyl-1H-imidazole]iridium(III) (abbreviation: [Ir(iPrpim)₃]) and tris[3-(2,6-dimethylphenyl)-7-methylimidazo[1,2-f]phenanthridinato]iridium(III) (abbreviation: [Ir(dmpimpt-Me)₃]); and organometallic complexes in which a phenylpyridine derivative having an electron-withdrawing group is a ligand, such as bis[2-(4′,6′-difluorophenyl)pyridinato-N,C^{2′}]iridium(III) tetrakis(1-pyrazolyl)borate (abbreviation: FIr6), bis[2-(4′,6′-difluorophenyl)pyridinato-N,C²′]iridium(III) picolinate (abbreviation: FIrpic), bis{2-[3′,5′-bis(trifluoromethyl)phenyl]pyridinato-N,C^{2′}}iridium(III) picolinate (abbreviation: [Ir(CF₃ppy)₂(pic)]), and bis[2-(4′,6′-difluorophenyl)pyridinato-N,C^{2′}]iridium(III) acetylacetonate (abbreviation: FIr(acac)).

<<Phosphorescent Substance (from 495 nm to 590 nm: Green or Yellow)>>

As examples of a phosphorescent substance which emits green or yellow light and whose emission spectrum has a peak wavelength of greater than or equal to 495 nm and less than or equal to 590 nm, the following substances can be given.

Examples of the phosphorescent substance include organometallic iridium complexes having a pyrimidine ring, such as tris(4-methyl-6-phenylpyrimidinato)iridium(III) (abbreviation: [Ir(mppm)₃]), tris(4-t-butyl-6-phenylpyrimidinato)iridium(III) (abbreviation: [Ir(tBuppm)₃]), (acetylacetonato)bis(6-methyl-4-phenylpyrimidinato)iridium(III) (abbreviation: [Ir(mppm)₂(acac)]), (acetylacetonato)bis(6-tert-butyl-4-phenylpyrimidinato)iridium(III) (abbreviation: [Ir(tBuppm)₂(acac)]), (acetylacetonato)bis[6-(2-norbornyl)-4-phenylpyrimidinato]iridium(III) (abbreviation: [Ir(nbppm)₂(acac)]), (acetylacetonato)bis[5-methyl-6-(2-methylphenyl)-4-phenylpyrimidinato]iridium(III) (abbreviation: [Ir(mpmppm)₂(acac)]), (acetylacetonato)bis{4,6-dimethyl-2-[6-(2,6-dimethylphenyl)-4-pyrimidinyl-κN³]phenyl-κC}iridium(III) (abbreviation: [Ir(dmppm-dmp)₂(acac)]), and (acetylacetonato)bis(4,6-diphenylpyrimidinato)iridium(III) (abbreviation: [Ir(dppm)₂(acac)]); organometallic iridium complexes having a pyrazine ring, such as (acetylacetonato)bis(3,5-dimethyl-2-phenylpyrazinato)iridium(III) (abbreviation: [Ir(mppr-Me)₂(acac)]) and (acetylacetonato)bis(5-isopropyl-3-methyl-2-phenylpyrazinato)iridium(III) (abbreviation: [Ir(mppr-iPr)₂(acac)]); organometallic iridium complexes having a pyridine ring, such as tris(2-phenylpyridinato-N,C^{2′})iridium(III) (abbreviation: [Ir(ppy)₃]), bis(2-phenylpyridinato-N,C^{2′})iridium(III) acetylacetonate (abbreviation: [Ir(ppy)₂(acac)]), bis(benzo[h]quinolinato)iridium(III) acetylacetonate (abbreviation: [Ir(bzq)₂(acac)]), tris(benzo[h]quinolinato)iridium(III) (abbreviation: [Ir(bzq)₃]), tris(2-phenylquinolinato-N,C^{2′})iridium(III) (abbreviation: [Ir(pq)₃]), bis(2-phenylquinolinato-N,C^{2′})iridium(III) acetylacetonate (abbreviation: [Ir(pq)₂(acac)]), bis[2-(2-pyridinyl-κN)phenyl-κC][2-(4-phenyl-2-pyridinyl-κN)phenyl-κC]iridium(III) (abbreviation: [Ir(ppy)₂(4dppy)]), bis[2-(2-pyridinyl-κN)phenyl-κC][2-(4-methyl-5-phenyl-2-pyridinyl-κN)phenyl-KC], [2-d3-methyl-8-(2-pyridinyl-κN)benzofuro[2,3-b]pyridine-κC]bis[2-(5-d3-methyl-2-pyridinyl-κN²)phenyl-κC]iridium(III) (abbreviation: [Ir(5mppy-d3)₂(mbfpypy-d3)]), {2-(methyl-d3)-8-[4-(1-methylethyl-1-d)-2-pyridinyl-κN]benzofuro[2,3-b]pyridin-7-yl-KC}bis{5-(methyl-d3)-2-[5-(methyl-d3)-2-pyridinyl-N]phenyl-κC}iridium(III) (abbreviation: Ir(5mtpy-d6)₂(mbfpypy-iPr-d4)), [2-d3-methyl-(2-pyridinyl-κN)benzofuro[2,3-b]pyridine-κC]bis[2-(2-pyridinyl-κN)phenyl-κC]iridium(III) (abbreviation: Ir(ppy)₂(mbfpypy-d3)), and [2-(4-methyl-5-phenyl-2-pyridinyl-κN)phenyl-κC]bis[2-(2-pyridinyl-κN)phenyl-κC]iridium(III) (abbreviation: Ir(ppy)₂(mdppy)); organometallic complexes such as bis(2,4-diphenyl-1,3-oxazolato-N,C^{2′})iridium(III) acetylacetonate (abbreviation: [Ir(dpo)₂(acac)]), bis{2-[4′-(perfluorophenyl)phenyl]pyridinato-N,C^{2′}}iridium(III) acetylacetonate (abbreviation: [Ir(p-PF-ph)₂(acac)]), and bis(2-phenylbenzothiazolato-N,C²′)iridium(III) acetylacetonate (abbreviation: [Ir(bt)₂(acac)]); and a rare earth metal complex such as tris(acetylacetonato) (monophenanthroline)terbium(III) (abbreviation: [Tb(acac)₃(Phen)]).

<<Phosphorescent Substance (from 570 nm to 750 nm: Yellow or Red)>>

As examples of a phosphorescent substance which emits yellow or red light and whose emission spectrum has a peak wavelength of greater than or equal to 570 nm and less than or equal to 750 nm, the following substances can be given.

Examples of a phosphorescent substance include organometallic complexes having a pyrimidine ring, such as (diisobutyrylmethanato)bis[4,6-bis(3-methylphenyl)pyrimidinato]iridium(III) (abbreviation: [Ir(5mdppm)₂(dibm)]), bis[4,6-bis(3-methylphenyl)pyrimidinato](dipivaloylmethanato)iridium(III) (abbreviation: [Ir(5mdppm)₂(dpm)]), and (dipivaloylmethanato)bis[4,6-di(naphthalen-1-yl)pyrimidinato]iridium(III) (abbreviation: [Ir(d1npm)₂(dpm)]); organometallic complexes having a pyrazine ring, such as (acetylacetonato)bis(2,3,5-triphenylpyrazinato)iridium(III) (abbreviation: [Ir(tppr)₂(acac)]), bis(2,3,5-triphenylpyrazinato)(dipivaloylmethanato)iridium(III) (abbreviation: [Ir(tppr)₂(dpm)]), bis{4,6-dimethyl-2-[3-(3,5-dimethylphenyl)-5-phenyl-2-pyrazinyl-N]phenyl-κC}(2,6-dimethyl-3,5-heptanedionato-κ²O,O′)iridium(III) (abbreviation: [Ir(dmdppr-P)₂(dibm)]), bis{4,6-dimethyl-2-[5-(4-cyano-2,6-dimethylphenyl)-3-(3,5-dimethylphenyl)-2-pyrazinyl-N]phenyl-κC}(2,2,6,6-tetramethyl-3,5-heptanedionato-x2O,O′)iridium(III) (abbreviation: [Ir(dmdppr-dmCP)₂(dpm)]), bis{2-[5-(2,6-dimethylphenyl)-3-(3,5-dimethylphenyl)-2-pyrazinyl-κN]-4,6-dimethylphenyl-κC}(2,2′,6,6′-tetramethyl-3,5-heptanedionato-κ2O,O′)iridium(III) (abbreviation: [Ir(dmdppr-dmp)₂(dpm)]), (acetylacetonato)bis(2-methyl-3-phenylquinoxalinato-N,C^{2′})iridium(III) (abbreviation: [Ir(mpq)₂(acac)]), (acetylacetonato)bis(2,3-diphenylquinoxalinato-N,C^{2′})iridium(III) (abbreviation: [Ir(dpq)₂(acac)]), and (acetylacetonato)bis[2,3-bis(4-fluorophenyl)quinoxalinato]iridium(III) (abbreviation: [Ir(Fdpq)₂(acac)]); organometallic complexes having a pyridine ring, such as tris(1-phenylisoquinolinato-N,C²′)iridium(III) (abbreviation: [Ir(piq)₃]), bis(1-phenylisoquinolinato-N,C^{2′})iridium(III) acetylacetonate (abbreviation: [Ir(piq)₂(acac)]), and bis[4,6-dimethyl-2-(2-quinolinyl-κN)phenyl-κC](2,4-pentanedionato-κ2O,O′)iridium(III) (abbreviation: [Ir(dmpqn)₂(acac)]); a platinum complex such as 2,3,7,8,12,13,17,18-octaethyl-21H,23H-porphyrin platinum(II) (abbreviation: [PtOEP]); and rare earth metal complexes such as tris(1,3-diphenyl-1,3-propanedionato)(monophenanthroline)europium(III) (abbreviation: [Eu(DBM)₃(Phen)]) and tris[1-(2-thenoyl)-3,3,3-trifluoroacetonato](monophenanthroline)europium(III) (abbreviation: [Eu(TTA)₃(Phen)]).

### <<Tadf Material>>

Any of materials described below can be used as the TADF material. The TADF material is a material that has a small difference between its S1 and T1 levels (preferably less than or equal to 0.20 eV), enables up-conversion of a triplet excited state into a singlet excited state (i.e., reverse intersystem crossing) using a little thermal energy, and efficiently exhibits light (fluorescent light) from the singlet excited state. The thermally activated delayed fluorescence is efficiently obtained under the condition where the difference in energy between the triplet excitation energy level and the singlet excitation energy level is greater than or equal to 0.00 eV and less than or equal to 0.20 eV, preferably greater than or equal to 0.00 eV and less than or equal to 0.10 eV. Note that delayed fluorescent light by the TADF material refers to light emission having a spectrum similar to that of normal fluorescent light and an extremely long lifetime. The lifetime is longer than or equal to 1×10⁻⁶ seconds, or longer than or equal to 1×10³ seconds.

Note that the TADF material can be also used as an electron-transport material, a hole-transport material, or a host material.

Examples of the TADF material include fullerene, a derivative thereof, an acridine derivative such as proflavine, and eosin. Other examples thereof include a metal-containing porphyrin such as a porphyrin containing magnesium (Mg), zinc (Zn), cadmium (Cd), tin (Sn), platinum (Pt), indium (In), or palladium (Pd). Examples of the metal-containing porphyrin include a protoporphyrin-tin fluoride complex (abbreviation: SnF₂(Proto IX)), a mesoporphyrin-tin fluoride complex (abbreviation: SnF₂(Meso IX)), a hematoporphyrin-tin fluoride complex (abbreviation: SnF₂(Hemato IX)), a coproporphyrin tetramethyl ester-tin fluoride complex (abbreviation: SnF₂(Copro III-4Me)), an octaethylporphyrin-tin fluoride complex (abbreviation: SnF₂(OEP)), an etioporphyrin-tin fluoride complex (abbreviation: SnF₂(Etio I)), and an octaethylporphyrin-platinum chloride complex (abbreviation: PtCl₂OEP).

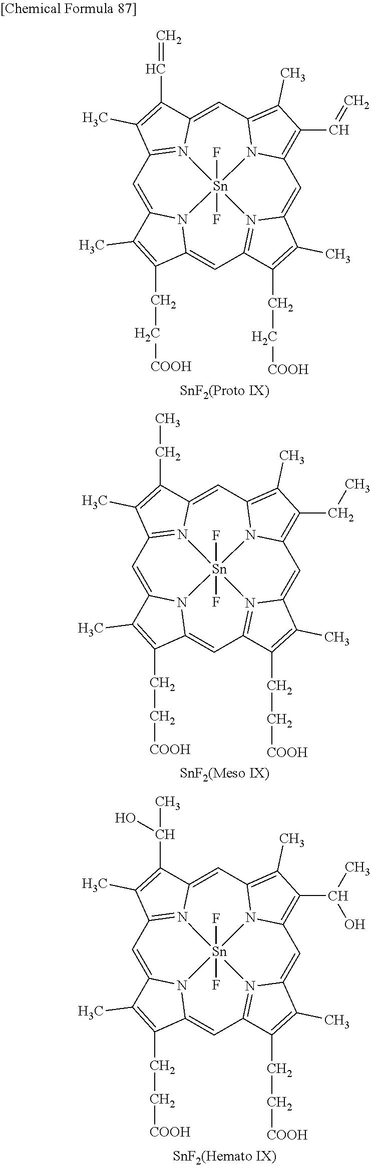

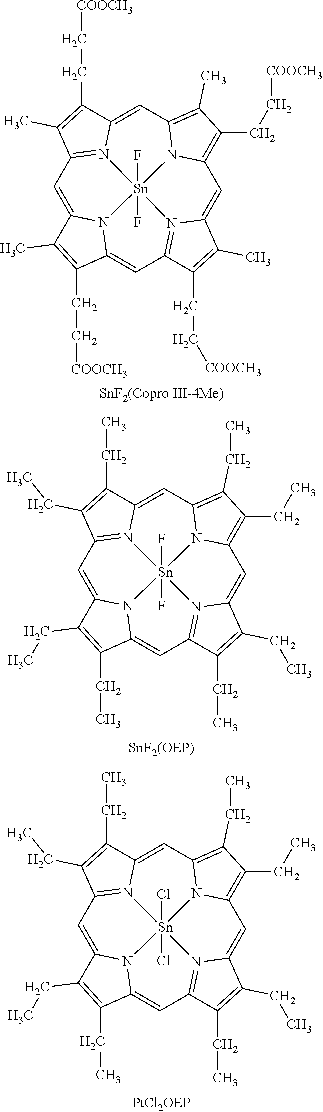

Additionally, a heteroaromatic compound having a π-electron rich heteroaromatic compound and a π-electron deficient heteroaromatic compound, such as 2-(biphenyl-4-yl)-4,6-bis(12-phenylindolo[2,3-a]carbazol-11-yl)-1,3,5-triazine (abbreviation: PIC-TRZ), 2-{4-[3-(N-phenyl-9H-carbazol-3-yl)-9H-carbazol-9-yl]phenyl}-4,6-diphenyl-1,3,5-triazine (abbreviation: PCCzPTzn), 2-[4-(10H-phenoxazin-10-yl)phenyl]-4,6-diphenyl-1,3,5-triazine (abbreviation: PXZ-TRZ), 3-[4-(5-phenyl-5,10-dihydrophenazin-10-yl)phenyl]-4,5-diphenyl-1,2,4-triazole (abbreviation: PPZ-3TPT), 3-(9,9-dimethyl-9H-acridin-10-yl)-9H-xanthen-9-one (abbreviation: ACRXTN), bis[4-(9,9-dimethyl-9,10-dihydroacridine)phenyl]sulfone (abbreviation: DMAC-DPS), 10-phenyl-10H,10′H-spiro[acridin-9,9′-anthracen]-10′-one (abbreviation: ACRSA), 4-(9′-phenyl-3,3′-bi-9H-carbazol-9-yl)benzofuro[3,2-d]pyrimidine (abbreviation: 4PCCzBfpm), 4-[4-(9′-phenyl-3,3′-bi-9H-carbazol-9-yl)phenyl]benzofuro[3,2-d]pyrimidine (abbreviation: 4PCCzPBfpm), or 9-[3-(4,6-diphenyl-1,3,5-triazin-2-yl)phenyl]-9′-phenyl-2,3′-bi-9H-carbazole (abbreviation: mPCCzPTzn-02) may be used.

Note that a substance in which a π-electron rich heteroaromatic compound is directly bonded to a π-electron deficient heteroaromatic compound is particularly preferable because both the donor property of the π-electron rich heteroaromatic compound and the acceptor property of the π-electron deficient heteroaromatic compound are improved and the energy difference between the singlet excited state and the triplet excited state becomes small. As the TADF material, a TADF material in which the singlet and triplet excited states are in thermal equilibrium (TADF100) may be used. Since such a TADF material enables a short emission lifetime (excitation lifetime), the efficiency of a light-emitting device in a high-luminance region can be less likely to decrease.

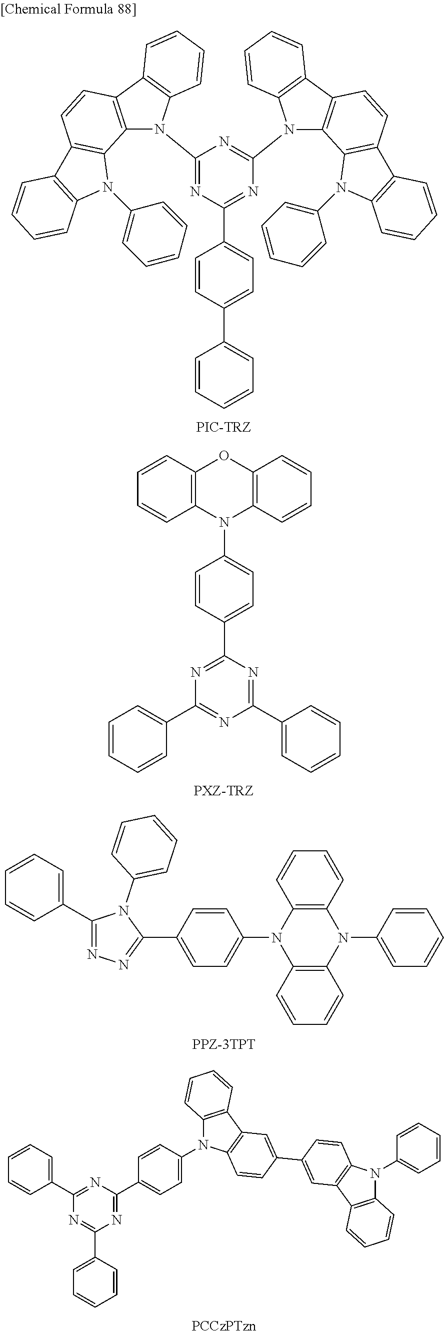

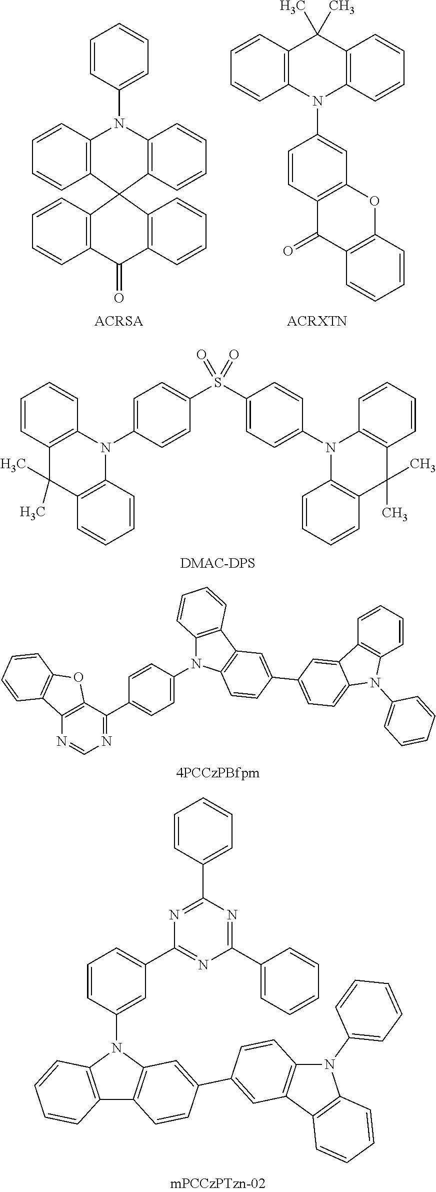 [SMILES: O=C1c2ccccc2C2(c3ccccc31)c1ccccc1N(c1ccccc1)c1ccccc12 ; CC1(C)c2ccccc2N(c2ccc3c(=O)c4ccccc4oc3c2)c2ccccc21 ; CC1(C)c2ccccc2N(c2ccc(S(=O)(=O)c3ccc(N4c5ccccc5C(C)(C)c5ccccc54)cc3)cc2)c2ccccc21 ; c1ccc(-n2c3ccccc3c3cc(-c4ccc5c(c4)c4ccccc4n5-c4ccc(-c5ncnc6c5oc5ccccc56)cc4)ccc32)cc1 ; c1ccc(-c2nc(-c3ccccc3)nc(-c3cccc(-n4c5ccccc5c5ccc(-c6ccc7c(c6)c6ccccc6n7-c6ccccc6)cc54)c3)n2)cc1]

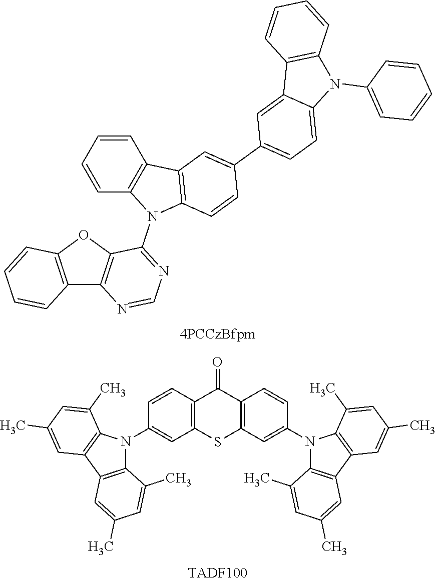

In addition to the above, another example of a material having a function of converting triplet excitation energy into light is a nano-structure of a transition metal compound having a perovskite structure. In particular, a nano-structure of a metal halide perovskite material is preferable. The nano-structure is preferably a nanoparticle or a nanorod.

As the organic compound (e.g., the host material) used in combination with the above-described light-emitting substance (guest material) in the light-emitting layers (113, 113a, and 113b), one or more kinds selected from substances having a larger energy gap than the light-emitting substance (guest material) can be used.

### <<Host Material for Fluorescent Light>>

In the case where the light-emitting substance used in the light-emitting layers (113, 113a, and 113b) is a fluorescent substance, an organic compound (host material) used in combination with the fluorescent substance is preferably an organic compound that has a high energy level in a singlet excited state and has a low energy level in a triplet excited state or an organic compound having a high fluorescence quantum yield. Thus, the hole-transport material (described above) and the electron-transport material (described below) shown in this embodiment, for example, can be used as long as they are organic compounds that satisfy such a condition. In addition, the organic compound described in Embodiment 1 can be used.

In terms of a preferable combination with the light-emitting substance (fluorescent substance), examples of the organic compound (host material), some of which overlap the above specific examples, include fused polycyclic aromatic compounds such as an anthracene derivative, a tetracene derivative, a phenanthrene derivative, a pyrene derivative, a chrysene derivative, and a dibenzo[g,p]chrysene derivative.

Specific examples of the organic compound (host material) that is preferably used in combination with the fluorescent substance include 9-phenyl-3-[4-(10-phenyl-9-anthryl)phenyl]-9H-carbazole (abbreviation: PCzPA), 3,6-diphenyl-9-[4-(10-phenyl-9-anthryl)phenyl]-9H-carbazole (abbreviation: DPCzPA), 3-[4-(1-naphthyl)-phenyl]-9-phenyl-9H-carbazole (abbreviation: PCPN), 9,10-diphenylanthracene (abbreviation: DPAnth), N,N-diphenyl-9-[4-(10-phenyl-9-anthryl)phenyl]-9H-carbazol-3-amine (abbreviation: CzA1PA), 4-(10-phenyl-9-anthryl)triphenylamine (abbreviation: DPhPA), YGAPA, PCAPA, N,9-diphenyl-N-{4-[4-(10-phenyl-9-anthryl)phenyl]phenyl}-9H-carbazol-3-amine (abbreviation: PCAPBA), N-(9,10-diphenyl-2-anthryl)-N,9-diphenyl-9H-carbazol-3-amine (abbreviation: 2PCAPA), 6,12-dimethoxy-5,11-diphenylchrysene, N,N,N,N,N′,N′,N″,N″-octaphenyldibenzo[g,p]chrysene-2,7,10,15-tetraamine (abbreviation: DBC1), 9-[4-(10-phenyl-9-anthracenyl)phenyl]-9H-carbazole (abbreviation: CzPA), 7-[4-(10-phenyl-9-anthryl)phenyl]-7H-dibenzo[c,g]carbazole (abbreviation: cgDBCzPA), 6-[3-(9,10-diphenyl-2-anthryl)phenyl]-benzo[b]naphtho[1,2-d]furan (abbreviation: 2mBnfPPA), 9-phenyl-10-[4′-(9-phenyl-9H-fluoren-9-yl)biphenyl-4-yl]anthracene (abbreviation: FLPPA), 9,10-bis(3,5-diphenylphenyl)anthracene (abbreviation: DPPA), 9,10-di(2-naphthyl)anthracene (abbreviation: DNA), 2-tert-butyl-9,10-di(2-naphthyl)anthracene (abbreviation: t-BuDNA), 9-(1-naphthyl)-10-(2-naphthyl)anthracene (abbreviation: α,β-ADN), 2-(10-phenylanthracen-9-yl)dibenzofuran, 2-(10-phenyl-9-anthracenyl)-benzo[b]naphtho[2,3-d]furan (abbreviation: Bnf(II)PhA), 9-(1-naphthyl)-10-[4-(2-naphthyl)phenyl]anthracene (abbreviation: α,N-PNPAnth), 2,9-di(1-naphthyl)-10-phenylanthracene (abbreviation: 2αN-αNPhA), 9-(1-naphthyl)-10-[3-(1-naphthyl)phenyl]anthracene (abbreviation: α,N-mαNPAnth), 9-(2-naphthyl)-10-[3-(1-naphthyl)phenyl]anthracene (abbreviation: βN-mαNPAnth), 9-(1-naphthyl)-10-[4-(1-naphthyl)phenyl]anthracene (abbreviation: αN-αNPAnth), 9-(2-naphthyl)-10-[4-(2-naphthyl)phenyl]anthracene (abbreviation: βN-βNPAnth), 2-(1-naphthyl)-9-(2-naphthyl)-10-phenylanthracene (abbreviation: 2αN-βNPhA), 9-(2-naphthyl)-10-[3-(2-naphthyl)phenyl]anthracene (abbreviation: βN-mβNPAnth), 1-{4-[10-(biphenyl-4-yl)-9-anthracenyl]phenyl}-2-ethyl-1H-benzimidazole (abbreviation: EtBImPBPhA), 9,9′-bianthryl (abbreviation: BANT), 9,9′-(stilbene-3,3′-diyl)diphenanthrene (abbreviation: DPNS), 9,9′-(stilbene-4,4′-diyl)diphenanthrene (abbreviation: DPNS2), 1,3,5-tri(1-pyrenyl)benzene (abbreviation: TPB3), 5,12-diphenyltetracene, and 5,12-bis(biphenyl-2-yl)tetracene.

### <<Host Material for Phosphorescent Light>>

In the case where the light-emitting substance used in the light-emitting layers (113, 113a, and 113b) is a phosphorescent substance, an organic compound having triplet excitation energy (an energy difference between a ground state and a triplet excited state) which is higher than that of the light-emitting substance is preferably selected as the organic compound (host material) used in combination with the phosphorescent substance. Note that when a plurality of organic compounds (e.g., a first host material and a second host material (or an assist material)) are used in combination with a light-emitting substance so that an exciplex is formed, the plurality of organic compounds are preferably mixed with the phosphorescent substance. In addition, the organic compound described in Embodiment 1 can be used.

With such a structure, light emission can be efficiently obtained by exciplex-triplet energy transfer (ExTET), which is energy transfer from an exciplex to a light-emitting substance. Note that a combination of the plurality of organic compounds that easily forms an exciplex is preferred, and it is particularly preferable to combine a compound that easily accepts holes (hole-transport material) and a compound that easily accepts electrons (electron-transport material).

In terms of a preferable combination with the light-emitting substance (phosphorescent substance), examples of the organic compounds (the host material and the assist material), some of which overlap the above specific examples, include an aromatic amine (an organic compound having an aromatic amine skeleton), a carbazole derivative (an organic compound having a carbazole ring), a dibenzothiophene derivative (an organic compound having a dibenzothiophene ring), a dibenzofuran derivative (an organic compound having a dibenzofuran ring), an oxadiazole derivative (an organic compound having an oxadiazole ring), a triazole derivative (an organic compound having an triazole ring), a benzimidazole derivative (an organic compound having an benzimidazole ring), a quinoxaline derivative (an organic compound having a quinoxaline ring), a dibenzoquinoxaline derivative (an organic compound having a dibenzoquinoxaline ring), a pyrimidine derivative (an organic compound having a pyrimidine ring), a triazine derivative (an organic compound having a triazine ring), a pyridine derivative (an organic compound having a pyridine ring), a bipyridine derivative (an organic compound having a bipyridine ring), a phenanthroline derivative (an organic compound having a phenanthroline ring), a furodiazine derivative (an organic compound having a furodiazine ring), and zinc- or aluminum-based metal complexes.

Among the above organic compounds, specific examples of the aromatic amine and the carbazole derivative, which are organic compounds having a high hole-transport property, are the same as the specific examples of the hole-transport materials described above, and those materials are preferable as the host material.

Among the above organic compounds, specific examples of the dibenzothiophene derivative and the dibenzofuran derivative, which are organic compounds having a high hole-transport property, include 4-{3-[3-(9-phenyl-9H-fluoren-9-yl)phenyl]phenyl}dibenzofuran (abbreviation: mmDBFFLBi-II), 4,4′,4″-(benzene-1,3,5-triyl)tri(dibenzofuran) (abbreviation: DBF3P-II), DBT3P-II, 2,8-diphenyl-4-[4-(9-phenyl-9H-fluoren-9-yl)phenyl]dibenzothiophene (abbreviation: DBTFLP-III), 4-[4-(9-phenyl-9H-fluoren-9-yl)phenyl]-6-phenyldibenzothiophene (abbreviation: DBTFLP-IV), and 4-[3-(triphenylen-2-yl)phenyl]dibenzothiophene (abbreviation: mDBTPTp-II). Such derivatives are preferable as the host material.

Other examples of preferable host materials include metal complexes having an oxazole-based or thiazole-based ligand, such as bis[2-(2-benzoxazolyl)phenolato]zinc(II) (abbreviation: ZnPBO) and bis[2-(2-benzothiazolyl)phenolato]zinc(II) (abbreviation: ZnBTZ).

Among the above organic compounds, specific examples of the oxadiazole derivative, the triazole derivative, the benzimidazole derivative, the quinoxaline derivative, the dibenzoquinoxaline derivative, the quinazoline derivative, and the phenanthroline derivative, which are organic compounds having a high electron-transport property, include: an organic compound including a heteroaromatic ring having a polyazole ring such as 2-(4-biphenylyl)-5-(4-tert-butylphenyl)-1,3,4-oxadiazole (abbreviation: PBD), 1,3-bis[5-(p-tert-butylphenyl)-1,3,4-oxadiazol-2-yl]benzene (abbreviation: OXD-7), 9-[4-(5-phenyl-1,3,4-oxadiazol-2-yl)phenyl]-9H-carbazole (abbreviation: COl1), 3-(4-biphenylyl)-4-phenyl-5-(4-tert-butylphenyl)-1,2,4-triazole (abbreviation: TAZ), 2,2′,2″-(1,3,5-benzenetriyl)tris(1-phenyl-1H-benzimidazole) (abbreviation: TPBI), 2-[3-(dibenzothiophen-4-yl)phenyl]-1-phenyl-1H-benzimidazole (abbreviation: mDBTBIm-II), or 4,4′-bis(5-methylbenzoxazol-2-yl)stilbene (abbreviation: BzOs); an organic compound including a heteroaromatic ring having a phenanthroline ring such as bathophenanthroline (abbreviation: Bphen), bathocuproine (abbreviation: BCP), 2,9-di(naphthalen-2-yl)-4,7-diphenyl-1,10-phenanthroline (abbreviation: NBphen), or 2,2′-(1,3-phenylene)bis(9-phenyl-1,10-phenanthroline) (abbreviation: mPPhen2P); and an organic compound including a heteroaromatic ring having a dibenzoquinoxaline ring such as 2-[3-(dibenzothiophen-4-yl)phenyl]dibenzo[f,h]quinoxaline (abbreviation: 2mDBTPDBq-II), 2-[3′-(dibenzothiophen-4-yl)biphenyl-3-yl]dibenzo[f,h]quinoxaline (abbreviation: 2mDBTBPDBq-II), 2-[3′-(9H-carbazol-9-yl)biphenyl-3-yl]dibenzo[f,h]quinoxaline (abbreviation: 2mCzBPDBq), 2-[4-(3,6-diphenyl-9H-carbazol-9-yl)phenyl]dibenzo[f,h]quinoxaline (abbreviation: 2CzPDBq-III), 7-[3-(dibenzothiophen-4-yl)phenyl]dibenzo[f,h]quinoxaline (abbreviation: 7mDBTPDBq-II), 6-[3-(dibenzothiophen-4-yl)phenyl]dibenzo[f,h]quinoxaline (abbreviation: 6mDBTPDBq-II), 2-{4-[9,10-di(2-naphthyl)-2-anthryl]phenyl}-1-phenyl-1H-benzimidazole (abbreviation: ZADN), or 2-4′-(9-phenyl-9H-carbazol-3-yl)-3,1′-biphenyl-1-yl]dibenzo[f;h]quinoxaline (abbreviation: 2mpPCBPDBq). Such organic compounds are preferable as the host material.

Among the above organic compounds, specific examples of the pyridine derivative, the diazine derivative (e.g., the pyrimidine derivative, the pyrazine derivative, and the pyridazine derivative), the triazine derivative, the furodiazine derivative, which are organic compounds having a high electron-transport property, include organic compounds including a heteroaromatic ring having a diazine ring such as 4,6-bis[3-(phenanthren-9-yl)phenyl]pyrimidine (abbreviation: 4,6mPnP2Pm), 4,6-bis[3-(4-dibenzothienyl)phenyl]pyrimidine (abbreviation: 4,6mDBTP2Pm-II), 4,6-bis[3-(9H-carbazol-9-yl)phenyl]pyrimidine (abbreviation: 4,6mCzP2Pm), 2-{4-[3-(N-phenyl-9H-carbazol-3-yl)-9H-carbazol-9-yl]phenyl}-4,6-diphenyl-1,3,5-triazine (abbreviation: PCCzPTzn), 9-[3-(4,6-diphenyl-1,3,5-triazin-2-yl)phenyl]-9′-phenyl-2,3′-bi-9H-carbazole (abbreviation: mPCCzPTzn-02), 3,5-bis[3-(9H-carbazol-9-yl)phenyl]pyridine (abbreviation: 35DCzPPy), 1,3,5-tri[3-(3-pyridyl)phenyl]benzene (abbreviation: TmPyPB), 9,9′-[pyrimidine-4,6-diylbis(biphenyl-3,3′-diyl)]bis(9H-carbazole) (abbreviation: 4,6mCzBP2Pm), 2-[3′-(9,9-dimethyl-9H-fluoren-2-yl)biphenyl-3-yl]-4,6-diphenyl-1,3,5-triazine (abbreviation: mFBPTzn), 8-(biphenyl-4-yl)-4-[3-(dibenzothiophen-4-yl)phenyl]-[1]benzofuro[3,2-d]pyrimidine (abbreviation: 8BP-4mDBtPBfpm), 9-[3′-(dibenzothiophen-4-yl)biphenyl-3-yl]naphtho[1′,2′: 4,5]furo[2,3-b]pyrazine (abbreviation: 9mDBtBPNfpr), 9-[(3′-(dibenzothiophen-4-yl)biphenyl-4-yl]naphtho[1′,2′: 4,5]furo[2,3-b]pyrazine (abbreviation: 9pmDBtBPNfpr), 11-[(3′-(dibenzothiophen-4-yl)biphenyl-3-yl]phenanthro[9′,10′: 4,5]furo[2,3-b]pyrazine (abbreviation: 11mDBtBPPnfpr), 11-[(3′-(dibenzothiophen-4-yl)biphenyl-4-yl]phenanthro[9′,10′: 4,5]furo[2,3-b]pyrazine, 11-[(3′-9H-carbazol-9-yl)biphenyl-3-yl]phenanthro[9′,10′: 4,5]furo[2,3-b]pyrazine, 12-(9′-phenyl-3,3′-bi-9H-carbazol-9-yl)phenanthro[9′,10′: 4,5]furo[2,3-b]pyrazine (abbreviation: 12PCCzPnfpr), 9-[(3′-9-phenyl-9H-carbazol-3-yl)biphenyl-4-yl]naphtho[1′,2′: 4,5]furo[2,3-b]pyrazine (abbreviation: 9pmPCBPNfpr), 9-(9′-phenyl-3,3′-bi-9H-carbazol-9-yl)naphtho[1′,2′: 4,5]furo[2,3-b]pyrazine (abbreviation: 9PCCzNfpr), 10-(9′-phenyl-3,3′-bi-9H-carbazol-9-yl)naphtho[1′,2′: 4,5]furo[2,3-b]pyrazine (abbreviation: 1OPCCzNfpr), 9-[3′-(6-phenylbenzo[b]naphtho[1,2-d]furan-8-yl)biphenyl-3-yl]naphtho[1′,2′: 4,5]furo[2,3-b]pyrazine (abbreviation: 9mBnfBPNfpr), 9-{3-[6-(9,9-dimethylfluoren-2-yl)dibenzothiophen-4-yl]phenyl}naphtho[1′,2′: 4,5]furo[2,3-b]pyrazine (abbreviation: 9mFDBtPNfpr), 9-[3′-(6-phenyldibenzothiophen-4-yl)biphenyl-3-yl]naphtho[1′,2′: 4,5]furo[2,3-b]pyrazine (abbreviation: 9mDBtBPNfpr-02), 9-[3-(9′-phenyl-3,3′-bi-9H-carbazol-9-yl)phenyl]naphtho[1′,2′: 4,5]furo[2,3-b]pyrazine (abbreviation: 9mPCCzPNfpr), 9-{(3′-[2,8-diphenyldibenzothiophen-4-yl)biphenyl-3-yl}naphtho[1′,2′: 4,5]furo[2,3-b]pyrazine, 11-{(3′-[2,8-diphenyldibenzothiophen-4-yl)biphenyl-3-yl]phenanthro[9′,10′: 4,5]furo[2,3-b]pyrazine, 5-[3-(4,6-diphenyl-1,3,5-triazin-2-yl)phenyl]-7,7-dimethyl-5H,7H-indeno[2,1-b]carbazole (abbreviation: mINc(II)PTzn), 2-[3′-(triphenylen-2-yl)biphenyl-3-yl]-4,6-diphenyl-1,3,5-triazine (abbreviation: mTpBPTzn), 2-(biphenyl-4-yl)-4-phenyl-6-(9,9′-spirobi[9H-fluoren]-2-yl)-1,3,5-triazine (abbreviation: BP-SFTzn), 2,6-bis(4-naphthalen-1-ylphenyl)-4-[4-(3-pyridyl)phenyl]pyrimidine (abbreviation: 2,4NP-6PyPPm), 3-[9-(4,6-diphenyl-1,3,5-triazin-2-yl)-2-dibenzofuranyl]-9-phenyl-9H-carbazole (abbreviation: PCDBfTzn), 2-(biphenyl-3-yl)-4-phenyl-6-{8-[(1,1′: 4′,1″-terphenyl)-4-yl]-1-dibenzofuranyl}-1,3,5-triazine (abbreviation: mBP-TPDBfTzn), 6-(biphenyl-3-yl)-4-[3,5-bis(9H-carbazol-9-yl)phenyl)-2-phenylpyrimidine (abbreviation: 6mBP-4Cz2PPm), and 4-[3,5-bis(9H-carbazol-9-yl)phenyl]-2-phenyl-6-(biphenyl-4-yl)pyrimidine (abbreviation: 6BP-4Cz2PPm), and those materials are preferable as the host material.

Among the above organic compounds, specific examples of metal complexes that are organic compounds having a high electron-transport property include zinc- or aluminum-based metal complexes, such as tris(8-quinolinolato)aluminum(III) (abbreviation: Alq), tris(4-methyl-8-quinolinolato)aluminum(III) (abbreviation: Almq₃), bis(10-hydroxybenzo[h]quinolinato)beryllium(II) (abbreviation: BeBq₂), bis(2-methyl-8-quinolinolato)(4-phenylphenolato)aluminum(III) (abbreviation: BAlq), and bis(8-quinolinolato)zinc(II) (abbreviation: Znq), and metal complexes having a quinoline ring or a benzoquinoline ring. Such metal complexes are preferable as the host material.

Moreover, high-molecular compounds such as poly(2,5-pyridinediyl) (abbreviation: PPy), poly[(9,9-dihexylfluorene-2,7-diyl)-co-(pyridine-3,5-diyl)](abbreviation: PF-Py), and poly[(9,9-dioctylfluorene-2,7-diyl)-co-(2,2′-bipyridine-6,6′-diyl)] (abbreviation: PF-BPy) are preferable as the host material.

Furthermore, the following organic compounds having a diazine ring, which have bipolar properties, a high hole-transport property, and a high electron-transport property, can be used as the host material: 9-phenyl-9′-(4-phenyl-2-quinazolinyl)-3,3′-bi-9H-carbazole (abbreviation: PCCzQz), 2-[4′-(9-phenyl-9H-carbazol-3-yl)-3,1′-biphenyl-1-yl]dibenzo[f,h]quinoxaline (abbreviation: 2mpPCBPDBq), 5-[3-(4,6-diphenyl-1,3,5-triazin-2-yl)phenyl]-7,7-dimethyl-5H,7H-indeno[2,1-b]carbazole (abbreviation: mINc(II)PTzn), 11-[4-(biphenyl-4-yl)-6-phenyl-1,3,5-triazin-2-yl]-11,12-dihydro-12-phenyl-indolo[2,3-a]carbazole (abbreviation: BP-Icz(II)Tzn), and 7-[4-(9-phenyl-9H-carbazol-2-yl)quinazolin-2-yl]-7H-dibenzo[c,g]carbazole (abbreviation: PC-cgDBCzQz).

### <Electron-Transport Layer>

The electron-transport layers (114, 114a, and 114b) transport the electrons, which are injected from the second electrode 102 and the charge-generation layers (106, 106a, and 106b) by electron-injection layers (115, 115a, and 115b) described later, to the light-emitting layers (113, 113a, and 113b). The heat resistance of the light-emitting device of one embodiment of the present invention can be improved by including the stacked electron-transport layers. The electron-transport material used in the electron-transport layers (114, 114a, and 114b) is preferably a substance having an electron mobility of 1×10¹ cm²/Vs or higher in the case where the square root of the electric field strength [V/cm] is 600. Note that any other substance can also be used as long as the substance has an electron-transport property higher than a hole-transport property. The electron-transport layers (114, 114a, and 114b) can function even with a single-layer structure and may have a stacked-layer structure including two or more layers. When a photolithography process is performed over the electron-transport layer including the above-described mixed material, which has heat resistance, an adverse effect of the thermal process on the device characteristics can be reduced.

### <<Electron-Transport Material>>

As the electron-transport material that can be used for the electron-transport layers (114, 114a, and 114b), an organic compound having a high electron-transport property can be used, and for example, a heteroaromatic compound can be used. The term heteroaromatic compound refers to a cyclic compound including at least two different kinds of elements in a ring. Examples of cyclic structures include a three-membered ring, a four-membered ring, a five-membered ring, a six-membered ring, and the like, among which a five-membered ring and a six-membered ring are particularly preferred. The elements included in the heteroaromatic compound are preferably one or more of nitrogen, oxygen, and sulfur, in addition to carbon. In particular, a heteroaromatic compound containing nitrogen (a nitrogen-containing heteroaromatic compound) is preferable, and any of materials having a high electron-transport property (electron-transport materials), such as a nitrogen-containing heteroaromatic compound and a π-electron deficient heteroaromatic compound including the nitrogen-containing heteroaromatic compound, is preferably used.

Note that the electron-transport material can be different from the materials used in the light-emitting layer. Not all excitons formed by recombination of carriers in the light-emitting layer can contribute to light emission and some excitons are diffused into a layer in contact with the light-emitting layer or a layer in the vicinity of the light-emitting layer. In order to avoid this phenomenon, the energy level (the lowest singlet excitation level or the lowest triplet excitation level) of a material used for the layer in contact with the light-emitting layer or the layer in the vicinity of the light-emitting layer is preferably higher than that of a material used for the light-emitting layer. Thus, when a material different from the material of the light-emitting layer is used as the electron-transport material, a device having high efficiency can be obtained.

The heteroaromatic compound is an organic compound including at least one heteroaromatic ring.

The heteroaromatic ring includes any one of a pyridine ring, a diazine ring, a triazine ring, a polyazole ring, an oxazole ring, a thiazole ring, and the like. A heteroaromatic ring having a diazine ring includes a heteroaromatic ring having a pyrimidine ring, a pyrazine ring, a pyridazine ring, or the like. A heteroaromatic ring having a polyazole ring includes a heteroaromatic ring having an imidazole ring, a triazole ring, or an oxadiazole ring.

The heteroaromatic ring includes a fused heteroaromatic ring having a fused ring structure. Examples of the fused heteroaromatic ring include a quinoline ring, a benzoquinoline ring, a quinoxaline ring, a dibenzoquinoxaline ring, a quinazoline ring, a benzoquinazoline ring, a dibenzoquinazoline ring, a phenanthroline ring, a furodiazine ring, and a benzimidazole ring.

Examples of the heteroaromatic compound having a five-membered ring structure, which is a heteroaromatic compound containing carbon and one or more of nitrogen, oxygen, and sulfur, include a heteroaromatic compound having an imidazole ring, a heteroaromatic compound having a triazole ring, a heteroaromatic compound having an oxazole ring, a heteroaromatic compound having an oxadiazole ring, a heteroaromatic compound having a thiazole ring, and a heteroaromatic compound having a benzimidazole ring.

Examples of the heteroaromatic compound having a six-membered ring structure, which is a heteroaromatic compound containing carbon and one or more of nitrogen, oxygen, and sulfur, include a heteroaromatic compound having a heteroaromatic ring, such as a pyridine ring, a diazine ring (a pyrimidine ring, a pyrazine ring, a pyridazine ring, or the like), a triazine ring, or a polyazole ring. Other examples include a heteroaromatic compound having a bipyridine structure, a heteroaromatic compound having a terpyridine structure, and the like, which are included in examples of a heteroaromatic compound in which pyridine rings are connected.

Examples of the heteroaromatic compound having a fused ring structure partly including the above six-membered ring structure include a heteroaromatic compound having a fused heteroaromatic ring such as a quinoline ring, a benzoquinoline ring, a quinoxaline ring, a dibenzoquinoxaline ring, a phenanthroline ring, a furodiazine ring (including a structure in which an aromatic ring is fused to a furan ring of a furodiazine ring), or a benzimidazole ring.

Specific examples of the above-described heteroaromatic compound having a five-membered ring structure (a polyazole ring (including an imidazole ring, a triazole ring, or an oxadiazole ring), an oxazole ring, a thiazole ring, or a benzimidazole ring) include 2-(4-biphenylyl)-5-(4-tert-butylphenyl)-1,3,4-oxadiazole (abbreviation: PBD), 1,3-bis[5-(p-tert-butylphenyl)-1,3,4-oxadiazol-2-yl]benzene (abbreviation: OXD-7), 9-[4-(5-phenyl-1,3,4-oxadiazol-2-yl)phenyl]-9H-carbazole (abbreviation: COl1), 3-(4-biphenylyl)-4-phenyl-5-(4-tert-butylphenyl)-1,2,4-triazole (abbreviation: TAZ), 3-(4-tert-butylphenyl)-4-(4-ethylphenyl)-5-(4-biphenylyl)-1,2,4-triazole (abbreviation: p-EtTAZ), 2,2′,2″-(1,3,5-benzenetriyl)tris(1-phenyl-1H-benzimidazole) (abbreviation: TPBI), 2-[3-(dibenzothiophen-4-yl)phenyl]-1-phenyl-1H-benzimidazole (abbreviation: mDBTBIm-II), and 4,4′-bis(5-methylbenzoxazol-2-yl)stilbene (abbreviation: BzOs).

Specific examples of the above-described heteroaromatic compound having a six-membered ring structure (including a heteroaromatic ring having a pyridine ring, a diazine ring, a triazine ring, or the like) include: a heteroaromatic compound including a heteroaromatic ring having a pyridine ring, such as 3,5-bis[3-(9H-carbazol-9-yl)phenyl]pyridine (abbreviation: 35DCzPPy) or 1,3,5-tri[3-(3-pyridyl)phenyl]benzene (abbreviation: TmPyPB); a heteroaromatic compound including a heteroaromatic ring having a triazine ring, such as 2-{4-[3-(N-phenyl-9H-carbazol-3-yl)-9H-carbazol-9-yl]phenyl}-4,6-diphenyl-1,3,5-triazine (abbreviation: PCCzPTzn), 9-[3-(4,6-diphenyl-1,3,5-triazin-2-yl)phenyl]-9′-phenyl-2,3′-bi-9H-carbazole (abbreviation: mPCCzPTzn-02), 5-[3-(4,6-diphenyl-1,3,5-triazin-2-yl)phenyl]-7,7-dimethyl-5H,7H-indeno[2,1-b]carbazole (abbreviation: mINc(II)PTzn), 2-[3′-(triphenylen-2-yl)biphenyl-3-yl]-4,6-diphenyl-1,3,5-triazine (abbreviation: mTpBPTzn), 2-(biphenyl-4-yl)-4-phenyl-6-(9,9′-spirobi[9H-fluoren]-2-yl)-1,3,5-triazine (abbreviation: BP-SFTzn), 2,6-bis(4-naphthalen-1-ylphenyl)-4-[4-(3-pyridyl)phenyl]pyrimidine (abbreviation: 2,4NP-6PyPPm), 3-[9-(4,6-diphenyl-1,3,5-triazin-2-yl)-2-dibenzofuranyl]-9-phenyl-9H-carbazole (abbreviation: PCDBfTzn), 2-(biphenyl-3-yl)-4-phenyl-6-{8-[(1,1′: 4′,1″-terphenyl)-4-yl]-1-dibenzofuranyl}-1,3,5-triazine (abbreviation: mBP-TPDBfTzn), 2-{3-[3-(dibenzothiophen-4-yl)phenyl]phenyl}-4,6-diphenyl-1,3,5-triazine (abbreviation: mDBtBPTzn), or mFBPTzn; and a heteroaromatic compound including a heteroaromatic ring having a diazine (pyrimidine) ring, such as 4,6-bis[3-(phenanthren-9-yl)phenyl]pyrimidine (abbreviation: 4,6mPnP2Pm), 4,6-bis[3-(4-dibenzothienyl)phenyl]pyrimidine (abbreviation: 4,6mDBTP2Pm-II), 4,6-bis[3-(9H-carbazol-9-yl)phenyl]pyrimidine (abbreviation: 4,6mCzP2Pm), 4,6mCzBP2Pm, 6-(biphenyl-3-yl)-4-[3,5-bis(9H-carbazol-9-yl)phenyl]-2-phenylpyrimidine (abbreviation: 6mBP-4Cz2PPm), 4-[3,5-bis(9H-carbazol-9-yl)phenyl]-2-phenyl-6-(biphenyl-4-yl)pyrimidine (abbreviation: 6BP-4Cz2PPm), 4-[3-(dibenzothiophen-4-yl)phenyl]-8-(naphthalen-2-yl)-[1]benzofuro[3,2-d]pyrimidine (abbreviation: 8βN-4mDBtPBfpm), 8BP-4mDBtPBfpm, 9mDBtBPNfpr, 9pmDBtBPNfpr, 3,8-bis[3-(dibenzothiophen-4-yl)phenyl]benzofuro[2,3-b]pyrazine (abbreviation: 3,8mDBtP2Bfpr), 4,8-bis[3-(dibenzothiophen-4-yl)phenyl]-[1]benzofuro[3,2-d]pyrimidine (abbreviation: 4,8mDBtP2Bfpm), 8-[3′-(dibenzothiophen-4-yl)biphenyl-3-yl]naphtho[1′,2′: 4,5]furo[3,2-d]pyrimidine (abbreviation: 8mDBtBPNfpm), or 8-[(2,2′-binaphthalen)-6-yl]-4-[3-(dibenzothiophen-4-yl)phenyl]-[1]benzofuro[3,2-d]pyrimidine (abbreviation: 8(βN2)-4mDBtPBfpm). Note that the above aromatic compounds including a heteroaromatic ring include a heteroaromatic compound having a fused heteroaromatic ring.

Other examples include heteroaromatic compounds including a heteroaromatic ring having a diazine (pyrimidine) ring, such as 2,2′-(pyridine-2,6-diyl)bis(4-phenylbenzo[h]quinazoline) (abbreviation: 2,6(P-Bqn)₂Py), 2,2′-(2,2′-bipyridine-6,6′-diyl)bis(4-phenylbenzo[h]quinazoline) (abbreviation: 6,6′(P-Bqn)₂BPy), 2,2′-(pyridine-2,6-diyl)bis{4-[4-(2-naphthyl)phenyl]-6-phenylpyrimidine} (abbreviation: 2,6(NP-PPm)₂Py), or 6-(biphenyl-3-yl)-4-[3,5-bis(9H-carbazol-9-yl)phenyl]-2-phenylpyrimidine (abbreviation: 6mBP-4Cz2PPm), and a heteroaromatic compound including a heteroaromatic ring having a triazine ring, such as 2,4,6-tris[3′-(pyridin-3-yl)biphenyl-3-yl]-1,3,5-triazine (abbreviation: TmPPPyTz), 2,4,6-tris(2-pyridyl)-1,3,5-triazine (abbreviation: 2Py3Tz), or 2-[3-(2,6-dimethyl-3-pyridyl)-5-(9-phenanthryl)phenyl]-4,6-diphenyl-1,3,5-triazine (abbreviation: mPn-mDMePyPTzn).

Specific examples of the above-described heteroaromatic compound having a fused ring structure partly including a six-membered ring structure (the heteroaromatic compound having a fused ring structure) include a heteroaromatic compound having a quinoxaline ring, such as bathophenanthroline (abbreviation: Bphen), bathocuproine (abbreviation: BCP), 2,9-di(naphthalen-2-yl)-4,7-diphenyl-1,10-phenanthroline (abbreviation: NBphen), 2,2′-(1,3-phenylene)bis(9-phenyl-1,10-phenanthroline) (abbreviation: mPPhen2P), 2,2′-(pyridin-2,6-diyl)bis(4-phenylbenzo[h]quinazoline) (abbreviation: 2,6(P-Bqn)₂Py), 2-[3-(dibenzothiophen-4-yl)phenyl]dibenzo[f,h]quinoxaline (abbreviation: 2mDBTPDBq-II), 2-[3′-(dibenzothiophen-4-yl)biphenyl-3-yl]dibenzo[f,h]quinoxaline (abbreviation: 2mDBTBPDBq-II), 2-[3′-(9H-carbazol-9-yl)biphenyl-3-yl]dibenzo[f,h]quinoxaline (abbreviation: 2mCzBPDBq), 2-[4-(3,6-diphenyl-9H-carbazol-9-yl)phenyl]dibenzo[f,h]quinoxaline (abbreviation: 2CzPDBq-III), 7-[3-(dibenzothiophen-4-yl)phenyl]dibenzo[f,h]quinoxaline (abbreviation: 7mDBTPDBq-II), 6-[3-(dibenzothiophen-4-yl)phenyl]dibenzo[f,h]quinoxaline (abbreviation: 6mDBTPDBq-II), and 2mpPCBPDBq.

For the electron-transport layers (114, 114a, and 114b), any of the metal complexes given below can be used as well as the heteroaromatic compounds described above. Examples of the metal complexes include a metal complex having a quinoline ring or a benzoquinoline ring, such as tris(8-quinolinolato)aluminum(III) (abbreviation: Alq₃), Almq₃, 8-quinolinolatolithium(I) (abbreviation: Liq), BeBq₂, bis(2-methyl-8-quinolinolato)(4-phenylphenolato)aluminum(III) (abbreviation: BAlq), or bis(8-quinolinolato)zinc(II) (abbreviation: Znq), and a metal complex having an oxazole ring or a thiazole ring, such as bis[2-(2-benzoxazolyl)phenolato]zinc(II) (abbreviation: ZnPBO), or bis[2-(2-benzothiazolyl)phenolato]zinc(II) (abbreviation: ZnBTZ).

High-molecular compounds such as poly(2,5-pyridinediyl) (abbreviation: PPy), poly[(9,9-dihexylfluorene-2,7-diyl)-co-(pyridine-3,5-diyl)] (abbreviation: PF-Py), and poly[(9,9-dioctylfluorene-2,7-diyl)-co-(2,2′-bipyridine-6,6′-diyl)] (abbreviation: PF-BPy) can be used as the electron-transport material.

Each of the electron-transport layers (114, 114a, and 114b) is not limited to a single layer and may be a stack of two or more layers each containing any of the above substances.

### <Electron-Injection Layer>

The electron-injection layers (115, 115a, and 115b) contain a substance having a high electron-injection property. The electron-injection layers (115, 115a, and 115b) are layers for increasing the efficiency of electron injection from the second electrode 102 and are preferably formed using a material whose value of the LUMO level has a small difference (0.50 eV or less) from the work function of a material used for the second electrode 102. Thus, the electron-injection layer 115 can be formed using an alkali metal, an alkaline earth metal, or a compound thereof, such as lithium, cesium, lithium fluoride (LiF), cesium fluoride (CsF), calcium fluoride (CaF₂), 8-quinolinolato-lithium (abbreviation: Liq), 2-(2-pyridyl)phenolatolithium (abbreviation: LiPP), 2-(2-pyridyl)-3-pyridinolatolithium (abbreviation: LiPPy), 4-phenyl-2-(2-pyridyl)phenolatolithium (abbreviation: LiPPP), an oxide of lithium (LiOₓ), or cesium carbonate. A rare earth metal or a compound of a rare earth metal, such as erbium fluoride (ErF₃) or ytterbium (Yb), can also be used. It is also possible to use a compound including a 1,3,4,6,7,8-tetrahydro-2H-pyrimido[1,2-a]pyrimidine skeleton, such as 1-(9,9′-spirobi[9H-fluoren]-2-yl)-1,3,4,6,7,8-hexahydro-2H-pyrimido[1,2-a]pyrimidine (abbreviation: 2hppSF), 1,1′-(9,9′-spirobi[9H-fluorene]-2,7-diyl)bis(1,3,4,6,7,8-hexahydro-2H-pyrimido[1,2-a]pyrimidine) (abbreviation: 2,7hpp2SF), or 1,1′-pyridine-2,6-diyl-bis(1,3,4,6,7,8-hexahydro-2H-pyrimido[1,2-a]pyrimidine) (abbreviation: hpp2Py). For the electron-injection layers (115, 115a, and 115b), a plurality of kinds of materials given above may be mixed or stacked as films. Electride may also be used for the electron-injection layers (115, 115a, and 115b). Examples of the electride include a substance in which electrons are added at high concentration to calcium oxide-aluminum oxide. Any of the substances used for the electron-transport layers (114, 114a, and 114b), which are given above, can also be used.

A mixed material in which an organic compound and an electron donor (donor) are mixed may also be used for the electron-injection layers (115, 115a, and 115b). Such a mixed material is excellent in an electron-injection property and an electron-transport property because electrons are generated in the organic compound by the electron donor. The organic compound here is preferably a material excellent in transporting the generated electrons; specifically, for example, the above-described electron-transport materials used for the electron-transport layers (114, 114a, and 114b), such as a metal complex and a heteroaromatic compound, can be used. As the electron donor, a substance showing an electron-donating property with respect to an organic compound can be used. Specifically, an alkali metal, an alkaline earth metal, and a rare earth metal are preferable, and lithium, cesium, magnesium, calcium, erbium, ytterbium, and the like are given. In addition, an alkali metal oxide and an alkaline earth metal oxide are preferable, and lithium oxide, calcium oxide, barium oxide, and the like are given. Alternatively, a Lewis base such as magnesium oxide can be used. Further alternatively, an organic compound such as tetrathiafulvalene (abbreviation: TTF) can be used. Alternatively, a stack of two or more of these materials may be used.

A mixed material in which an organic compound and a metal are mixed may also be used for the electron-injection layers (115, 115a, and 115b). The organic compound used here preferably has a LUMO level higher than or equal to −3.60 eV and lower than or equal to −2.30 eV. Moreover, a material having an unshared electron pair is preferable.

Thus, as the organic compound used in the above mixed material, a mixed material obtained by mixing a metal and the heteroaromatic compound given above as the material that can be used for the electron-transport layer may be used. Preferable examples of the heteroaromatic compound include materials having an unshared electron pair, such as a heteroaromatic compound having a five-membered ring structure (e.g., an imidazole ring, a triazole ring, an oxazole ring, an oxadiazole ring, a thiazole ring, or a benzimidazole ring), a heteroaromatic compound having a six-membered ring structure (e.g., a pyridine ring, a diazine ring (including a pyrimidine ring, a pyrazine ring, a pyridazine ring, or the like), a triazine ring, a bipyridine ring, or a terpyridine ring), and a heteroaromatic compound having a fused ring structure partly including a six-membered ring structure (e.g., a quinoline ring, a benzoquinoline ring, a quinoxaline ring, a dibenzoquinoxaline ring, or a phenanthroline ring). Since the materials are specifically described above, description thereof is omitted here.

As a metal used for the above mixed material, a transition metal that belongs to Group 5, Group 7, Group 9, or Group 11 or a material that belongs to Group 13 in the periodic table is preferably used, and examples include Ag, Cu, Al, and In. Here, the organic compound forms a singly occupied molecular orbital (SOMO) with the transition metal.

To amplify light obtained from the light-emitting layer 113b, for example, the optical path length between the second electrode 102 and the light-emitting layer 113b is preferably less than one fourth of the wavelength k of light emitted from the light-emitting layer 113b. In that case, the optical path length can be adjusted by changing the thickness of the electron-transport layer 114b or the electron-injection layer 115b.

When the two EL layers (103a and 103b) are provided and the charge-generation layer 106 is provided between the plurality of EL layers as in the light-emitting device in FIG. 3E, a structure in which a plurality of EL layers are stacked between the pair of electrodes (the structure is also referred to as a tandem structure) can be obtained.

### <Charge-Generation Layer>

The charge-generation layer 106 has a function of injecting electrons into the EL layer 103a and injecting holes into the EL layer 103b when voltage is applied between the first electrode (anode) 101 and the second electrode (cathode) 102. The charge-generation layer 106 may be either a p-type layer in which an electron acceptor (acceptor) is added to a hole-transport material or an electron-injection buffer layer in which an electron donor (donor) is added to an electron-transport material. Alternatively, both of these structures may be stacked. Furthermore, an electron-relay layer may be provided between the p-type layer and the electron-injection buffer layer. Note that forming the charge-generation layer 106 with the use of any of the above materials can inhibit an increase in driving voltage caused by the stack of the EL layers.

In the case where the charge-generation layer 106 is a p-type layer in which an electron acceptor is added to a hole-transport material, which is an organic compound, any of the materials described in this embodiment can be used as the hole-transport material. Examples of the electron acceptor include 7,7,8,8-tetracyano-2,3,5,6-tetrafluoroquinodimethane (abbreviation: F₄-TCNQ) and chloranil. Other examples include oxides of metals that belong to Group 4 to Group 8 of the periodic table. Specific examples include vanadium oxide, niobium oxide, tantalum oxide, chromium oxide, molybdenum oxide, tungsten oxide, manganese oxide, and rhenium oxide. Any of the above-described acceptor materials may be used. Furthermore, a mixed film obtained by mixing materials of a p-type layer or a stack of films containing the respective materials may be used.

In the case where the charge-generation layer 106 is an electron-injection buffer layer in which an electron donor is added to an electron-transport material, any of the materials described in this embodiment can be used as the electron-transport material. As the electron donor, it is possible to use an alkali metal, an alkaline earth metal, a rare earth metal, a metal belonging to Group 2 or Group 13 of the periodic table, or an oxide or a carbonate thereof. Specifically, lithium (Li), cesium (Cs), magnesium (Mg), calcium (Ca), ytterbium (Yb), indium (In), lithium oxide (Li₂O), cesium carbonate, or the like is preferably used. An alkali metal compound such as Liq may be used. An organic compound such as tetrathianaphthacene may be used as the electron donor. An organic compound including a 1,3,4,6,7,8-tetrahydro-2H-pyrimido[1,2-a]pyrimidine skeleton, such as 2hppSF, 2,7hpp2SF, or hpp2Py may be used as the electron donor. When any of these organic compounds is used as the electron donor, the electron-transport material to be combined with the electron donor is preferably an organic compound including a heteroaromatic ring having a phenanthroline ring, such as bathophenanthroline (abbreviation: Bphen), bathocuproine (abbreviation: BCP), 2,9-di(naphthalen-2-yl)-4,7-diphenyl-1,10-phenanthroline (abbreviation: NBphen), or 2,2′-(1,3-phenylene)bis(9-phenyl-1,10-phenanthroline) (abbreviation: mPPhen2P), in which case driving voltage of the light-emitting device can be decreased.

When an electron-relay layer is provided between a p-type layer and an electron-injection buffer layer in the charge-generation layer 106, the electron-relay layer contains at least a substance having an electron-transport property and has a function of preventing an interaction between the electron-injection buffer layer and the p-type layer and transferring electrons smoothly. The LUMO level of the substance having an electron-transport property in the electron-relay layer is preferably between the LUMO level of the acceptor substance in the p-type layer and the LUMO level of the substance having an electron-transport property in the electron-transport layer in contact with the charge-generation layer 106. Specifically, the LUMO level of the substance having an electron-transport property in the electron-relay layer can be higher than or equal to −5.00 eV, preferably higher than or equal to −5.00 eV and lower than or equal to −3.00 eV. Note that as the substance having an electron-transport property in the electron-relay layer, a phthalocyanine-based material or a metal complex having a metal-oxygen bond and an aromatic ligand is preferably used.

Note that in terms of light extraction efficiency, the charge-generation layer 106 preferably has a property of transmitting visible light (specifically, the charge-generation layer 106 preferably has a visible light transmittance of 40% or more). The charge-generation layer 106 functions even if it has lower conductivity than the first electrode 101 or the second electrode 102.

Although FIG. 3E illustrates the structure in which two EL layers 103 are stacked, three or more EL layers may be stacked with charge-generation layers each provided between two adjacent EL layers.

### <Cap Layer>

Although not illustrated in FIGS. 3A to 3F, a cap layer may be provided over the second electrode 102 of the light-emitting device. For example, a material with high refractive index can be used for the cap layer. When the cap layer is provided over the second electrode 102, extraction efficiency of light emitted from the second electrode 102 can be improved.

Specific examples of a material that can be used for the cap layer include 5,5′-diphenyl-2,2′-di-5H-[1]benzothieno[3,2-c]carbazole (abbreviation: BisBTc), and 4,4′,4″-(benzene-1,3,5-triyl)tri(dibenzothiophene) (abbreviation: DBT3P-II).

### <Substrate>

The light-emitting device described in this embodiment can be formed over a variety of substrates. Note that the type of substrate is not limited to a certain type. Examples of the substrate include semiconductor substrates (e.g., a single crystal substrate and a silicon substrate), an SOI substrate, a glass substrate, a quartz substrate, a plastic substrate, a metal substrate, a stainless steel substrate, a substrate including stainless steel foil, a tungsten substrate, a substrate including tungsten foil, a flexible substrate, an attachment film, and paper or a base material film including a fibrous material.

Examples of the glass substrate include a barium borosilicate glass substrate, an aluminoborosilicate glass substrate, and a soda lime glass substrate. Examples of the flexible substrate, the attachment film, and the base material film include plastics typified by polyethylene terephthalate (PET), polyethylene naphthalate (PEN), and polyether sulfone (PES), a synthetic resin such as acrylic resin, polypropylene, polyester, polyvinyl fluoride, polyvinyl chloride, polyamide, polyimide, aramid, epoxy resin, an inorganic vapor deposition film, and paper.

For manufacturing the light-emitting device in this embodiment, a gas phase method such as an evaporation method or a liquid phase method such as a spin coating method or an ink-jet method can be used. When an evaporation method is used, a physical vapor deposition method (PVD method) such as a sputtering method, an ion plating method, an ion beam evaporation method, a molecular beam evaporation method, or a vacuum evaporation method, a chemical vapor deposition method (CVD method), or the like can be used. Specifically, the layers having various functions (the hole-injection layer 111, the hole-transport layer 112, the light-emitting layer 113, the electron-transport layer 114, and the electron-injection layer 115) included in the EL layers of the light-emitting device can be formed by an evaporation method (e.g., a vacuum evaporation method), a coating method (e.g., a dip coating method, a die coating method, a bar coating method, a spin coating method, or a spray coating method), a printing method (e.g., an ink-jet method, screen printing (stencil), offset printing (planography), flexography (relief printing), gravure printing, or micro-contact printing), or the like.

In the case where a film formation method such as the coating method or the printing method is employed, a high-molecular compound (e.g., an oligomer, a dendrimer, or a polymer), a middle-molecular compound (a compound between a low-molecular compound and a high-molecular compound with a molecular weight of 400 to 4000), an inorganic compound (e.g., a quantum dot material), or the like can be used. The quantum dot material can be a colloidal quantum dot material, an alloyed quantum dot material, a core-shell quantum dot material, a core quantum dot material, or the like.

Materials that can be used for the layers (the hole-injection layer 111, the hole-transport layer 112, the light-emitting layer 113, the electron-transport layer 114, and the electron-injection layer 115) included in the EL layer 103 of the light-emitting device described in this embodiment are not limited to the materials described in this embodiment, and other materials can be used in combination as long as the functions of the layers are fulfilled.

The structures described in this embodiment can be used in combination with any of the structures described in the other embodiments as appropriate.

### Embodiment 4

This embodiment will describe a light-emitting and light-receiving apparatus 700 as a specific example of a light-emitting apparatus of one embodiment of the present invention and an example of the manufacturing method. Note that the light-emitting and light-receiving apparatus 700 includes both a light-emitting device and a light-receiving device, and can also be referred to as a light-emitting apparatus including a light-receiving device or a light-receiving apparatus including a light-emitting device. In addition, the light-emitting and light-receiving apparatus 700 can be used for a display portion of an electronic appliance or the like, and thus can also be referred to as a display panel or a display apparatus.

### <Structure Example of Light-Emitting and Light-Receiving Apparatus 700>

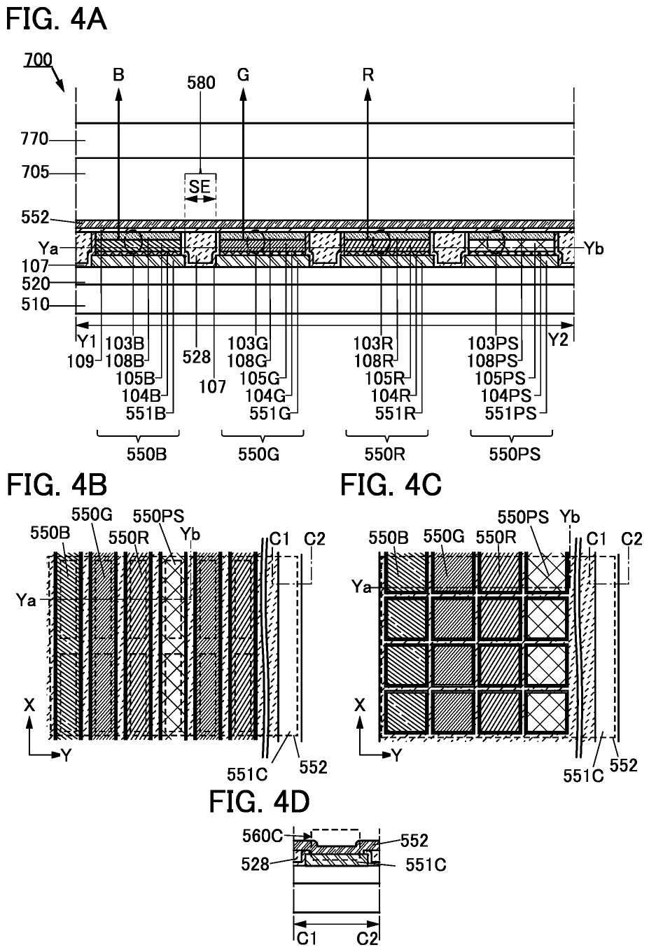

The light-emitting and light-receiving apparatus 700 illustrated in FIG. 4A includes a light-emitting device 550B, a light-emitting device 550G, a light-emitting device 550R, and a light-receiving device 550PS that are formed over a functional layer 520 over a first substrate 510. The functional layer 520 includes, for example, driver circuits such as a gate driver and a source driver that are composed of a plurality of transistors, and wirings that electrically connect these components. These driver circuits are electrically connected to the light-emitting device 550B, the light-emitting device 550G, the light-emitting device 550R, and the light-receiving device 550PS, for example, to drive them. The light-emitting and light-receiving apparatus 700 includes an insulating layer 705 over the functional layer 520 and the devices (the light-emitting devices and the light-receiving device), and the insulating layer 705 has a function of attaching a second substrate 770 and the functional layer 520.

At least one of the light-emitting devices 550B, 550G, and 550R has the device structure described in the foregoing embodiment. In addition, the structure of the EL layer 103 (see FIG. 3A) differs between the light-emitting devices. For example, alight-emitting layer 105B of the EL layer 103B can emit blue light, a light-emitting layer 105G of an EL layer 103G can emit green light, and a light-emitting layer 105R of an EL layer 103R can emit red light. For example, the light-emitting layer 105B can have the structure of the light-emitting layer described in the foregoing embodiment, and the light-emitting layers 105G and 105R can be formed using known materials.

Although the case where the devices (a plurality of light-emitting devices and a light-receiving device) are formed separately is described in this embodiment, part of an EL layer of a light-emitting device (a hole-injection layer, a hole-transport layer, and an electron-transport layer) and part of an active layer of a light-receiving device (the hole-injection layer, the hole-transport layer, and the electron-transport layer) may be formed using the same material at the same time in the manufacturing process.

In this specification and the like, a structure in which light-emitting layers in light-emitting devices of different colors (e.g., blue (B), green (G), and red (R)) and a light-receiving layer in a light-receiving device are separately formed or separately patterned is sometimes referred to as a side-by-side (SBS) structure. Although the light-emitting device 550B, the light-emitting device 550G, the light-emitting device 550R, and the light-receiving device 550PS are arranged in this order in the light-emitting and light-receiving apparatus 700 illustrated in FIG. 4A, one embodiment of the present invention is not limited to this structure.

In FIG. 4A, the light-emitting device 550B includes an electrode 551B, an electrode 552, and the EL layer 103B interposed between the electrode 551B and the electrode 552. The light-emitting device 550G includes an electrode 551G, the electrode 552, and the EL layer 103G interposed between the electrode 551G and the electrode 552. The light-emitting device 550R includes an electrode 551R, the electrode 552, and the EL layer 103R interposed between the electrode 551R and the electrode 552. The EL layers (103B, 103G, and 103R) each have a stacked-layer structure of layers having different functions including their respective light-emitting layers (105B, 105G, and 105R). Note that a specific structure of each layer of the light-emitting device is described in the foregoing embodiment, but is not limited.

In FIG. 4A, the light-receiving device 550PS includes an electrode 551PS, the electrode 552, and a light-receiving layer 103PS interposed between the electrode 551PS and the electrode 552. The light-receiving layer 103PS has a stacked-layer structure of layers having different functions including an active layer 105PS. The active layer 105PS contains a semiconductor. Examples of the semiconductor include inorganic semiconductors such as silicon and organic semiconductors such as organic compounds. Specific structures of other layers in the light-receiving device can be similar to the structures of the corresponding layers in the light-emitting device.

FIG. 4A illustrates a case where the EL layer 103B includes a hole-injection/transport layer 104B, the light-emitting layer 105B, an electron-transport layer 108B, and an electron-injection layer 109; the EL layer 103G includes a hole-injection/transport layer 104G, the light-emitting layer 105G, an electron-transport layer 108G, and the electron-injection layer 109; the EL layer 103R includes a hole-injection/transport layer 104R, the light-emitting layer 105R, an electron-transport layer 108R, and the electron-injection layer 109; and the light-receiving layer 103PS includes a hole-injection/transport layer 104PS, the active layer 105PS, an electron-transport layer 108PS, and the electron-injection layer 109. However, the present invention is not limited thereto.

In FIG. 4A, the electron-injection layer 109 and the electrode 552 are layers (common layers) shared by the devices (the light-emitting device 550B, the light-emitting device 550G, the light-emitting device 550R, and the light-receiving device 550PS).

Hereinafter, for simplicity, the light-emitting device 550B, the light-emitting device 550G, and the light-emitting device 550R are collectively referred to as a light-emitting device 550; the electrode 551B, the electrode 551G, and the electrode 551R are collectively referred to as an electrode 551; the EL layer 103B, the EL layer 103G, and the EL layer 103R are collectively referred to as the EL layer 103; the hole-injection/transport layer 104B, the hole-injection/transport layer 104G, and the hole-injection/transport layer 104R are collectively referred to as a hole-injection/transport layer 104; the light-emitting layer 105B, the light-emitting layer 105G, and the light-emitting layer 105R are collectively referred to as a light-emitting layer 105; and the electron-transport layer 108B, the electron-transport layer 108G, and the electron-transport layer 108R are collectively referred to as an electron-transport layer 108, in some cases.

As illustrated in FIG. 4A, an insulating layer 107 may be formed on the side surfaces (or end portions) of the hole-injection/transport layer 104, the light-emitting layer 105, and the electron-transport layer 108 included in the EL layer 103, and the side surfaces (or end portions) of the hole-injection/transport layer 104PS, the active layer 105PS, and the electron-transport layer 108PS included in the light-receiving layer 103PS. The insulating layer 107 is formed in contact with the side surfaces (or end portions) of the EL layer 103 and the light-receiving layer 103PS. This can inhibit entry of oxygen, moisture, or constituent elements thereof into the inside through the side surfaces of the EL layer 103 and the light-receiving layer 103PS. Note that the insulating layer 107 continuously covers the side surfaces of part of the EL layer 103 and part of the light-receiving layer 103PS of adjacent devices. For example, in FIG. 4A, the side surfaces of parts of the EL layer 103B of the light-emitting device 550B and the EL layer 103G of the light-emitting device 550G are covered with the continuous insulating layer 107.

As illustrated in FIG. 4A, a partition 528 is provided between the devices. Note that the electron-injection layer 109 and the electrode 552 that are common layers shared by the devices are provided continuously without being divided by the partition 528. Thus, the partition 528 can be regarded as being provided in a region surrounded by the electron-injection layer 109 and the insulating layer 107. In addition, the partitions 528 are positioned along the side surfaces (or end portions) of the electrode 551, part of the EL layer 103 (the hole-injection/transport layer 104, the light-emitting layer 105, and the electron-transport layer 108), and part of the light-receiving layer 103PS (the hole-injection/transport layer 104, the active layer 105PS, and the electron-transport layer 108) with the insulating layer 107 therebetween.

In each of the EL layer 103 and the light-receiving layer 103PS, particularly the hole-injection layer, which is included in the hole-transport region between the anode and the light-emitting layer and between the anode and the active layer, often has high conductivity; thus, a hole-injection layer formed as a layer shared by adjacent devices might cause crosstalk. Thus, as described in this structure example, part of the EL layer 103 (the hole-injection/transport layer 104, the light-emitting layer 105, and the electron-transport layer 108) and part of the light-receiving layer 103PS (the hole-injection/transport layer 104PS, the active layer 105PS, and the electron-transport layer 108PS) are separated, and the insulating layer 107 and the partition 528 are provided therebetween, so that crosstalk between adjacent devices can be inhibited.

Furthermore, a depression portion generated between adjacent devices can be flattened by provision of the partition 528. When the depression portion is flattened, disconnection of the electron-injection layer 109 and the electrode 552 formed over the EL layer 103 and the light-receiving layer 103PS can be inhibited.

For the insulating layer 107, aluminum oxide, magnesium oxide, hafnium oxide, gallium oxide, indium gallium zinc oxide, silicon nitride, or silicon nitride oxide can be used, for example. Some of the above-described materials may be stacked to form the insulating layer 107. The insulating layer 107 can be formed by a sputtering method, a CVD method, a molecular beam epitaxy (MBE) method, a pulsed laser deposition (PLD) method, an atomic layer deposition (ALD) method, or the like and is formed preferably by an ALD method, which achieves good coverage.

Examples of an insulating material used to form the partition 528 include organic materials such as an acrylic resin, a polyimide resin, an epoxy resin, an imide resin, a polyamide resin, a polyimide-amide resin, a silicone resin, a siloxane resin, a benzocyclobutene-based resin, a phenol resin, and precursors of these resins. Other examples include organic materials such as polyvinyl alcohol (PVA), polyvinyl butyral, polyvinyl pyrrolidone, polyethylene glycol, polyglycerin, pullulan, water-soluble cellulose, and an alcohol-soluble polyamide resin. A photosensitive resin such as a photoresist can also be used. Examples of the photosensitive resin include positive-type materials and negative-type materials.

With the use of the photosensitive resin, the partition 528 can be formed by only light exposure and developing steps. The partition 528 may be formed using a negative photosensitive resin (e.g., a resist material). In the case where an insulating layer containing an organic material is used as the partition 528, a material absorbing visible light is suitably used. When such a material absorbing visible light is used for the partition 528, light emission from the EL layer can be absorbed by the partition 528, leading to a reduction in light leakage (stray light) to an adjacent EL layer or light-receiving layer. Thus, a light-emitting and light-receiving apparatus having high display quality can be provided.

For example, the difference between the top-surface level of the partition 528 and the top-surface level of the EL layer 103 or the light-receiving layer 103PS is preferably 0.5 times or less, further preferably 0.3 times or less the thickness of the partition 528. The partition 528 may be provided such that the top-surface level of the EL layer 103 or the light-receiving layer 103PS is higher than the top-surface level of the partition 528, for example. Alternatively, the partition 528 may be provided such that the top-surface level of the partition 528 is higher than the top-surface level of the active layer of the EL layer 103B, the EL layer 103G, and the EL layer 103R or the active layer of the light-receiving layer 103PS, for example.

When crosstalk occurs between devices in a light-emitting and light-receiving apparatus with a high resolution exceeding 1000 ppi, a color gamut that the light-emitting and light-receiving apparatus can reproduce is narrowed. In a light-emitting and light-receiving apparatus with a high resolution more than 1000 ppi, preferably more than 2000 ppi, further preferably more than 5000 ppi, the insulating layer 107 and the partition 528 are provided between part of the EL layer 103 (the hole-injection/transport layer 104, the light-emitting layer 105, and the electron-transport layer 108) and part of the light-receiving layer 103PS (the hole-injection/transport layer 104, the active layer 105PS, and the electron-transport layer 108), whereby the light-emitting and light-receiving apparatus can display bright colors.

FIGS. 4B and 4C are each a schematic top view of the light-emitting and light-receiving apparatus 700 taken along the dashed-dotted line Ya-Yb in the cross-sectional view of FIG. 4A. That is, the devices are arranged in a matrix. Note that FIG. 4B illustrates what is called a stripe arrangement, in which the light-emitting devices of the same color or the light-receiving devices are arranged in the X-direction. FIG. 4C illustrates a structure in which the light-emitting devices of the same color or the light-receiving devices are arranged in the X-direction and separated by patterning for each pixel. Note that the arrangement method of the light-emitting devices is not limited thereto; another method such as a delta, zigzag, PenTile, or diamond arrangement can also be used.

Note that part of the EL layer 103 (the hole-injection/transport layer 104, the light-emitting layer 105, and the electron-transport layer 108) and part of the light-receiving layer 103PS (the hole-injection/transport layer 104PS, the active layer 105PS, and the electron-transport layer 108PS) are processed by patterning using a photolithography method for separation, so that a light-emitting and light-receiving apparatus (display panel) with a high resolution can be manufactured. The end portions (side surfaces) of the layers of the EL layer 103 and the layers of the light-receiving layer 103PS processed by patterning using a photolithography method have substantially one surface (or are positioned on substantially the same plane). In this case, the widths (SE) of spaces 580 between the EL layers and between the EL layer and the light-receiving layer are each preferably 5 μm or less, further preferably 1 μm or less.

FIG. 4D is a schematic cross-sectional view taken along the dashed-dotted line C1-C2 in FIGS. 4B and 4C. FIG. 4D illustrates a connection portion 560C where a connection electrode 551C and the electrode 552 are electrically connected to each other. In the connection portion 560C, the electrode 552 is provided over and in contact with the connection electrode 551C. The partition 528 is provided to cover an end portion of the connection electrode 551C.

### <Manufacturing Method Example of Light-Emitting and Light-Receiving Apparatus>

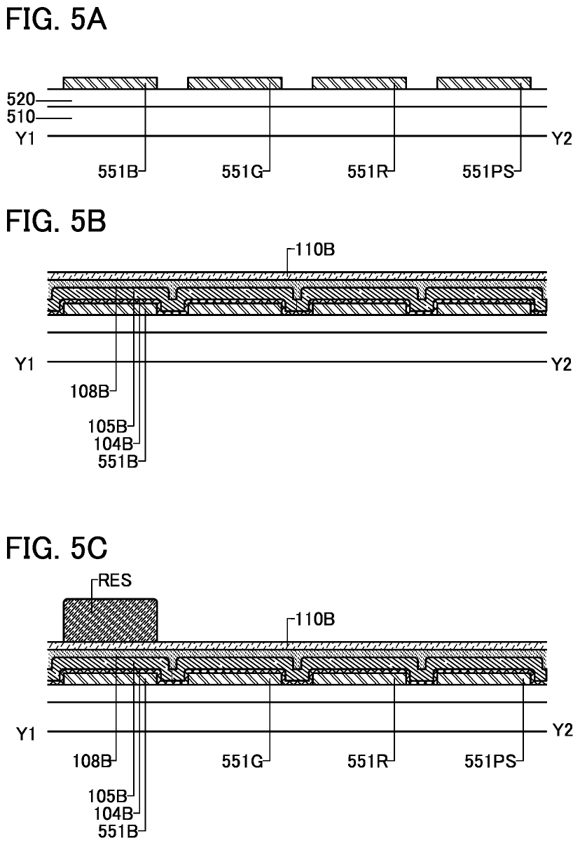

The electrode 551B, the electrode 551G, the electrode 551R, and the electrode 551PS are formed as illustrated in FIG. 5A. For example, a conductive film is formed over the functional layer 520 over the first substrate 510 and processed into predetermined shapes by a photolithography method.

The conductive film can be formed by any of a sputtering method, a CVD method, an MBE method, a vacuum evaporation method, a PLD method, an ALD method, and the like. Examples of the CVD method include a plasma-enhanced chemical vapor deposition (PECVD) method and a thermal CVD method. An example of a thermal CVD method is a metal organic CVD (MOCVD) method.

The conductive film may be processed by a nanoimprinting method, a sandblasting method, a lift-off method, or the like as well as a photolithography method described above. Alternatively, island-shaped thin films may be directly formed by a film formation method using a shielding mask such as a metal mask.

There are two typical examples of photolithography methods. In one of the methods, a resist mask is formed over a thin film that is to be processed, the thin film is processed by etching or the like, and then the resist mask is removed. In the other method, a photosensitive thin film is formed and then processed into a desired shape by light exposure and development. The former method involves heat treatment steps such as pre-applied bake (PAB) after resist application and post-exposure bake (PEB) after light exposure. In one embodiment of the present invention, a lithography method is used not only for processing of a conductive film but also for processing of a thin film used for formation of an EL layer (a film made of an organic compound or a film partly including an organic compound).

As light for exposure in a photolithography method, it is possible to use light with the i-line (wavelength: 365 nm), light with the g-line (wavelength: 436 nm), light with the h-line (wavelength: 405 nm), or light in which the i-line, the g-line, and the h-line are mixed. Alternatively, ultraviolet light, KrF laser light, ArF laser light, or the like can be used. Exposure may be performed by liquid immersion exposure technique. As the light for exposure, extreme ultraviolet (EUV) light or X-rays may also be used. Instead of the light for exposure, an electron beam can be used. It is preferable to use EUV, X-rays, or an electron beam because extremely minute processing can be performed. Note that a photomask is not needed when light exposure is performed by scanning with a beam such as an electron beam.

For etching of a thin film using a resist mask, a dry etching method, a wet etching method, a sandblasting method, or the like can be used.

Subsequently, as illustrated in FIG. 5B, the hole-injection/transport layer 104B, the light-emitting layer 105B, and the electron-transport layer 108B are formed over the electrode 551i, the electrode 551G, the electrode 551R, and the electrode 551PS. Note that the hole-injection/transport layer 104B, the light-emitting layer 105B, and the electron-transport layer 108B can be formed by a vacuum evaporation method, for example. Furthermore, a sacrificial layer 110B is formed over the electron-transport layer 108B. For the formation of the hole-injection/transport layer 104B, the light-emitting layer 105B, and the electron-transport layer 108B, any of the materials described in the above embodiments can be used, for example.

For the sacrificial layer 110B, it is preferable to use a film highly resistant to etching treatment performed on the hole-injection/transport layer 104B, the light-emitting layer 105B, and the electron-transport layer 108B, i.e., a film having high etching selectivity with respect to the hole-injection/transport layer 104B, the light-emitting layer 105B, and the electron-transport layer 108B. The sacrificial layer 110B preferably has a stacked-layer structure of a first sacrificial layer and a second sacrificial layer that have different etching selectivities. For the sacrificial layer 110B, it is possible to use a film that can be removed by a wet etching method, which causes less damage to the EL layer 103B. In wet etching, oxalic acid or the like can be used as an etching material. Note that in this specification and the like, a sacrificial layer may be called a mask layer.

For the sacrificial layer 110B, an inorganic film such as a metal film, an alloy film, a metal oxide film, a semiconductor film, or an inorganic insulating film can be used, for example. The sacrificial layer 110B can be formed by any of a variety of film formation methods such as a sputtering method, an evaporation method, a CVD method, and an ALD method.

For the sacrificial layer 110B, a metal material such as gold, silver, platinum, magnesium, nickel, tungsten, chromium, molybdenum, iron, cobalt, copper, palladium, titanium, aluminum, yttrium, zirconium, or tantalum or an alloy material containing the metal material can be used, for example. It is particularly preferable to use a low-melting-point material such as aluminum or silver.

A metal oxide such as indium gallium zinc oxide (also referred to as In—Ga—Zn oxide or IGZO) can be used for the sacrificial layer 110B. It is also possible to use indium oxide, indium zinc oxide (In—Zn oxide), indium tin oxide (In—Sn oxide), indium titanium oxide (In—Ti oxide), indium tin zinc oxide (In—Sn—Zn oxide), indium titanium zinc oxide (In—Ti—Zn oxide), indium gallium tin zinc oxide (In—Ga—Sn—Zn oxide), or the like. Alternatively, indium tin oxide containing silicon can also be used, for example.

An element M (M is one or more of aluminum, silicon, boron, yttrium, copper, vanadium, beryllium, titanium, iron, nickel, germanium, zirconium, molybdenum, lanthanum, cerium, neodymium, hafnium, tantalum, tungsten, and magnesium) may be used instead of gallium. In particular, M is preferably one or more of gallium, aluminum, and yttrium.

For the sacrificial layer 110B, an inorganic insulating material such as aluminum oxide, hafnium oxide, or silicon oxide can be used.

The sacrificial layer 110B is preferably formed using a material that can be dissolved in a solvent chemically stable with respect to at least the electron-transport layer 108B that is in the uppermost position. Specifically, a material that can be dissolved in water or alcohol can be suitably used for the sacrificial layer 110B. In formation of the sacrificial layer 110B, it is preferable that application of such a material dissolved in a solvent such as water or alcohol be performed by a wet process and followed by heat treatment for evaporating the solvent. At this time, the heat treatment is preferably performed under a reduced-pressure atmosphere, in which case the solvent can be removed at a low temperature in a short time and thermal damage to the hole-injection/transport layer 104B, the light-emitting layer 105B, and the electron-transport layer 108B can be accordingly reduced.

In the case where the sacrificial layer 110B having a stacked-layer structure is formed, the stacked-layer structure can include the first sacrificial layer formed using any of the above-described materials and the second sacrificial layer thereover.

The second sacrificial layer in that case is a film used as a hard mask for etching of the first sacrificial layer. In processing the second sacrificial layer, the first sacrificial layer is exposed. Thus, the combination of films having high etching selectivity therebetween is selected for the first sacrificial layer and the second sacrificial layer. Thus, a film that can be used for the second sacrificial layer can be selected in accordance with the etching conditions of the first sacrificial layer and those of the second sacrificial layer.

For example, in the case where the second sacrificial layer is etched by dry etching involving a fluorine-containing gas (also referred to as a fluorine-based gas), the second sacrificial layer can be formed using silicon, silicon nitride, silicon oxide, tungsten, titanium, molybdenum, tantalum, tantalum nitride, an alloy containing molybdenum and niobium, an alloy containing molybdenum and tungsten, or the like. Here, a film of a metal oxide such as IGZO or ITO can be given as an example of a film having a high etching selectivity with respect to the second sacrificial layer (i.e., a film with a low etching rate) in the dry etching involving the fluorine-based gas, and can be used for the first sacrificial layer.

Note that the material for the second sacrificial layer is not limited to the above and can be selected from a variety of materials in accordance with the etching conditions of the first sacrificial layer and those of the second sacrificial layer. For example, any of the films that can be used for the first sacrificial layer can be used for the second sacrificial layer.

For the second sacrificial layer, a nitride film can be used, for example. Specifically, it is possible to use a nitride such as silicon nitride, aluminum nitride, hafnium nitride, titanium nitride, tantalum nitride, tungsten nitride, gallium nitride, or germanium nitride.

Alternatively, an oxide film can be used for the second sacrificial layer. Typically, it is possible to use a film of an oxide or an oxynitride such as silicon oxide, silicon oxynitride, aluminum oxide, aluminum oxynitride, hafnium oxide, or hafnium oxynitride.

Next, as illustrated in FIG. 5C, a resist is applied onto the sacrificial layer 110B, and the resist having a desired shape (a resist mask RES) is formed by a photolithography method. Such a method involves heat treatment steps such as pre-applied bake (PAB) after the resist application and post-exposure bake (PEB) after light exposure. The temperature reaches approximately 100° C. during the PAB, and approximately 120° C. during the PEB, for example. Thus, the light-emitting device should be resistant to such high treatment temperatures.

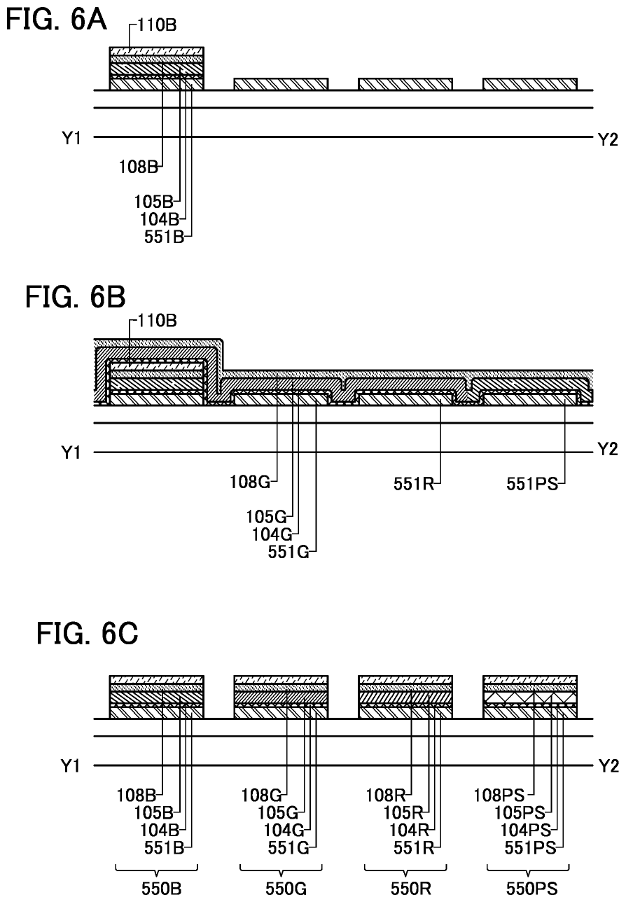

Next, part of the sacrificial layer 110B that is not covered with the resist mask RES is removed by etching using the resist mask RES, the resist mask RES is removed, and then the hole-injection/transport layer 104B, the light-emitting layer 105B, and the electron-transport layer 108B that are not covered with the sacrificial layer 110B are removed by etching, so that the hole-injection/transport layer 104B, the light-emitting layer 105B, and the electron-transport layer 108B are processed to have side surfaces (or have their side surfaces exposed) over the electrode 551B or have belt-like shapes extending in the direction intersecting the sheet of the diagram. Note that dry etching is preferably employed for the etching. In the case where the sacrificial layer 110B has the aforementioned stacked-layer structure of the first sacrificial layer and the second sacrificial layer, the hole-injection/transport layer 104B, the light-emitting layer 105B, and the electron-transport layer 108B may be processed into a predetermined shape in the following manner: part of the second sacrificial layer is etched using the resist mask RES, the resist mask RES is then removed, and part of the first sacrificial layer is etched using the second sacrificial layer as a mask. The structure illustrated in FIG. 6A is obtained through these etching steps.

Subsequently, as illustrated in FIG. 6B, the hole-injection/transport layer 104G, the light-emitting layer 105G, and the electron-transport layer 108G are formed over the sacrificial layer 110B, the electrode 551G, the electrode 551R, and the electrode 551PS. The hole-injection/transport layer 104G, the light-emitting layer 105G, and the electron-transport layer 108G can be formed using any of the materials described in the above embodiments, for example. Note that the hole-injection/transport layer 104G, the light-emitting layer 105G, and the electron-transport layer 108G can be formed by a vacuum evaporation method, for example.

Hereinafter, in a manner similar to formation of the hole-injection/transport layer 104B, the light-emitting layer 105B, the electron-transport layer 108B, and the sacrificial layer 110B, the hole-injection/transport layer 104G, the light-emitting layer 105G, the electron-transport layer 108G, and a sacrificial layer 110G are formed over the electrode 551G, the hole-injection/transport layer 104R, the light-emitting layer 105R, the electron-transport layer 108R, and a sacrificial layer 110R are formed over the electrode 551R, and the hole-injection/transport layer 104PS, the active layer 105PS, the electron-transport layer 108PS, and a sacrificial layer 110PS are formed over the electrode 551PS, whereby the structure illustrated in FIG. 6C is obtained.

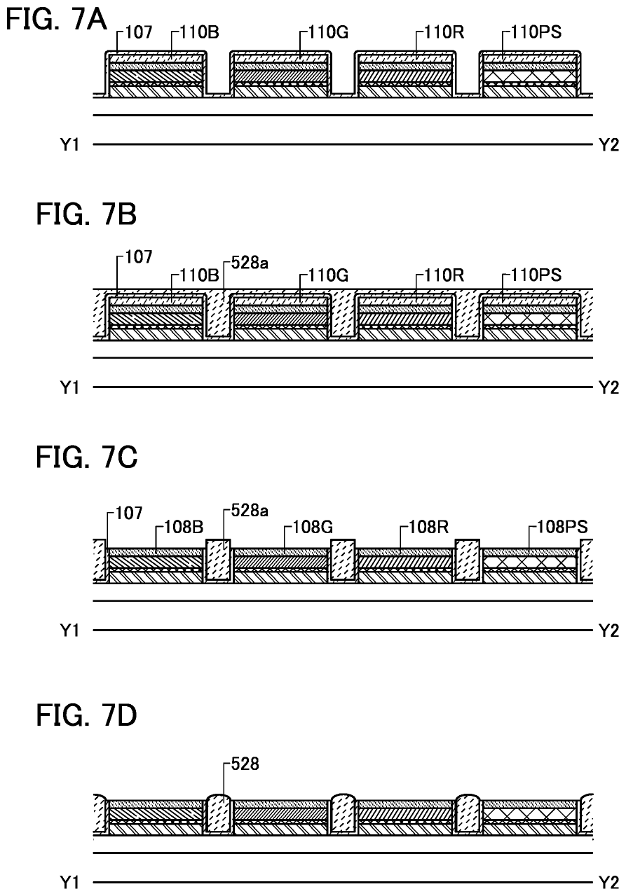

Next, as illustrated in FIG. 7A, the insulating layer 107 is formed over the sacrificial layers 110B, 110G, 110R, and 110PS.

Note that the insulating layer 107 can be formed by an ALD method, for example. In this case, as illustrated in FIG. 7A, the insulating layer 107 is formed to be in contact with the side surfaces (end portions) of the hole-injection/transport layers (104B, 104G, 104R, and 104PS), the light-emitting layers (105B, 105G, and 105R), the active layer 105PS, and the electron-transport layers (108B, 108G, 108R, and 108PS) of the devices. This can inhibit entry of oxygen, moisture, or constituent elements thereof into the inside through the side surfaces of the layers.

Next, as illustrated in FIG. 7B, a resin film 528a is formed over the insulating layer 107. As the resin film 528a, for example, a negative photosensitive resin or a positive photosensitive resin can be used.

Then, as illustrated in FIG. 7C, part of the resin film 528a, part of the insulating layer 107, and the sacrificial layers (110B, 110G, 110R, and 110PS) are removed to expose the top surfaces of the electron-transport layers (108B, 108G, 108R, and 108PS).

Next, heat treatment is performed to process an upper edge portion of the resin film 528a into a curved shape, so that the partition 528 is formed, as illustrated in FIG. 7D. When the upper edge portion of the partition 528 has a curved shape, good coverage with the electron-injection layer 109 to be formed later can be obtained. For example, in the case of using a positive photosensitive acrylic resin as a material for the resin film 528a, the partition 528 is preferably formed so as to have a curved surface with a curvature radius (greater than or equal to 0.2 μm and less than or equal to 3 μm) at the upper edge portion.

Next, the electron-injection layer 109 is formed over the insulating layer 107, the electron-transport layers (108B, 108G, 108R, and 108PS), and the partition 528. The electron-injection layer 109 can be formed using any of the materials described in the above embodiments. The electron-injection layer 109 is formed by a vacuum evaporation method, for example.

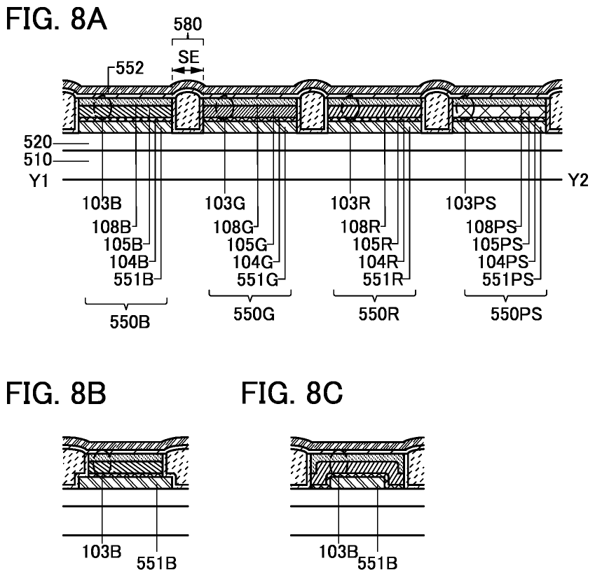

Next, as illustrated in FIG. 8A, the electrode 552 is formed over the electron-injection layer 109. The electrode 552 is formed by a vacuum evaporation method, for example.

Through the above steps, the EL layer 103B, the EL layer 103G, the EL layer 103R, and the light-receiving layer 103PS in the light-emitting device 550B, the light-emitting device 550G, the light-emitting device 550R, and the light-receiving device 550PS can be processed to be separated from each other.

Pattern formation by a photolithography method is performed in separate processing of the EL layer 103 and the light-receiving layer 103PS in the above manner, so that a light-emitting and light-receiving apparatus (display panel) with a high resolution can be manufactured. The end portions (side surfaces) of the layers of the EL layer and the light-receiving layer processed by patterning using a photolithography method have substantially one surface (or are positioned on substantially the same plane). The pattern formation by a photolithography method can inhibit crosstalk between adjacent light-emitting devices and between the light-emitting device and the light-receiving device. In addition, the space 580 is provided between adjacent devices processed by patterning using a photolithography method. In FIG. 8C, when the space 580 is denoted by a distance SE between the EL layers of adjacent light-emitting devices, decreasing the distance SE can increase the aperture ratio and the resolution. By contrast, as the distance SE is increased, the effect of the difference in the manufacturing process between the adjacent light-emitting devices becomes permissible, which leads to an increase in manufacturing yield. Since the light-emitting device manufactured according to this specification is suitable for a miniaturization process, the distance SE between the EL layers of adjacent light-emitting devices can be longer than or equal to 0.5 μm and shorter than or equal to 5 μm, preferably longer than or equal to 1 μm and shorter than or equal to 3 μm, further preferably longer than or equal to 1 μm and shorter than or equal to 2.5 μm, still further preferably longer than or equal to 1 μm and shorter than or equal to 2 μm. Typically, the distance SE is preferably longer than or equal to 1 μm and shorter than or equal to 2 μm (e.g., 1.5 μm or a neighborhood thereof).

In this specification and the like, a device formed using a metal mask or a fine metal mask (FMM) is sometimes referred to as a device having a metal mask (MM) structure. In this specification and the like, a device formed without using a metal mask or an FMM is sometimes referred to as a device having a metal maskless (MML) structure. Since a light-emitting and light-receiving apparatus having the MML structure is formed without using a metal mask, the pixel arrangement, the pixel shape, and the like can be designed more flexibly than in a light-emitting and light-receiving apparatus having the FMM structure or the MM structure.

Note that the island-shaped EL layers of the light-emitting and light-receiving apparatus having the MML structure are formed by not patterning using a metal mask but processing after formation of an EL layer. Thus, a light-emitting and light-receiving apparatus with a higher resolution or a higher aperture ratio than a conventional one can be achieved. Moreover, EL layers can be formed separately for each color, which enables extremely clear images; thus, a light-emitting and light-receiving apparatus with a high contrast and high display quality can be achieved. Furthermore, provision of a sacrificial layer over an EL layer can reduce damage on the EL layer during the manufacturing process and increase the reliability of the light-emitting device.

In FIG. 4A and FIG. 8A, the width of the EL layer 103 is substantially equal to that of the electrode 551 in the light-emitting device 550, and the width of the light-receiving layer 103PS is substantially equal to that of the electrode 551PS in the light-receiving device 550PS; however, one embodiment of the present invention is not limited thereto.

In the light-emitting device 550, the width of the EL layer 103 may be smaller than that of the electrode 551. In the light-receiving device 550PS, the width of the light-receiving layer 103PS may be smaller than that of the electrode 551PS. FIG. 8B illustrates an example in which the width of the EL layer 103B is smaller than that of the electrode 551B in the light-emitting device 550B.

In the light-emitting device 550, the width of the EL layer 103 may be larger than that of the electrode 551. In the light-receiving device 550PS, the width of the light-receiving layer 103PS may be larger than that of the electrode 551PS. FIG. 8C illustrates an example in which the width of the EL layer 103B is larger than that of the electrode 551B in the light-emitting device 550B.

The structures described in this embodiment can be used in combination with any of the structures described in the other embodiments as appropriate.

### Embodiment 5

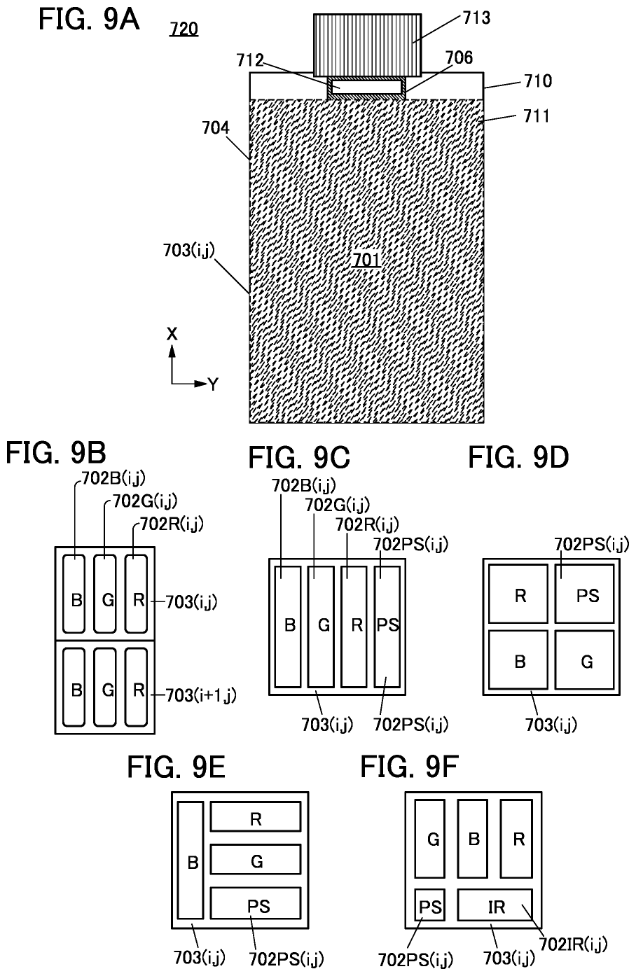

In this embodiment, an apparatus 720 and a light-emitting and light-receiving apparatus 700 are described with reference to FIGS. 9A to 9F, FIGS. 10A to 10C, and FIG. 11. The apparatuses illustrated in FIGS. 9A to 9F, FIGS. 10A to 10C, and FIG. 11 include any of the light-emitting devices described in Embodiments 1 and 2 and thus are light-emitting apparatuses. Furthermore, the apparatus 720 described in this embodiment can be used in a display unit of an electronic appliance or the like and thus can also be referred to as a display panel or a display device. Moreover, when the apparatus 720 includes the light-emitting device as a light source and a light-receiving device that can receive light from the light-emitting device, the apparatus 720 can be referred to as a light-emitting and light-receiving apparatus. Note that the light-emitting apparatus, the display panel, the display device, and the light-emitting and light-receiving apparatus each include at least a light-emitting device.

Furthermore, the light-emitting apparatus, the display panel, the display device, and the light-emitting and light-receiving apparatus of this embodiment can each have high definition or a large size. Thus, the light-emitting apparatus, the display panel, the display device, and the light-emitting and light-receiving apparatus of this embodiment can be used, for example, in display units of electronic appliances such as a digital camera, a digital video camera, a digital photo frame, a mobile phone, a portable game console, a smart phone, a wristwatch terminal, a tablet terminal, a portable information terminal, and an audio reproducing apparatus, in addition to display units of electronic appliances with a relatively large screen, such as a television device, a desktop or laptop personal computer, a monitor of a computer or the like, digital signage, and a large game machine such as a pachinko machine.

FIG. 9A is a top view of the apparatus 720 (e.g., the light-emitting apparatus, the display panel, the display device, and the light-emitting and light-receiving apparatus).

In FIG. 9A, the apparatus 720 has a structure in which a substrate 710 and a substrate 711 are attached to each other. In addition, the apparatus 720 includes a display region 701, a circuit 704, and a wiring 706. Note that the display region 701 includes a plurality of pixels. As illustrated in FIG. 9B, a pixel 703(i, j) illustrated in FIG. 9A is adjacent to a pixel 703(i+1, j).

Furthermore, in the example of the apparatus 720 illustrated in FIG. 9A, the substrate 710 is provided with an integrated circuit (IC) 712 by a chip on glass (COG) method, a chip on film (COF) method, or the like. As the IC 712, an IC including a scan line driver circuit, a signal line driver circuit, or the like can be used, for example. In the example illustrated in FIG. 9A, an IC including a signal line driver circuit is used as the IC 712, and a scan line driver circuit is used as the circuit 704.

The wiring 706 has a function of supplying signals and power to the display region 701 and the circuit 704. The signals and power are input to the wiring 706 from the outside through a flexible printed circuit (FPC) 713 or to the wiring 706 from the IC 712. Note that the apparatus 720 is not necessarily provided with the IC. The IC may be mounted on the FPC by a COF method or the like.

FIG. 9B illustrates the pixel 703(i, j) and the pixel 703(i+1, j) of the display region 701. A plurality of kinds of subpixels including light-emitting devices that emit different color light from each other can be included in the pixel 703(i, j). In addition to the above, a plurality of subpixels including light-emitting devices that emit light of the same color may be included. For example, the pixel can include three kinds of subpixels. The three subpixels can be of three colors of red (R), green (G), and blue (B) or of three colors of yellow (Y), cyan (C), and magenta (M), for example. Alternatively, the pixel can include four kinds of subpixels. The four subpixels can be of four colors of R, G, B, and white (W) or of four colors of R, G, B, and Y, for example. Specifically, the pixel 703(i, j) can consist of a subpixel 702B(i,j) for blue display, a subpixel 702G(i, j) for green display, and a subpixel 702R(i,j) for red display.

The subpixel may include not only a light-emitting device but also a light-receiving device. Note that when the subpixel includes a light-receiving device, the apparatus 720 is also referred to as a light-emitting and light-receiving apparatus.

FIGS. 9C to 9F illustrate various layout examples of the pixel 703(i,j) including a subpixel 702PS(i,j) including alight-receiving device. The pixel arrangement in FIG. 9C is stripe arrangement, and the pixel arrangement in FIG. 9D is matrix arrangement. The pixel arrangement in FIG. 9E has a structure where three subpixels (the subpixels R, G, and PS) are vertically arranged next to one subpixel (the subpixel B). In the pixel arrangement in FIG. 9F, the vertically oriented three subpixels G, B, and R are arranged laterally, and the subpixel PS and the horizontally oriented subpixel IR are arranged laterally below the three subpixels. Note that the wavelength of light detected by the subpixel 702PS(i, j) is not particularly limited; however, the light-receiving device included in the subpixel 702PS(i, j) preferably has sensitivity to light emitted from the light-emitting device included in the subpixel 702R(i, j), the subpixel 702G(i, j), the subpixel 702B(i, j), or the subpixel 702IR(i, j). For example, the light-receiving device preferably detects one or more kinds of light in blue, violet, bluish violet, green, yellowish green, yellow, orange, red, and infrared wavelength ranges, for example.

Furthermore, as illustrated in FIG. 9F, a subpixel 702IR(i, j) that emits infrared rays may be added to any of the above-described sets of subpixels in the pixel 703(i, j). Specifically, the subpixel that emits light including light with a wavelength ranging from 650 nm to 1000 nm, inclusive, may be used in the pixel 703(i,j).

Note that the arrangement of subpixels is not limited to the structures illustrated in FIG. 9 and a variety of arrangement methods can be employed. The arrangement of subpixels may be stripe arrangement, S stripe arrangement, matrix arrangement, delta arrangement, Bayer arrangement, or pentile arrangement, for example.

Furthermore, top surfaces of the subpixels may have a triangular shape, a quadrangular shape (including a rectangular shape and a square shape), a polygonal shape such as a pentagonal shape, a polygonal shape with rounded corners, an elliptical shape, or a circular shape, for example. The top surface shape of a subpixel herein refers to a top surface shape of a light-emitting region of a light-emitting device.

Furthermore, in the case where not only a light-emitting device but also a light-receiving device is included in a pixel, the pixel has a light-receiving function and thus can detect a contact or approach of an object while displaying an image. For example, an image can be displayed by using all the subpixels included in a light-emitting apparatus; or light can be emitted by some of the subpixels as a light source and an image can be displayed by using the remaining subpixels.

Note that the light-receiving area of the subpixel 702PS(i, j) is preferably smaller than the light-emitting areas of the other subpixels. A smaller light-receiving area leads to a narrower image-capturing range, inhibits a blur in a captured image, and improves the definition. Thus, by using the subpixel 702PS(i, j), high-resolution or high-definition image capturing is possible. For example, image capturing for personal authentication with the use of a fingerprint, a palm print, the iris, the shape of a blood vessel (including the shape of a vein and the shape of an artery), a face, or the like is possible by using the subpixel 702PS(i,j).

Moreover, the subpixel 702PS(i, j) can be used in a touch sensor (also referred to as a direct touch sensor), a near touch sensor (also referred to as a hover sensor, a hover touch sensor, a contactless sensor, or a touchless sensor), or the like. For example, the subpixel 702PS(i, j) preferably detects infrared light. Thus, touch sensing is possible even in a dark place.

Here, the touch sensor or the near touch sensor can detect an approach or contact of an object (e.g., a finger, a hand, or a pen). The touch sensor can detect the object when the light-emitting and light-receiving apparatus and the object come in direct contact with each other. Furthermore, the near touch sensor can detect the object even when the object is not in contact with the light-emitting and light-receiving apparatus. For example, the light-emitting and light-receiving apparatus can preferably detect the object when the distance between the light-emitting and light-receiving apparatus and the object is more than or equal to 0.1 mm and less than or equal to 300 mm, preferably more than or equal to 3 mm and less than or equal to 50 mm. With this structure, the light-emitting and light-receiving apparatus can be controlled without the object directly contacting with the light-emitting and light-receiving apparatus. In other words, the light-emitting and light-receiving apparatus can be controlled in a contactless (touchless) manner. With the above-described structure, the light-emitting and light-receiving apparatus can be controlled with a reduced risk of being dirty or damaged, or without direct contact between the object and a dirt (e.g., dust, bacteria, or a virus) attached to the light-emitting and light-receiving apparatus.

For high-resolution image capturing, the subpixel 702PS(i, j) is preferably provided in every pixel included in the light-emitting and light-receiving apparatus. Meanwhile, in the case where the subpixel 702PS(i, j) is used in a touch sensor, a near touch sensor, or the like, high accuracy is not required as compared to the case of capturing an image of a fingerprint or the like; accordingly, the subpixel 702PS(i, j) is provided in some subpixels in the light-emitting and light-receiving apparatus. When the number of subpixels 702PS(i, j) included in the light-emitting and light-receiving apparatus is smaller than the number of subpixels 702R(i, j) or the like, higher detection speed can be achieved.

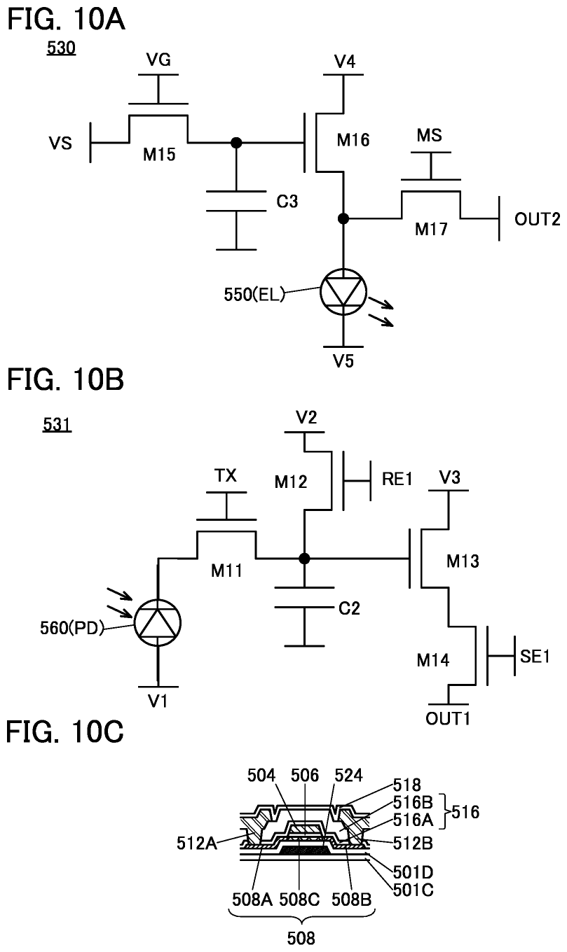

Next, an example of a pixel circuit of a subpixel included in the light-emitting device is described with reference to FIG. 10A. A pixel circuit 530 illustrated in FIG. 10A includes a light-emitting device (EL) 550, a transistor M15, a transistor M16, a transistor M17, and a capacitor C3. Note that a light-emitting diode can be used as the light-emitting device (EL) 550. In particular, the light-emitting device described in Embodiment 1 is preferably used as the light-emitting device (EL) 550.

In FIG. 10A, a gate of the transistor M15 is electrically connected to a wiring VG, one of a source and a drain of the transistor M15 is electrically connected to a wiring VS, and the other of the source and the drain of the transistor M15 is electrically connected to one electrode of the capacitor C3 and a gate of the transistor M16. One of a source and a drain of the transistor M16 is electrically connected to a wiring V4, and the other is electrically connected to an anode of the light-emitting device (EL) 550 and one of a source and a drain of the transistor M17. A gate of the transistor M17 is electrically connected to a wiring MS, and the other of the source and the drain of the transistor M17 is electrically connected to a wiring OUT2. A cathode of the light-emitting device (EL) 550 is electrically connected to a wiring V5.

A constant potential is supplied to the wiring V4 and the wiring V5. In the light-emitting device (EL) 550, the anode side can have a high potential and the cathode side can have a lower potential than the anode side. The transistor M15 is controlled by a signal supplied to the wiring VG and functions as a selection transistor for controlling a selection state of the pixel circuit 530. The transistor M16 functions as a driving transistor that controls a current flowing through the light-emitting device (EL) 550 in accordance with a potential supplied to the gate of the transistor M16. When the transistor M15 is on, a potential supplied to the wiring VS is supplied to the gate of the transistor M16, and the luminance of the light-emitting device (EL) 550 can be controlled in accordance with the potential. The transistor M17 is controlled by a signal supplied to the wiring MS and has a function of outputting a potential between the transistor M16 and the light-emitting device (EL) 550 to the outside through the wiring OUT2.

Here, a transistor in which a metal oxide (an oxide semiconductor) is used in a semiconductor layer where a channel is formed is preferably used as the transistors M11, M12, M13, and M14 included in the pixel circuit 531 in FIG. 10B and the transistors M15, M16, and M17 included in the pixel circuit 530 in FIG. 10A.

A transistor using a metal oxide having a wider band gap and a lower carrier density than silicon can achieve an extremely low off-state current. Such a low off-state current enables retention of charges accumulated in a capacitor that is connected in series to the transistor for a long time. Thus, it is particularly preferable to use a transistor containing an oxide semiconductor as the transistors M11, M12, and M15 each of which is connected in series to a capacitor C2 or the capacitor C3. When each of the other transistors also includes an oxide semiconductor, manufacturing cost can be reduced.

Alternatively, transistors using silicon as a semiconductor in which a channel is formed can be used as the transistors M11 to M17. In particular, it is preferable to use silicon with high crystallinity such as single crystal silicon or polycrystalline silicon because high field-effect mobility can be achieved and higher-speed operation can be performed.

Alternatively, a transistor containing an oxide semiconductor may be used as at least one of the transistors M11 to M17, and transistors containing silicon may be used as the other transistors.

Next, an example of a pixel circuit of a subpixel including a light-receiving device is described with reference to FIG. 10B. The pixel circuit 531 illustrated in FIG. 10B includes a light-receiving device (PD) 560, the transistor M11, the transistor M12, the transistor M13, the transistor M14, and the capacitor C2. For example, a photodiode is used as the light-receiving device (PD) 560.

In FIG. 10B, an anode of the light-receiving device (PD) 560 is electrically connected to a wiring V1, and a cathode of the light-receiving device (PD) 560 is electrically connected to one of a source and a drain of the transistor M11. A gate of the transistor M11 is electrically connected to a wiring TX, and the other of the source and the drain of the transistor M11 is electrically connected to one electrode of the capacitor C2, one of a source and a drain of the transistor M12, and a gate of the transistor M13. A gate of the transistor M12 is electrically connected to a wiring REl, and the other of the source and the drain of the transistor M12 is electrically connected to a wiring V2. One of a source and a drain of the transistor M13 is electrically connected to a wiring V3, and the other of the source and the drain of the transistor M13 is electrically connected to one of a source and a drain of the transistor M14. A gate of the transistor M14 is electrically connected to a wiring SE1, and the other of the source and the drain of the transistor M14 is electrically connected to a wiring OUT1.

A constant potential is supplied to the wiring V1, the wiring V2, and the wiring V3. When the light-receiving device (PD) 560 is driven with a reverse bias, the wiring V2 is supplied with a potential higher than the potential of the wiring V1. The transistor M12 is controlled by a signal supplied to the wiring REl and has a function of resetting the potential of a node connected to the gate of the transistor M13 to a potential supplied to the wiring V2. The transistor M11 is controlled by a signal supplied to the wiring TX and has a function of controlling the timing at which the potential of the node changes, in accordance with a current flowing through the light-receiving device (PD) 560. The transistor M13 functions as an amplifier transistor for outputting a signal corresponding to the potential of the node. The transistor M14 is controlled by a signal supplied to the wiring SE1 and functions as a selection transistor for reading an output corresponding to the potential of the node by an external circuit connected to the wiring OUT1.

Although n-channel transistors are illustrated in FIGS. 10A and 10B, p-channel transistors can be used instead.

The transistors included in the pixel circuit 530 and the transistors included in the pixel circuit 531 are preferably formed side by side over the same substrate. Preferably, the transistors included in the pixel circuit 530 and the transistors included in the pixel circuit 531 are periodically arranged in one region, in particular.

One or more layers including the transistor and/or the capacitor are preferably provided to overlap with the light-receiving device (PD) 560 or the light-emitting device (EL) 550. Thus, the effective area of each pixel circuit can be reduced, and a high-resolution light-receiving unit or display unit can be achieved.

FIG. 10C illustrates an example of a specific structure of a transistor that can be used in the pixel circuit described with reference to FIGS. 10A and 10B. As the transistor, a bottom-gate transistor, a top-gate transistor, or the like can be used as appropriate.

The transistor illustrated in FIG. 10C includes a semiconductor film 508, a conductive film 504, an insulating film 506, a conductive film 512A, and a conductive film 512B. The transistor is formed over an insulating film 501C, for example. The transistor also includes an insulating film 516 (an insulating film 516A and an insulating film 516B) and an insulating film 518.

The semiconductor film 508 includes a region 508A electrically connected to the conductive film 512A and a region 508B electrically connected to the conductive film 512B. The semiconductor film 508 includes a region 508C between the region 508A and the region 508B.

The conductive film 504 includes a region overlapping with the region 508C and has a function of a gate electrode.

The insulating film 506 includes a region positioned between the semiconductor film 508 and the conductive film 504. The insulating film 506 has a function of a first gate insulating film.

The conductive film 512A has one of a function of a source electrode and a function of a drain electrode, and the conductive film 512B has the other thereof.

A conductive film 524 can be used in the transistor. The semiconductor film 508 is sandwiched between the conductive film 504 and a region included in the conductive film 524. The conductive film 524 has a function of a second gate electrode. An insulating film 501D is positioned between the semiconductor film 508 and the conductive film 524 and has a function of a second gate insulating film.

The insulating film 516 functions as, for example, a protective film covering the semiconductor film 508. Specifically, a film including a silicon oxide film, a silicon oxynitride film, a silicon nitride oxide film, a silicon nitride film, an aluminum oxide film, a hafnium oxide film, an yttrium oxide film, a zirconium oxide film, a gallium oxide film, a tantalum oxide film, a magnesium oxide film, a lanthanum oxide film, a cerium oxide film, or a neodymium oxide film can be used as the insulating film 516, for example.

For the insulating film 518, a material that has a function of inhibiting diffusion of oxygen, hydrogen, water, an alkali metal, an alkaline earth metal, and the like is preferably used. Specifically, the insulating film 518 can be formed using silicon nitride, silicon oxynitride, aluminum nitride, or aluminum oxynitride, for example. In each of silicon oxynitride and aluminum oxynitride, the number of nitrogen atoms contained is preferably larger than the number of oxygen atoms contained.

Note that in a step of forming the semiconductor film used in the transistor of the pixel circuit, the semiconductor film used in the transistor of the driver circuit can be formed. A semiconductor film having the same composition as the semiconductor film used in the transistor of the pixel circuit can be used in the driver circuit, for example.

The semiconductor film 508 preferably contains indium, M (M is one or more of gallium, aluminum, silicon, boron, yttrium, tin, copper, vanadium, beryllium, titanium, iron, nickel, germanium, zirconium, molybdenum, lanthanum, cerium, neodymium, hafnium, tantalum, tungsten, and magnesium), and zinc, for example. Specifically, M is preferably one or more of aluminum, gallium, yttrium, and tin.

In particular, an oxide containing In, Ga, and Zn (also referred to as IGZO) is preferably used as the semiconductor film 508. Alternatively, it is preferable to use an oxide containing In, Sn, and Zn. Further alternatively, it is preferable to use an oxide containing In, Ga, Sn, and Zn. Further alternatively, it is preferable to use an oxide containing In, Al, and Zn (also referred to as IAZO). Further alternatively, it is preferable to use an oxide containing In, Al, Ga, and Zn (also referred to as IAGZO).

When the semiconductor film is an In-M-Zn oxide, the atomic ratio of In is preferably greater than or equal to the atomic ratio of Min the In-M-Zn oxide. Examples 10 of the atomic ratio of the metal elements in such an In-M-Zn oxide are In:M:Zn=1:1:1, 1:1:1.2, 1:3:2, 1:3:4, 2:1:3, 3:1:2, 4:2:3, 4:2:4.1, 5:1:3, 5:1:6, 5:1:7, 5:1:8, 6:1:6, and 5:2:5 and a composition in the vicinity of any of the above atomic ratios. Note that the vicinity of the atomic ratio includes ±30% of an intended atomic ratio.

For example, when the atomic ratio is described as In:Ga:Zn=4:2:3 or a composition in the vicinity thereof, the case is included where the atomic proportion of Ga is greater than or equal to 1 and less than or equal to 3 and the atomic proportion of Zn is greater than or equal to 2 and less than or equal to 4 with the atomic proportion of In being 4. In addition, when the atomic ratio is described as In:Ga:Zn=5:1:6 or a composition in the vicinity thereof, the case is included where the atomic proportion of Ga is greater than 0.1 and less than or equal to 2 and the atomic proportion of Zn is greater than or equal to 5 and less than or equal to 7 with the atomic proportion of In being 5. Furthermore, when the atomic ratio is described as In:Ga:Zn=1:1:1 or a composition in the vicinity thereof, the case is included where the atomic proportion of Ga is greater than 0.1 and less than or equal to 2 and the atomic proportion of Zn is greater than 0.1 and less than or equal to 2 with the atomic proportion of In being 1.

There is no particular limitation on the crystallinity of a semiconductor material used in the transistor, and an amorphous semiconductor or a semiconductor having crystallinity (a microcrystalline semiconductor, a polycrystalline semiconductor, a single crystal semiconductor, or a semiconductor partly including crystal regions) can be used. It is preferable to use a semiconductor having crystallinity, in which case deterioration of transistor characteristics can be suppressed.

In the case of using a metal oxide in a semiconductor film 508, the apparatus 720 includes a light-emitting device including a metal oxide in its semiconductor film and having a metal maskless (MML) structure. With this structure, the leakage current that might flow through the transistor and the leakage current that might flow between adjacent light-emitting devices (also referred to as a lateral leakage current, a side leakage current, or the like) can become extremely low. With the structure, a viewer can observe any one or more of the image crispness, the image sharpness, a high chroma, and a high contrast ratio in an image displayed on the display device. When the leakage current that might flow through the transistor and the lateral leakage current that might flow between light-emitting devices are extremely low, display with little leakage of light at the time of black display (what is called black floating) (such display is also referred to as deep black display) can be achieved.

Alternatively, silicon may be used for the semiconductor film 508. Examples of silicon include single crystal silicon, polycrystalline silicon, and amorphous silicon. In particular, a transistor containing low-temperature polysilicon (LTPS) in its semiconductor layer (hereinafter also referred to as an LTPS transistor) is preferably used. The LTPS transistor has high field-effect mobility and excellent frequency characteristics.

With the use of transistors using silicon such as LTPS transistors, a circuit required to be driven at a high frequency (e.g., a source driver circuit) can be formed on the same substrate as the display unit. This allows simplification of an external circuit mounted on the light-emitting apparatus and a reduction in costs of parts and mounting costs.

The structure of the transistors used in the display panel may be selected as appropriate depending on the size of the screen of the display panel. For example, single crystal Si transistors can be used in the display panel with a screen diagonal greater than or equal to 0.1 inches and less than or equal to 3 inches. In addition, LTPS transistors can be used in the display panel with a screen diagonal greater than or equal to 0.1 inches and less than or equal to 30 inches, preferably greater than or equal to 1 inch and less than or equal to 30 inches. In addition, an LTPO structure (where an LTPS transistor and an OS transistor are used in combination) can be used for the display panel with a screen diagonal greater than or equal to 0.1 inches and less than or equal to 50 inches, preferably greater than or equal to 1 inch and less than or equal to 50 inches. In addition, OS transistors (transistors each including a metal oxide in a semiconductor where a channel is formed) can be used in the display panel with a screen diagonal greater than or equal to 0.1 inches and less than or equal to 200 inches, preferably greater than or equal to 50 inches and less than or equal to 100 inches.

With the use of single crystal Si transistors, an increase in screen size is extremely difficult due to the size of a single crystal Si substrate. Furthermore, since a laser crystallization apparatus is used in the manufacturing process, LTPS transistors are unlikely to respond to an increase in screen size (typically to a screen diagonal greater than 30 inches). By contrast, since the manufacturing process does not necessarily require a laser crystallization apparatus or the like or can be performed at a relatively low temperature (typically, lower than or equal to 450° C.), OS transistors can be applied to a display panel with a relatively large area (typically, a screen diagonal greater than or equal to 50 inches and less than or equal to 100 inches). In addition, LTPO can be applied to a display panel with a size (typically, a screen diagonal greater than or equal to 1 inch and less than or equal to 50 inches) midway between the size of a display panel using LTPS transistors and the size of a display panel using OS transistors.

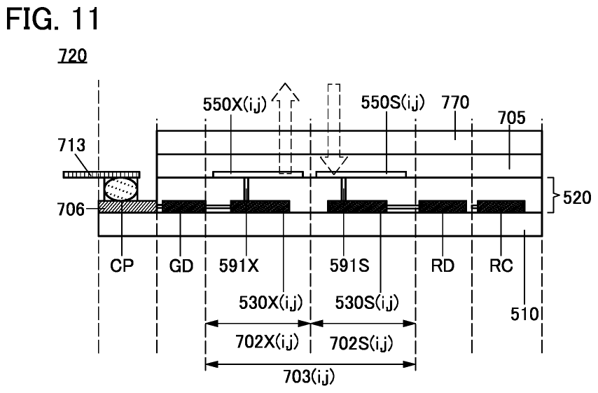

Next, a cross-sectional view of a light-emitting and light-receiving apparatus is shown. FIG. 11 is a cross-sectional view of the apparatus illustrated in FIG. 9A.

FIG. 11 is a cross-sectional view of part of a region including the FPC 713 and the wiring 706 and part of the display region 701 including the pixel 703(i,j).

In FIG. 11, the apparatus 720 includes the functional layer 520 between the first substrate 510 and the second substrate 770. The functional layer 520 includes, as well as the above-described transistors (M11, M12, M13, M14, M15, M16, and M17), the capacitors (C2 and C3), and the like described with reference to FIGS. 10A to 10C, wirings (VS, VG, V1, V2, V3, V4, and V5) electrically connected to these components, for example. FIG. 11 illustrates a non-limiting example of the functional layer 520 that includes a pixel circuit 530X(i,j), a pixel circuit 530S(i,j), and a circuit GD.

Furthermore, each pixel circuit included in the functional layer 520 is electrically connected to a light-emitting device or a light-receiving device. For example, in FIG. 11, the pixel circuit 530X(i, j) and the pixel circuit 530S(i, j) included in the functional layer 520 are electrically connected to a light-emitting device 550X(i,j) and a light-receiving device 550S(i,j), respectively, in FIG. 11 formed over the functional layer 520. Concretely, the light-emitting device 550X(i, j) is electrically connected to the pixel circuit 530X(i,j) through a wiring 591X, and the light-receiving device 550S(i, j) is electrically connected to the pixel circuit 530S(i, j) through a wiring 591S. The insulating layer 705 is provided over the functional layer 520, the light-emitting devices, and the light-receiving device, and has a function of attaching the second substrate 770 and the functional layer 520.

As the second substrate 770, a substrate where touch sensors are arranged in a matrix can be used. For example, a substrate provided with capacitive touch sensors or optical touch sensors can be used as the second substrate 770. Thus, the light-emitting and light-receiving apparatus of one embodiment of the present invention can be used as a touch panel.

The structures described in this embodiment can be used in appropriate combination with any of the structures described in the other embodiments.

### Embodiment 6

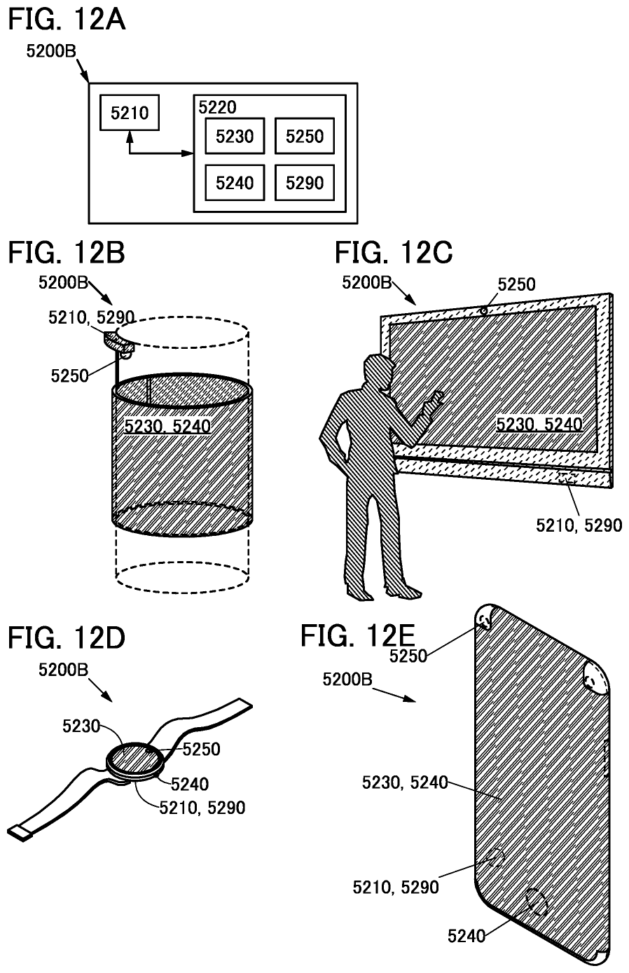

This embodiment will describe structures of electronic appliances of embodiments of the present invention with reference to FIGS. 12A to 12E, FIGS. 13A to 13E, and FIGS. 14A and 14B.

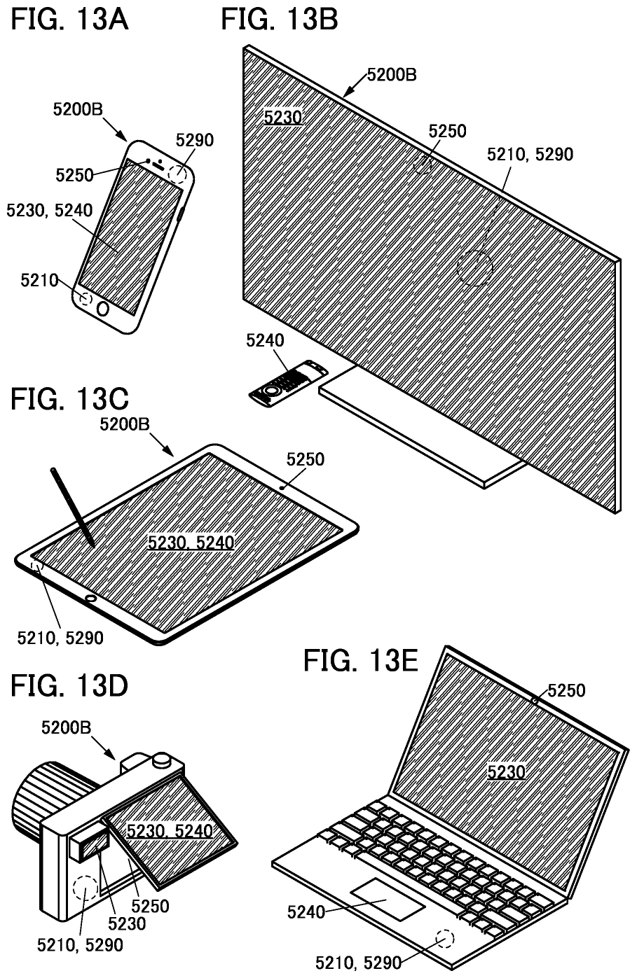

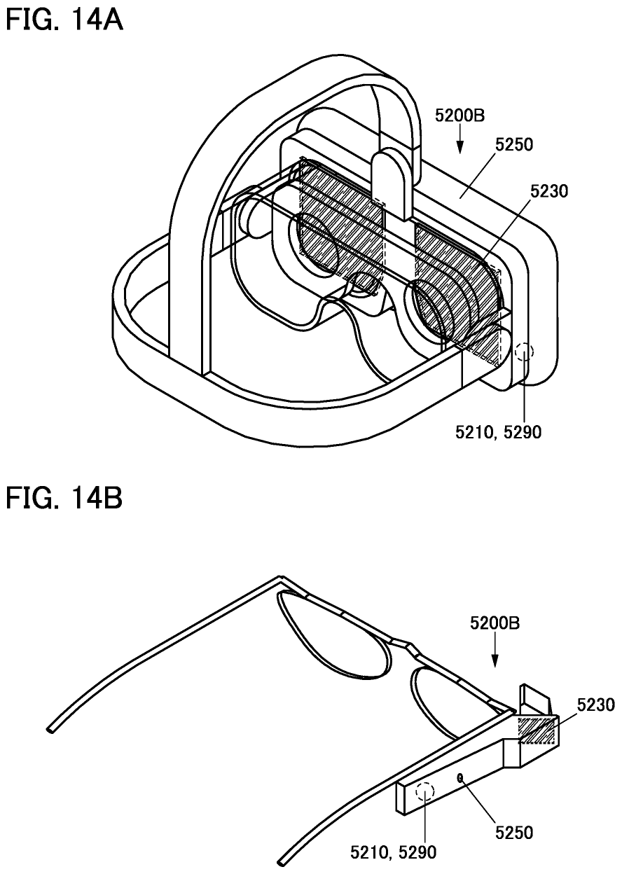

FIGS. 12A to 12E, FIGS. 13A to 13E, and FIGS. 14A and 14B each illustrate a structure of an electronic appliance of one embodiment of the present invention. FIG. 12A is a block diagram of an electronic appliance and FIGS. 12B to 12E are perspective views illustrating structures of electronic appliances. FIGS. 13A to 13E are perspective views illustrating structures of electronic appliances. FIGS. 14A and 14B are perspective views illustrating structures of electronic appliances.

An electronic appliance 5200B described in this embodiment includes an arithmetic device 5210 and an input/output device 5220 (see FIG. 12A).

The arithmetic device 5210 has a function of receiving handling data and a function of supplying image data on the basis of the handling data.

The input/output device 5220 includes a display unit 5230, an input unit 5240, a sensor unit 5250, and a communication unit 5290, and has a function of supplying handling data and a function of receiving image data. The input/output device 5220 also has a function of supplying sensing data, a function of supplying communication data, and a function of receiving communication data.

The input unit 5240 has a function of supplying handling data. For example, the input unit 5240 supplies handling data on the basis of handling by a user of the electronic appliance 5200B.

Specifically, a keyboard, a hardware button, a pointing device, a touch sensor, an illuminance sensor, an imaging device, an audio input device, an eye-gaze input device, an attitude sensing device, or the like can be used as the input unit 5240.

The display unit 5230 includes a display panel and has a function of displaying image data. For example, the display panel described in Embodiment 4 can be used for the display unit 5230.

The sensor unit 5250 has a function of supplying sensing data. For example, the sensor unit 5250 has a function of sensing a surrounding environment where the electronic appliance is used and supplying the sensing data.

Specifically, an illuminance sensor, an imaging device, an attitude sensing device, a pressure sensor, a human motion sensor, or the like can be used as the sensor unit 5250.

The communication unit 5290 has a function of receiving and supplying communication data. For example, the communication unit 5290 has a function of being connected to another electronic appliance or a communication network by wireless communication or wired communication. Specifically, the communication unit 5290 has a function of wireless local area network communication, telephone communication, near field communication, or the like.

FIG. 12B illustrates an electronic appliance having an outer shape along a cylindrical column or the like. An example of such an electronic appliance is digital signage. The display panel of one embodiment of the present invention can be used for the display unit 5230. The electronic appliance may have a function of changing its display method in accordance with the illuminance of a usage environment. The electronic appliance has a function of changing the displayed content when sensing the existence of a person. Thus, for example, the electronic appliance can be provided on a column of a building. The electronic appliance can display advertising, guidance, or the like.

FIG. 12C illustrates an electronic appliance having a function of generating image data on the basis of the path of a pointer used by the user. Examples of such an electronic appliance include an electronic blackboard, an electronic bulletin board, and digital signage. Specifically, a display panel with a diagonal size of 20 inches or longer, preferably 40 inches or longer, further preferably 55 inches or longer can be used. A plurality of display panels can be arranged and used as one display region. Alternatively, a plurality of display panels can be arranged and used as a multiscreen.

FIG. 12D illustrates an electronic appliance that is capable of receiving data from another device and displaying the data on the display unit 5230. An example of such an electronic appliance is a wearable electronic appliance. Specifically, the electronic appliance can display several options, and the user can choose some from the options and send a reply to the data transmitter. As another example, the electronic appliance has a function of changing its display method in accordance with the illuminance of a usage environment. Thus, for example, power consumption of the wearable electronic appliance can be reduced. As another example, the wearable electronic appliance can display an image so as to be suitably used even in an environment under strong external light, e.g., outdoors in fine weather.

FIG. 12E illustrates an electronic appliance including the display unit 5230 having a surface gently curved along a side surface of a housing. An example of such an electronic appliance is a mobile phone. The display unit 5230 includes a display panel that has a function of displaying images on the front surface, the side surfaces, the top surface, and the rear surface, for example. Thus, a mobile phone can display data on not only its front surface but also its side surfaces, top surface, and rear surface, for example.

FIG. 13A illustrates an electronic appliance that is capable of receiving data via the Internet and displaying the data on the display unit 5230. An example of such an electronic appliance is a smartphone. For example, the user can check a created message on the display unit 5230 and send the created message to another device. As another example, the electronic appliance has a function of changing its display method in accordance with the illuminance of a usage environment. Thus, power consumption of the smartphone can be reduced. As another example, it is possible to obtain a smartphone which can display an image such that the smartphone can be suitably used in an environment under strong external light, e.g., outdoors in fine weather.

FIG. 13B illustrates an electronic appliance that can use a remote controller as the input unit 5240. An example of such an electronic appliance is a television system. For example, data received from a broadcast station or via the Internet can be displayed on the display unit 5230. The electronic appliance can take an image of the user with the sensor unit 5250 and transmit the image of the user. The electronic appliance can acquire a viewing history of the user and provide it to a cloud service. The electronic appliance can acquire recommendation data from a cloud service and display the data on the display unit 5230. A program or a moving image can be displayed on the basis of the recommendation data. As another example, the electronic appliance has a function of changing its display method in accordance with the illuminance of a usage environment. Accordingly, for example, it is possible to obtain a television system which can display an image such that the television system can be suitably used even under strong external light entering the room from the outside in fine weather.

FIG. 13C illustrates an electronic appliance that is capable of receiving an educational material via the Internet and displaying it on the display unit 5230. An example of such an electronic appliance is a tablet computer. The user can input an assignment with the input unit 5240 and send it via the Internet. The user can obtain a corrected assignment or the evaluation from a cloud service and have it displayed on the display unit 5230. The user can select a suitable educational material on the basis of the evaluation and have it displayed.

For example, an image signal can be received from another electronic appliance and displayed on the display unit 5230. When the electronic appliance is placed on a stand or the like, the display unit 5230 can be used as a sub-display. Thus, for example, it is possible to obtain a tablet computer which can display an image such that the tablet computer is favorably used even in an environment under strong external light, e.g., outdoors in fine weather.

FIG. 13D illustrates an electronic appliance including a plurality of display units 5230. An example of such an electronic appliance is a digital camera. For example, the display unit 5230 can display an image that the sensor unit 5250 is capturing. A captured image can be displayed on the sensor unit. A captured image can be decorated using the input unit 5240. A message can be attached to a captured image. A captured image can be transmitted via the Internet. The electronic appliance has a function of changing shooting conditions in accordance with the illuminance of a usage environment. Accordingly, for example, it is possible to obtain a digital camera that can display a subject such that an image is favorably viewed even in an environment under strong external light, e.g., outdoors in fine weather.

FIG. 13E illustrates an electronic appliance in which the electronic appliance of this embodiment is used as a master to control another electronic appliance used as a slave. An example of such an electronic appliance is a portable personal computer. For example, part of image data can be displayed on the display unit 5230 and another part of the image data can be displayed on a display unit of another electronic appliance. Image signals can be supplied. Data written from an input unit of another electronic appliance can be obtained with the communication unit 5290. Thus, a large display region can be utilized in the case of using a portable personal computer, for example.

FIG. 14A illustrates an electronic appliance including the sensor unit 5250 that senses an acceleration or a direction. An example of such an electronic appliance is a goggles-type electronic appliance. The sensor unit 5250 can supply data on the position of the user or the direction in which the user faces. The electronic appliance can generate image data for the right eye and image data for the left eye in accordance with the position of the user or the direction in which the user faces. The display unit 5230 includes a display region for the right eye and a display region for the left eye. Thus, a virtual reality image that gives the user a sense of immersion can be displayed on the goggles-type electronic appliance, for example.

FIG. 14B illustrates an electronic appliance including an imaging device and the sensor unit 5250 that senses an acceleration or a direction. An example of such an electronic appliance is a glasses-type electronic appliance. The sensor unit 5250 can supply data on the position of the user or the direction in which the user faces. The electronic appliance can generate image data in accordance with the position of the user or the direction in which the user faces. Accordingly, the data can be shown together with a real-world scene, for example. Alternatively, an augmented reality image can be displayed on the glasses-type electronic appliance.

Note that this embodiment can be combined with any of the other embodiments in this specification as appropriate.

### Embodiment 7

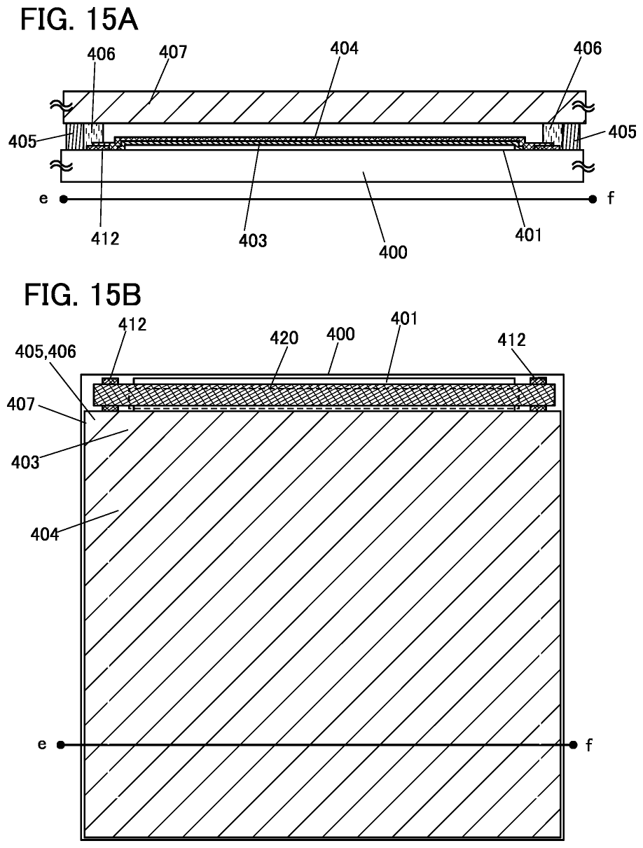

In this embodiment, a structure in which the light-emitting device described in Embodiment 1 is used in a lighting device will be described with reference to FIGS. 15A and 15B. FIG. 15A shows a cross section taken along the line e-f in a top view of the lighting device in FIG. 15B.

In the lighting device in this embodiment, a first electrode 401 is formed over a substrate 400 that is a support and has a light-transmitting property. The first electrode 401 corresponds to the first electrode 101 in Embodiment 1. When light is extracted from the first electrode 401 side, the first electrode 401 is formed using a material having a light-transmitting property.

A pad 412 for applying voltage to a second electrode 404 is provided over the substrate 400.

An EL layer 403 is formed over the first electrode 401. The structure of the EL layer 403 corresponds to, for example, the structure of the EL layer 103 in Embodiment 3. Refer to the corresponding description for these structures.

The second electrode 404 is formed to cover the EL layer 403. The second electrode 404 corresponds to the second electrode 102 in Embodiment 1. The second electrode 404 is formed using a material having high reflectance when light is extracted from the first electrode 401 side. The second electrode 404 is connected to the pad 412 so that voltage is applied to the second electrode 404.

As described above, the lighting device described in this embodiment includes a light-emitting device including the first electrode 401, the EL layer 403, and the second electrode 404. Since the light-emitting device has high emission efficiency, the lighting device in this embodiment can have low power consumption.

The substrate 400 provided with the light-emitting device having the above structure and a sealing substrate 407 are fixed and sealed with sealing materials (405 and 406), whereby the lighting device is completed. It is possible to use only either the sealing material 405 or the sealing material 406. In addition, the inner sealing material 406 (not illustrated in FIG. 15B) can be mixed with a desiccant that enables moisture to be adsorbed, increasing the reliability.

When parts of the pad 412 and the first electrode 401 are extended to the outside of the sealing materials 405 and 406, the extended parts can serve as external input terminals. An IC chip 420 mounted with a converter or the like may be provided over the external input terminals.

### Embodiment 8

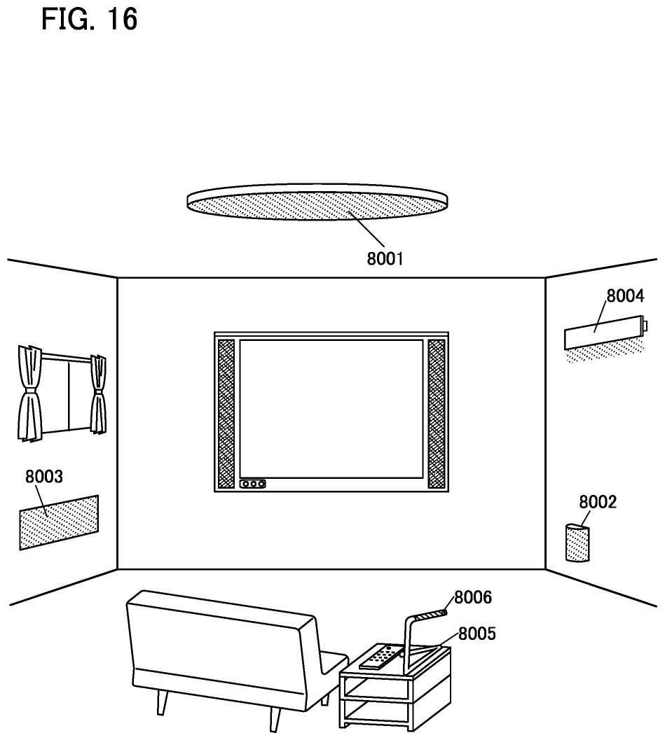

This embodiment will describe application examples of lighting devices manufactured using the light-emitting apparatus of one embodiment of the present invention or the light-emitting device, which is part of the light-emitting apparatus with reference to FIG. 16.

A ceiling light 8001 can be used as an indoor lighting device. Examples of the ceiling light 8001 include a direct-mount light and an embedded light. Such lighting devices are manufactured using the light-emitting apparatus and a housing and a cover in combination. Application to a cord pendant light (light that is suspended from a ceiling by a cord) is also possible.

A foot light 8002 lights a floor so that safety on the floor can be improved. For example, it can be effectively used in a bedroom, on a staircase, and on a passage. In such cases, the size and shape of the foot light can be changed in accordance with the dimensions and structure of a room. The foot light can be a stationary lighting device using the light-emitting apparatus and a support in combination.

A sheet-like lighting 8003 is a thin sheet-like lighting device. The sheet-like lighting, which is attached to a wall when used, is space-saving and thus can be used for a wide variety of uses. Furthermore, the area of the sheet-like lighting can be easily increased. The sheet-like lighting can also be used on an object such as a wall or a housing that has a curved surface.

A lighting device 8004 in which the direction of light from a light source is controlled to be only a desired direction can be used.

A desk lamp 8005 includes a light source 8006. As the light source 8006, the light-emitting apparatus of one embodiment of the present invention or the light-emitting device, which is part of the light-emitting apparatus, can be used.

Besides the above examples, when the light-emitting apparatus of one embodiment of the present invention or the light-emitting device, which is part of the light-emitting apparatus, is used as part of furniture in a room, a lighting device that functions as the furniture can be obtained.

As described above, a variety of lighting devices that include the light-emitting apparatus can be obtained. Note that these lighting devices are also embodiments of the present invention.

The structures described in this embodiment can be used in appropriate combination with any of the structures described in the other embodiments.

### Example 1

### Synthesis Example 1

In this example, a synthesis method of N-[4-(2,2′-binaphthyl-6-yl)phenyl]-N-(biphenyl-4-yl)benzo[b]naphtho[1,2-d]furan-6-amine (abbreviation: (βN2)PBiBnf(6)) (Structural Formula (103)), which is an organic compound of one embodiment of the present invention, is described. The structural formula of (βN2)PBiBnf(6) is shown below.

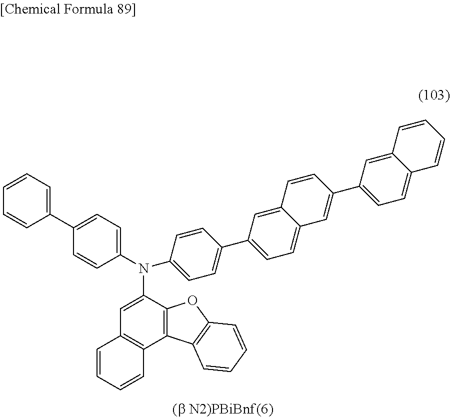 [SMILES: c1ccc(-c2ccc(N(c3ccc(-c4ccc5cc(-c6ccc7ccccc7c6)ccc5c4)cc3)c3cc4ccccc4c4c3oc3ccccc34)cc2)cc1]

### Step 1: Synthesis of N-[4-(2,2′-binaphthyl-6-yl)phenyl]biphenyl-4-amine

Into a 200-mL three-neck flask were put 4.3 g (13 mmol) of N-(4-bromophenyl)-4-biphenylamine, 5.0 g (13 mmol) of 2-(2,2′-binaphthyl-6-yl)-4,4,5,5-tetramethyl-1,3,2-dioxaborolane, 5.58 g (40 mmol) of potassium carbonate (abbreviation: K₂CO₃), 0.10 g (0.26 mmol) of tri(o-tolyl)phosphine (commonly known name: P(o-tolyl)₃), 20 mL of water, 13 mL of ethanol, and 67 mL of toluene. This mixture was degassed by being stirred under reduced pressure. To this mixture was added 46 mg (204 μmol) of palladium(II) acetate (abbreviation: Pd(OAc)₂), and stirring was performed at 90° C. for two hours. The precipitated solid was collected by suction filtration and washed repeatedly with toluene, ethanol, and water. This mixture was dispersed in hot toluene, and suction filtration was carried out to give 5.74 g of a shiny light gray solid containing the target substance in a yield of 87%. Synthesis Scheme (s-1) of Step 1 is shown below.

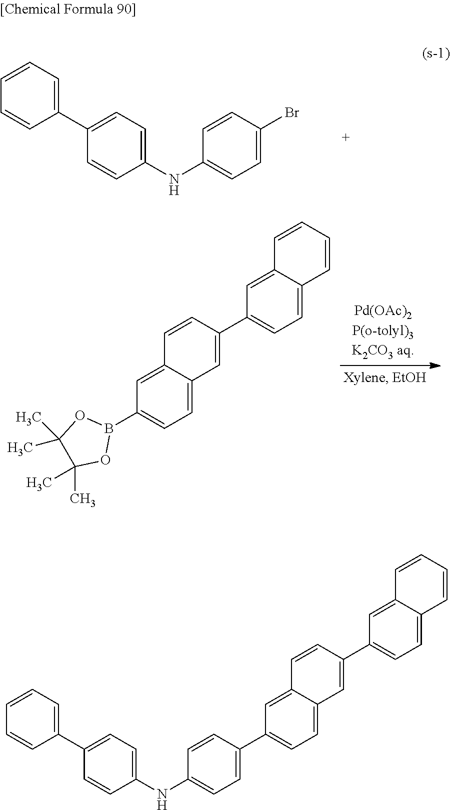 [SMILES: Brc1ccc(Nc2ccc(-c3ccccc3)cc2)cc1 ; CC1(C)OB(c2ccc3cc(-c4ccc5ccccc5c4)ccc3c2)OC1(C)C ; c1ccc(-c2ccc(Nc3ccc(-c4ccc5cc(-c6ccc7ccccc7c6)ccc5c4)cc3)cc2)cc1]

### Step 2: Synthesis of (βN2)PBiBnf(6)

Into a 200-mL three-neck flask were put 2.2 g (4.4 mmol) of N-[4-(2,2′-binaphthyl-6-yl)phenyl]biphenyl-4-amine obtained in Step 1 and 1.5 g (4.4 mmol) of 6-iodobenzo[b]naphtho[1,2-d]furan. After the air in the flask was replaced with nitrogen, 1.3 g (13 mmol) of sodium tert-butoxide (abbreviation: tBuONa) and 42 mL of xylene were added thereto. This mixture was degassed by being stirred under reduced pressure. To this mixture were added 0.25 mL (88 μmol) of tri-tert-butylphosphine (abbreviation: P(tBu)₃) (10 wt % hexane solution) and 41 mg (17 μmol) of bis(dibenzylideneacetone)palladium(0), and stirring was performed at 150° C. for four hours. After that, 0.16 g (0.47 mmol) of 6-iodobenzo[b]naphtho[1,2-d]furan and 9.8 mg (44 μmol) of Pd(dba)₂ were added, and stirring was performed at 150° C. for two hours. After the stirring, toluene was added to the obtained mixture, and stirring was performed under heating at 100° C. This mixture was suction-filtered at 100° C. through alumina, Celite (Catalog No. 537-02305 produced by FUJIFILM Wako Pure Chemical Corporation), and Florisil (Catalog No. 066-05265 produced by FUJIFILM Wako Pure Chemical Corporation). A solid obtained by concentration of the resulting filtrate was recrystallized with toluene to give 1.2 g of the target white solid in a yield of 39%.

The obtained solid was purified by a train sublimation method. In the purification by sublimation, the solid was heated at 385° C. for 90 hours under a pressure of 3.34 Pa with an argon flow rate of 10 mL/min. After the purification by sublimation, 0.9 g of the target white solid was obtained at a collection rate of 75%. Synthesis Scheme (s-2) of Step 2 is shown below.

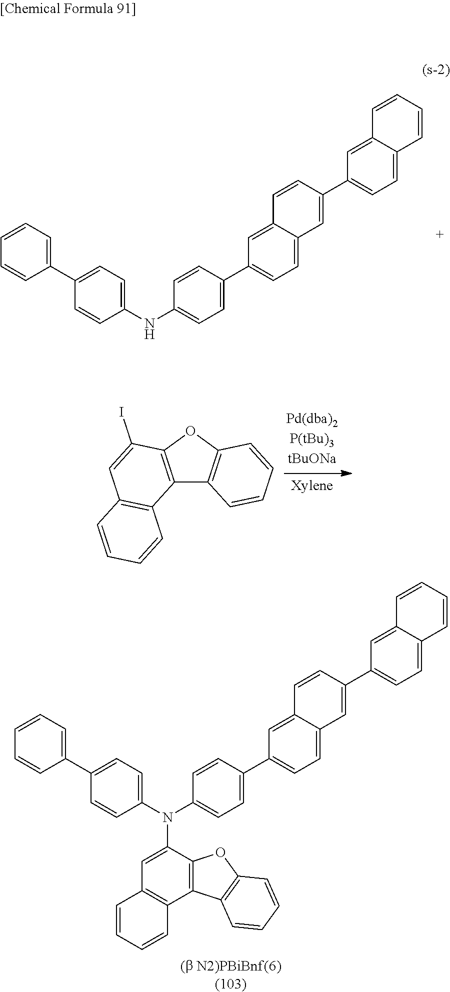 [SMILES: c1ccc(-c2ccc(Nc3ccc(-c4ccc5cc(-c6ccc7ccccc7c6)ccc5c4)cc3)cc2)cc1 ; Ic1cc2ccccc2c2c1oc1ccccc12 ; c1ccc(-c2ccc(N(c3ccc(-c4ccc5cc(-c6ccc7ccccc7c6)ccc5c4)cc3)c3cc4ccccc4c4c3oc3ccccc34)cc2)cc1]

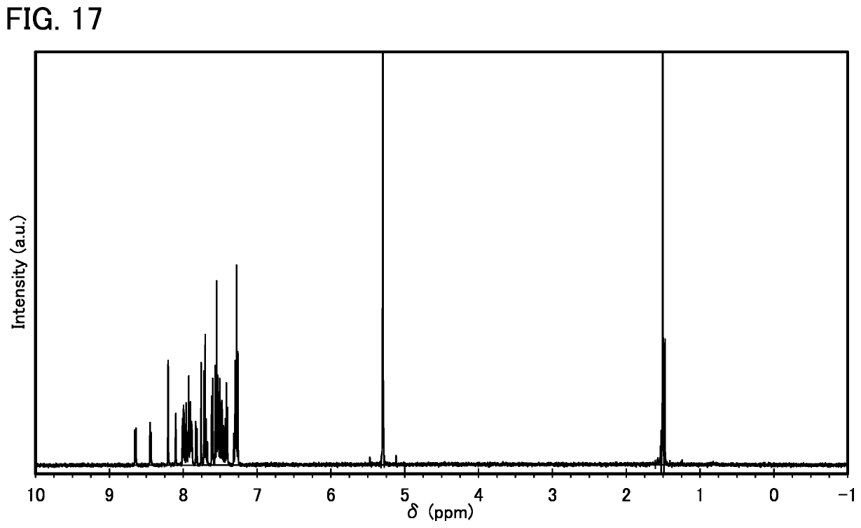

The results of ¹H NMR measurement of the obtained substance is given below. FIG. 17 shows a ¹H NMR spectrum. This shows that (βN2)PBiBnf(6) was obtained in this synthesis example.

¹H NMR (dichloromethane-d₂, 500 MHz): δ=8.65 (d, J=8.5 Hz, 1H), 8.44 (dd, J=7.5 Hz, 2.5 Hz, 1H), 8.20 (s, 2H), 8.10 (s, 1H), 8.01-7.88 (m, 8H), 7.82 (dd, J=8.7 Hz, 2.0 Hz, 1H), 7.76 (s, 1H), 7.72-7.67 (m, 3H), 7.62-7.40 (m, 12H), 7.32-7.26 (m, 5H)

### <Measurement of Physical Properties>

Next, the ultraviolet-visible absorption spectra (hereinafter, simply referred to as absorption spectra) and emission spectra (photoluminescence (PL) spectra, hereinafter referred to as PL spectra) of a toluene solution and a solid thin film of (βN2)PBiBnf(6) were measured.

The absorption spectrum of the solution was measured with an ultraviolet-visible spectrophotometer (V-770DS, manufactured by JASCO Corporation), and the absorption spectrum of the thin film was measured with an ultraviolet-visible spectrophotometer (U-4100, manufactured by Hitachi, Ltd.). To calculate the absorption spectrum of (βN2)PBiBnf(6) in a toluene solution, the absorption spectrum of toluene put in a quartz cell was measured and then subtracted from the absorption spectrum of the toluene solution of (βN2)PBiBnf(6) put in a quartz cell. The emission spectrum was measured with a fluorescence spectrophotometer (FP-8600DS, manufactured by JASCO Corporation).

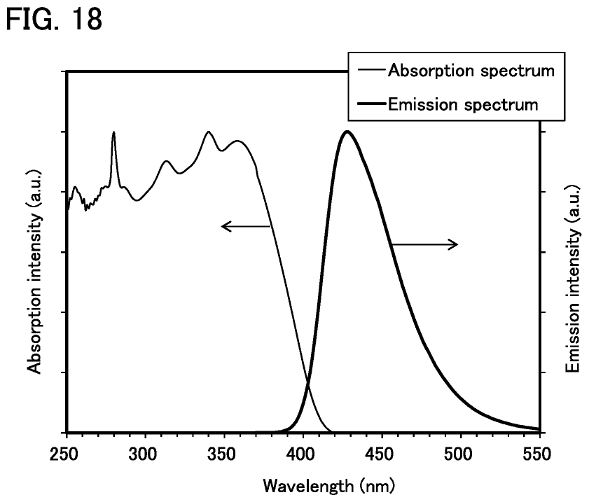

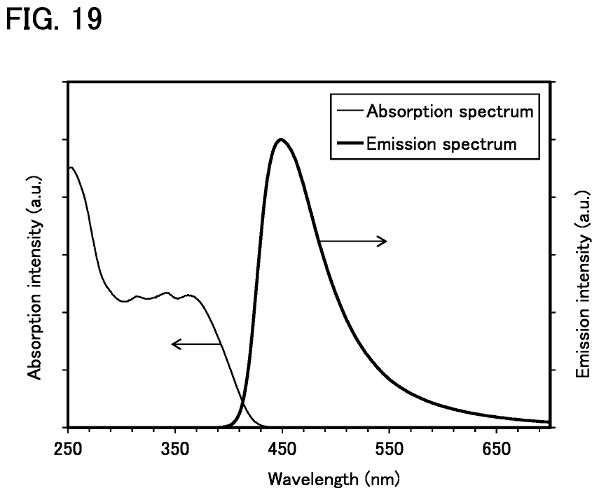

FIG. 18 shows the measurement results of the absorption spectrum and the emission spectrum of the toluene solution of (βN2)PBiBnf(6), and FIG. 19 shows the measurement results of the absorption spectrum and the emission spectrum of the solid thin film of (βN2)PBiBnf(6). As shown in the measurement results in FIG. 18, the toluene solution of (βN2)PBiBnf(6) had the maximum absorption peak at around 363 nm, and it was found that (βN2)PBiBnf(6) is a material that does not absorb light with a wavelength longer than 430 nm. This indicates that (βN2)PBiBnf(6) can be suitably used for a transport layer of a light-emitting device. Furthermore, the toluene solution of (βN2)PBiBnf(6) had an emission peak at around 428 nm (excitation wavelength: 358 nm). As shown in the measurement results in FIG. 19, the thin film of (βN2)PBiBnf(6) had the maximum absorption peak at around 362 nm, and it was found that (βN2)PBiBnf(6) is a material that does not absorb light with a wavelength longer than 430 nm. This indicates that (βN2)PBiBnf(6) can be suitably used for a transport layer of a light-emitting device. Furthermore, the thin film of (βN2)PBiBnf(6) had an emission peak at around 449 nm (excitation wavelength: 370 nm).

The thermogravimetry-differential thermal analysis (TG-DTA) of (βN2)PBiBnf(6) was performed. The measurement was conducted using a high vacuum differential type differential thermal balance (TG-DTA 2410SA, manufactured by Bruker AXS K.K.). The measurement was performed under an atmospheric pressure at a temperature rising rate of 10° C./min under a nitrogen stream (flow rate: 200 mL/min).

In the TG-DTA of (βN2)PBiBnf(6), the temperature (sublimation or decomposition temperature) at which the weight obtained by thermogravimetry was reduced by 5% of the weight at the beginning of the measurement was 495° C., indicating that (βN2)PBiBnf(6) has high heat resistance.

Differential scanning calorimetry (DSC) measurement of (βN2)PBiBnf(6) was performed with DSC8500 manufactured by PerkinElmer, Inc. The DSC measurement was performed in the following manner: the temperature was raised from −10° C. to 350° C. at a temperature rising rate of 40° C./min and held for two minutes, and then the temperature was decreased to −10° C. at a temperature decreasing rate of 100° C./min. This operation was performed three times in succession.

The DSC measurement results of the first cycle show that the glass transition temperature of (βN2)PBiBnf(6) is 137° C., indicating that using (βN2)PBiBnf(6) enables providing an organic semiconductor element having high heat resistance.

### Example 2

### Synthesis Example 2

In this synthesis example, a synthesis method of N-[4-(2,2′-binaphthyl-6-yl)phenyl]-N-(biphenyl-4-yl)benzo[b]naphtho[1,2-d]furan-8-amine (abbreviation: (βN2)PBiBnf(8)) (Structural Formula (108)), which is an organic compound of one embodiment of the present invention, is described. The structural formula of (βN2)PBiBnf(8) is shown below.

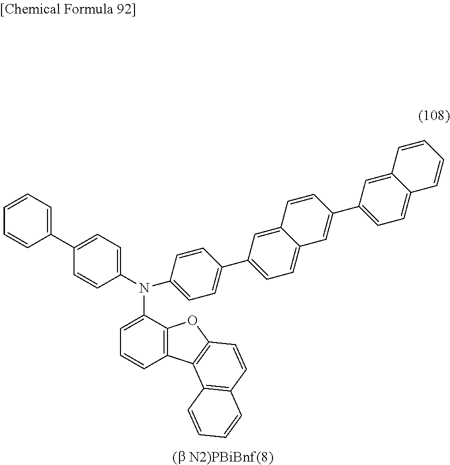 [SMILES: c1ccc(-c2ccc(N(c3ccc(-c4ccc5cc(-c6ccc7ccccc7c6)ccc5c4)cc3)c3cccc4c3oc3ccc5ccccc5c34)cc2)cc1]

### Step 1: Synthesis of (βN2)PBiBnf(8)

Into a 200-mL three-neck flask were put 3.0 g (6.0 mmol) of N-[4-(2,2′-binaphthyl-6-yl)phenyl]biphenyl-4-amine synthesized in Step 1 in Synthesis Example 1, 1.5 g (6.0 mmol) of 8-chlorobenzo[b]naphtho[1,2-d]furan, and 57 mg (0.14 mmol) of 2-dicyclohexylphosphino-2′,6′-dimethoxybiphenyl (commonly known name: SPhos). After the air in the flask was replaced with nitrogen, 1.8 g (18 mmol) of sodium tert-butoxide (abbreviation: tBuONa) and 62 mL of xylene were added thereto. This mixture was degassed by being stirred under reduced pressure. To this mixture was added 33 mg (60 μmol) of bis(dibenzylideneacetone)palladium(0) (commonly known name: Pd(dba)₂), and stirring was performed at 120° C. for seven hours. After the stirring, ethanol was added to the obtained mixture, and the precipitated solid was collected by suction filtration to give 4.4 g of a gray solid containing the target substance.

The obtained solid was purified by a train sublimation method. In the purification by sublimation, the solid was heated at 340° C. for 18 hours under a pressure of 1.4 Pa with an argon flow rate of 10 mL/min. After the purification by sublimation, 1.4 g of the target pale yellow solid was obtained at a collection rate of 32%. Synthesis Scheme (s-3) of Step 1 is shown below.

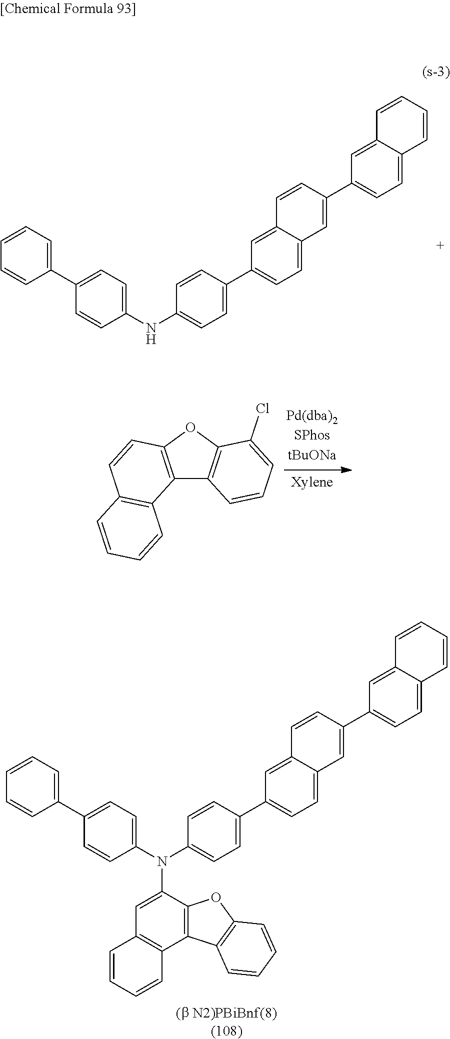 [SMILES: c1ccc(-c2ccc(Nc3ccc(-c4ccc5cc(-c6ccc7ccccc7c6)ccc5c4)cc3)cc2)cc1 ; Clc1cccc2c1oc1ccc3ccccc3c12 ; c1ccc(-c2ccc(N(c3ccc(-c4ccc5cc(-c6ccc7ccccc7c6)ccc5c4)cc3)c3cc4ccccc4c4c3oc3ccccc34)cc2)cc1]

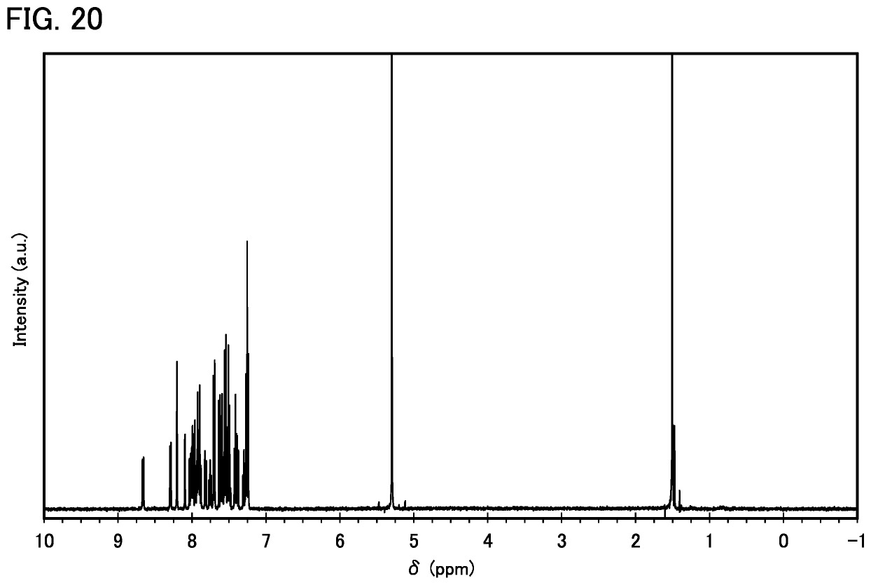

The results of ¹H NMR measurement of the obtained substance is given below. FIG. 20 shows a ¹H NMR spectrum. This shows that (βN2)PBiBnf(8) was obtained in this synthesis example.

¹H NMR (dichloromethane-d₂, 500 MHz): δ=8.66 (d, J=8.0 Hz, 1H), 8.29 (dd, J=7.5 Hz, 1.5 Hz, 1H), 8.21 (s, 2H), 8.10 (s, 1H), 8.04-7.88 (m, 9H), 7.82 (dd, J=8.5 Hz, 2.0 Hz, 1H), 7.76 (dt, J=7.3 Hz, 1.5 Hz, 1H), 7.71-7.69 (m, 2H), 7.63 (d, J=9.0 Hz, 1H), 7.62-7.48 (m, 8H), 7.43-7.37 (m, 3H), 7.31-7.23 (m, 5H)

### <Measurement of Physical Properties>

Next, absorption spectra and emission spectra of a toluene solution and a solid thin film of (βN2)PBiBnf(8) were measured by a method similar to that in Example 1.

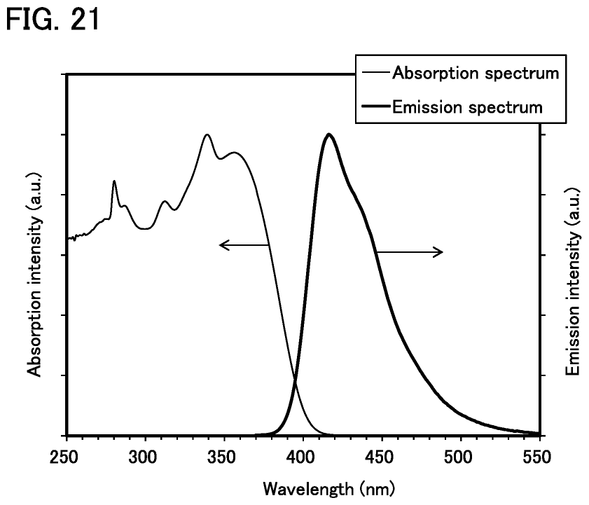

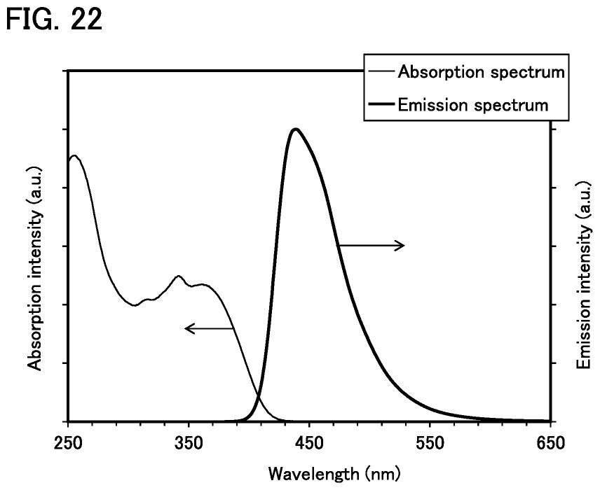

FIG. 21 shows the measurement results of the absorption spectrum and the emission spectrum of the toluene solution of (βN2)PBiBnf(8), and FIG. 22 shows the measurement results of the absorption spectrum and the emission spectrum of the solid thin film of (βN2)PBiBnf(8). As shown in the measurement results in FIG. 21, the toluene solution of (βN2)PBiBnf(8) had the maximum absorption peak at around 359 nm, and it was found that (βN2)PBiBnf(8) is a material that does not absorb light with a wavelength longer than 430 nm. This indicates that (βN2)PBiBnf(8) can be suitably used for a transport layer of a light-emitting device. Furthermore, the toluene solution of (βN2)PBiBnf(8) had an emission spectrum peak at around 416 nm (excitation wavelength: 356 nm). As shown in the measurement results in FIG. 22, the thin film of (βN2)PBiBnf(8) had the maximum absorption peak at around 361 nm, and it was found that (βN2)PBiBnf(8) is a material that does not absorb light with a wavelength longer than 430 nm. This indicates that (βN2)PBiBnf(8) can be suitably used for a transport layer of a light-emitting device. Furthermore, the thin film of (βN2)PBiBnf(8) had an emission peak at around 439 nm (excitation wavelength: 370 nm).

In addition, the TG-DTA of (βN2)PBiBnf(8) was performed by the method similar to that in Example 1. As a result, the temperature (sublimation or decomposition temperature) at which the weight obtained by thermogravimetry was reduced by 5% of the weight at the beginning of the measurement was greater than or equal to 500° C., indicating that (βN2)PBiBnf(8) has high heat resistance.

In addition, the DSC measurement of (βN2)PBiBnf(8) was performed by the method similar to that in Example 1. The DSC measurement results of the second cycle show that the glass transition temperature of (βN2)PBiBnf(8) is 134° C., indicating that using (βN2)PBiBnf(8) enables providing an organic semiconductor element having high heat resistance.

Note that (βN2)PBiBnf(6) whose synthesis method is described in Example 1 and (βN2)PBiBnf(8) whose synthesis method is described in this synthesis example are different from each other in the position to which the nitrogen (N^{A}) at the center of the triarylamine is bonded; N^{A} is bonded to the 6-position of the benzo[b]naphtho[1,2-d]furan ring in (βN2)PBiBnf(6) and N^{A} is bonded to the 8-position of the benzo[b]naphtho[1,2-d]furan ring in (βN2)PBiBnf(8). However, these compounds are represented by the same compositional formula and have the same molecular weight. From the fact that the T_{g} of (βN2)PBiBnf(6) was 137° C., and the T_{g} of (βN2)PBiBnf(8) was 134° C., it was found that (βN2)PBiBnf(6) has higher heat resistance than (βN2)PBiBnf(8) because of the nitrogen (N^{A}) bonded to the 6-position of the benzo[b]naphtho[1,2-d]furan ring in (βN2)PBiBnf(6) although (βN2)PBiBnf(6) and (βN2)PBiBnf(8) have the same molecular weight.

### Example 3

### Synthesis Example 3

In this example, a synthesis method of N-[4-(2,2′-binaphthyl-6-yl)phenyl]-N-(biphenyl-2-yl)benzo[b]naphtho[1,2-d]furan-6-amine (abbreviation: (βN2)PoBiBnf(6)) (Structural Formula (118)), which is an organic compound of one embodiment of the present invention, is described. The structural formula of (βN2)PoBiBnf(6) is shown below.

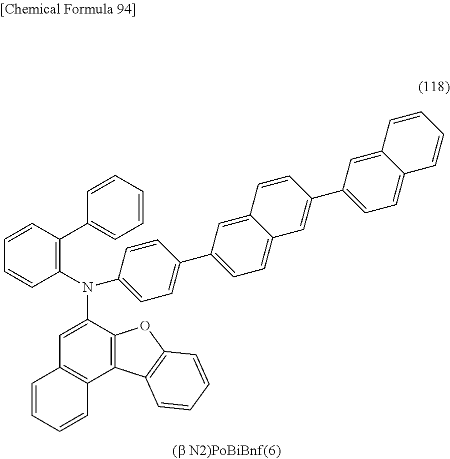 [SMILES: c1ccc(-c2ccccc2N(c2ccc(-c3ccc4cc(-c5ccc6ccccc6c5)ccc4c3)cc2)c2cc3ccccc3c3c2oc2ccccc23)cc1]

### Step 1: Synthesis of N-(biphenyl-2-yl)-1-chlorophenyl-4-amine

Into a 1-L three-neck flask were put 18 g (95 mmol) of 1-bromo-4-chlorobenzene and 16 g (95 mmol) of ortho-biphenylamine. After the air in the flask was replaced with nitrogen, 18 g (0.19 mol) of sodium tert-butoxide and 0.47 L of toluene were added thereto. This mixture was degassed by being stirred under reduced pressure. To this mixture were added 5.0 mL (1.9 mmol) of tri-tert-butylphosphine (commonly known name: P(tBu)₃) (10 wt % hexane solution) and 0.28 g (0.95 mmol) of bis(dibenzylideneacetone)palladium(0) (commonly known name: Pd(dba)₂), and stirring was performed at 120° C. for five hours. After that, 12 g (49 mmol) of 1-chloro-4-iodobenzene, 2.5 mL (0.97 mmol) of P(tBu)₃, and 0.27 g (0.47 mmol) of Pd(dba)₂ were added, and stirring was performed at 120° C. for six hours. After that, 4.8 mL (1.9 mmol) of P(tBu)₃ and 0.25 g (0.44 mmol) of Pd(dba)₂ were added, and stirring was performed at 120° C. for three hours. After that, 4.0 mL (1.6 mmol) of P(tBu)₃ and 0.26 g (0.46 mmol) of Pd(dba)₂ were added, and stirring was performed at 120° C. for five hours. After the stirring, water was added to the obtained mixture, separation was performed, and then the obtained organic layer was dried with magnesium sulfate. Suction filtration was performed, and the resulting filtrate was concentrated to give an reddish brown oily substance. The obtained oily substance was purified by silica gel column chromatography (developing solvent: a mixed solvent of hexane and ethyl acetate in a ratio of 10:1), whereby 16 g of the target pale yellow oily substance was obtained in a yield of 60%. Synthesis Scheme (s-4) of Step 1 is shown below.

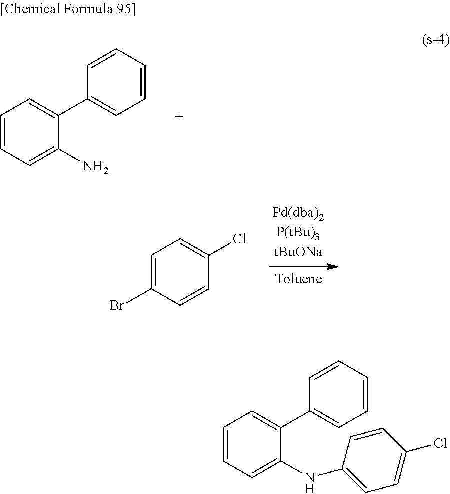 [SMILES: Nc1ccccc1-c1ccccc1 ; Clc1ccc(Br)cc1 ; Clc1ccc(Nc2ccccc2-c2ccccc2)cc1]

### Step 2: Synthesis of N-[4-(2,2′-binaphthyl-6-yl)phenyl]-N-(biphenyl-2-yl)amine

Into a 300-mL three-neck flask were put 5.8 g (21 mmol) of N-(biphenyl-2-yl)-1-chlorophenyl-4-amine obtained in Step 1, 7.9 g (21 mmol) of 2-(2,2′-binaphthyl-6-yl)-4,4,5,5-tetramethyl-1,3,2-dioxaborolane, 8.6 g (62 mmol) of potassium carbonate, 0.15 g (0.41 mmol) of di-1-adamantyl-n-butylphosphine (commonly known name: cataCXium®A), 31 mL of water, 32 mL of ethanol, and 100 mL of toluene. This mixture was degassed by being stirred under reduced pressure, and then the air in the flask was replaced with nitrogen. To this mixture were added 75 mg (0.33 mmol) of palladium(II) acetate (abbreviation: Pd(OAc)₂), and stirring was performed at 90° C. for three hours. To this mixture was added 4.2 g (30 mmol) of potassium carbonate, 0.12 g (0.34 mmol) of cataCXium®A, and 40 mg (0.17 mmol) of Pd(OAc)₂, and stirring was performed at 90° C. for three hours. To this mixture were added 3.2 g (21 mmol) of cesium fluoride, 0.11 g (0.29 mmol) of cataCXium®A, and 54 mg (0.24 mmol) of Pd(OAc)₂, and stirring was performed at 90° C. for seven hours. After the stirring, water was added to the reaction mixture, and the obtained aqueous layer was subjected to extraction with toluene. The obtained organic layer was washed twice with water, washed with saturated brine, and then dried with anhydrous magnesium sulfate. The obtained mixture was suction-filtered through alumina, Celite (Catalog No. 537-02305 produced by FUJIFILM Wako Pure Chemical Corporation), and Florisil (Catalog No. 066-05265 produced by FUJIFILM Wako Pure Chemical Corporation). The resulting filtrate was concentrated to give a solid. The solid was dissolved in a small amount of toluene, and ethanol was added. The precipitated solid was collected by suction filtration to give 2.9 g of the target gray solid in a yield of 29%. Synthesis Scheme (s-5) of Step 2 is shown below.

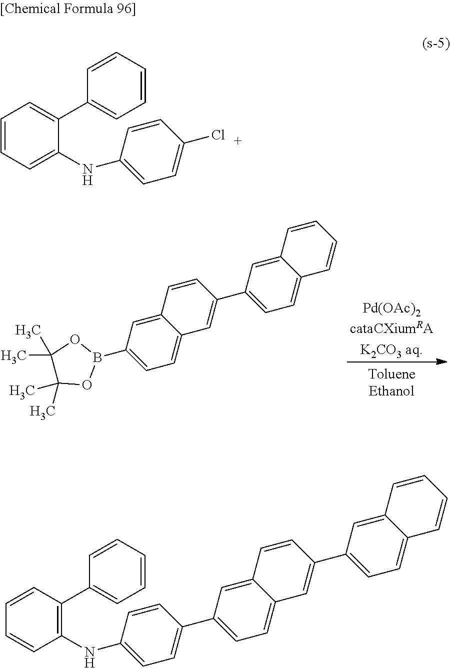 [SMILES: Clc1ccc(Nc2ccccc2-c2ccccc2)cc1 ; CC1(C)OB(c2ccc3cc(-c4ccc5ccccc5c4)ccc3c2)OC1(C)C ; c1ccc(-c2ccccc2Nc2ccc(-c3ccc4cc(-c5ccc6ccccc6c5)ccc4c3)cc2)cc1]

### Step 3: Synthesis of (βN2)PoBiBnf(6)

Into a 200-mL three-neck flask were put 2.0 g (4.0 mmol) of N-[4-(2,2′-binaphthyl-6-yl)phenyl]-N-(biphenyl-2-yl)amine obtained in Step 2 and 1.4 g (4.0 mmol) of 6-iodobenzo[b]naphtho[1,2-d]furan. After the air in the flask was replaced with nitrogen, 1.2 g (12 mmol) of sodium tert-butoxide (abbreviation: tBuONa) and 40 mL of toluene were added thereto. This mixture was degassed by being stirred under reduced pressure. To this mixture were added 0.21 mL (80 μmol) of tri-tert-butylphosphine (abbreviation: P(tBu)₃) (10 wt % hexane solution) and 30 mg (51 μmol) of bis(dibenzylideneacetone)palladium(0) (abbreviation: Pd(dba)₂), and stirring was performed at 120° C. for two hours. After the stirring, toluene was added to the obtained mixture, and stirring was performed under heating at 100° C. This mixture was suction-filtered at 100° C. through alumina, Celite (Catalog No. 537-02305 produced by FUJIFILM Wako Pure Chemical Corporation), and Florisil (Catalog No. 066-05265 produced by FUJIFILM Wako Pure Chemical Corporation). The resulting filtrate was concentrated to give 2.6 g of the target pale yellow solid in a yield of 90%.

By a train sublimation method, 0.66 g of the obtained solid was purified. In the purification by sublimation, the solid was heated at 330° C. for 16 hours under a pressure of 1.4 Pa with an argon flow rate of 10 mL/min. After the purification by sublimation, 0.37 g of the target pale yellow solid was obtained at a collection rate of 56%. Synthesis scheme (s-6) of Step 3 is shown below.

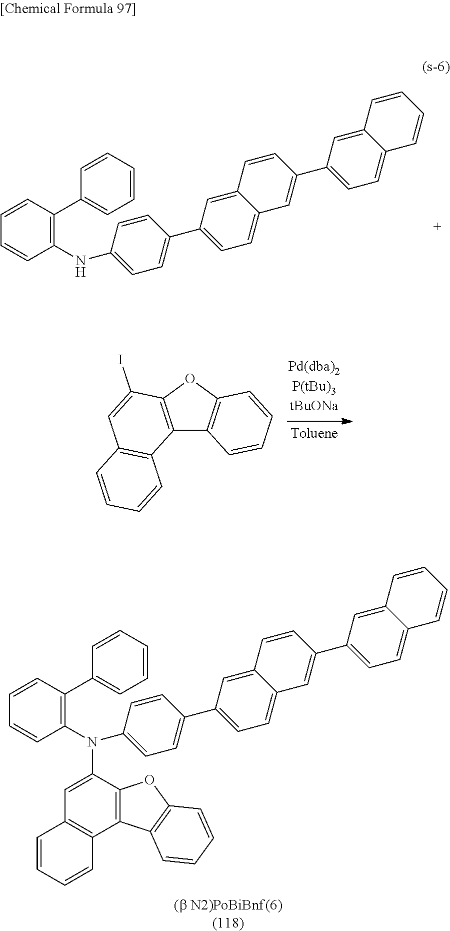 [SMILES: c1ccc(-c2ccccc2Nc2ccc(-c3ccc4cc(-c5ccc6ccccc6c5)ccc4c3)cc2)cc1 ; Ic1cc2ccccc2c2c1oc1ccccc12 ; c1ccc(-c2ccccc2N(c2ccc(-c3ccc4cc(-c5ccc6ccccc6c5)ccc4c3)cc2)c2cc3ccccc3c3c2oc2ccccc23)cc1]

In the above manner, (βN2)PoBiBnf(6) represented by Structural Formula (118) in Embodiment was synthesized.

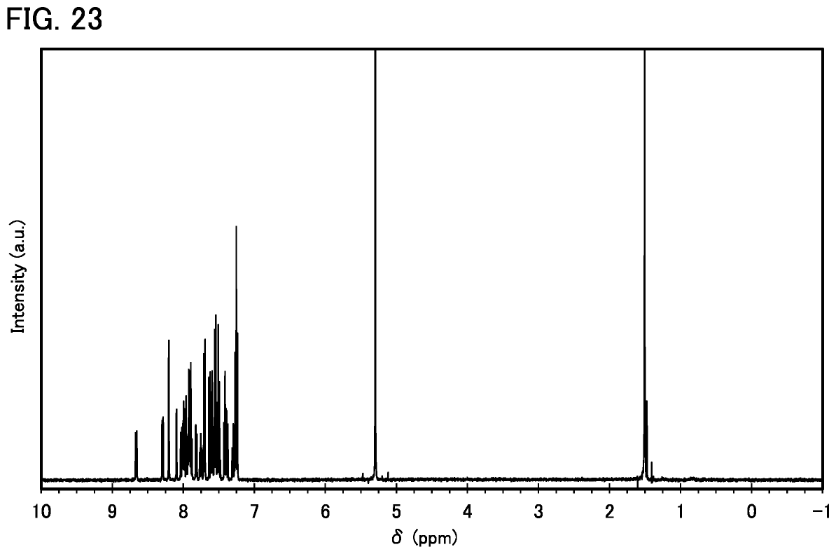

The results of ¹H NMR measurement of the obtained substance is given below. FIG. 23 shows a ¹H NMR spectrum. This shows that (βN2)PoBiBnf(6) was obtained in this synthesis example.

¹H NMR (dichloromethane-d₂, 500 MHz): δ=8.49 (d, J=8.5 Hz, 1H), 8.34-8.32 (m, 1H), 8.20 (d, J=5.0 Hz, 2H), 8.05 (s, 1H), 8.00-7.88 (m, 7H), 7.78 (dd, J=8.5 Hz, 2.0 Hz, 1H), 7.71 (d, J=8.5 Hz, 1H), 7.59-7.34 (m, 13H), 7.30 (s, 1H), 7.27 (d, J=8.0 Hz, 2H), 7.00 (d, J=8.0 Hz, 2H), 6.88 (t, J=8.0 Hz, 2H), 6.75 (t, J=7.5 Hz, 1H),

### <Measurement of Physical Properties>

Next, absorption spectra and emission spectra of a toluene solution and a solid thin film of (βN2)PoBiBnf(6) were measured by a method similar to that in Example 1.

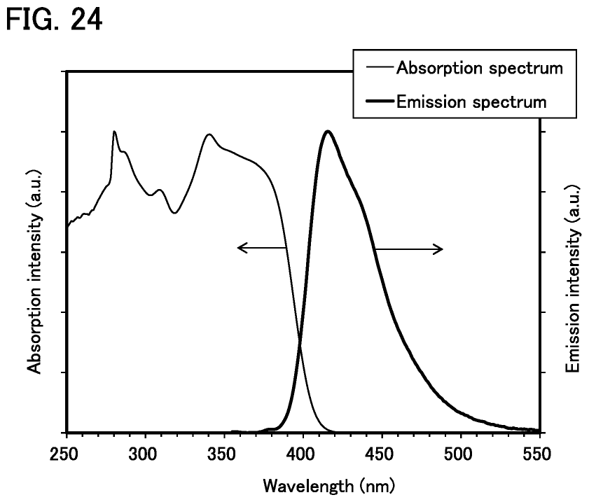

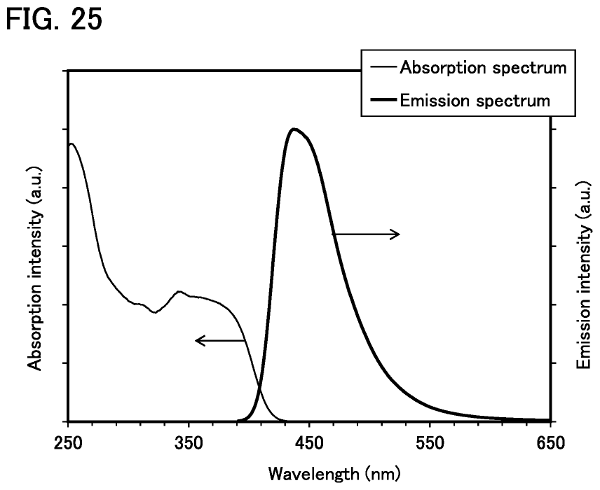

FIG. 24 shows the measurement results of the absorption spectrum and the emission spectrum of the toluene solution of (βN2)PoBiBnf(6), and FIG. 25 shows the measurement results of the absorption spectrum and the emission spectrum of the solid thin film of (βN2)PoBiBnf(6). As shown in the measurement results in FIG. 24, the toluene solution of (βN2)PoBiBnf(6) had the maximum absorption peak at around 380 nm, and it was found that (βN2)PoBiBnf(6) is a material that does not absorb light with a wavelength longer than 430 nm. This indicates that (βN2)PoBiBnf(6) can be suitably used for a transport layer of a light-emitting device. Furthermore, the toluene solution of (βN2)PoBiBnf(6) had an emission spectrum peak at around 416 nm (excitation wavelength: 340 nm). As shown in the measurement results in FIG. 25, the thin film of (βN2)PoBiBnf(6) had a shoulder peak at around 375 nm, and it was found that (βN2)PoBiBnf(6) is a material that does not absorb light with a wavelength longer than 430 nm. This indicates that (βN2)PoBiBnf(6) can be suitably used for a transport layer of a light-emitting device. Furthermore, the thin film of (βN2)PoBiBnf(6) had an emission peak at around 437 nm (excitation wavelength: 371 nm).

In addition, the TG-DTA of (βN2)PoBiBnf(6) was performed by the method similar to that in Example 1. As a result, the temperature (sublimation or decomposition temperature) at which the weight obtained by thermogravimetry was reduced by 5% of the weight at the beginning of the measurement was 469° C., indicating that (βN2)PoBiBnf(6) has high heat resistance.

In addition, the DSC measurement of (βN2)PoBiBnf(6) was performed by the method similar to that in Example 1. The DSC measurement results of the second cycle show that the glass transition temperature of (βN2)PoBiBnf(6) is 132° C., indicating that using (βN2)PoBiBnf(6) enables providing an organic semiconductor element having high heat resistance.

A difference between (βN2)PBiBnf(6) whose synthesis method is shown in Example 2 and (βN2)PoBiBnf(6) whose synthesis method is shown in this synthesis example is the substitution site of the biphenyl group bonded to N^{A}. When these compounds are compared for their T_{g}, (βN2)PBiBnf(6) has a higher T_{g}, which is 137° C. Thus, it was found that when Ar¹ of an organic compound of one embodiment of the present invention is a biphenyl group, the organic compound can have higher heat resistance in the case where the biphenyl group is a p-biphenyl group than in the case where the biphenyl group is an o-biphenyl group.

### Example 4

### Synthesis Example 4

In this synthesis example, a synthesis method of N-[4-(2,2′-binaphthyl-6-yl)phenyl]-N-(biphenyl-2-yl)benzo[b]naphtho[1,2-d]furan-8-amine (abbreviation: (βN2)PoBiBnf(8)) (Structural Formula (117)), which is an organic compound of one embodiment of the present invention, is described. The structural formula of (βN2)PoBiBnf(8) is shown below.

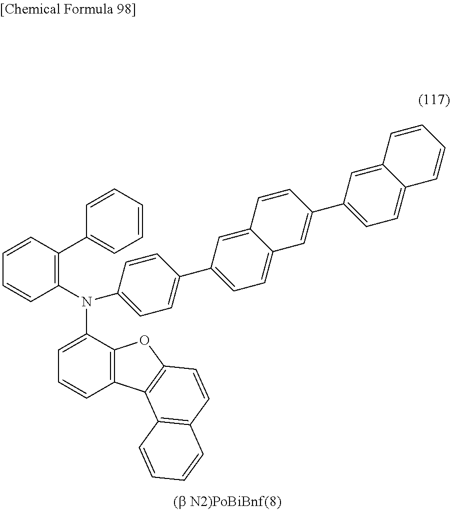 [SMILES: c1ccc(-c2ccccc2N(c2ccc(-c3ccc4cc(-c5ccc6ccccc6c5)ccc4c3)cc2)c2cccc3c2oc2ccc4ccccc4c23)cc1]

### Step 1A: Synthesis 1 of (βN2)PoBiBnf(8)

Into a 200-mL three-neck flask were put 1.2 g (2.4 mmol) of N-[4-(2,2′-binaphthyl-6-yl)phenyl]-N-(biphenyl-2-yl)amine synthesized in Step 2 in Synthesis Example 3, 0.61 g (2.4 mmol) of 8-chlorobenzo[b]naphtho[1,2-d]furan, and 20 mg (48 μmol) of 2-dicyclohexylphosphino-2′,6′-dimethoxybiphenyl (commonly known name: SPhos). After the air in the flask was replaced with nitrogen, 0.71 g (7.4 mmol) of sodium tert-butoxide (abbreviation: tBuONa) and 25 mL of toluene were added thereto. This mixture was degassed by being stirred under reduced pressure. To this mixture was added 21 mg (37 μmol) of bis(dibenzylideneacetone)palladium(0) (commonly known name: Pd(dba)₂), and stirring was performed at 120° C. for seven hours. After the stirring, 0.32 g (1.3 mmol) of 8-chlorobenzo[b]naphtho[1,2-d]furan, 23 mg (56 μmol) of SPhos, 0.30 g (3.1 mmol) of tBuONa, and 14 mg (24 μmol) of Pd(dba)₂ were added, and stirring was performed at 120° C. for six hours. After the stirring, toluene was added to the obtained mixture, and stirring was performed under heating at 100° C. This mixture was suction-filtered at 100° C. through alumina, Celite (Catalog No. 537-02305 produced by FUJIFILM Wako Pure Chemical Corporation), and Florisil (Catalog No. 066-05265 produced by FUJIFILM Wako Pure Chemical Corporation). The resulting filtrate was concentrated to give 1.8 g of a pale yellow solid containing the target substance. This solid was purified by silica gel column chromatography (while the ratio of hexane to toluene in the developing solvent was changed from 5:1 to 3:1) to give 0.82 g of the target white solid in a yield of 48%. Synthesis Scheme (s-7) of Step 1A is shown below.

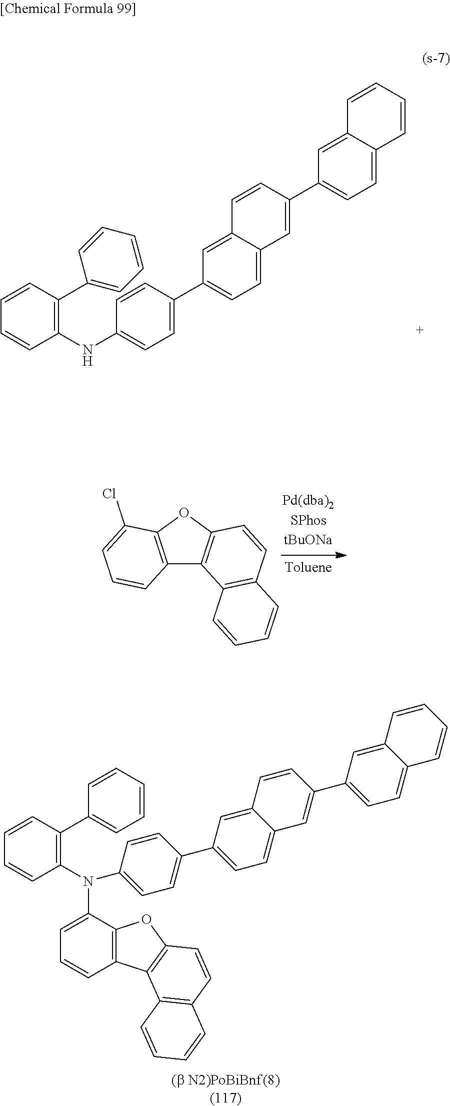 [SMILES: c1ccc(-c2ccccc2Nc2ccc(-c3ccc4cc(-c5ccc6ccccc6c5)ccc4c3)cc2)cc1 ; Clc1cccc2c1oc1ccc3ccccc3c12 ; c1ccc(-c2ccccc2N(c2ccc(-c3ccc4cc(-c5ccc6ccccc6c5)ccc4c3)cc2)c2cccc3c2oc2ccc4ccccc4c23)cc1]

Note that (βN2)PoBiBnf(8) can also be synthesized by a method other than that in Step 1A. Next, a synthesis method of (βN2)PoBiBnf(8) different from that in Step 1A is described.

### Step 1B: Synthesis of N-(biphenyl-2-yl)-N-(1-chlorophenyl-4-yl)benzo[b]naphto[1,2-d]furan-8-amine

Into a 200-mL three-neck flask were put 2.8 g (10 mmol) of N-(biphenyl-2-yl)-1-chlorophenyl-4-amine, 6.37 g (25 mmol) of 8-chlorobenzo[b]naphtho[1,2-d]furan, and 87 mg (0.21 mmol) of 2-dicyclohexylphosphino-2′,6′-dimethoxybiphenyl (commonly known name: SPhos). After the air in the flask was replaced with nitrogen, 2.9 g (30 mmol) of sodium tert-butoxide (abbreviation: tBuONa) and 70 mL of toluene were added thereto. This mixture was degassed by being stirred under reduced pressure. To this mixture was added 61 mg (0.11 mmol) of bis(dibenzylideneacetone)palladium(0) (commonly known name: Pd(dba)₂) was added, and stirring was performed at 120° C. for three hours. After the stirring, toluene was added to the obtained mixture, and stirring was performed under heating at 100° C. This mixture was suction-filtered at 100° C. through alumina, Celite (Catalog No. 537-02305 produced by FUJIFILM Wako Pure Chemical Corporation), and Florisil (Catalog No. 066-05265 produced by FUJIFILM Wako Pure Chemical Corporation). The resulting filtrate was concentrated to give 8.4 g of a pale yellow solid containing the target substance. This solid was purified by silica gel column chromatography (while the ratio of hexane to toluene in developing solvent was changed from 5:1 to 3:1) to give 1.6 g of the target white solid in a yield of 31%. Synthesis Scheme (s-8) of Step 1B is shown below.

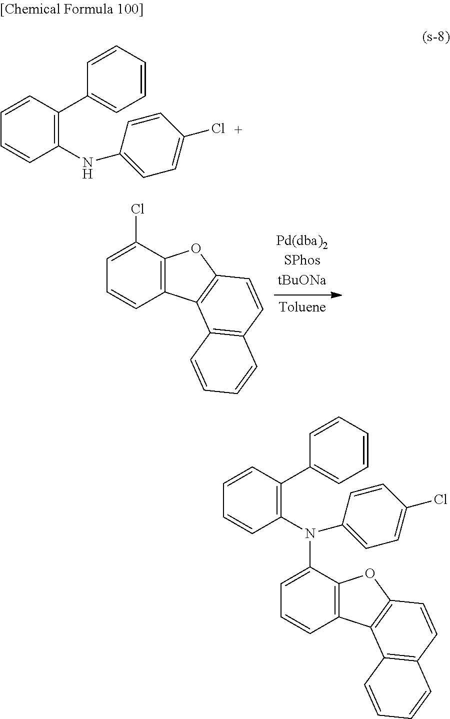

### Step 2B: Synthesis 2 of (βN2)PoBiBnf(8)

Into a 200-mL three-neck flask were put 1.6 g (3.2 mmol) of N-(biphenyl-2-yl)-N-(1-chlorophenyl-4-yl)benzo[b]naphtho[1,2-d]furan-8-amine, 1.2 g (3.2 mmol) of 2-(2,2′-binaphthyl-6-yl)-4,4,5,5-tetramethyl-1,3,2-dioxaborolane, 1.5 g (10 mmol) of cesium fluoride, 25 mg (69 μmol) of di-1-adamantyl-n-butylphosphine (commonly known name: cataCXium®A), 6.0 mL of water, 10 mL of ethanol, and 30 mL of toluene. This mixture was degassed by being stirred under reduced pressure, and then the air in the flask was replaced with nitrogen. To this mixture was added 20 mg (89 μmol) of palladium(II) acetate (abbreviation: Pd(OAc)₂), and stirring was performed at 90° C. for one hour. After the stirring, 1.6 g (11 mmol) of cesium fluoride, 24 mg (67 μmol) of cataCXium®A, and 21 mg (95 μmol) of Pd(OAc)₂ were added, and stirring was performed at 90° C. for seven hours. After the stirring, 21 mg (60 μmol) of cataCXium®A and 7.2 mg (95 μmol) of Pd(OAc)₂ were added, and stirring was performed at 90° C. for eight hours. After the stirring, 0.61 mg (1.6 mmol) of 2-(2,2′-binaphthyl-6-yl)-4,4,5,5-tetramethyl-1,3,2-dioxaborolane, 19 mg (52 μmol) of cataCXium®A, and 16 mg (72 μmol) of Pd(OAc)₂ were added, and stirring was performed at 90° C. for four hours. After the stirring, 11 mg (32 μmol) of cataCXium®A and 29 mg (0.13 mmol) of Pd(OAc)₂ were added, and stirring was performed at 90° C. for eight hours. After the stirring, 1.4 g (9.4 mmol) of cesium fluoride, 25 mg (70 μmol) of cataCXium®A, and 6.4 mg (28 μmol) of Pd(OAc)₂ were added, and stirring was performed at 90° C. for two hours. After the stirring, water was added to the reaction mixture, and the obtained aqueous layer was subjected to extraction with toluene. The obtained organic layer was washed twice with water and then dried with anhydrous magnesium sulfate. The obtained mixture was suction-filtered through Celite (Catalog No. 537-02305 produced by FUJIFILM Wako Pure Chemical Corporation). The resulting filtrate was concentrated to give 2.4 g of a blackish-brown viscous solid containing the target substance. This solid was purified by silica gel column chromatography (developing solvent: toluene) and high performance liquid chromatography to give 0.75 g of the target pale yellow solid in a yield of 33%. Synthesis Scheme (s-9) of Step 2B is shown below.

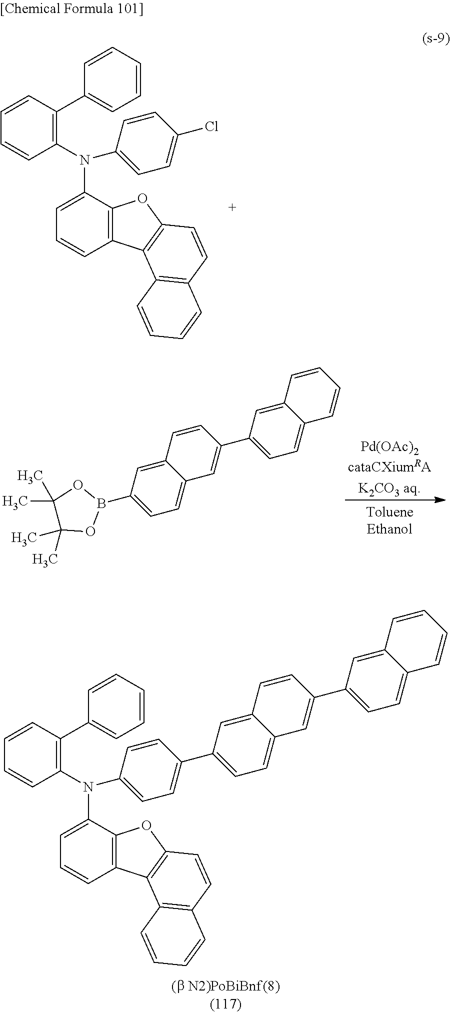 [SMILES: Clc1ccc(N(c2ccccc2-c2ccccc2)c2cccc3c2oc2ccc4ccccc4c23)cc1 ; CC1(C)OB(c2ccc3cc(-c4ccc5ccccc5c4)ccc3c2)OC1(C)C ; c1ccc(-c2ccccc2N(c2ccc(-c3ccc4cc(-c5ccc6ccccc6c5)ccc4c3)cc2)c2cccc3c2oc2ccc4ccccc4c23)cc1]

In the above manner, (βN2)PoBiBnf(8) represented by Structural Formula (117) in Embodiment was synthesized.

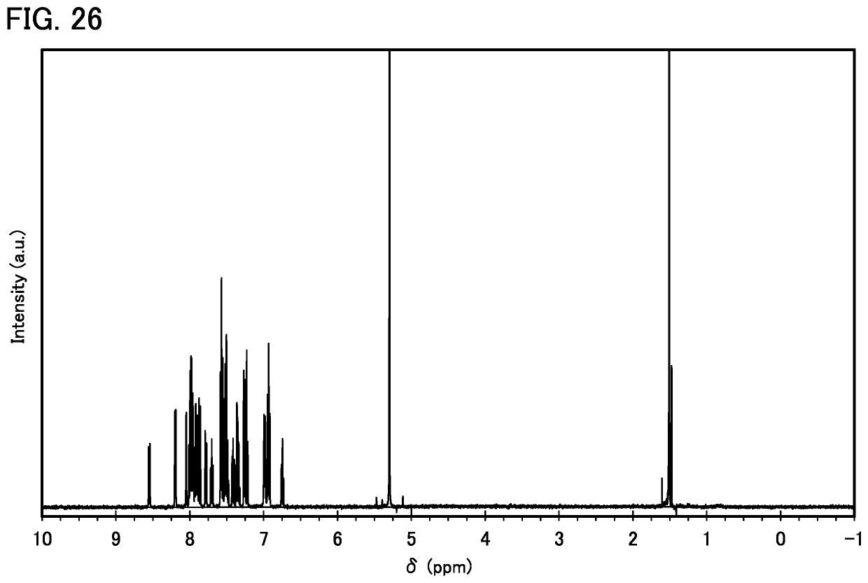

The results of ¹H NMR measurement of the obtained substance is given below. FIG. 26 shows a ¹H NMR spectrum. This shows that (βN2)PBiBnf(8) was obtained in this synthesis example.

¹H NMR (dichloromethane-d2, 500 MHz): δ=8.55 (d, J=8.0 Hz, 1H), 8.20 (dd, J=5.5 Hz, 1.5 Hz, 2H), 8.05 (d, J=1.5 Hz, 1H), 8.02-7.86 (m, 10H), 7.78 (dd, J=8.5 Hz, 1.5 Hz, 1H), 7.71 (dt, J=8.0 Hz, 1.0 Hz, 1H), 7.59-7.47 (m, 7H), 7.44-7.70 (m, 1H), 7.38-7.32 (m, 2H), 7.27-7.21 (m, 3H), 6.99 (d, J=8.5 Hz, 2H), 6.93 (m, 3H), 6.75 (dt, J=7.5 Hz, 1.0 Hz, 1H)

### <Measurement of Physical Properties>

Next, absorption spectra and emission spectra of a toluene solution and a solid thin film of (βN2)PoBiBnf(8) were measured by a method similar to that in Example 1.

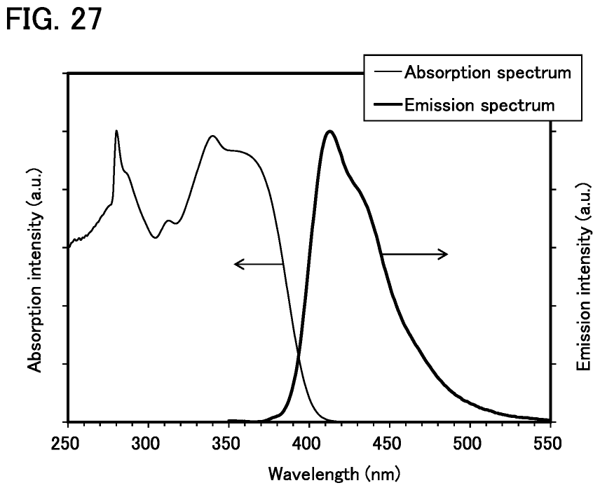

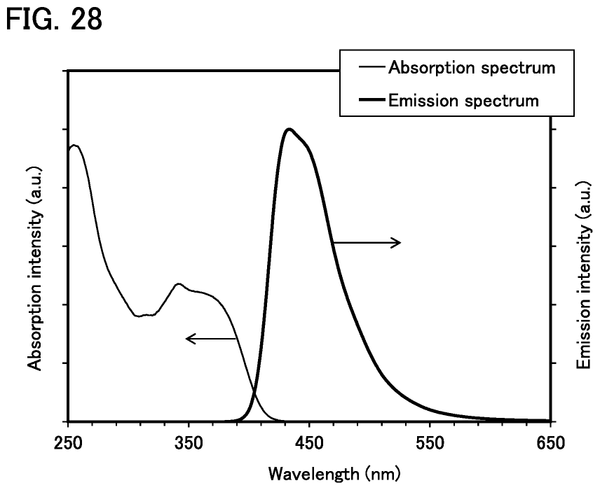

FIG. 27 shows the measurement results of the absorption spectrum and the emission spectrum of the toluene solution of (βN2)PoBiBnf(8), and FIG. 28 shows the measurement results of the absorption spectrum and the emission spectrum of the solid thin film of (βN2)PoBiBnf(8). As shown in the measurement results in FIG. 27, the toluene solution of (βN2)PoBiBnf(8) had the maximum absorption peak at around 365 nm, and it was found that (βN2)PoBiBnf(8) is a material that does not absorb light with a wavelength longer than 430 nm. This indicates that (βN2)PoBiBnf(8) can be suitably used for a transport layer of a light-emitting device. Furthermore, the toluene solution of (βN2)PoBiBnf(8) had an emission spectrum peak at around 413 nm (excitation wavelength: 340 nm). As shown in the measurement results in FIG. 28, the solid thin film of (βN2)PoBiBnf(8) had a shoulder peak at around 371 nm, and it was found that (βN2)PoBiBnf(8) is a material that does not absorb light with a wavelength longer than 430 nm. This indicates that (βN2)PoBiBnf(8) can be suitably used for a transport layer of a light-emitting device. Furthermore, the thin film of (βN2)PoBiBnf(8) had an emission peak at around 433 nm (excitation wavelength: 368 nm).

In addition, the TG-DTA of (βN2)PoBiBnf(8) was performed by the method similar to that in Example 1. As a result, the temperature (sublimation or decomposition temperature) at which the weight obtained by thermogravimetry was reduced by 5% of the weight at the beginning of the measurement was 491° C., indicating that (βN2)PoBiBnf(8) has high heat resistance.

In addition, the DSC measurement of (βN2)PoBiBnf(8) was performed by the method similar to that in Example 1. The DSC measurement results of the second cycle show that the glass transition temperature of (βN2)PBiBnf(8) is 127° C., indicating that using (βN2)PoBiBnf(8) enables providing an organic semiconductor element having high heat resistance.

A difference between (βN2)PBiBnf(8) whose synthesis method is shown in Example 2 and (βN2)PoBiBnf(8) whose synthesis method is shown in this synthesis example is the substitution site of the biphenyl group bonded to N^{A}. When these compounds are compared for their T_{g}, (βN2)PBiBnf(8) has a higher T_{g}, which is 137° C. Thus, it was found that when Ar¹ of an organic compound of one embodiment of the present invention is a biphenyl group, the organic compound can have higher heat resistance in the case where the biphenyl group is a p-biphenyl group than in the case where the biphenyl group is an o-biphenyl group. As described above, there was the same tendency in the comparison of (βN2)PBiBnf(6) and (βN2)PoBiBnf(6).

Note that even in the case where Ar¹ of an organic compound of one embodiment of the present invention is an o-biphenyl group as in the case of (βN2)PoBiBnf(8) and (βN2)PoBiBnf(6), the compound has an extremely high T_{g}exceeding 120° C., and it can be said that the compound has sufficiently high heat resistance.

### <Cv Measurement>

The HOMO levels and the LUMO levels of (βN2)PBiBnf(6), (βN2)PBiBnf(8), (βN2)PoBiBnf(6), and (βN2)PoBiBnf(8) were obtained by a cyclic voltammetry (CV) measurement. The calculation method is shown below.

An electrochemical analyzer (ALS model 600A or 600C, manufactured by BAS Inc.) was used as a measurement apparatus. To prepare a solution for the CV measurement, dehydrated dimethylformamide (DMF; produced by Sigma-Aldrich Inc., 99.8%, catalog No. 22705-6) was used as a solvent, and tetra-n-butylammonium perchlorate (commonly known name: n-Bu₄NClO₄; produced by Tokyo Chemical Industry Co., Ltd., catalog No. T0836) as a supporting electrolyte was dissolved at a concentration of 100 mmol/L. Furthermore, the object to be measured was also dissolved at a concentration of 2 mmol/L.

A platinum electrode (PTE platinum electrode, manufactured by BAS Inc.) was used as a working electrode, another platinum electrode (Pt counter electrode for VC-3 (5 cm), manufactured by BAS Inc.) was used as an auxiliary electrode, and an Ag/Ag⁺ electrode (RE7 reference electrode for nonaqueous solvent, manufactured by BAS Inc.) was used as a reference electrode. Note that the measurement was performed at room temperature (20° C. to 25° C.).

The scan speed in the CV measurement was fixed to 0.1 V/see, and an oxidation potential Ea [V] and a reduction potential Ec [V] with respect to the reference electrode were measured. The potential Ea is an intermediate potential of an oxidation-reduction wave, and the potential Ec is an intermediate potential of a reduction-oxidation wave. Here, since the potential energy of the reference electrode used in this example with respect to the vacuum level is known to be −4.94 [eV], the HOMO level and the LUMO level can be calculated by the following formulae: HOMO level [eV]=−4.94−Ea and LUMO level [eV]−4.94−Ec.

The CV measurement was repeated 100 times, and the oxidation-reduction wave in the hundredth cycle was compared with the oxidation-reduction wave in the first cycle to examine the electrical stability of the compound.

As a result, in the measurement of the oxidation potential Ea [V] performed on (βN2)PBiBnf(6), (βN2)PBiBnf(8), (βN2)PoBiBnf(6), and (βN2)PoBiBnf(8), the HOMO levels of (βN2)PBiBnf(6), (βN2)PBiBnf(8), (βN2)PoBiBnf(6), and (βN2)PoBiBnf(8) were found to be −5.53 eV, −5.53 eV, −5.54 eV, and −5.55 eV, respectively. On the other hand, in the measurement of the reduction potential Ec [V], the LUMO levels of (βN2)PBiBnf(6), (βN2)PBiBnf(8), (βN2)PoBiBnf(6), and (βN2)PoBiBnf(8) were found to be −2.48 eV, −2.48 eV, −2.45 eV, and −2.44 eV, respectively.

The oxidation-reduction wave was repeatedly measured, and the waveform in the hundredth cycle was compared with that in the first cycle; in the Ea measurement of (βN2)PBiBnf(6), (βN2)PBiBnf(8), (βN2)PoBiBnf(6), and (βN2)PoBiBnf(8), 86%, 85%, 87%, and 91% of the peak intensities were maintained, respectively; in the Ec measurement of (βN2)PBiBnf(6), (βN2)PBiBnf(8), (βN2)PoBiBnf(6), and (βN2)PoBiBnf(8), 99%, 97%, 97%, and 93% of the peak intensities were maintained, respectively. Accordingly, (βN2)PBiBnf(6), (βN2)PBiBnf(8), (βN2)PoBiBnf(6), and (βN2)PoBiBnf(8) are highly resistant to repetitive oxidation and reduction. In particular, their resistance to reduction is excellent; thus, (βN2)PBiBnf(6), (βN2)PBiBnf(8), (βN2)PoBiBnf(6), and (βN2)PoBiBnf(8) can be suitably used for an electron-blocking layer in contact with a light-emitting layer in a light-emitting device.

### Example 5

This example describes results of fabricating light-emitting devices 1 to 4 of embodiments of the present invention with the use of organic compounds of embodiments of the present invention. The structural formulae of the organic compounds used for the light-emitting devices 1 to 4 are shown below. In addition, structures of the light-emitting devices 1 to 4 are shown.

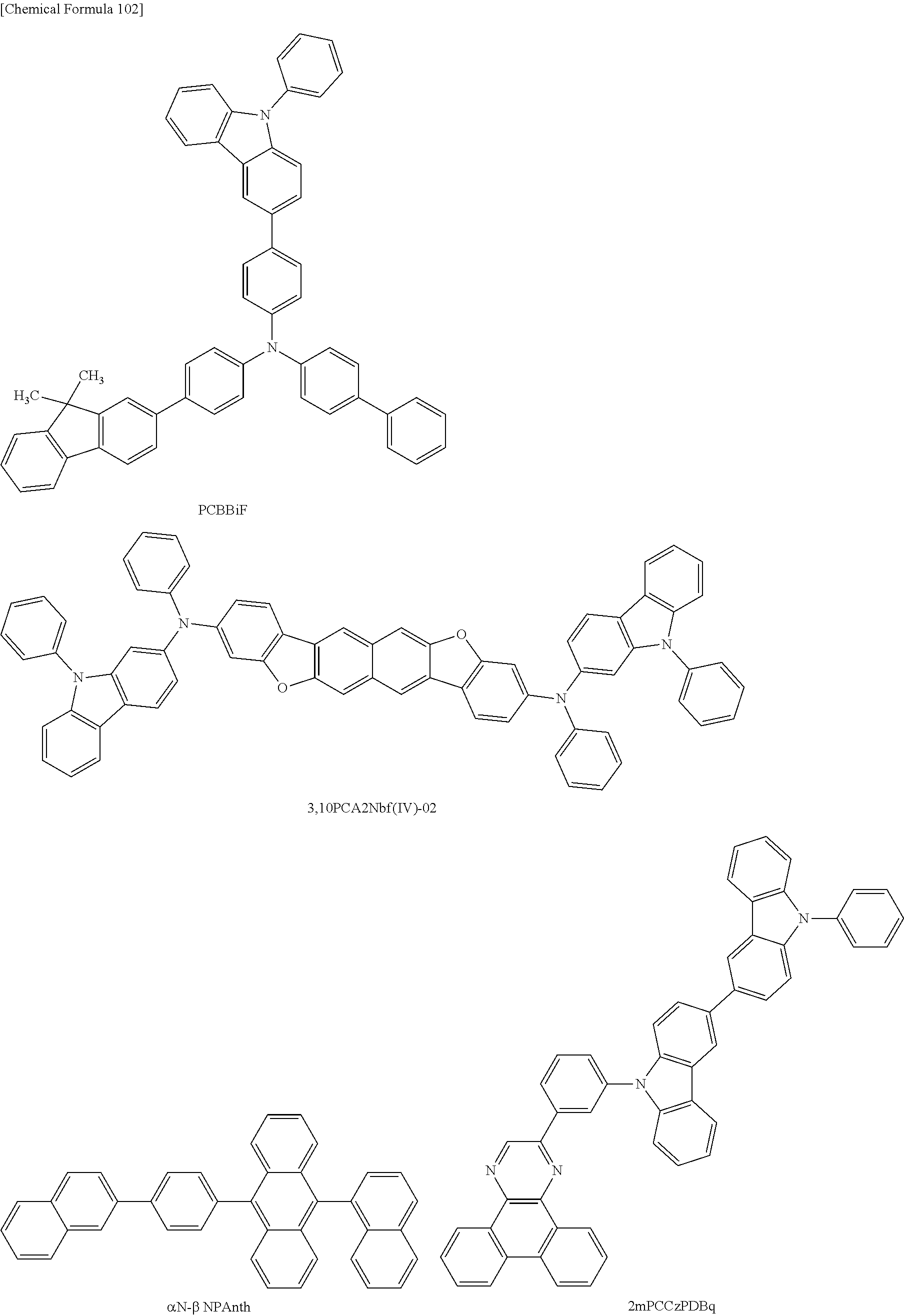

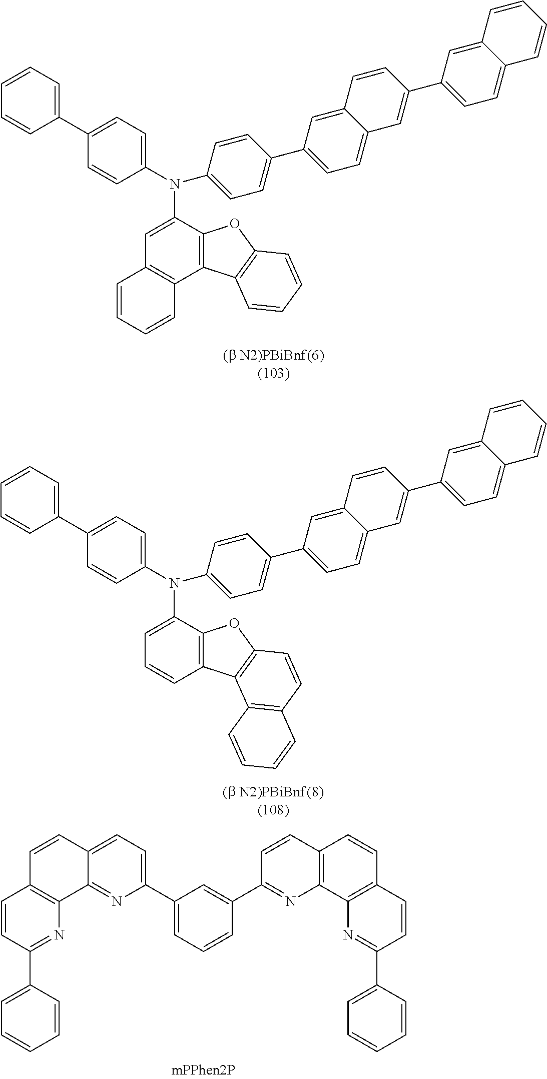 [SMILES: c1ccc(-c2ccc(N(c3ccc(-c4ccc5cc(-c6ccc7ccccc7c6)ccc5c4)cc3)c3cc4ccccc4c4c3oc3ccccc34)cc2)cc1 ; c1ccc(-c2ccc(N(c3ccc(-c4ccc5cc(-c6ccc7ccccc7c6)ccc5c4)cc3)c3cccc4c3oc3ccc5ccccc5c34)cc2)cc1 ; c1ccc(-c2ccc3ccc4ccc(-c5cccc(-c6ccc7ccc8ccc(-c9ccccc9)nc8c7n6)c5)nc4c3n2)cc1]

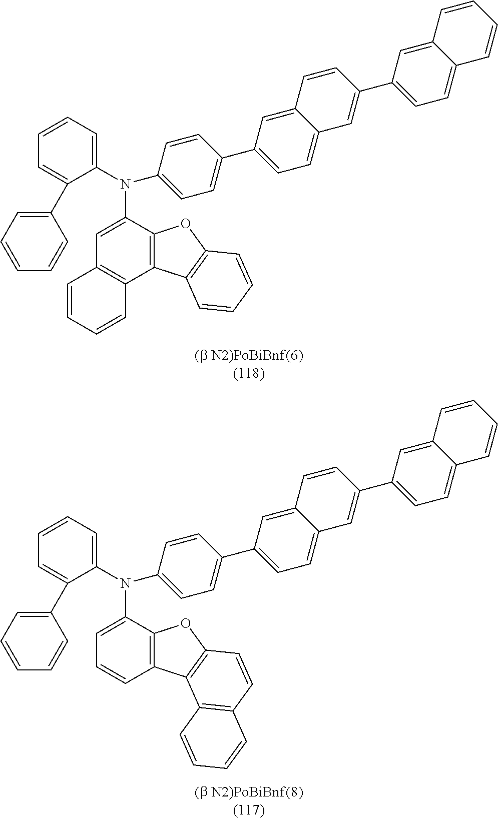 [SMILES: c1ccc(-c2ccccc2N(c2ccc(-c3ccc4cc(-c5ccc6ccccc6c5)ccc4c3)cc2)c2cc3ccccc3c3c2oc2ccccc23)cc1 ; c1ccc(-c2ccccc2N(c2ccc(-c3ccc4cc(-c5ccc6ccccc6c5)ccc4c3)cc2)c2cccc3c2oc2ccc4ccccc4c23)cc1]

TABLE 1

| | Thickness | Light-emitting | Light-emitting | Light-emitting | Light-emitting |
|---|---|---|---|---|---|
| | (nm) | device 1 | device 2 | device 3 | device 4 |
| Second electrode | 200 | Al | | | |
| Electron-injection layer | 1 | LiF | | | |
| Electron-transport layer | 15 | mPPhen2P | | | |
| Hole-blocking layer | 10 | 2mPCCzPDBq | | | |
| Light-emitting layer | 25 | αN-βNPAnth:3,10PCA2Nbf(IV)-02 (1:0.015) | | | |
| Electron-blocking layer | 10 | (βN2)PBiBnf(6) | (βN2)PBiBnf(8) | (βN2)PoBiBnf(6) | (βN2)PoBiBnf(8) |

| | | |
|---|---|---|
| Hole-transport layer | 20 | PCBBiF |
| Hole-injection layer | 10 | PCBBiF:OCHD-003 (1:0.03) |
| First electrode | 70 | ITSO |

### <<Fabrication of Light-Emitting Device 1>>

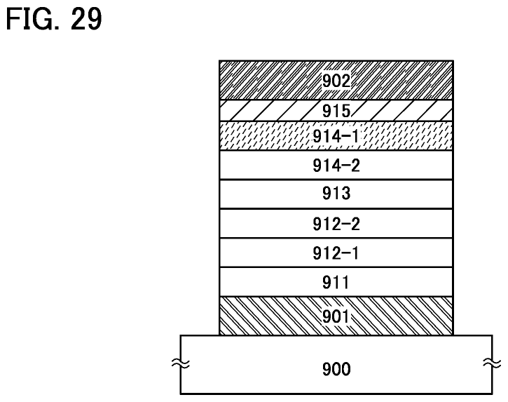

In the light-emitting device 1 described in this example, as illustrated in FIG. 29, a hole-injection layer 911, a hole-transport layer 912-1, an electron-blocking layer 912-2, a light-emitting layer 913, a hole-blocking layer 914-2, an electron-transport layer 914-1, and an electron-injection layer 915 are stacked in this order over a first electrode 901 formed over a substrate 900, and a second electrode 902 is stacked over the electron-injection layer 915.

First, the first electrode 901 was formed over the substrate 900. The electrode area was set to 4 mm² (2 mm×2 mm). A glass substrate was used as the substrate 900. As the first electrode 901, 70-nm-thick indium tin oxide containing silicon oxide (ITSO) was deposited by a sputtering method. In this example, the first electrode 901 functions as an anode.

For pretreatment, a surface of the substrate was washed with water, baking was performed at 200° C. for one hour, and then UV ozone treatment was performed for 370 seconds. After that, the substrate was transferred into a vacuum evaporation apparatus where the pressure was reduced to approximately 10⁻⁴ Pa, vacuum baking was performed at 170° C. for 30 minutes in a heating chamber of the vacuum evaporation apparatus, and then the substrate was cooled down for approximately 30 minutes.

Next, the hole-injection layer 911 was formed over the first electrode 901. The hole-injection layer 911 was formed in the following manner: the pressure in the vacuum evaporation apparatus was reduced to 10⁻⁴ Pa, and then N-(biphenyl-4-yl)-N-[4-(9-phenyl-9H-carbazol-3-yl)phenyl]-9,9-dimethyl-9H-fluoren-2-amine (abbreviation: PCBBiF) and an electron acceptor material (OCHD-003) that contains fluorine and has a molecular weight of 672 were deposited by co-evaporation to a thickness of 10 nm in a weight ratio of PCBBiF:OCHD-003=1:0.03.

Then, the hole-transport layer 912-1 was formed over the hole-injection layer 911. The hole-transport layer 912-1 was formed to a thickness of 20 nm by evaporation of PCBBiF.

Next, the electron-blocking layer 912-2 was formed over the hole-transport layer 912-1. The electron-blocking layer 912-2 was formed to a thickness of 10 nm by evaporation of (βN2)PBiBnf(6) (Structural Formula (103)) which is an organic compound of one embodiment of the present invention.

Then, the light-emitting layer 913 was formed over the electron-blocking layer 912-2. The light-emitting layer 913 was formed to a thickness of 25 nm by co-evaporation of 9-(1-naphthyl)-10-[4-(2-naphthyl)phenyl]anthracene (abbreviation: αN-βNPAnth) and 3,10-bis[N-(9-phenyl-9H-carbazol-2-yl)-N-phenylamino]naphtho[2,3-b;6,7-b′]bisbenzofuran (abbreviation: 3,1OPCA2Nbf(IV)-02) such that the weight ratio of αN-βNPAnth to 3,1OPCA2Nbf(IV)-02 was 1:0.015.

Next, the hole-blocking layer 914-2 was formed over the light-emitting layer 913. The hole-blocking layer 914-2 was formed to a thickness of 10 nm by evaporation of 2-{3-[3-(N-phenyl-9H-carbazol-3-yl)-9H-carbazol-9-yl]phenyl}dibenzo[f,h]quinoxaline (abbreviation: 2mPCCzPDBq).

Then, the electron-transport layer 914-1 was formed over the hole-blocking layer 914-2. The electron-transport layer 914-1 was formed to a thickness of 15 nm by evaporation of 2,2′-(1,3-phenylene)bis(9-phenyl-1,10-phenanthroline) (abbreviation: mPPhen2P).

Then, the electron-injection layer 915 was formed over the electron-transport layer 914-1. The electron-injection layer 915 was formed to a thickness of 1 nm by evaporation of lithium fluoride (LiF).

After that, the second electrode 902 was formed over the electron-injection layer 915. As the second electrode 902, aluminum (Al) was deposited by evaporation to a thickness of 200 nm. In this example, the second electrode 902 functions as a cathode.

Through the above process, the light-emitting device 1 was fabricated. Next, methods of fabricating the light-emitting devices 2 to 4 are described.

### <<Fabrication of Light-Emitting Device 2>>

The light-emitting device 2 is different from the light-emitting device 1 in that (βN2)PBiBnf(6) used for the electron-blocking layer 912-2 in the light-emitting device 1 is replaced with (βN2)PBiBnf(8) (Structural Formula (108)) which is an organic compound of one embodiment of the present invention. The other components were formed in the same manner as those in the light-emitting device 1.

### <<Fabrication of Light-Emitting Device 3>>

The light-emitting device 3 is different from the light-emitting device 1 in that (βN2)PBiBnf(6) used for the electron-blocking layer 912-2 in the light-emitting device 1 is replaced with (βN2)PoBiBnf(6) (Structural Formula (118)) which is an organic compound of one embodiment of the present invention. The other components were formed in the same manner as those in the light-emitting device 1.

### <<Fabrication of Light-Emitting Device 4>>

The light-emitting device 4 is different from the light-emitting device 1 in that (βN2)PBiBnf(6) used for the electron-blocking layer 912-2 in the light-emitting device 1 is replaced with (βN2)PoBiBnf(8) (Structural Formula (117)) which is an organic compound of one embodiment of the present invention. The other components were formed in the same manner as those in the light-emitting device 1.

The light-emitting devices were sealed using a glass substrate in a glove box containing a nitrogen atmosphere so as not to be exposed to the air (a sealing material was applied to surround the device and UV treatment and heat treatment at 80° C. for one hour were performed at the time of sealing). Then, the initial characteristics of the light-emitting devices were measured.

Table 2 shows the main characteristics of the light-emitting devices 1 to 4 at a luminance of about 1000 cd/m². Table 3 shows a time LT₉₀ taken for the luminance to drop to 90% of its initial value at a constant current density of 50 mA/cm², which were obtained under the condition where the light-emitting devices 2 to 4 each emitted light. The luminance, CIE chromaticity, and emission spectra were measured with a spectroradiometer (SR-UL1R, TOPCON TECHNOHOUSE CORPORATION). The external quantum efficiency was calculated from the luminance and the emission spectra measured with the spectroradiometer, on the assumption that the light-emitting devices had Lambertian light-distribution characteristics.

TABLE 2

| | | | | | | | External |
|---|---|---|---|---|---|---|---|
| | | | Current | | | Current | quantum |
| | Voltage | Current | density | | | efficiency | efficiency |
| | (V) | (mA) | (mA/cm²) | Chromaticity x | Chromaticity y | (cd/A) | (%) |
| Light-emitting device 1 | 4.0 | 0.3414 | 8.54 | 0.138 | 0.122 | 773 | 9.05 |
| Light-emitting device 2 | 4.0 | 0.4158 | 10.40 | 0.138 | 0.105 | 924 | 8.89 |
| Light-emitting device 3 | 4.2 | 0.5163 | 12.91 | 0.135 | 0.124 | 1273 | 9.86 |
| Light-emitting device 4 | 4.0 | 0.3622 | 9.05 | 0.136 | 0.111 | 893 | 9.86 |

TABLE 3

| | LT₉₀ (h) |
|---|---|
| Light-emitting device 2 | 108 |
| Light-emitting device 3 | 72 |
| Light-emitting device 4 | 63 |

Current density: 50 mA/cm²

Table 2 shows that the light-emitting devices 2 to 4 each have favorable initial characteristics. Table 3 shows that the light-emitting devices 2 to 4 each have a long LT₉₀ and a small luminance change over driving time. From the comparison between the light-emitting device 2 and the light-emitting device 4, it was found that when Ar¹ is a biphenyl group, the light-emitting devices have longer LT₉₀ in the case where the biphenyl group is a p-biphenyl group than in the case where the biphenyl group is an o-biphenyl group. The LT₉₀ of the light-emitting device 3 is equivalent to that of the light-emitting device 4; accordingly, it can be said that the devices have excellent properties in both of the case where N^{A} is bonded at the 6-position of the benzo[b]naphtho[1,2-d]furan ring and the case where N^{A} is bonded at the 8-position of the benzo[b]naphtho[1,2-d]furan ring.

From the above results, it was revealed that a light-emitting device with excellent initial characteristics and a long driving lifetime can be fabricated with the use of an organic compound of one embodiment of the present invention for an electron-blocking layer.

This application is based on Japanese Patent Application Serial No. 2023-048370 filed with Japan Patent Office on Mar. 24, 2023, the entire contents of which are hereby incorporated by reference.

What is claimed is:

1. An organic compound represented by General Formula (G1): 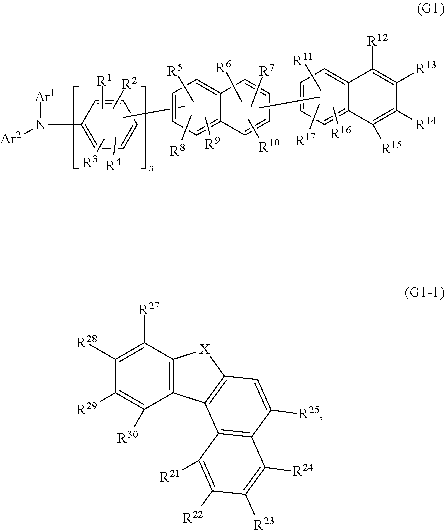
wherein, in General Formula (G1):
Ar¹ represents a substituted or unsubstituted aryl group having 6 to 30 carbon atoms or a substituted or unsubstituted heteroaryl group having 2 to 30 carbon atoms;
Ar² is a group represented by General Formula (G1-1);
R¹ to R¹⁷ each independently represent any one of hydrogen, a substituted or unsubstituted alkyl group having 1 to 6 carbon atoms, and a substituted or unsubstituted cycloalkyl group having 3 to 10 carbon atoms; and
n represents an integer of 0 to 3,
wherein, in General Formula (G1-1):
X represents oxygen or sulfur;
any one of R²¹ to R³⁰ is bonded to nitrogen in General Formula (G1); and
the others of R²¹ to R³⁰ are each independently represent any one of hydrogen, a halogen, a cyano group, a substituted or unsubstituted alkyl group having 1 to 6 carbon atoms, a substituted or unsubstituted alkenyl group having 2 to 6 carbon atoms, a substituted or unsubstituted alkynyl group having 2 to 6 carbon atoms, a substituted or unsubstituted cycloalkyl group having 3 to 10 carbon atoms, a substituted or unsubstituted alkoxy group having 1 to 6 carbon atoms, a trialkylsilyl group having 3 to 10 carbon atoms, a substituted or unsubstituted aryl group having 6 to 30 carbon atoms, and a substituted or unsubstituted heteroaryl group having 2 to 30 carbon atoms.

2. An organic compound represented by General Formula (G2): 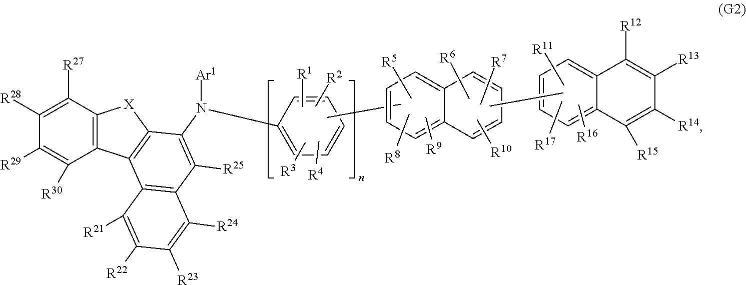
wherein Ar¹ represents a substituted or unsubstituted aryl group having 6 to 30 carbon atoms or a substituted or unsubstituted heteroaryl group having 2 to 30 carbon atoms,
wherein R¹ to R¹⁷ each independently represent any one of hydrogen, a substituted or unsubstituted alkyl group having 1 to 6 carbon atoms, and a substituted or unsubstituted cycloalkyl group having 3 to 10 carbon atoms,
wherein X represents oxygen or sulfur,
wherein R²¹ to R²⁵ and R²⁷ to R³⁰ each independently represent any one of hydrogen, a halogen, a cyano group, a substituted or unsubstituted alkyl group having 1 to 6 carbon atoms, a substituted or unsubstituted alkenyl group having 2 to 6 carbon atoms, a substituted or unsubstituted alkynyl group having 2 to 6 carbon atoms, a substituted or unsubstituted cycloalkyl group having 3 to 10 carbon atoms, a substituted or unsubstituted alkoxy group having 1 to 6 carbon atoms, a trialkylsilyl group having 3 to 10 carbon atoms, a substituted or unsubstituted aryl group having 6 to 30 carbon atoms, and a substituted or unsubstituted heteroaryl group having 2 to 30 carbon atoms, and wherein n represents an integer of 0 to 3.

3. An organic compound represented by General Formula (G3): 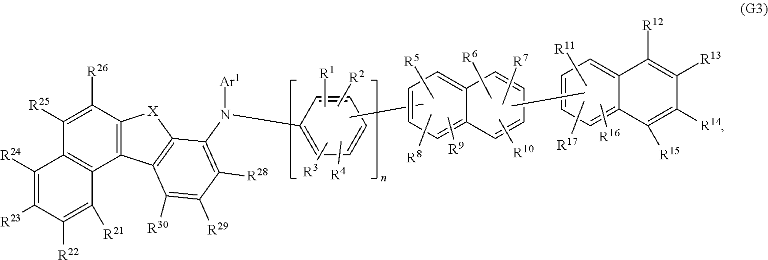
wherein, Ar¹ represents a substituted or unsubstituted aryl group having 6 to 30 carbon atoms or a substituted or unsubstituted heteroaryl group having 2 to 30 carbon atoms,
wherein R¹ to R¹⁷ each independently represent any one of hydrogen, a substituted or unsubstituted alkyl group having 1 to 6 carbon atoms, and a substituted or unsubstituted cycloalkyl group having 3 to 10 carbon atoms,
wherein X represents oxygen or sulfur,
wherein R²¹ to R²⁶ and R²⁸ to R³⁰ each independently represent any one of hydrogen, a halogen, a cyano group, a substituted or unsubstituted alkyl group having 1 to 6 carbon atoms, a substituted or unsubstituted alkenyl group having 2 to 6 carbon atoms, a substituted or unsubstituted alkynyl group having 2 to 6 carbon atoms, a substituted or unsubstituted cycloalkyl group having 3 to 10 carbon atoms, a substituted or unsubstituted alkoxy group having 1 to 6 carbon atoms, a trialkylsilyl group having 3 to 10 carbon atoms, a substituted or unsubstituted aryl group having 6 to 30 carbon atoms, and a substituted or unsubstituted heteroaryl group having 2 to 30 carbon atoms, and
wherein n represents an integer of 0 to 3.

4. The organic compound according to claim 2, wherein the organic compound is represented by General Formula (G4): 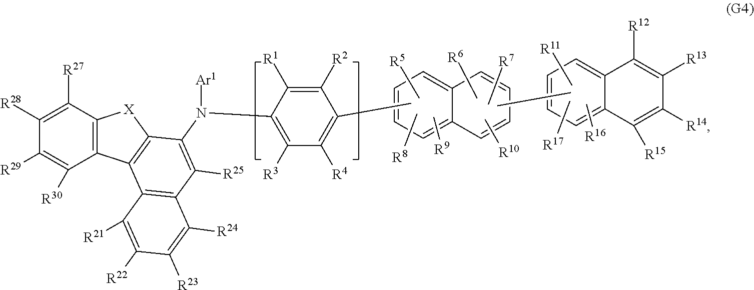
wherein Ar¹ represents a substituted or unsubstituted aryl group having 6 to 30 carbon atoms or a substituted or unsubstituted heteroaryl group having 2 to 30 carbon atoms,
wherein R¹ to R¹⁷ each independently represent any one of hydrogen, a substituted or unsubstituted alkyl group having 1 to 6 carbon atoms, and a substituted or unsubstituted cycloalkyl group having 3 to 10 carbon atoms,
wherein X represents oxygen or sulfur,
wherein R²¹ to R²⁵ and R²⁷ to R³⁰ each independently represent any one of hydrogen, a halogen, a cyano group, a substituted or unsubstituted alkyl group having 1 to 6 carbon atoms, a substituted or unsubstituted alkenyl group having 2 to 6 carbon atoms, a substituted or unsubstituted alkynyl group having 2 to 6 carbon atoms, a substituted or unsubstituted cycloalkyl group having 3 to 10 carbon atoms, a substituted or unsubstituted alkoxy group having 1 to 6 carbon atoms, a trialkylsilyl group having 3 to 10 carbon atoms, a substituted or unsubstituted aryl group having 6 to 30 carbon atoms, and a substituted or unsubstituted heteroaryl group having 2 to 30 carbon atoms, and
wherein n represents an integer of 1 to 3.

5. The organic compound according to claim 3, wherein the organic compound is represented by General Formula (G5): 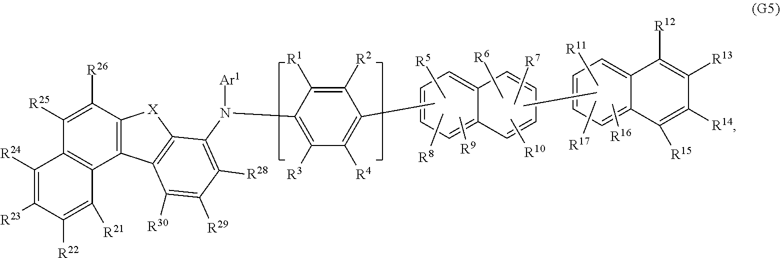
wherein Ar¹ represents a substituted or unsubstituted aryl group having 6 to 30 carbon atoms or a substituted or unsubstituted heteroaryl group having 2 to 30 carbon atoms,
wherein R¹ to R¹⁷ each independently represent any one of hydrogen, a substituted or unsubstituted alkyl group having 1 to 6 carbon atoms, and a substituted or unsubstituted cycloalkyl group having 3 to 10 carbon atoms,
wherein X represents oxygen or sulfur,
wherein R²¹ to R²⁶ and R²⁸ to R³⁰ each independently represent any one of hydrogen, a halogen, a cyano group, a substituted or unsubstituted alkyl group having 1 to 6 carbon atoms, a substituted or unsubstituted alkenyl group having 2 to 6 carbon atoms, a substituted or unsubstituted alkynyl group having 2 to 6 carbon atoms, a substituted or unsubstituted cycloalkyl group having 3 to 10 carbon atoms, a substituted or unsubstituted alkoxy group having 1 to 6 carbon atoms, a trialkylsilyl group having 3 to 10 carbon atoms, a substituted or unsubstituted aryl group having 6 to 30 carbon atoms, and a substituted or unsubstituted heteroaryl group having 2 to 30 carbon atoms, and
wherein n represents an integer of 1 to 3.

6. The organic compound according to claim 2, wherein the organic compound is represented by General Formula (G6): 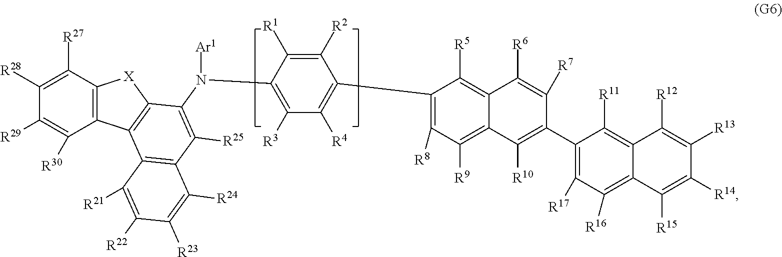
wherein Ar¹ represents a substituted or unsubstituted aryl group having 6 to 30 carbon atoms or a substituted or unsubstituted heteroaryl group having 2 to 30 carbon atoms,
wherein R¹ to R¹⁷ each independently represent any one of hydrogen, a substituted or unsubstituted alkyl group having 1 to 6 carbon atoms, and a substituted or unsubstituted cycloalkyl group having 3 to 10 carbon atoms,
wherein X represents oxygen or sulfur,
wherein R²¹ to R²⁵ and R²⁷ to R³⁰ each independently represent any one of hydrogen, a halogen, a cyano group, a substituted or unsubstituted alkyl group having 1 to 6 carbon atoms, a substituted or unsubstituted alkenyl group having 2 to 6 carbon atoms, a substituted or unsubstituted alkynyl group having 2 to 6 carbon atoms, a substituted or unsubstituted cycloalkyl group having 3 to 10 carbon atoms, a substituted or unsubstituted alkoxy group having 1 to 6 carbon atoms, a trialkylsilyl group having 3 to 10 carbon atoms, a substituted or unsubstituted aryl group having 6 to 30 carbon atoms, and a substituted or unsubstituted heteroaryl group having 2 to 30 carbon atoms, and wherein n represents an integer of 0 to 3.

7. The organic compound according to claim 3, wherein the organic compound is represented by General Formula (G7): 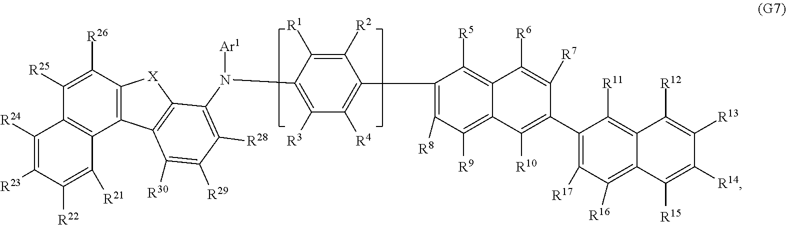
wherein Ar¹ represents a substituted or unsubstituted aryl group having 6 to 30 carbon atoms or a substituted or unsubstituted heteroaryl group having 2 to 30 carbon atoms,
wherein R¹ to R¹⁷ each independently represent any one of hydrogen, a substituted or unsubstituted alkyl group having 1 to 6 carbon atoms, and a substituted or unsubstituted cycloalkyl group having 3 to 10 carbon atoms,
wherein X represents oxygen or sulfur,
wherein R²¹ to R²⁶ and R²⁸ to R³⁰ each independently represent any one of hydrogen, a halogen, a cyano group, a substituted or unsubstituted alkyl group having 1 to 6 carbon atoms, a substituted or unsubstituted alkenyl group having 2 to 6 carbon atoms, a substituted or unsubstituted alkynyl group having 2 to 6 carbon atoms, a substituted or unsubstituted cycloalkyl group having 3 to 10 carbon atoms, a substituted or unsubstituted alkoxy group having 1 to 6 carbon atoms, a trialkylsilyl group having 3 to 10 carbon atoms, a substituted or unsubstituted aryl group having 6 to 30 carbon atoms, and a substituted or unsubstituted heteroaryl group having 2 to 30 carbon atoms, and
wherein n represents an integer of 0 to 3.

8. The organic compound according to claim 1, wherein n represents 1.

9. The organic compound according to claim 1, wherein the organic compound is represented by Structural Formula (103), (108), (117), or (118):

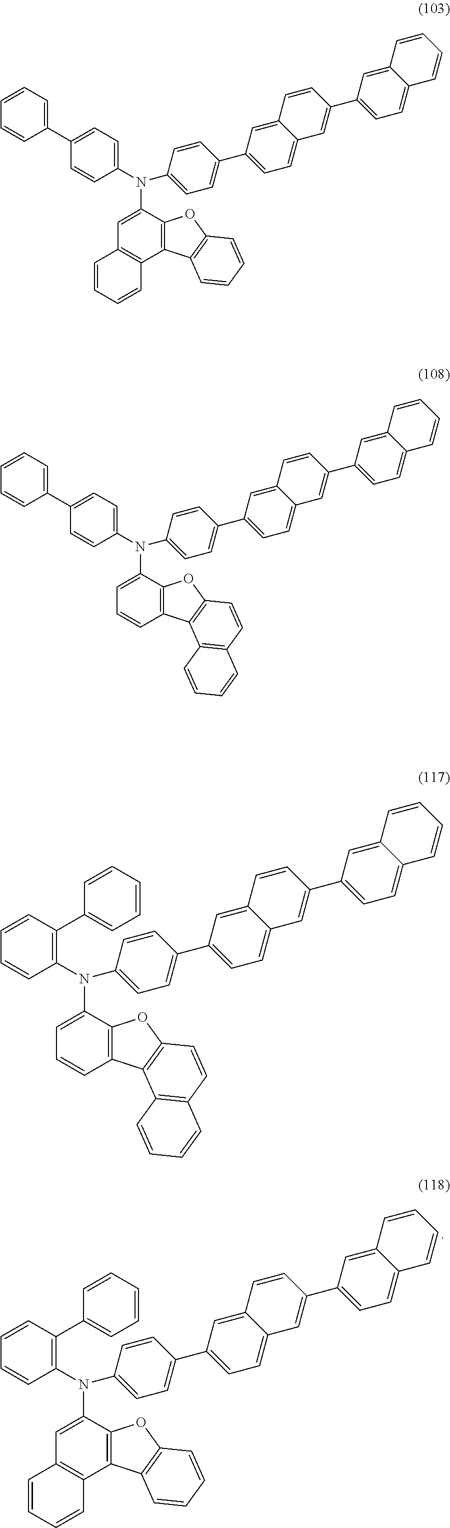 [SMILES: c1ccc(-c2ccc(N(c3ccc(-c4ccc5cc(-c6ccc7ccccc7c6)ccc5c4)cc3)c3cc4ccccc4c4c3oc3ccccc34)cc2)cc1 ; c1ccc(-c2ccc(N(c3ccc(-c4ccc5cc(-c6ccc7ccccc7c6)ccc5c4)cc3)c3cccc4c3oc3ccc5ccccc5c34)cc2)cc1 ; c1ccc(-c2ccccc2N(c2ccc(-c3ccc4cc(-c5ccc6ccccc6c5)ccc4c3)cc2)c2cccc3c2oc2ccc4ccccc4c23)cc1 ; c1ccc(-c2ccccc2N(c2ccc(-c3ccc4cc(-c5ccc6ccccc6c5)ccc4c3)cc2)c2cc3ccccc3c3c2oc2ccccc23)cc1]

10. An organic semiconductor device comprising the organic compound according to claim 1.

11. An electronic appliance comprising the organic semiconductor device according to claim 10.

12. The organic compound according to claim 2, wherein n represents 1.

13. An organic semiconductor device comprising the organic compound according to claim 2.

14. An electronic appliance comprising the organic semiconductor device according to claim 13.

15. The organic compound according to claim 3, wherein n represents 1.

16. An organic semiconductor device comprising the organic compound according to claim 3.

17. An electronic appliance comprising the organic semiconductor device according to claim 16.
